(19) United States
(12) Patent Application Publication
(10) Pub. No.: US 2026/0308536 A1
(43) Pub. Date: Oct. 8, 2026

(54) NOVEL URICASE VARIANTS AND METHODS OF USE THEREOF

(71) Applicant: Insmed Incorporated, Bridgewater, NJ (US)

(72) Inventors: Chris BAILEY-KELLOGG, Bridgewater, NJ (US); Karl GRISWOLD, Bridgewater, NJ (US); Claire GODBERSEN-PALMER, Bridgewater, NJ (US); Hao CHENG, Bridgewater, NJ (US); Yongliang FANG, Bridgewater, NJ (US); Matthew MILLER, Bridgewater, NJ (US); Adam PLAUNT, Bridgewater, NJ (US); Sasha ROSE, Bridgewater, NJ (US)

(73) Assignee: Insmed Incorporated, Bridgewater, NJ (US)

(21) Appl. No.: 19/480,640

(22) PCT Filed: May 3, 2024
(86) PCT No.: PCT/US2024/027796

§ 371 Date: Oct. 31, 2025

Related U.S. Application Data
(60) Provisional application No. 63/500,537, filed on May 5, 2023.

(51) Int. Cl. C12N 9/06; A61K 38/00

(52) U.S. Cl. CPC C12N 9/0048; C12Y 107/03003; A61K 38/00

(57) ABSTRACT

The present disclosure relates to chimeric pig-baboon uricase variants, Arthrobacter globiformis uricase variants, Aspergillus flavus uricase variants, and Candida utilis uricase variants, and methods of using the same to treat gout, tumor lysis syndrome, and other diseases associated with elevated uric acid levels in body fluids, including blood. Methods of producing the uricase variants disclosed herein are also provided.

### CROSS-REFERENCE TO RELATED APPLICATIONS

This application claims priority from U.S. Provisional Application Ser. No. 63/500,537, filed May 5, 2023, the disclosure of which is incorporated by reference herein in its entirety.

### GOVERNMENT STATEMENT

This invention was made with government support under Grant Number 1R43AI149839-01 awarded by U.S. National Institute of Allergy and Infectious Diseases of the National Institutes of Health. The U.S. government has certain rights in the invention.

### REFERENCE TO AN ELECTRONIC SEQUENCE LISTING

The contents of the electronic sequence listing (INMD_190_01WO_SeqList_ST26.xml; Size: 394,209 bytes; and Date of Creation: Apr. 23, 2024) are herein incorporated by reference in their entirety.

### BACKGROUND OF THE INVENTION

In humans, uric acid is produced following breakdown of purines. Accumulation of uric acid in the blood (hyperuricemia) is manifested in diseases, such as gout and tumor lysis syndrome.

Gout is a common and complex form of arthritis characterized by sudden, severe attacks of pain, swelling, redness and tenderness in one or more joints. Gout is caused by accumulation of uric acid crystals in the joints, leading to inflammation of intense pain. Uric acid crystals are formed when high levels of uric acid are present in the blood.

Tumor lysis syndrome is a complication from the treatment of cancer, e.g., lymphomas, leukemias, including non-Hodgkin lymphoma, acute myeloid leukemia, and acute lymphoblastic leukemia. Tumor lysis syndrome occurs as large numbers of tumor cells are lysed, releasing their contents into the bloodstream. Tumor lysis syndrome is characterized by high blood uric acid (hyperuricemia), as well as high blood potassium (hyperkalemia), high blood phosphate (hyperphosphatemia), low blood calcium (hypocalcemia), and higher than normal levels of blood urea nitrogen (BUN). The metabolic abnormalities seen in tumor lysis syndrome can ultimately result in serious complications, such as acute uric acid nephropathy, acute kidney failure, seizures, cardiac arrhythmias, and death.

Uricases are enzymes catalyzing the oxidation of uric acid to a more soluble product, allantoin, a purine metabolite which is more readily excreted. Because humans do not produce enzymatically active uricase, due to several mutations in the gene for uricase acquired during the evolution of higher primates, exogenously administered uricase provides a therapy for diseases manifesting hyperuricemia, such as gout and tumor lysis syndrome.

The present invention addresses the need for deimmunized uricases for the treatment of gout, tumor lysis syndrome, and other diseases associated with elevated uric acid levels in body fluids, including blood.

### SUMMARY OF THE INVENTION

In one aspect, the present disclosure provides uricase variants of the chimeric pig-baboon uricase of SEQ ID NO:5. In one embodiment, the chimeric pig-baboon uricase variant comprises the following amino acid substitutions relative to the chimeric pig-baboon uricase of SEQ ID NO:5: F75Q; K79E; T83Q; E90H; F96H; I100H; Y105D; V119K; Y127H; F172Y; and L260N. In a further embodiment, the chimeric pig-baboon uricase variant comprises an amino acid sequence of SEQ ID NO:1.

In one embodiment, the chimeric pig-baboon uricase variant comprises the following amino acid substitutions relative to the chimeric pig-baboon uricase of SEQ ID NO:5: F75Q; K79E; T83Q; E90H; F96H; I100H; Y105D; V119K; Y127H; K155N; F172Y; and L260N. In a further embodiment, the chimeric pig-baboon uricase variant comprises an amino acid sequence of SEQ ID NO:2.

In one embodiment, the chimeric pig-baboon uricase variant comprises the following amino acid substitutions relative to the chimeric pig-baboon uricase of SEQ ID NO:5: F75Q; K76N; K79N; T83Q; E90H; F96H; I100E; Y105D; V119K; F172Y; L260N; and I262T. In a further embodiment, the chimeric pig-baboon uricase variant comprises an amino acid sequence of SEQ ID NO:3.

In one embodiment, the chimeric pig-baboon uricase variant comprises the following amino acid substitutions relative to the chimeric pig-baboon uricase of SEQ ID NO:5: F75Q; K76N; K79N; T83Q; E90H; F96H; I100E; Y105D; V119K; K155N; F172Y; L260N; and I262T. In a further embodiment, the chimeric pig-baboon uricase variant comprises an amino acid sequence of SEQ ID NO:4.

In one embodiment, a chimeric pig-baboon uricase variant disclosed herein further comprises an N-terminal methionine residue.

In one aspect, the present disclosure provides variants of the Arthrobacter globiformis uricase of SEQ ID NO:6. In one embodiment, the Arthrobacter globiformis uricase variant comprises an amino acid sequence selected from SEQ ID NOs: 12-196. In a further embodiment, the Arthrobacter globiformis uricase variant comprises an amino acid sequence selected from SEQ ID NOs: 77, 80, 95, 134, 144, 161, and 164. In one embodiment, an Arthrobacter globiformis uricase variant disclosed herein does not include an N-terminal methionine residue. In a further embodiment, the N-terminal methionine residue of the Arthrobacter globiformis uricase variant has been removed by post-translational modification.

In one aspect, the present disclosure provides variants of the Aspergillus flavus uricase of SEQ ID NO:7. In one embodiment, the Aspergillus flavus uricase variant comprises an amino acid sequence selected from SEQ ID NOs: 197-273. In a further embodiment, the Aspergillus flavus uricase variant comprises an amino acid sequence selected from SEQ ID NOs: 229, 236, and 267-273. In one embodiment, an Aspergillus flavus uricase variant disclosed herein further comprises an N-terminal methionine residue.

In one aspect, the present disclosure provides variants of the Candida utilis uricase of SEQ ID NO:8. In one embodiment, the Candida utilis uricase variant comprises an amino acid sequence selected from SEQ ID NOs: 274-341. In a further embodiment, the Candida utilis uricase variant comprises an amino acid sequence selected from SEQ ID NOs: 276, 307, 327, 334, 340, and 341. In one embodiment, a Candida utilis uricase variant disclosed herein does not include an N-terminal methionine residue. In a further embodiment, the N-terminal methionine residue of the Candida utilis uricase variant has been removed by post-translational modification.

In one embodiment of a uricase variant disclosed herein, the uricase variant is isolated. In another embodiment, the uricase variant is purified. In another embodiment, the uricase variant is isolated and purified.

In one embodiment of a uricase variant disclosed herein, the uricase variant is a homotetramer. In another embodiment, the uricase variant is a homodimer. In another embodiment, the uricase variant is non-PEGylated. In another embodiment, the uricase variant is PEGylated, e.g., at one or more lysine residues. In a further embodiment, the uricase variant is a homotetramer and each of the uricase monomers of the homotetramer is PEGylated.

In another aspect, the present disclosure provides an isolated nucleic acid which encodes one of the uricase variants disclosed herein.

In another aspect, the present disclosure provides a nucleic acid vector comprising a nucleic acid which encodes one of the uricase variants disclosed herein.

In another aspect, the present disclosure provides a host cell comprising a nucleic acid vector disclosed herein. In a further embodiment, the host cell is capable of producing one or more of the uricase variants disclosed herein.

In another aspect, the present disclosure provides a pharmaceutical composition comprising one or more of the uricase variants disclosed herein, and a pharmaceutically acceptable carrier.

In another aspect, the present disclosure provides a method of reducing elevated uric acid levels in a subject in need of treatment. The method includes administering to the subject an effective amount of one of the uricase variants disclosed herein, or a pharmaceutical composition comprising the same. In one embodiment of the method, the subject is a human patient. In one embodiment of the method, the uric acid levels are reduced in the plasma or blood of the subject. In one embodiment of the method, the administering is carried out parenterally, e.g., via intravenous or subcutaneous administration. In one embodiment of the method, the subject is a gout patient. In a further embodiment, the subject is a refractory gout patient. In one embodiment of the method, the subject has been diagnosed with tumor lysis syndrome.

In another aspect, the present disclosure provides a method of treating gout in a subject in need of treatment. The method includes administering to the subject an effective amount of one of the uricase variants disclosed herein, or a pharmaceutical composition comprising the same. In one embodiment of the method, the gout treated is refractory gout. In one embodiment of the method, the subject is a human patient. In one embodiment of the method, the administering is carried out parenterally, e.g., via intravenous or subcutaneous administration.

In yet another aspect, the present disclosure provides a method of treating tumor lysis syndrome in a subject in need of treatment. The method includes administering to the subject an effective amount of one of the uricase variants disclosed herein, or a pharmaceutical composition comprising the same. In one embodiment of the method, the subject is a human patient. In one embodiment of the method, the administering is carried out parenterally, e.g., via intravenous or subcutaneous administration.

In even yet another aspect, the present disclosure provides a method of recombinantly producing one of the uricase variants disclosed herein. The method comprises (i) culturing a host cell comprising a nucleic acid vector comprising a nucleic acid sequence encoding the uricase variant disclosed herein, wherein the nucleic acid sequence is operatively linked to a heterologous promoter under conditions to allow for expression of the nucleic acid sequence encoding the uricase variant and recombinant production of the uricase variant by the host cell; and (ii) isolating the recombinantly produced uricase variant.

### BRIEF DESCRIPTION OF THE FIGURES

FIG. 1A shows the first stage in the uricase deimmunization workflow, in which immunodominant T cell epitopes in wild type chimeric pig-baboon uricase of which pegloticase (trade name KRYSTEXXA®) is comprised (top) were targeted for mutational silencing using substitutions predicted to maintain protein structure and function (middle). Sets of mutations were computationally optimized to yield diverse combinatorial libraries that differentially balanced reduction in immunogenicity vs. retention of function (bottom).

FIG. 1B shows the second stage in the uricase deimmunization workflow, in which one library was constructed and screened using a high throughput halo assay that reports uricase activity (top). Promising clones were picked into deep 96-well plates (middle), enabling medium throughput analysis of expression-normalized enzymatic activity in solution (bottom). The fastest enzymes were purified and analyzed in more detail.

FIG. 1C shows the third stage in the uricase deimmunization workflow, in which sensitive cellular immunoassays quantified antigen-specific proliferation of T cells among peripheral blood mononuclear cells (PBMCs) from a panel of genetically diverse donors. The immunoassays validated the computational prediction of reduced immunogenicity for each analyzed variant, and the data was used to guide lead candidate selection.

FIG. 2 is a graph showing predicted epitopes in the wild type chimeric pig-baboon uricase vs. the deimmunized library. Wild type chimeric pig-baboon uricase primary sequence position is along the x-axis and number of MHC-II alleles predicted to bind the 15mer peptide starting at each position is indicated by grey bars and height on the y-axis. Higher bars are indicative of greater immunogenicity risk. Mutated positions in library are shown as arrow heads on the x-axis, and the number of MHC-II alleles predicted to bind the mutated peptides are shown as shaded markers for the mean, median, top quartile, and single best member(s) of the library population. Note the dramatically reduced MHC-II binding potential of library members versus the wild type enzyme.

FIG. 3A is a picture showing growth of E. coli expressing the combinatorial deimmunized uricase (dURC) library on agar containing uric acid, and halos created by active clones converting insoluble uric acid into soluble allantoin.

FIG. 3B is a graphic representation showing absorbance at 293 nm over time of whole cell lysates from cultured colonies incubated with uric acid solution. Conversion of uric acid to allantoin results in decreasing absorbance, and specific activity is calculated from slopes.

FIG. 3C is a graph showing the predicted function score vs. T cell epitope score for individual variants. In the graph, selected clones with larger and darker circles indicate higher experimentally determined activity for the variant.

FIG. 3D is the residual T cell epitope map of variant 1 (dURC1), with the sequence position on the x-axis and T cell epitopes marked as bars on the y-axis. Mutated positions in the variant are shown as arrow heads on the x-axis. Epitopes deleted by the variants' mutations are light grey and residual epitopes in the variant are dark grey.

FIG. 3E is the residual T cell epitope map of variant 2 (dURC2), with the sequence position on the x-axis and T cell epitopes marked as bars on the y-axis. Mutated positions in the variant are shown as arrow heads on the x-axis. Epitopes deleted by the variants' mutations are light grey and residual epitopes in the variant are dark grey.

FIG. 3F is the residual T cell epitope map of variant 3 (dURC3), with the sequence position on the x-axis and T cell epitopes marked as bars on the y-axis. Mutated positions in the variant are shown as arrow heads on the x-axis. Epitopes deleted by the variants' mutations are light grey and residual epitopes in the variant are dark grey.

FIG. 3G is the residual T cell epitope map of variant 4 (dURC4), with the sequence position on the x-axis and T cell epitopes marked as bars on the y-axis. Mutated positions in the variant are shown as arrow heads on the x-axis. Epitopes deleted by the variants' mutations are light grey and residual epitopes in the variant are dark grey.

FIG. 4A is a graph showing the chimeric pig-baboon uricase variant expression yields as % of the wild type chimeric pig-baboon uricase (WT), following purification to >95%. Variants 1, 3, and 4 exceeded the performance specification of 20%, shown as dotted line.

FIG. 4B is a graph showing rates of uric acid conversion over a range of substrate concentrations for variants 1~4 and the wild type chimeric pig-baboon uricase (WT).

FIG. 4C is a graph showing enzyme resistance to thermal inactivation measured by heat stress activity assay, with resistance performance of candidate variants 1~4 quantified relative to the wild type chimeric pig-baboon uricase (WT). The performance specification was >90%, shown as dotted line.

FIG. 4D is a graph showing enzyme specific activity as a percentage of initial activity after the indicated number of freeze-thaw cycles. In each freeze-thaw cycle data set, four bars from left to right represent the wild type chimeric pig-baboon uricase (WT), variant 1, variant 2, and variant 3, respectively.

FIG. 5A is a graph showing specific activity of the wild type chimeric pig-baboon uricase (WT) measured by velocity at various intervals following storage at 4 or −20° C., with −20° C. samples stored with or without glycerol cryoprotectant.

FIG. 5B is a graph showing specific activity of variant 1 measured by velocity at various intervals following storage at 4 or −20° C., with −20° C. samples stored with or without glycerol cryoprotectant.

FIG. 5C is a graph showing specific activity of variant 2 measured by velocity at various intervals following storage at 4 or −20° C., with −20° C. samples stored with or without glycerol cryoprotectant.

FIG. 5D is a graph showing specific activity of variant 3 measured by velocity at various intervals following storage at 4 or −20° C., with −20° C. samples stored with or without glycerol cryoprotectant.

FIG. 6A is a graph showing the percentage of proliferating CD3⁺Ki67⁺ T cells following a 7-day expansion and measured by flow cytometry, in the presence of peptide fragments corresponding to all epitope hotspots of the wild type chimeric pig-baboon uricase (WT), or matched peptides from variant 1, 2, 3 or 4. The percentage values for each uricase enzyme were following background subtraction, with both mean and standard deviation presented and with individual donors shown as markers. The hashed horizontal line represents the 2-fold reduction specification relative to WT.

FIG. 6B is a graph showing the percentage of proliferating CD3⁺Ki67⁺ T cells following a 14-day expansion and measured by flow cytometry, in the presence of peptide fragments corresponding to all epitope hotspots of the wild type chimeric pig-baboon uricase (WT), or matched peptides from variant 1, 2, 3 or 4. The percentage values for each uricase enzyme were following background subtraction, with both mean and standard deviation presented and with individual donors shown as markers. The hashed horizontal line represents the 2-fold reduction specification relative to WT.

FIG. 6C is a graph showing the percentage of proliferating CD3⁺Ki67⁺ T cells for individual donors following 7-day expansion, in the presence of peptide fragments corresponding to all epitope hotspots of the WT, or matched peptides from variant 3 (dURC3). Donor MHC-II genotype is shown on the x-axis. Donors were classified as responders if their proliferation signal was >1% above background (hashed horizontal line).

FIG. 6D is a graph showing the percentage of proliferating CD3⁺Ki67⁺ T cells for individual donors following 14-day expansion, in the presence of peptide fragments corresponding to all epitope hotspots of the WT, or matched peptides from variant 3 (dURC3). Donor MHC-II genotype is shown on the x-axis. Donors were classified as responders if their proliferation signal was >1% above background (hashed horizontal line).

FIG. 7A is a schematic of the study design for assessing the immunogenicity of PEGylated dURC3 and PEGylated wild type chimeric pig-baboon uricase in humanized HLA transgenic mouse strain DR4.

FIG. 7B is a line graph showing the relationship between the antidrug antibody titer determined by ELISA absorbance and plasma dilution on a log scale, using day 42 plasma collected from humanized DR4 HLA transgenic mice immunized with PEGylated dURC3 or PEGylated wild type chimeric pig-baboon uricase (WT). There were 10 mice in each group, and each data point is presented as mean±SEM. Abs=absorbance.

FIG. 8A is a graph showing Arthrobacter globiformis uricase variant expression yields as % of the wild type Arthrobacter globiformis uricase (WT). All of the test variants exceeded the performance specification of 20%, shown as dotted line. Abbreviated code names of the test variants are presented for brevity, and their corresponding full code names are: B7=MedU_1B7; B8=MedU_5B8; C2=MedU_1C2; C9=MedU_5C9; E6=MedU_5E6; E9=MedU_5E9; F1=MedU_1F1.

FIG. 8B is a graph showing enzyme resistance to thermal inactivation measured by heat stress activity assay, with resistance performance of the indicated Arthrobacter globiformis uricase variants quantified relative to the wild type Arthrobacter globiformis uricase (WT). The performance specification was >0.9 (or 90%), shown as dotted line. Abbreviated code names of the test variants are presented for brevity, and their corresponding full code names are: B7=MedU_1B7; B8=MedU_5B8; C2=MedU_1C2; C9=MedU_5C9; E9=MedU_5E9; F1=MedU_1F1.

FIG. 9A is a graph showing Aspergillus flavus uricase variant expression yields as % of the wild type Aspergillus flavus uricase (WT). The target performance specification was 20%, shown as dotted line. For brevity, the prefix “RasU_” for the code name of each test variant is omitted.

FIG. 9B is a graph showing enzyme resistance to thermal inactivation measured by heat stress activity assay, with resistance performance of the indicated Aspergillus flavus uricase variants quantified relative to the wild type Aspergillus flavus uricase (WT). The performance specification was >0.9 (or 90%), shown as dotted line. For brevity, the prefix “RasU_” for the code name of each test variant is omitted.

FIG. 10 is a graph showing the soluble enzyme activity, measured with whole cell lysate of induced E. coli cells, of the indicated Candida utilis uricase variants as % of that of the wild type Candida utilis uricase (WT). For brevity, the prefix “SelU_” for the code name of each test variant is omitted.

### DETAILED DESCRIPTION OF THE INVENTION

Humans and higher primates lack the uricase enzyme, i.e., the enzyme capable of oxidizing uric acid. As a result, humans have high serum uric acid levels. In some people, uric acid levels rise above the solubility limit, resulting in crystallization in joints. Acute inflammation in response to those crystals causes severe pain; a condition known as gout. Treatment for severe gout includes injection of non-human uricase to reduce serum uric acid levels. Nyborg et al., PLOS One, 2016, DOI: 10.1371/journal.pone.0167935.

There are two clinically approved uricase-based therapies: KRYSTEXXA® (pegloticase), approved for application against chronic refractory gout (Sundy et al., Jama, 2011, 306(7): p. 711-20), and ELITEK® (rasburicase), approved for treatment of tumor lysis syndrome (Coiffier et al., J Clin Oncol, 2003, 21(23): p. 4402-4406). Pegloticase is a homotetrameric uricase protein comprised of four pig and baboon uricase chimera monomers, each having the amino acid sequence of SEQ ID NO:5. Pegloticase is hyper-PEGylated via random lysine conjugation to reduce immunogenicity and extend half-life. Rasburicase is a modified recombinant Aspergillus flavus uricase (Nyborg et al., PLOS One, 2016, DOI: 10.1371/journal.pone.0167935). Because both enzymes are non-human in nature, they evoke strong immune responses, resulting in black box warnings from the US FDA, along with fairly rapid loss of therapeutic efficacy for many pegloticase patients due to the problems resulting from the development of antidrug antibodies (Baraf et al., J Clin Rheumatol, 2014, 20(8): p. 427-32; Garay, et al., Joint Bone Spine, 2012, 79(3): p. 237-42; Verhoef et al., Drug Discov Today, 2014, 19(12): p. 1945-52). 92% of patients develop antidrug antibodies to pegloticase, highlighting a need for more effective uricase-based therapies.

Besides pegloticase and rasburicase, uricases from other species have been identified, including uricase from Arthrobacter globiformis (Juan et al., Acta Crystallogr D Biol Crystallogr. 2008 D64 (Pt 8): 815-22; Nyborg et al. (2016). PLOS One DOI: 10.1371/journal.pone.0167935), and uricase from Candida utilis (Koyama et al., J Biochem. 1996, 120(5): 969-73). A recombinant, PEGylated uricase derived from Arthrobacter globiformis (code named HZN-003, formerly MEDI-4945) is in the preclinical phase of development by Horizon Therapeutics, Dublin, Ireland (Jenkins et al., Front Pharmacol., 2022, 23; 13:925219). Pegadricase is a PEGylated, recombinant uricase derived from Candida utilis, currently in the clinical phase of development by Selecta Biosciences, Watertown, MA, USA (Sands et al., Nat Commun., 2022, 13, 272; Jenkins et al., Front Pharmacol., 2022, 23; 13:925219). As with pegloticase and rasburicase, immunogenicity of the uricases derived from Arthrobacter globiformis and Candida utilis may drastically reduce the utility of the enzymes.

Aspects of the present disclosure relate primarily to a recombinant uricase variant protein comprising amino acid mutations in T-cell epitopes such that the T-cell epitopes in the uricase variant protein are depleted, as compared to a counterpart uricase protein, e.g., a wild type (WT) uricase protein. Such uricase protein variants are referred to herein in some instances, as a “deimmunized uricase variant.” The use of such variants and methods of manufacturing the variants are also described herein.

“Recombinant protein,” as used herein, refers to any artificially produced protein and is distinguished from naturally produced proteins (i.e., proteins are produced in tissues of an animal that possesses only the natural gene for the specific protein of interest).

The term “variant” refers to a protein that is distinguished from a counterpart protein, such as the WT form of the protein, on the basis of the presence of one or more amino acid modifications, such as, for example, one or more amino acid substitutions, insertions, deletions, or a combination thereof. The term “mutant gene” is a term of the art and refers to a gene that is distinguished from the WT form of the gene on the basis of the presence of one or more nucleic acid modifications, such as, for example, one or more nucleic acid substitutions, insertions, deletions, or a combination thereof. In some embodiments, a mutant gene encodes a variant protein. However, a mutation in a gene may also be a silent mutation, i.e., a mutation that does not affect the amino acid sequence in the protein that is encoded by the gene. A mutant/variant may occur in nature or may be an engineered mutant/variant.

As used herein, the term “wild type” (abbreviated “WT”), unless otherwise specified, refers to the most prevalent form of an organism, strain, gene, protein, or characteristic as it occurs in nature and/or is distinguished from mutant or variant forms.

An amino acid modification may be an amino acid substitution, amino acid deletion and/or amino acid insertion. An amino acid substitution may be a conservative amino acid substitution or a non-conservative amino acid substitution. An amino acid substitution at a specific position on the protein sequence is denoted herein in the following manner: “one letter code of the WT amino acid residue-amino acid position-one letter code of the amino acid residue that replaces this WT residue.” For example, a uricase variant which has an amino acid substitution of W22L refers to a uricase variant protein in which the wild type residue at the 22^{nd} position (W or tryptophan) is replaced by L or leucine.

A “T-cell epitope” refers to a peptide epitope that binds to an MHCII molecule and subsequently forms a ternary complex with a cognate T-cell receptor (TCR). As used herein, one or more T-cell epitopes are “depleted” from a uricase variant if a peptide originating from the uricase variant is unable to bind an MHCII molecule, or if the peptide binds the MHCII molecule but subsequently does not bind a TCR, e.g., because of the lack of affinity to the MHCII molecule.

It is to be understood that the terminology used herein is for the purpose of describing particular embodiments only and is not intended to be limiting.

Unless defined otherwise, all technical and scientific terms used herein have the same meaning as commonly understood to one of ordinary skill in the art to which the present application belongs. Although any methods and materials similar or equivalent to those described herein can be used in the practice or testing of the present application, representative methods and materials are herein described.

Following long-standing patent law convention, the terms “a”, “an”, and “the” refer to “one or more” when used in this application, including the claims. Thus, for example, reference to “a carrier” includes mixtures of one or more carriers, two or more carriers, and the like and reference to “the method” includes reference to equivalent steps and/or methods known to those skilled in the art, and so forth.

Unless otherwise indicated, all numbers expressing quantities of ingredients, reaction conditions, and so forth used in the specification and claims are to be understood as being modified in all instances by the term “about”. Accordingly, unless indicated to the contrary, the numerical parameters set forth in the present specification and attached claims are approximations that can vary depending upon the desired properties sought to be obtained by the present application. Generally, the term “about”, as used herein in references to a measurable value such as an amount of weight, time, dose, etc. is meant to encompass values within an acceptable degree of variability in the art. In some embodiments, degree of variability is based on FDA guidelines.

Also as used herein, “and/or” refers to and encompasses any and all possible combinations of one or more of the associated listed items, as well as the lack of combinations when interpreted in the alternative (“or”).

The term “pharmaceutically acceptable”, unless otherwise noted, is used to characterize a moiety (e.g., a salt, dosage form, or excipient) as being appropriate for use in accordance with sound medical judgment. In general, a pharmaceutically acceptable moiety has one or more benefits that outweigh any deleterious effect that the moiety may have. Deleterious effects may include, for example, excessive toxicity, irritation, allergic response, and other problems and complications.

“Operably linked” refers to an arrangement of elements wherein the components so described are configured so as to perform their usual function. In the case of a promoter, a promoter that is operably linked to a coding sequence affects the expression of a coding sequence. The promoter or other control elements need not be contiguous with the coding sequence, so long as they function to direct the expression thereof. For example, intervening untranslated yet transcribed sequences can be present between the promoter sequence and the coding sequence, and the promoter sequence can still be considered “operably linked” to the coding sequence.

In one aspect, the present disclosure provides a uricase variant of the chimeric pig-baboon uricase of SEQ ID NO:5. Pegloticase is a homotetramer and each of its monomers comprises the chimeric pig-baboon uricase protein sequence of SEQ ID NO:5. In the present disclosure, a chimeric pig-baboon uricase protein of SEQ ID NO:5 serves as the benchmark enzyme for the uricase variants and is thus referred to as the wild type chimeric pig-baboon uricase. Compared to the wild type chimeric pig-baboon uricase, the chimeric pig-baboon uricase variants provided herein lack dominant immunogenic T cell epitopes while maintaining high stability and enzymatic activity, i.e., they are T-cell epitope depleted.

In one embodiment, the chimeric pig-baboon uricase variant comprises the following amino acid substitutions relative to the chimeric pig-baboon uricase of SEQ ID NO:5: F75Q; K79E; T83Q; E90H; F96H; I100H; Y105D; V119K; Y127H; F172Y; and L260N. In a further embodiment, the uricase variant comprises an amino acid sequence of SEQ ID NO: 1.

In one embodiment, the chimeric pig-baboon uricase variant comprises the following amino acid substitutions relative to the chimeric pig-baboon uricase of SEQ ID NO:5: F75Q; K79E; T83Q; E90H; F96H; I100H; Y105D; V119K; Y127H; K155N; F172Y; and L260N. In a further embodiment, the uricase variant comprises an amino acid sequence of SEQ ID NO:2.

In one embodiment, the chimeric pig-baboon uricase variant comprises the following amino acid substitutions relative to the chimeric pig-baboon uricase of SEQ ID NO:5: F75Q; K76N; K79N; T83Q; E90H; F96H; I100E; Y105D; V119K; F172Y; L260N; and I262T. In a further embodiment, the uricase variant comprises an amino acid sequence of SEQ ID NO:3.

In one embodiment, the chimeric pig-baboon uricase variant comprises the following amino acid substitutions relative to the chimeric pig-baboon uricase of SEQ ID NO:5: F75Q; K76N; K79N; T83Q; E90H; F96H; I100E; Y105D; V119K; K155N; F172Y; L260N; and I262T. In a further embodiment, the uricase variant comprises an amino acid sequence of SEQ ID NO: 4.

In another aspect, the present disclosure provides a variant of the Aspergillus flavus uricase of SEQ ID NO:7. Compared to the wild type Aspergillus flavus uricase of SEQ ID NO: 7, the Aspergillus flavus uricase variants provided herein are T cell epitope depleted while maintaining high or comparable enzymatic activity.

In one embodiment, the Aspergillus flavus uricase variant comprises an amino acid sequence selected from SEQ ID NOs: 197-273. Tables 10 and 11A of the present application shows the amino acid substitutions in the exemplary Aspergillus flavus uricase variants relative to the wild type Aspergillus flavus uricase of SEQ ID NO:7. In a further embodiment, the Aspergillus flavus uricase variant comprises an amino acid sequence selected from SEQ ID NOs: 229, 236, and 267-273.

In one aspect, the present disclosure provides a variant of the Arthrobacter globiformis uricase of SEQ ID NO:6. In one embodiment, the Arthrobacter globiformis uricase variant is created by introducing amino acid substitutions into the R49S variant Arthrobacter globiformis uricase of SEQ ID NO:6 disclosed in Nyborg et al., PLOS One, 2016, DOI: 10.1371/journal.pone.0167935, incorporated herein by reference in its entirety. For ease of description, the R49S variant Arthrobacter globiformis uricase of SEQ ID NO:6 is referred to as the wild type Arthrobacter globiformis uricase herein, as it serves as the enzyme being mutated to arrive at the variant disclosed herein. Compared to the wild type Arthrobacter globiformis uricase of SEQ ID NO:6, the Arthrobacter globiformis uricase variants provided herein are T cell epitope depleted, while maintaining high or comparable stability, enzymatic activity, and yield.

In one embodiment, the Arthrobacter globiformis uricase variant comprises an amino acid sequence selected from SEQ ID NOs: 12-196. Table 7 of the present application shows the amino acid substitutions in the exemplary Arthrobacter globiformis uricase variants relative to the wild type Arthrobacter globiformis uricase of SEQ ID NO:6. In a further embodiment, the Arthrobacter globiformis uricase variant comprises an amino acid sequence selected from SEQ ID NOs: 77, 80, 95, 134, 144, 161, and 164.

In one aspect, the present disclosure provides a variant of the Candida utilis uricase of SEQ ID NO:8. Pegadricase is a PEGylated uricase and comprises the Candida utilis uricase of SEQ ID NO:8. Compared to the wild type Candida utilis uricase of SEQ ID NO:8, the Candida utilis uricase variants provided are T-cell epitope depleted while maintaining high or comparable enzymatic activity and expression level.

In one embodiment, the Candida utilis uricase variant comprises an amino acid sequence selected from SEQ ID NOs: 274-341. Table 13 of the present application shows the amino acid substitutions in the exemplary Candida utilis uricase variants relative to the wild type Candida utilis uricase of SEQ ID NO:8. In a further embodiment, the Candida utilis uricase variant comprises an amino acid sequence selected from SEQ ID NOs: 276, 307, 327, 334, 340, and 341.

In the present disclosure, if an N-terminal methionine residue is not present in a uricase variant, alternative embodiments include uricase variants of the same sequences that have the N-terminal methionine residue present, as is the case for the chimeric pig-baboon uricase variants and the Aspergillus flavus uricase variants disclosed herein. In these instances, as the alternative variants comprise an amino acid sequence that starts with an N-terminal methionine residue while the comparator wild type uricase sequence of SEQ ID NO:5 or SEQ ID NO:7 does not include an N-terminal methionine residue, the numbering of amino acids in the comparator will be increased by 1 to compensate for the additional methionine residue in the alternative variant. One of ordinary skill in the art will readily understand that the amino acid numbering provided herein is based on the respective wild type sequences that may or may not include an N-terminal methionine residue. As such, one of ordinary skill in the art will readily understand that the numbering of amino acids in the respective alternative variant protein will be adjusted +1 if the alternative variant includes an N-terminal methionine residue and the comparator does not. Conversely, as applicable to alternative embodiments of the Arthrobacter globiformis uricase variants and Candida utilis uricase variants disclosed below, the numbering of the amino acids in the respective variant protein will be adjusted-1 if the variant does not include an N-terminal methionine residue and the comparator includes an N-terminal methionine residue. In some embodiments of the uricase variants with an N-terminal methionine residue, the N-terminal methionine residue is removed by post-translational modification. The N-terminal methionine residue is removed, in one embodiment, by endogenous bacterial methionine aminopeptidase after the uricase variant is produced in bacteria, e.g., in E. coli.

In one embodiment, if an N-terminal methionine residue is present in a uricase variant, the present disclosure also encompasses alternative uricase variants of the same sequences that do not include the N-terminal methionine residue, e.g., because it is removed after translation, as is the case for the Arthrobacter globiformis uricase variants and the Candida utilis uricase variants disclosed herein. In these instances, as the alternative variants comprise an amino acid sequence without an N-terminal methionine residue while the comparator wild type uricase sequence of SEQ ID NO:6 or SEQ ID NO:8 includes an N-terminal methionine residue, the numbering of the amino acids in the respective alternative variant protein will be adjusted-1, as discussed above.

In one embodiment of a uricase variant disclosed herein, the uricase variant is isolated. In another embodiment, the uricase variant is purified. In some embodiments, the uricase variant comprises a purification tag at the C-terminus, the N-terminus, or both the N- and C-terminus to allow for purification by affinity chromatography. In one embodiment, the purification tag is a polyhistidine tag (also referred to as a “his-tag”). The his-tag, in one embodiment, comprises six (6) histidine residues. In another embodiment, the purification tag is a his-glu tag (HQ tag). In a further embodiment, the HQ tag has the amino acid sequence of HQHQHQ (SEQ ID NO:9). In another embodiment, the purification tag is a his-asp tag (HN tag). In a further embodiment, the HN tag has the amino acid sequence of HNHNHINHNHNHN (SEQ ID NO:10). In another embodiment, the purification tag is a histidine affinity tag (HAT). In a further embodiment, the HAT has the amino acid sequence of KDHLIHNVHKEEHAHAHNK (SEQ ID NO:11). In another embodiment, a uricase variant provided herein is isolated and purified.

The uricase variants disclosed herein encompass monomer subunits as well as multimers (e.g., a homodimer, a homotetratmer, a heterodimer or a heterotetramer). In one embodiment, the uricase variant provided herein is a monomer. In one embodiment, the uricase variant provided herein is a homodimer. In another embodiment, the uricase variant provided herein is a homotrimer. In another embodiment, the uricase variant provided herein is a homotetramer. In one embodiment, a uricase variant disclosed herein is a heterodimer, a heterotrimer or a heterotetramer.

It is noted that the amino acid positions and mutations described herein are provided for uricase protein monomers. However, embodiments of the invention are also directed to multimeric, e.g., dimeric and tetrameric, forms of uricase. For example, in the case of homodimers and homotetratmers, the amino acid mutations set forth for the monomeric protein will also be present in the remaining protein subunits. However, in embodiments where a heterodimer and/or heterotetramer uricase variant is provided, mutations in one subunit may or may not be present in the remaining uricase subunits.

In one embodiment, the uricase variant disclosed herein is non-PEGylated. In another embodiment, the uricase variant is PEGylated, i.e., the uricase variant is covalently conjugated to polyethylene glycol (PEG), e.g., at one or more lysine residues. In a further embodiment, each of the uricase monomers of a multimeric uricase variant, e.g., a homotetramer uricase variant, is PEGylated. In one embodiment, the uricase variant is covalently conjugated to one or more molecules of monomethoxypoly(ethylene glycol) (mPEG). In a further embodiment, each mPEG molecule is about 10 kDa in molecular weight. A uricase variant monomer may be conjugated to any desired number of PEG or mPEG molecules, such as 1, 2, 3, 4, 5, 6, 7, 8, 9, 10, 11, 12, 13, or 14. In one embodiment, a uricase variant monomer is conjugated to from about 8 to about 11 PEG or mPEG molecules, about 9 PEG or mPEG molecules, or about 10 PEG or mPEG molecules. In one embodiment, the average total molecular weight of the PEG moiety of a PEGylated monomeric uricase variant is about 10 kDa to about 150 kDa, about 30 kDa to 120 kDa, or about 50 kDa to about 100 kDa.

In one embodiment, the uricase variant disclosed herein is covalently bonded to PEG, e.g., mPEG, via a biocompatible linking group, using methods known in the art, as described, for example, by Park et al, Anticancer Res., 1981, 1:373-376; and Zaplipsky and Lee, Polyethylene Glycol Chemistry: Biotechnical and Biomedical Applications, J. M. Harris, ed., 1992, Plenum Press, New York, Chapter 21, the disclosures of each of which are incorporated by reference herein in their entireties. A biocompatible linking group is non-toxic and can be used in vitro or in vivo without causing injury, sickness, disease, or death. In some embodiments, PEG can be bonded to the linking group, for example, via an ether bond, an ester bond, a thiol bond or an amide bond. Suitable biocompatible linking groups include, for example, an ester group, an amide group, an imide group, a carbamate group, a carboxyl group, a hydroxyl group, a carbohydrate, a succinimide group, an epoxide group, an oxycarbonylimidazole group, a nitro phenyl group, a trysylate group, an aldehyde group, an isocyanate group, a vinylsulfone group, a tyrosine group, a cysteine group, a histidine group or a primary amine.

In another embodiment, the uricase variant disclosed herein is conjugated without a linking group to PEG, e.g., mPEG, through an amino group, a sulfhydral group, a hydroxyl group or a carboxyl group. In one embodiment, PEG is conjugated to one or more lysine residues on the uricase variant. In one embodiment, PEG is conjugated to one or more cysteine residues on the uricase variant. In one embodiment, PEG is conjugated to one or more serine residues on the uricase variant.

In another aspect, the present disclosure provides an isolated nucleic acid which encodes a uricase variant disclosed herein. An isolated nucleic acid is removed from its natural environment, and may additionally be in substantially pure, e.g., at least 90% pure, or in homogeneous form. The isolated nucleic acid may be, for example, a synthetic DNA, a non-naturally occurring mRNA, or a cDNA. Methods of producing the disclosed nucleic acids are well known to the one of skill in the art. See, e.g., Maniatis, T., 1990, Molecular Cloning, A Laboratory Manual, 2d ed., Cold Spring Harbor Laboratory, Cold Spring Harbor, N.Y., incorporated herein by reference in its entirety. Additionally or alternatively, the disclosed nucleic acids may be produced by introducing one or more mutations to a related nucleic acid with a similar nucleic acid sequence using site-directed mutagenesis techniques known in the art, such as extension of overlapping gene segments by PCR, disclosed in Heckman et al., Nat Protoc 2007, 2, 924-932, incorporated herein by reference in its entirety. The disclosed nucleic acids can be cleaved at appropriate sites with restriction endonuclease(s), followed by further enzymatic modification if desired, isolated, and ligated in vitro.

In another aspect, the present disclosure provides a nucleic acid vector comprising a nucleic acid encoding a uricase variant disclosed herein. In one embodiment, the nucleic acid coding for a uricase variant is inserted into a vector, e.g., a plasmid, for multiplication of the nucleic acid. In another embodiment, the nucleic acid coding for a uricase variant is inserted into an appropriate expression vector, i.e., a vector which contains the necessary elements, e.g., promoter sequences, terminator sequences, polyadenylation sequences, and/or enhancer sequences, for the transcription and translation of the inserted uricase variant-coding sequence. Vectors may be plasmids, phage, phagemids, adenoviral, AAV, lentiviral, for example. A variety of host-vector systems may be utilized to express the uricase variant-coding sequence. Exemplary systems include mammalian cell systems infected with virus (e.g., vaccinia virus, adenovirus, etc.); insect cell systems infected with virus (e.g., baculovirus); microorganisms such as yeast containing yeast vectors, or bacteria transformed with bacteriophage DNA, plasmid DNA, or cosmid DNA. The expression elements of these vectors vary in their strengths and specificities. In one embodiment, a bacterial expression vector is used. In a further embodiment, the bacterial expression vector is for use with E. coli. In one embodiment, the promoter in the bacterial expression vector is a T5 promoter inducible by isopropyl β-D-1 thiogalactopyranoside (IPTG) and repressible by glucose, a T7 promoter inducible by IPTG and repressible by glucose, a rhamnose (rham) promoter inducible by rhamnose and repressible by glucose, or an alkaline phosphatase (phoA) promoter inducible by phosphate starvation and repressible by the presence of phosphate.

Any of the methods known for the insertion of DNA fragments into a vector may be used to construct expression vectors containing a nucleic acid comprising appropriate regulatory elements (e.g., transcriptional/translational control signals) and a uricase variant coding sequence operably linked thereto. These methods may include in vitro recombinant DNA and synthetic techniques and in vivo recombination (genetic recombination). Expression of a nucleic acid sequence encoding a uricase variant may be regulated by a second nucleic acid sequence so that the uricase variant protein is expressed in a host transformed with the recombinant DNA molecule. For example, expression of a uricase variant may be controlled by any promoter/enhancer element known in the art. In some embodiments, the nucleic acid comprises a nucleic acid sequence encoding the uricase variant operatively linked to a heterologous promoter. Exemplary promoters which may be used to control uricase variant expression using a mammalian expression vector include the simian virus 40 (SV40) early promoter region, the promoter contained in the 3′ long terminal repeat of Rous sarcoma virus, the herpes thymidine kinase promoter, and the regulatory sequences of the metallothionine gene. Exemplary promoters useful for prokaryotic expression vectors include the β-lactamase promoter, the tac promoter, and the osmoregulated osmB promoter.

In another aspect, the present disclosure provides a host cell comprising a nucleic acid vector disclosed herein. In a further embodiment, the host cell is capable of producing a uricase variant disclosed herein. Appropriate cell lines or host systems can be chosen based on the desired expression level and/or post-translational processing and modification (e.g., glycosylation, cleavage) of the uricase variant protein. Suitable host cells include bacteria (e.g., E. coli), mammalian cells, plant cells, insect cells, fungi, yeast and transgenic plants and animals. Exemplary mammalian cell lines available in the art for expression of a heterologous protein, such as a uricase variant disclosed herein, include Chinese hamster ovary (CHO) cells, HeLa cells, baby hamster kidney cells, mouse melanoma cells, rat myeloma cells, human embryonic kidney cells, and human embryonic retina cells. Introducing the vector into a host cell can be accomplished using techniques well known in the art. For eukaryotic cells, suitable techniques may include calcium phosphate transfection, diethylaminoethyl (DEAE)-dextran, electroporation, liposome-mediated transfection, and transduction using retroviruses or other viruses, for example. For bacterial cells, suitable techniques may include calcium chloride transformation, electroporation, and transfection using bacteriophage. The introduction may be followed by causing or allowing expression from the nucleic acid, e.g., by culturing host cells under conditions for expression of the nucleic acid sequence encoding the uricase variant to produce the uricase variant protein. In one embodiment, the nucleic acid encoding the uricase variant is integrated into the genome, e.g., chromosome, of the host cell. Integration may be promoted by inclusion of sequences which promote recombination with the genome, in accordance with standard techniques.

In one embodiment, the host cell is a mammalian cell, e.g., a Chinese hamster ovary (CHO) cell, or a human embryonic kidney cell. In a further embodiment, the expression vector for use in the mammalian cell comprises the actin (e.g., chicken β-actin), cytomegalovirus (CMV), CMV enhancer/elongation factor (CEF), CMV early enhancer/chicken β-actin (CAG), hybrid CMV enhancer/chicken β-actin (CBh), elongation factor-la (EFlalpha), glyceraldehyde-3-phosphate dehydrogenase (GAPDH), or simian virus 40 (SV40) promoter.

In another embodiment, the host cell is a yeast cell. In a further embodiment, the expression vector for use in the yeast cell comprises the alcohol oxidase (AOX), glyceraldehyde-3-phosphate dehydrogenase (GAP), alcohol dehydrogenase (ADH), or galactokinase 1 (GAL1) promoter.

In another embodiment, the host cell is an insect cell infected with baculovirus. In a further embodiment, the expression vector for use with the baculovirus comprises the polyhedrin gene promoter.

In another embodiment, the host cell is bacteria, e.g., E. coli. In a further embodiment, the expression vector for use in bacteria comprises the osmB promoter, the T7-lac promoter (Shilling et al., Commun Biol 3, 214 (2020)), the pBAD promoter, the Tac promoter, a tet-inducible promoter, the cold-shock Protein A (cspA) promoter, or the inducible promoter of the alkaline phosphatase gene (phoA) derived from E. coli.

In another aspect, the present disclosure provides a method of recombinantly producing a uricase variant disclosed herein. The method includes (i) culturing a host cell comprising a nucleic acid vector comprising a nucleic acid sequence encoding a uricase variant disclosed herein, wherein the nucleic acid sequence is operatively linked to a heterologous promoter under conditions to allow for expression of the nucleic acid sequence encoding the uricase variant and recombinant production of the uricase variant by the host cell; and (ii) isolating the recombinantly produced uricase variant.

The uricase variants may be produced in any suitable cell-culture system including prokaryotic cells, e.g., E. coli, BL21 or JM83, or eukaryotic cells, e.g., Pichia pastoris yeast strain X-33 or CHO cells. Further suitable cell lines known in the art are obtainable from cell line depositories, such as the American Type Culture Collection (ATCC). The uricase variants can be isolated from the growth medium, cellular lysates or cellular membrane fractions. The isolation and purification of the recombinantly produced uricase variants of the invention may be performed by any conventional means, including ammonium sulphate precipitation, affinity columns, column chromatography, gel electrophoresis and the like and may involve the use of monoclonal or polyclonal antibodies directed, e.g., against a tag fused with the biologically active protein of the invention. For example, the protein can be purified via the Strep-tag II using streptavidin affinity chromatography (Skerra and Schmidt (2000). Methods Enzymol 326, pp. 271-304).

In one embodiment, the vector is under the control of an osmotic pressure sensitive promoter. An osmotic pressure sensitive promoter initiates transcription as a result of increased osmotic pressure as sensed by the cell. In one embodiment, the host cell is E. coli, and the promoter is the osmB promoter, the T7-lac promoter, the pBAD promoter, the Tac promoter, a tet-inducible promoter, the cold-shock Protein A (cspA) promoter, or the inducible promoter of the alkaline phosphatase gene (phoA) derived from E. coli.

In one embodiment, the uricase variant is isolated using a cationic surfactant, for example, cetyl pyridinium chloride (CPC). In one embodiment, the method further comprises purifying the recombinantly produced uricase variant using, for example, affinity chromatography, ammonium sulfate fractionation, or sodium dodecyl sulfate-polyacrylamide gel (SDS-PAGE) electrophoresis. For example, the uricase variant can be made according to the methods described in International Patent Application Publication No. WO 2000/008196, incorporated herein by reference in its entirety. In one embodiment, the uricase variant is produced in bacteria and, following isolation, subjected to endotoxin mitigation using methods known in the art, such as one disclosed in the examples of the present application.

In another aspect, the present disclosure provides a pharmaceutical composition comprising one or more of the uricase variants disclosed herein. The pharmaceutical composition may further comprise a pharmaceutically acceptable excipient, carrier, buffer, stabilizer or other materials well known to those skilled in the art. Such materials are non-toxic and do not interfere with the efficacy of the uricase variants, which are the active ingredients. Such materials may include, for example, solvents, dispersion media, antibacterial and antifungal agents, and/or isotonic and absorption delaying agents. Some examples of pharmaceutically acceptable carriers are water, saline, phosphate buffered saline, dextrose, glycerol, and ethanol, as well as combinations thereof. In one embodiment, the pharmaceutical composition includes an isotonic agent, for example, a sugar, and/or a polyalcohol, such as mannitol or sorbitol, or sodium chloride. Additional examples of pharmaceutically acceptable substances are wetting agents or auxiliary substances, such as emulsifying agents, preservatives or buffers, which increase the shelf life or effectiveness.

In one embodiment, the pharmaceutical composition comprising the uricase variant disclosed herein may be formulated in liquid, semi-solid or solid forms, such as liquid solutions (e.g., injectable and infusible solutions), dispersions or suspensions, powders, liposomes, and suppositories. The preferred form depends on the intended mode of administration, therapeutic application, the physicochemical properties of the variant, and the route of delivery. Formulations may include excipients, or combinations of excipients, for example: sugars, amino acids and surfactants. Liquid formulations may include a wide range of uricase variant concentrations and pH. Solid formulations may be produced by lyophilization, spray drying, or drying by supercritical fluid technology, for example.

For intravenous injection, or injection at the site of affliction, the active ingredient may be in a parenterally acceptable aqueous solution which is pyrogen-free and has suitable pK, isotonicity, and stability. Those of relevant skill in the art are well able to prepare suitable solutions using, for example, isotonic vehicles such as sodium chloride solution, Ringer's solution, and a lactated Ringer's solution. Preservatives, stabilizers, buffers, antioxidants and/or other additives may be included.

In some embodiments, the pharmaceutical composition is formulated as a solution, microemulsion, dispersion, liposome, or other ordered structure suitable to contain a high uricase variant concentration. Sterile injectable solutions can be prepared by incorporating a uricase variant in an appropriate solvent with one or a combination of ingredients enumerated above, followed by filtered sterilization. Generally, dispersions are prepared by incorporating the uricase variant into a sterile vehicle that contains a dispersion medium and other ingredients from those enumerated above. In the case of sterile powders for the preparation of sterile injectable solutions, the methods of preparation include vacuum drying and freeze-drying that yields a powder of the active ingredient plus any additional desired ingredient from a previously sterile-filtered solution thereof. The proper fluidity of a solution can be maintained, for example, by using a coating such as lecithin, by maintaining the particle size of a dispersion, or by using surfactants. Prolonged absorption of injectable compositions can be brought about by including in the composition an agent that delays absorption, for example, monostearate salts and gelatin.

In some embodiments, the pharmaceutical composition may be prepared with a carrier that protects the uricase variant against rapid release, such as a controlled release formulation, including implants, transdermal patches, and microencapsulated delivery systems. Biodegradable, biocompatible polymers can be used, such as ethylene vinyl acetate, polyanhydrides, polyglycolic acid, collagen, polyorthoesters, and polylactic acid.

In one embodiment, the pharmaceutical composition is a solution of a uricase variant, e.g., a phosphate buffered saline solution containing a uricase variant. In a further embodiment, the solution is sterile and suitable for injection, e.g., intravenous injection or subcutaneous injection.

In another aspect, the present disclosure provides a method of reducing elevated uric acid levels in a subject in need of treatment. The method includes administering to the subject an effective amount of one of the uricase variants disclosed herein, or a pharmaceutical composition comprising the same.

As used herein, “treatment” or “treating,” or “ameliorating” and variations thereof are used interchangeably. These terms refer to an approach for obtaining beneficial or desired results including but not limited to a therapeutic benefit and/or a prophylactic benefit. Therapeutic benefit refers to any therapeutically relevant improvement in or effect on one or more diseases, conditions, or symptoms under treatment. The term “treating” in one embodiment, includes: (1) preventing or delaying the appearance of clinical symptoms of the state, disorder or condition developing in the patient that may be afflicted with or predisposed to the state, disorder or condition but does not yet experience or display clinical or subclinical symptoms of the state, disorder or condition; (2) inhibiting the state, disorder or condition (e.g., arresting, reducing or delaying the development of the disease, or a relapse thereof in case of maintenance treatment, of at least one clinical or subclinical symptom thereof); (3) relieving the condition (for example, by causing regression, or reducing the severity of the state, disorder or condition or at least one of its clinical or subclinical symptoms).

The term “effective amount” or “therapeutically effective amount” refers to the amount of an agent, e.g., a uricase variant disclosed in the present application, or a pharmaceutical composition comprising a uricase variant of the present disclosure, that is sufficient to achieve an outcome, for example, to effect beneficial or desired results. The therapeutically effective amount may vary depending upon one or more of: the subject and disease condition being treated, the weight and age of the subject, the severity of the disease condition, the manner of administration and the like.

The terms “subject,” “individual,” and “patient” are used interchangeably herein to refer to a vertebrate, such as a mammal. The mammal may be, for example, a mouse, a rat, a rabbit, a cat, a dog, a pig, a sheep, a horse, a non-human primate (e.g., cynomolgus monkey, chimpanzee), or a human. A subject's tissues, cells, or derivatives thereof, obtained in vivo or cultured in vitro are also encompassed. A human subject may be an adult, a teenager, a child (2 years to 14 years of age), an infant (1 month to 24 months), or a neonate (up to 1 month). In some embodiments, the adults are seniors about 65 years or older, or about 60 years or older. In some embodiments, the subject is a pregnant woman or a woman intending to become pregnant.

In one embodiment of the method, the subject is a human patient. In a further embodiment, the human patient is an adult human patient.

In one embodiment of the method, the uric acid levels are reduced in the plasma or blood of the subject. In one embodiment, suitable indicators for assessing effectiveness of the method include normalization or lowering of plasma uric acid levels (PUA), e.g., lowering or maintenance of PUA to 6.8 mg/dL or less, or 6 mg/dL or less in a human patient.

In some embodiments of the method, administration of the uricase variant or a pharmaceutical composition disclosed herein is carried out parenterally, e.g., via intramuscular, intrathecal, subcutaneous, or intravenous administration. In one embodiment, the administration is intravenous (IV) administration. In another embodiment, the administration is subcutaneous (SC) administration. In another embodiment, the administration is rectal, topical, or pulmonary administration.

In one embodiment, the subject is a gout patient, e.g., a gout human patient. In one embodiment, the gout is recurrent gout. In another embodiment, the gout is advanced gout, with deposits of uric acid crystals forming under the skin in nodules called tophi. In a human patient, tophi can develop in several areas, such as fingers, hands, feet, elbows or Achilles tendons along the backs of ankles. In another embodiment, the gout patient has kidney stones, which are uric acid crystals collected in the urinary tracts.

In one embodiment, the subject is a refractory gout patient, i.e., refractory to a prior different treatment. The prior treatments include, but are not limited to, treatments with nonsteroidal anti-inflammatory drugs (NSAIDs), colchicine, corticosteroids, allopurinol, febuxostat, probenecid, KRYSTEXXA (pegloticase), and a combination of the foregoing. In one embodiment, refractory gout is a chronic condition characterized by high serum uric acid levels, recurrent gout flares, chronic arthritis, and progressive tophaceous deposition. In another embodiment, refractory gout is associated with high rates of cardiovascular and renal comorbidities.

In one embodiment, the subject has been diagnosed with tumor lysis syndrome. In some embodiments, the subject diagnosed with tumor lysis syndrome has a lymphoma (e.g., a Burkitt's lymphoma, a non-Hodgkin's lymphoma), acute lymphoblastic leukemia, or acute myeloid leukemia. In one embodiment, the subject diagnosed with tumor lysis syndrome is a human patient with a plasma uric acid concentration of >8 mg/dL. In another embodiment, the subject diagnosed with tumor lysis syndrome is a human patient with a plasma uric acid concentration of >15 mg/dL (hyperuricemia).

In another aspect, the present disclosure provides a method of treating gout in a subject in need of treatment. The method includes administering to the subject an effective amount of one of the uricase variants disclosed herein, or a pharmaceutical composition comprising the same. In one embodiment, the gout treated is refractory gout, as described above. In one embodiment, the gout treated is recurrent gout. In another embodiment, the gout treated is advanced gout, characterized by deposits of uric acid crystals forming under the skin in nodules called tophi. In another embodiment, the subject with gout has kidney stones. In one embodiment, the subject is a human patient. In a further embodiment, the human patient is an adult human patient.

In some embodiments of the method, administration of the uricase variant or the pharmaceutical composition disclosed herein is carried out parenterally, e.g., via intramuscular, intrathecal, SC, or IV administration. In one embodiment, the administration is IV administration. In another embodiment, the administration is SC administration. In another embodiment, the administration is rectal, topical, or pulmonary administration.

In another aspect, the present disclosure provides a method of treating tumor lysis syndrome in a subject in need of treatment. The method includes administering to the subject an effective amount of one of the uricase variants disclosed herein, or a pharmaceutical composition comprising the same. In one embodiment of the method, the subject is a human patient. In a further embodiment, the human patient is an adult human patient.

In some embodiments of the method, administration of the uricase variant or the pharmaceutical composition disclosed herein is carried out parenterally, e.g., via intramuscular, intrathecal, SC, or IV administration. In one embodiment, the administration is IV administration. In another embodiment, the administration is SC administration. In another embodiment, the administration is rectal, topical, or pulmonary administration. In some embodiments, the subject with tumor lysis syndrome has a lymphoma (e.g., a Burkitt's lymphoma, a non-Hodgkin's lymphoma), acute lymphoblastic leukemia, or acute myeloid leukemia. In one embodiment, the subject with tumor lysis syndrome is a human patient with a plasma uric acid concentration of >8 mg/dL. In another embodiment, the subject with tumor lysis syndrome is a human patient with a plasma uric acid concentration of >15 mg/dL (hyperuricemia).

In another aspect, the present disclosure provides a method for metabolizing uric acid using one of the uricase variants disclosed herein.

In another aspect, the present disclosure provides use of a composition comprising one or more of the uricase variants disclosed herein for reducing uric acid levels in a biological fluid. In one embodiment, the biological fluid is from a gout patient or a refractory gout patient. In another embodiment, the biological fluid is from a patient diagnosed with tumor lysis syndrome. In a further embodiment, the biological fluid comprises blood.

### EXAMPLES

The present invention is further illustrated by reference to the following Examples. However, it should be noted that these Examples, like the embodiments described above, are illustrative and are not to be construed as restricting the scope of the invention in any way.

### Example 1—Creation and Characterization of Non-PEGylated Deimmunized Chimeric Pig-Baboon Uricase Variants

This example describes the development of highly engineered, deimmunized chimeric pig-baboon uricase variants by utilizing a computationally-driven protein engineering platform. The variants exhibited activity greater than the activity of the wild type chimeric pig-baboon uricase of SEQ ID NO:5, of which pegloticase is comprised, while having reduced immunogenic potential via mutagenic silencing (depletion) of constituent T cell epitopes. The deimmunized chimeric pig-baboon uricase variants and the comparator wild type chimeric pig-baboon uricase described in this example are non-PEGylated, unless otherwise specified.

### Methods

### 1. Soluble Enzyme Activity Assay

Small volume cultures of E. coli transformed with the expression vector pET-26b(+) containing the cDNA sequence encoding the wild type chimeric pig-baboon uricase or its variant were grown and induced in deep 96-well plates. The expression vector pET-26b(+) encodes a His tag containing 6 histidine residues at the C-terminus of the inserted uricase gene. Induced E. coli cells were pelleted by centrifugation. Cell pellets were resuspended in BUGBUSTER® HT Protein Extraction Reagent (MilliporeSigma, MA, USA), pelleted by centrifugation, and the insoluble pellet was resolubilized in an alkaline carbonate/bicarbonate buffer (pH 10.5). The resolubilized sample was combined in a UV transparent 96-well plate with a uric acid substrate solution (0.1 M boric acid, 0.12 mM uric acid, pH 9.0), and absorbance at 293 nm was measured as a function of time. Uricase activity was measured as the slope of the absorbance at 293 nm vs time curve, with steeper negative slopes indicative of higher activity. As such, the slopes were quantified as raw activity measures in arbitrary units.

### 2. Expression Assay

The expression assay is based on measuring total activity in the soluble supernatant following lysis of E. coli cell pellets in the alkaline carbonate/bicarbonate buffer (pH 10.5), as described above, and separately measuring activity of immobilized uricase enzyme (on a 96-well Ni-NTA HisSorb plate (Qiagen)) from the soluble supernatant (described below), as the wild type chimeric pig-baboon uricase or its variant produced in E. coli contained a His tag at the C-terminus. In theory the HisSorb immobilized surface is saturated with enzyme, and thus dividing total activity by this saturated immobilized activity yields a relative expression level.

To measure the activity of immobilized uricase enzyme from the soluble supernatant, small volume cultures of E. coli transformed with the expression vector pET-26b(+) containing the cDNA sequence encoding the wild type chimeric pig-baboon uricase or its variant were grown and induced in deep 96-well plates. The induced E. coli cells were pelleted by centrifugation. Cell pellets were resuspended in BUGBUSTER® HT Protein Extraction Reagent, pelleted by centrifugation, and the insoluble pellet was resolubilized in an alkaline carbonate/bicarbonate buffer (pH 10.5). The resolubilized sample was collected and transferred to a 96-well Ni-NTA HisSorb plate (Qiagen) bearing immobilized metal ion affinity ligands on the bottom of the wells. The HisSorb plate was incubated for one hour to bind the His-tagged uricase enzymes to the wells, followed by the removal of the supernatant from the wells and washing of the wells five times with the alkaline carbonate/bicarbonate buffer (pH 10.5). Thereafter, a uric acid substrate solution (0.1 M boric acid, 0.12 mM uric acid, pH 9.0) was added to the HisSorb plate, and absorbance at 293 nm was measured as a function of time. Uricase activity was measured as the slope of the absorbance at 293 nm vs time curve, with steeper negative slopes indicative of higher activity. Relative expression level was calculated as the activity of the total soluble enzyme fraction divided by the activity of the HisSorb-bound enzyme fraction.

### 3. Differential Scanning Fluorimetry (DSF)

DSF was performed according to Niesen et al., Nat Protoc. 2007, 2(9):2212-21, incorporated herein by reference in its entirety, to determine the melting temperature of the wild type chimeric pig-baboon uricase and its variants using a C1000 thermal cycler in conjunction with a CFX96 real-time system (Bio-Rad). Proteins were diluted to a final concentration of 66 μg/mL in PBS buffer containing SYPRO Orange dye at a final concentration of 5×. Triplicate samples of each protein were exposed to a temperature gradient from 40° C. to 99° C. Melting temperatures were calculated using gain of fluorescence of SYPRO orange and loss of fluorescence using PRISM software.

### 4. Heat Stress Activity Assay

The temperatures at which 50% of normal activity was lost after heating for the wild type chimeric pig-baboon uricase and its variants were determined by heat stress activity assay with the following procedure:

(1) Uricase in alkaline carbonate/bicarbonate buffer (pH 10.5) was incubated for 30 min at a range of temperatures of 25° C. (room temperature), 30° C., 32° C., 34° C., 36° C., 38° C., 40° C., 42° C., 44° C., 46° C., or 48° C., and were then cooled to the room temperature.

(2) 2 μg of heat treated uricase was combined with a uric acid substrate solution (0.1 M boric acid, 0.12 mM uric acid, pH 9.0) and absorbance measurements at 293 nm were captured every 30 seconds for a period of 20 min. Uricase activity was measured as the slope of the absorbance at 293 nm vs time curve, with steeper negative slopes indicative of higher activity. The temperature resulting in 50% normal activity was calculated by non-linear regression.

### 5. Determination of Reaction Velocity for the Wild Type Chimeric Pig-Baboon Uricase and its Variants

The reaction velocity for the wild type chimeric pig-baboon uricase and its variants was determined by the following procedure:
(1) diluting a uricase protein sample to 0.2 mg/mL in a buffer containing 0.1M carbonate-bicarbonate, pH 10.5;
(2) in a 96-well plate, aliquoting 10 μL of the diluted protein sample per well;
(3) adding 190 μL of a uric acid substrate solution containing 0.125 mM uric acid to each well;
(4) loading the 96-well plate onto a plate reader;
(5) measuring absorbance at 293 nm every 30 seconds for 20 minutes under the controlled temperature of 25° C. on the plate reader;
(6) calculating Vₘₐₓ values using the BIOTEK Gen5 software.

### 6. Determination of Michaelis Menten Kinetics for the Wild Type Chimeric Pig-Baboon Uricase and its Variants

The Michaelis Menten kinetics for the wild type chimeric pig-baboon uricase and its variants were determined by the following procedure:
(1) diluting a uricase protein sample to 0.2 mg/mL in a buffer containing 0.1M carbonate-bicarbonate, pH 10.5;
(2) into a plurality of wells of a 96-well plate, aliquoting 10 μL of the diluted protein sample per well;
(3) adding to each well containing the diluted protein sample 40 μL each of uric acid substrate solutions containing uric acid at the following concentrations: 2000 μM, 1700 μM, 1500 μM, 1200 μM, 1000 μM, 750 μM, 500 μM, 375 μM, 250 μM, 187 μM, 125 μM, and 93 μM;
(4) loading the 96-well plate onto a plate reader;
(5) measuring absorbance at 293 nm every 30 seconds for 20 minutes under the controlled temperature of 25° C. on the plate reader;
(6) calculating Vₘₐₓ values using the BIOTEK Gen5™ software;
(7) Using the Michaelis-Menten function of the PRISM software, analyzing the Vₘₐₓ values as a function of the uric acid concentration to derive the Michaelis Menten kinetics parameters.

### Results

The monomeric form of the wild type chimeric pig-baboon uricase comprises the amino acid sequence of SEQ ID NO:5, where threonine is residue #1. The specified mutations in the chimeric pig-baboon uricase variants of this example are indexed to threonine as residue #1, as in SEQ ID NO:5. However, in this example, the wild type chimeric pig-baboon uricase and its variants expressed in E. coli possessed a methionine (preceding the threonine) as residue #1, with the threonine as residue #2, due to the presence of an Ndel restriction site encoding methionine in-frame with the wild type and the variant chimeric pig-baboon uricase genes in the expression vector pET-26b(+) (MilliporeSigma, MA, USA). The expression vector pET-26b(+) encodes a His tag containing 6 histidine residues at the C-terminus of the inserted uricase gene.

(wild type chimeric pig-baboon uricase)
SEQ ID NO: 5
TYKKNDEVEF VRTGYGKDMI KVLHIQRDGK YHSIKEVATT VQLTLSSKKD YLHGDNSDVI
PTDTIKNTVN VLAKFKGIKS IETFAVTICE HFLSSFKHVI RAQVYVEEVP WKRFEKNGVK
HVHAFIYTPT GTHFCEVEQI RNGPPVIHSG IKDLKVLKTT QSGFEGFIKD QFTTLPEVKD
RCFATQVYCK WRYHQGRDVD FEATWDTVRS IVLQKFAGPY DKGEYSPSVQ KTLYDIQVLT
LGQVPEIEDM EISLPNIHYL NIDMSKMGLI NKEEVLLPLD NPYGKITGTV KRKLSSRL

### 1. Deimmunized Library Design

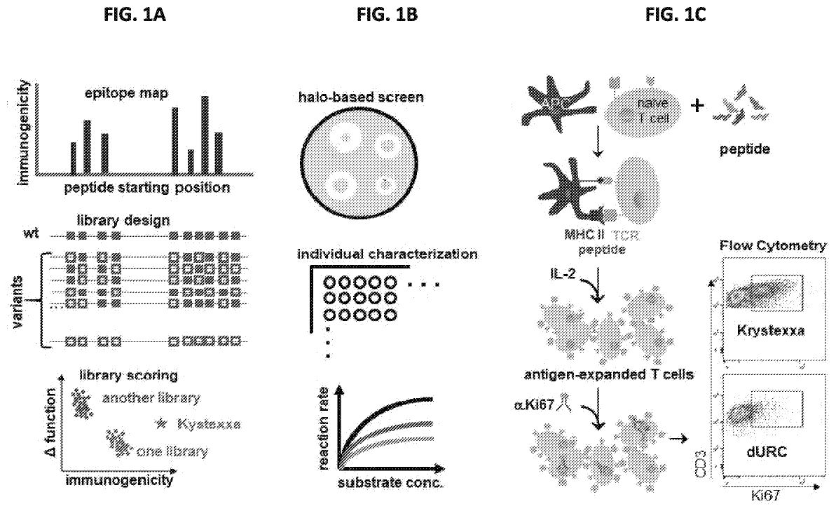

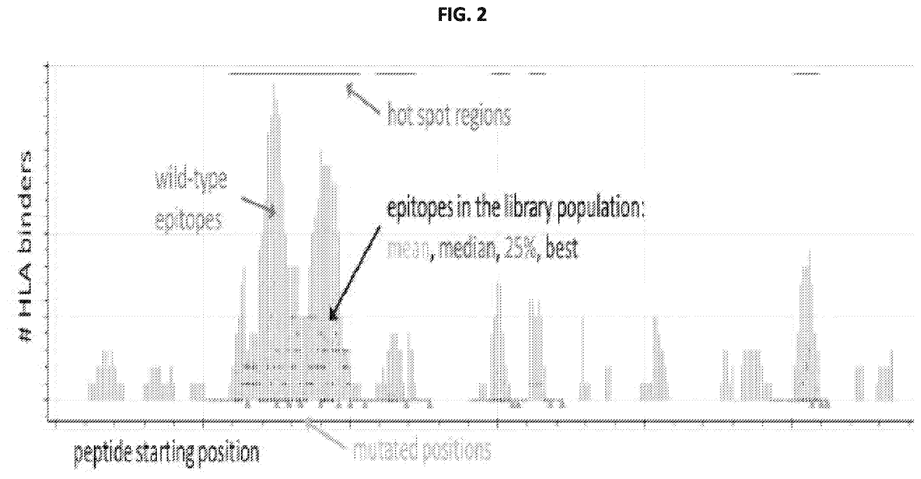

Deimmunized library design was carried out to computationally optimize uricase variant libraries and select one library for experimental construction. As illustrated in FIG. 1A, using proprietary design algorithms, mutations (both positions and position-specific substitutions) to the wild type chimeric pig-baboon uricase defining combinatorial libraries predicted to be enriched in clones with high function and low immunogenicity were selected. In designing a library, the algorithms model the entire protein and evaluate the interrelated effects of combinations of mutations on both epitope content and protein stability and function. The algorithms generated hundreds of Pareto optimal and near-optimal designs, striking different levels of aggressiveness in deleting epitopes vs. maintaining function. The designs explored different population sizes (500,000 to 1.5 million members), different numbers of mutational target sites (15 to 20), and different numbers of substitutions per site (1 to 4). Subsequent filtering and inspection of epitope and function scores led to the selection of a 17-site, ~1 million-member library (FIG. 2). The library members had reduced MHC-II binding potential as compared to the wild type chimeric pig-baboon uricase. The variants of this library design had the designed amino acids summarized in Table 1. The amino acid positions are indexed to threonine as residue #1, as in SEQ ID NO:5.

TABLE 1
Designed amino acids in chimeric pig-baboon uricase variants
according to the selected deimmunized library design

| Target | Wild Type (WT) | Variant Amino |
|---|---|---|
| Site | Amino Acid | Acid |
| 65 | I | IQ |
| 75 | F | FKQ |
| 79 | K | EKN |
| 83 | T | EQT |
| 90 | E | EHT |
| 96 | F | FH |
| 100 | I | EHI |
| 105 | Y | DY |
| 119 | V | KV |
| 127 | Y | HY |
| 155 | K | KT |
| 157 | L | KL |
| 168 | I | HI |
| 172 | F | FY |
| 257 | I | IK |
| 260 | L | LN |
| 262 | I | IT |

Upon selection of this design, functional chimeric pig-baboon uricase variants were identified via high throughput screening (FIG. 1B), with most or all immunodominant epitopes in the protein sequence silenced (FIGS. 1C and 2), as described further below.

### 2. Library Screening and Functional Evaluation.

Library screening and functional evaluation were performed to experimentally identify and characterize a diverse set of high-functioning uricase (dURC) candidates by (i) employing a high-throughput halo-based assay to select active variants, and (ii) purification and detailed analysis of kinetics and stability for the top 10-20 candidates.

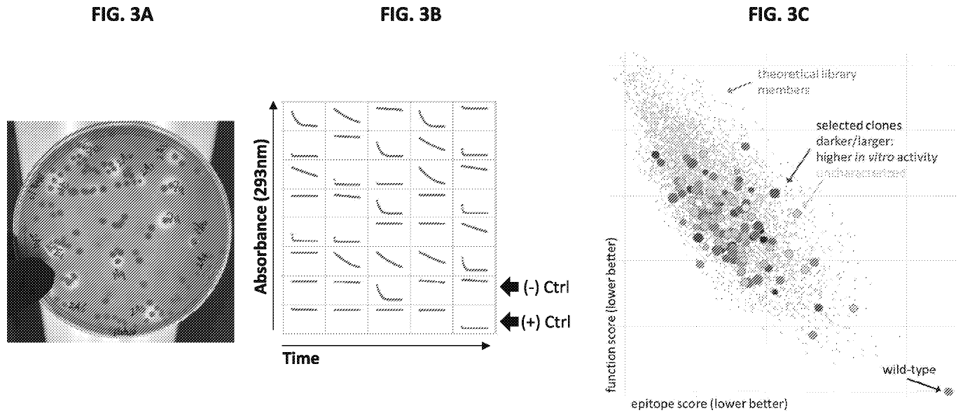

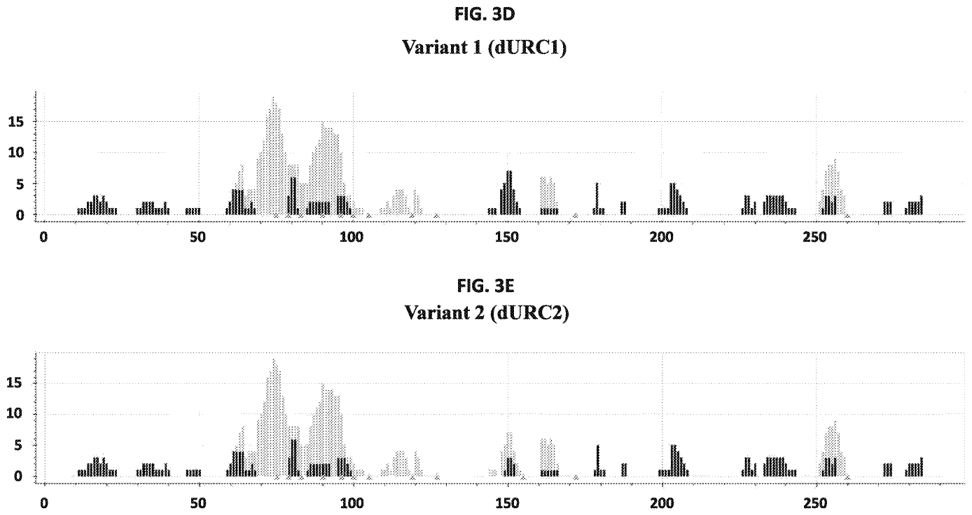

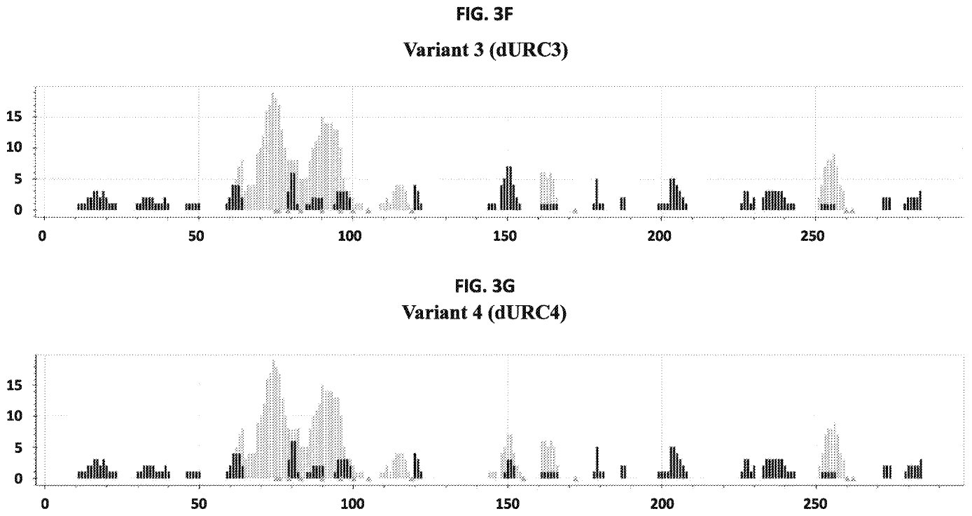

Primary screening of the combinatorial dURC library was conducted by uricase halo assay. Specifically, E. coli transformed with the expression vector pET-26b(+) containing the cDNA sequence encoding the wild type chimeric pig-baboon uricase or its variant was inoculated onto LB agar plates containing uric acid. Bacterial colonies expressing active uricase converted the suspended uric acid into water soluble allantoin, such that a clear zone (halo) around target bacteria indicates production of an active enzyme (FIG. 3A). The diameter of each clear zone was recorded as a semi-quantitative measure of enzyme expression and activity. The variants selected based on halo size were inoculated into deep 96-well plates, grown, induced, and cell pellets resuspended in lysis buffer. The uricase enzyme activity was quantified (i) in the alkaline-soluble lysate (pH 10.5 carbonate/bicarbonate buffer), and (ii) on enzyme immobilized from the alkaline-soluble lysate onto Ni-NTA HisSorb plates (Qiagen). A pseudo-estimate of expression level was calculated as the quotient of total lysate activity to immobilized lysate activity, as described in the “Methods” section above. Solution phase kinetic analysis on the whole cell lysates allowed for stringent selection of clones exhibiting the fastest kinetics (FIG. 3B). Genes from highly active variants were then sequenced and the predicted T cell epitope content for each encoded enzyme was computed. FIG. 3C is a graph showing the predicted function score vs. T cell epitope score for individual variants.

Variants 1 (dURC1) and 3 (dURC3) were identified as possible lead candidates. Besides the mutations according to the library design, i.e., the designed mutations of F75Q; K79N; T83Q; E90H; F96H; I100E; Y105D; V119K; F172Y; L260N; and I262T, dURC3 comprised an off-position amino acid mutation K76N. An off-position amino acid mutation is defined as having a non-designed amino acid at a non-target site. Since neither variant possessed mutations targeting the predicted epitope cluster in the 150 region, “polishing” mutations designed to mitigate this residual hotspot were evaluated. Specifically, in the dURC1 backbone, the following mutations were tested separately: L154T; K155D; K155N; L157H; L157T; T159E; T159Q; and S162D; and in the dURC3 backbone, the K155N mutation was tested. Those results yielded four candidate variants (variants 1-4, or dURC1-4) demonstrating high activity and large reductions in predicted T cell epitopes (FIGS. 3D-3G). The characteristics, including yield, activity, melting temperature measured by DSF, temperature at which 50% of normal activity was lost after heating measured by heat stress activity assay, Michaelis-Menten kinetics parameters, of variants 1, 2, 3, and 4 (i.e., dURC1, dURC2, dURC3 and dURC4), as well as the wild type chimeric pig-baboon uricase, are summarized in Table 2. The amino acid sequences of dURC1, dURC2, dURC3 and dURC4 are shown as SEQ ID NO: 1, 2, 3, and 4, respectively.

TABLE 2
Characteristics of wild type chimeric pig-baboon uricase and variants 1, 2, 3, and 4

| | | Mutations | | | | | | | | |
|---|---|---|---|---|---|---|---|---|---|---|
| Variant or | | relative to | | | | | | | | |
| wild type | | wild type | | | | | | | | |
| chimeric | | chimeric | | | | | | PBS | | |
| pig- | | pig-baboon | | | | | Temp | | Apparent | Apparent |
| baboon | | uricase of | Yield | | Activity | Tₘ by | Activity | | k_{cat} | k_{cat}/Kₘ |
| uricase | Code | SEQ ID | (μg/mL culture) | | by | DSF | Lost | Kₘ | (arbitrary | (arbitrary |
| (SEQ ID NO) | name | NO: 5 | Mean | SD | HisSorb | (° C.) | (° C.) | (μM) | units) | units) |
| Variant 1 | dURC1 | F75Q; K79E; | 125 | 64 | 2.1 | N/A | 41.9 | 678 | 42.5 | 15.9 |
| (SEQ ID NO: 1) | | T83Q; E90H; | | | | | | | | |
| | | F96H; I100H; | | | | | | | | |
| | | Y105D; V119K; | | | | | | | | |
| | | Y127H; F172Y; | | | | | | | | |
| | | L260N | | | | | | | | |
| Variant 2 | dURC2 | F75Q; K79E; | 52 | 26 | N/A | N/A | 38.6 | 467 | 34.4 | 13.6 |
| (SEQ ID NO: 2) | | T83Q; E90H; | | | | | | | | |
| | | F96H; I100H; | | | | | | | | |
| | | Y105D; V119K; | | | | | | | | |
| | | Y127H; K155N; | | | | | | | | |
| | | F172Y; L260N | | | | | | | | |
| Variant 3 | dURC3 | F75Q; K76N; | 140 | 80 | 1.6 | N/A | 36.5 | 595 | 40.4 | 14.7 |
| (SEQ ID NO: 3) | | K79N; T83Q; | | | | | | | | |
| | | E90H; F96H; | | | | | | | | |
| | | I100E; Y105D; | | | | | | | | |
| | | V119K; F172Y; | | | | | | | | |
| | | L260N; I262T | | | | | | | | |
| Variant 4 | dURC4 | F75Q; K76N; | 167 | 48 | N/A | N/A | 36 | 347 | 27.6 | 12.6 |
| (SEQ ID NO: 4) | | K79N; T83Q; | | | | | | | | |
| | | E90H; F96H; | | | | | | | | |
| | | I100E; Y105D; | | | | | | | | |
| | | V119K; K155N; | | | | | | | | |
| | | F172Y; L260N; | | | | | | | | |
| | | I262T | | | | | | | | |
| Wild Type | N/A | N/A | 375 | 38 | N/A | 44 | 40.9 | 397 | 29.6 | 13.4 |
| (SEQ ID NO: 5) | | | | | | | | | | |

Tₘ = melting temperature;
DSF = differential scanning fluorimetry

(dURC1, with the mutations to the wild type chimeric pig-baboon
uricase depicted by italics and boldface)
SEQ ID NO: 1
TYKKNDEVEF VRTGYGKDMI KVLHIQRDGK YHSIKEVATT VQLTLSSKKD YLHGDNSDVI
PTDTIKNTVN VLAKQKGIES IEQFAVTICH HFLSSHKHVH RAQVDVEEVP WKRFEKNGKK
HVHAFIHTPT GTHFCEVEQI RNGPPVIHSG IKDLKVLKTT QSGFEGFIKD QYTTLPEVKD
RCFATQVYCK WRYHQGRDVD FEATWDTVRS IVLQKFAGPY DKGEYSPSVQ KTLYDIQVLT
LGQVPEIEDM EISLPNIHYN NIDMSKMGLI NKEEVLLPLD NPYGKITGTV KRKLSSRL
(dURC2, with the mutations to the wild type chimeric pig-baboon
uricase depicted by italics and boldface)
SEQ ID NO: 2
TYKKNDEVEF VRTGYGKDMI KVLHIQRDGK YHSIKEVATT VQLTLSSKKD YLHGDNSDVI
PTDTIKNTVN VLAKQKGIES IEQFAVTICH HFLSSHKHVH RAQVDVEEVP WKRFEKNGKK
HVHAFIHTPT GTHFCEVEQI RNGPPVIHSG IKDLNVLKTT QSGFEGFIKD QYTTLPEVKD
RCFATQVYCK WRYHQGRDVD FEATWDTVRS IVLQKFAGPY DKGEYSPSVQ KTLYDIQVLT
LGQVPEIEDM EISLPNIHYN NIDMSKMGLI NKEEVLLPLD NPYGKITGTV KRKLSSRL
(dURC3, with the mutations to the wild type chimeric pig-baboon
uricase depicted by italics and boldface)
SEQ ID NO: 3
TYKKNDEVEF VRTGYGKDMI KVLHIQRDGK YHSIKEVATT VQLTLSSKKD YLHGDNSDVI
PTDTIKNTVN VLAKQNGINS IEQFAVTICH HFLSSHKHVE RAQVDVEEVP WKRFEKNGKK
HVHAFIYTPT GTHFCEVEQI RNGPPVIHSG IKDLKVLKTT QSGFEGFIKD QYTTLPEVKD
RCFATQVYCK WRYHQGRDVD FEATWDTVRS IVLQKFAGPY DKGEYSPSVQ KTLYDIQVLT
LGQVPEIEDM EISLPNIHYN NTDMSKMGLI NKEEVLLPLD NPYGKITGTV KRKLSSRL
(dURC4, with the mutations to the wild type chimeric pig-baboon
uricase depicted by italics and boldface)
SEQ ID NO: 4
TYKKNDEVEF VRTGYGKDMI KVLHIQRDGK YHSIKEVATT VQLTLSSKKD YLHGDNSDVI
PTDTIKNTVN VLAKQNGINS IEQFAVTICH HFLSSHKHVE RAQVDVEEVP WKRFEKNGKK
HVHAFIYTPT GTHFCEVEQI RNGPPVIHSG IKDLNVLKTT QSGFEGFIKD QYTTLPEVKD
RCFATQVYCK WRYHQGRDVD FEATWDTVRS IVLQKFAGPY DKGEYSPSVQ KTLYDIQVLT
LGQVPEIEDM EISLPNIHYN NTDMSKMGLI NKEEVLLPLD NPYGKITGTV KRKLSSRL

dURC Functional Evaluation

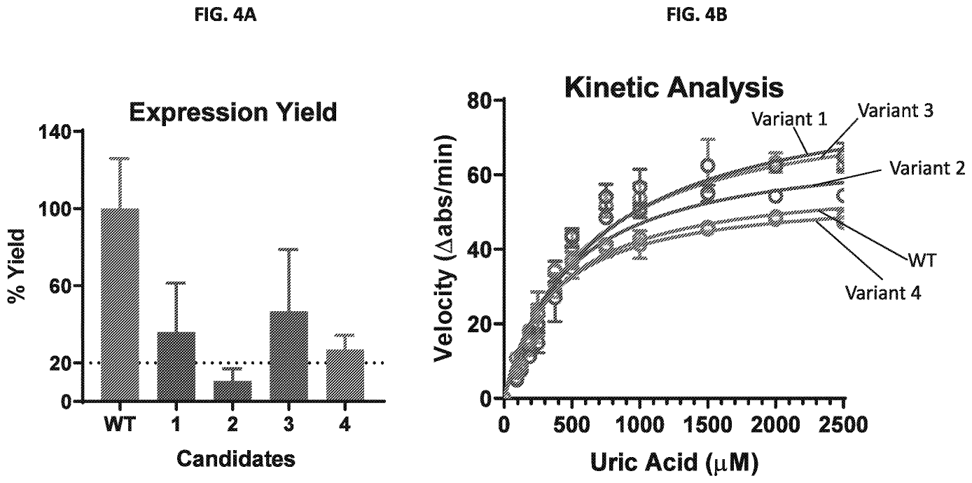

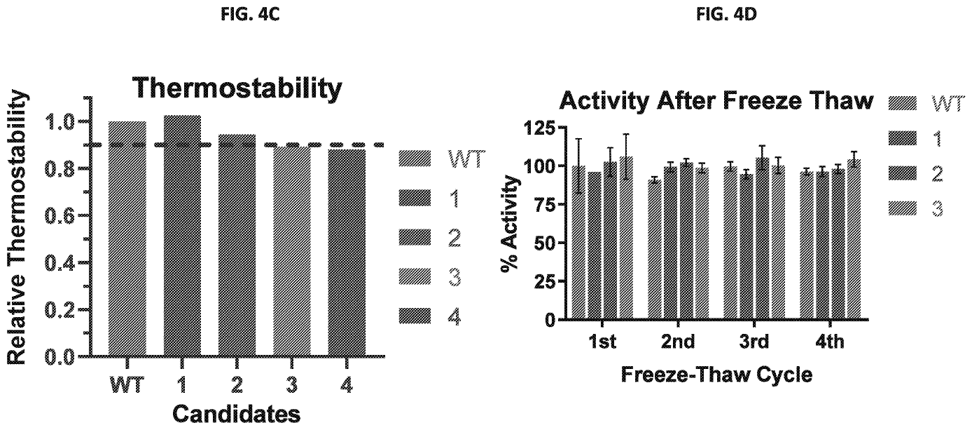

Each of candidate variants 1~4 was then expressed in large-volume batch cultures and purified. Purified protein yields were quantitatively determined for each enzyme and are shown as a percentage of the wild type chimeric pig-baboon uricase (FIG. 4A). Three of the candidates (variants 1, 3, and 4) had yields of ≥20% of that of the wild type, with variant 3 (dURC3) achieving ~50% of the wild type yield.

The activity of the purified enzymes was quantified by Michaelis Menten kinetic analysis, using the solution phase microplate assay across a range of substrate concentrations (FIG. 4B). Table 3 shows the kinetic parameters (apparent k_{cat} and K_{M}) determined for each enzyme by non-linear regression. All candidates had ≥33% of the wild type activity, with three of the four candidates manifesting faster kinetics than the wild type chimeric pig-baboon uricase. dURC3 had a 33% higher apparent k_{cat} compared to the wild type.

TABLE 3
Kinetic parameters of wild type chimeric
pig-baboon uricase and variants

| | Apparent k_{cat} | |
|---|---|---|
| Uricase Enzyme | (arbitrary units) | K_{M} (μM) |
| Variant 1 (dURC1) | 43 ± 2 | 700 ± 100 |
| Variant 3 (dURC3) | 40 ± 2 | 600 ± 70 |
| Variant 2 (dURC2) | 34 ± 1 | 470 ± 60 |
| Wild type chimeric pig- | 30 ± 1 | 400 ± 50 |
| baboon uricase(WT) | | |
| Variant 4 (dURC4) | 28.0 ± 0.5 | 350 ± 20 |

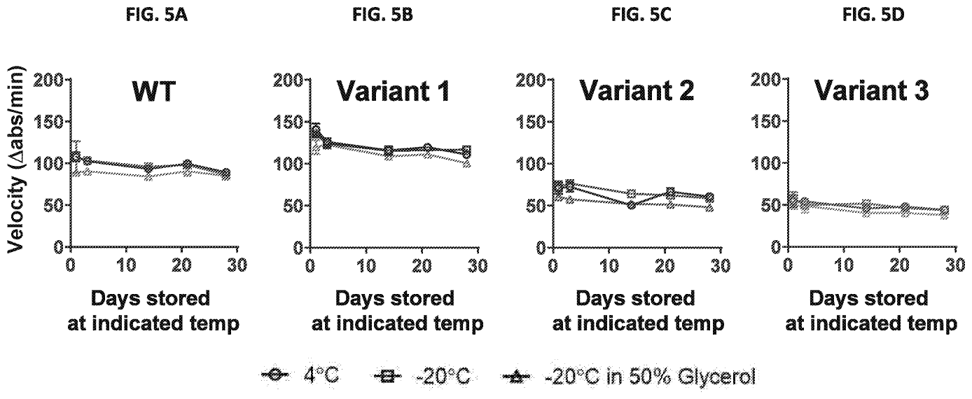

Protein stability was quantified by a number of metrics. Resistance to heat inactivation was measured by heat stress activity assay (FIG. 4C), and three of the four candidate variants had >90% wild type stability. Stability during repeated freeze-thaw cycles was quantified by measuring specific activity during temperature cycling. Variants 1-3 exhibited wild type levels of stability, with no statistically significant loss of activity through at least four cycles of freeze-thaw (FIG. 4D). Finally, the activity of variants 1-3 was tracked during storage at 4° C. and −20° C., the latter with or without glycerol cryoprotectant. None of the enzymes exhibited significant loss in activity during at least a month of storage (FIGS. 5A-5D).

### 3. Immunogenicity Analysis

An assessment of immunogenicity of the uricase (dURC) candidate variants relative to the wild type chimeric pig-baboon uricase was made by ex vivo cellular immunoassays using human peripheral blood mononuclear cells (PBMCs). The details of the immunoassays are described in Salvat et al., Proc Natl Acad Sci. USA, 2017, 114(26):E5085-E5093, incorporated herein by reference in its entirety. A flow cytometric approach was adopted to enable initial immunogenicity assessment of the four candidate variants. Based on the experimental immunogenicity data, a final lead candidate was selected.

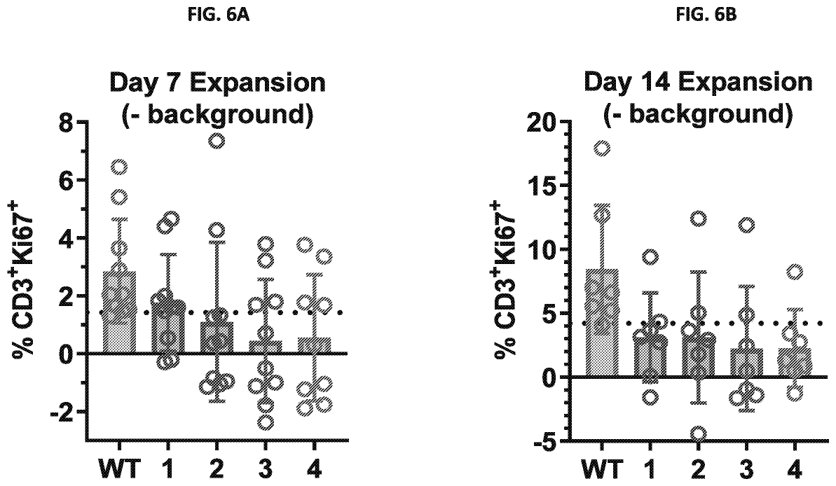

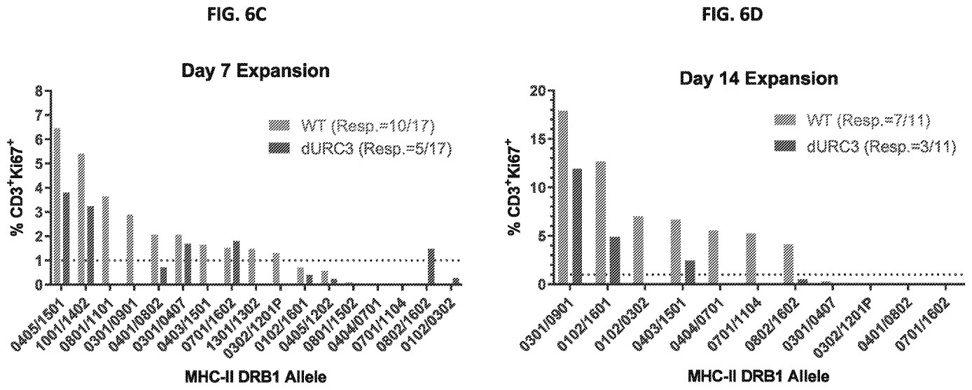

To assess the immunostimulatory potential of the enzymes, a well-validated flow cytometry protocol was employed to quantify antigen specific T cell stimulation by fluorescent staining of the Ki67 proliferation marker. PBMCs from 20 human donors were expanded in the presence of peptide fragments corresponding to all epitope hotspots of the wild type chimeric pig-baboon uricase or matched peptides from each of the dURC variants 1-4. The percentage of proliferating (i.e., Ki67⁺) antigen-specific T cells was evaluated on days 7 and 14. All dURC variants showed reduced proliferative response in comparison to the wild type chimeric pig-baboon uricase at both day 7 and day 14 (FIGS. 6A and 6B). Variant dURC3 was selected as the final lead candidate based on the magnitude of its reduction in T cell stimulation relative to the wild type chimeric pig-baboon uricase (6-fold on day 7, and 4-fold on day 14). The proportion of responding donors following stimulation with the wild type chimeric pig-baboon uricase (WT) or dURC3 was evaluated next, where responders were defined as donors with >1% increase in CD3⁺Ki67⁺ T cells above the vehicle control condition. Seventeen of the 20 donors met inclusion criteria for analysis on day 7 and eleven of the 20 donors on day 14. WT manifested 10/17 responding donors on day 7 and 7/11 on day 14, compared to 5/17 and 3/11, respectively, for dURC3. Thus, dURC3 exhibited >2-fold reduction in proportion of responding donors for both the day 7 and day 14 timepoints (FIGS. 6C and D). The donor cohort exhibited diverse MHC-II genotypes, and this data provides evidence that dURC3 is deimmunized for a broad patient population.

### Example 2—Creation and Characterization of PEGylated Chimeric Pig-Baboon Uricase Variant dURC3

In this example, the chimeric pig-baboon uricase variant dURC3, as well as the wild type chimeric pig-baboon uricase, expressed in E. coli as described in Example 1, was purified and conjugated to polyethylene glycol (PEG). The activity, thermostability, and immunogenicity of the resultant PEGylated dURC3 and PEGylated wild type chimeric pig-baboon uricase were determined.

### Methods

### 1. Purification

The wild type chimeric pig-baboon uricase and its variants expressed in E. coli were subject to multi-step purifications by various means, as detailed below.

### 1.1. Inclusion Body Purification

E. coli cells were lysed with 50 ml B-PER bacterial protein extraction reagent (ThermoFisher Scientific) per liter of culture with the addition of 1000 U/L BENZONASE® endonuclease (MilliporeSigma). Specifically, the cell pellet was resuspended in B-PER using a Dounce homogenizer followed by incubation at room temperature for one hour on a nutator. The suspension was centrifuged at 25,000 rpm for 30 min at 4° C. The supernatant was removed and discarded, and the pellet was resuspended using a Dounce homogenizer in 50 ml/L load buffer containing 100 mM sodium bicarbonate (pH 10.5) and 10 mM imidazole. After incubation at room temperature for one hour on a nutator, the suspension was centrifuged at 25,000 rpm for 30 min at 4° C., and the supernatant was removed and reserved for subsequent chromatography.

### 1.2. Chromatographic Purification

The reserved supernatant from Section 1.1 above was loaded onto a HISPREP FF 16/10 column at 5 ml/min. The column was then washed with 2 column volumes (cv) of load buffer containing 1% TRITON-114 followed by 10 cv of load buffer, followed by elution with 5 cv of 100 mM sodium bicarbonate (pH 10.5) and 500 mM imidazole. The eluted protein was analyzed by SDS-PAGE, activity, DSF, liquid chromatography-mass spectrometry (LC/MS), and dynamic light scattering (DLS). The elution was subsequently concentrated to 15 ml using stirred cell with 50 kDa filter to a concentration not exceeding 3 mg/ml. The concentrated elution was loaded onto a SUPERDEX 200 HILOAD 26/600 column, followed by elution with 100 mM sodium bicarbonate (pH 10.5). Fractions containing tetrameric uricase as identified by DLS as being monodisperse were pooled, and the pooled tetrameric uricase fractions were analyzed by SDS-PAGE and DLS. The pooled tetrameric uricase fractions were then concentrated using stirred cell with 50 kDa filter to 3 mg/ml.

### 1.3. Endotoxin Mitigation

Endotoxin in a preparation of the wild type chimeric pig-baboon uricase and its variant was removed by the following procedure:
(1) adding to the preparation 1% TRITON-114 followed by incubation for 30 minutes at 4° C. and then 30 minutes at 37° C.;
(2) centrifuging the mixture in pyrogen free tubes for 30 minutes at 37° C.;
(3) decanting the top layer to fresh pyrogen free tubes;
(4) repeating steps (1)-(3) as needed to lower endotoxin level to <0.5 EU/ml;
(5) repeating steps (2) and (3) one final time to remove residual TRITON-114;
(6) testing endotoxin using limulus amebocyte lysate (LAL) assay (Charles River) by diluting the test sample 1:20 in endotoxin free water and applying 25 μl to each sample well.

### 2. PEGylation of the Wild Type Chimeric Pig-Baboon Uricase and its Variants

PEGylation of the wild type chimeric pig-baboon uricase or its variants was performed by the following procedure:
(1) adding 50 fold molar excess methoxy-PEG-(CH₂)₅COO—NHS of 10 kDa in molecular weight (Chemical Name: Poly(oxy-1,2-ethanediyl), α-methyl-ω-{2-[(2,5-dioxo-1-pyrrolidinyl)oxy]-6-oxohexyloxy}; NOF America Corporation, Cat #SUNBRIGHT ME-100HS) to purified uricase protein on ice and incubating with stirring for one hour;
(2) verifying radius by DLS (14-15 nm radius ~=wild type);
(3) concentrating PEGylated protein using stirred cell with 50 kDa filter to 15 ml;
(4) loading the concentrated PEGylated protein onto a SUPERDEX 200 HILOAD 26/600 column and eluting with PBS;
(5) pooling fractions identified as being mono-disperse by DLS;
(6) concentrating the pooled fractions using a stirred cell with 50 kDa filter to 5 mg/ml.

### 3. Titer Determination for Antidrug Antibody in Mouse Plasma Using ELISA

A high-binding 96-well ELISA plate (COSTAR #2592) was coated with a 5 μg/mL solution containing either PEGylated chimeric pig-baboon uricase variant dURC3 protein, or PEGylated wild type chimeric pig-baboon uricase protein, in bicarbonate buffer overnight at 4° C. The coating solution was then removed, and the plate was washed three times with phosphate buffer containing 0.1% Tween 20 detergent. The washed plate was blocked with 1× superblock buffer (ThermoFisher #37515) for 15 minutes at 37° C., followed by removal of the superblock buffer. Serial dilutions of 1:20 to 1:12,207 of mouse plasma in LOWCROSS buffer (Fisher Scientific #NC9831360) were added to the wells, and the plate was incubated at 37° C. for 60 minutes. Plasma dilutions were removed, and the plate was washed three times with phosphate buffer containing 0.1% Tween 20 detergent. The plate was then incubated for 30 minutes at 37° C. with a 1:10,000 dilution of an anti-mouse secondary antibody conjugated with horseradish peroxidase, diluted in LOWCROSS buffer. The secondary antibody was then removed, and the plate was washed four times with phosphate buffer containing 0.1% Tween 20 detergent. Antidrug antibodies were then quantified by detection with TNB (3,3′,5,5′-tetramethylbenzidine) substrate (Bethyl #E102), measuring absorbance at 450 nm and subtracting background absorbance at a reference wavelength of 620 nm.

### Results

dURC3 and the wild type chimeric pig-baboon uricase were successfully PEGylated, with an average of about 10 PEG molecules conjugated to a uricase monomer, based on SDS-PAGE and size exclusion chromatography with multi-angle static light scattering (SEC-MALS) analysis. The activity quantified by Michaelis Menten kinetic analysis, as well as the melting temperature quantified by differential scanning fluorimetry, of PEGylated dURC3 and PEGylated wild type chimeric pig-baboon uricase was determined using the methods described in Example 1. The data are shown in Table 4.

TABLE 4
Kinetic parameters and melting temperatures of PEGylated
dURC3 and PEGylated wild type chimeric pig-baboon uricase

| | PEGylated | PEGylated |
|---|---|---|
| | dURC3 | wild type |
| Specific Activity (arbitrary units/second) | 47.5 | 150 |
| Vₘₐₓ (Δabs/min) | 67 | 170 |
| Apparent k_{cat}/Kₘ (arbitrary units) | 0.135 | 0.203 |
| Melting temperature (° C.) | 55 | 62.5 |

The immunogenicity of PEGylated dURC3 and PEGylated wild type chimeric pig-baboon uricase was assessed in humanized HLA transgenic mouse strain DR4. Humanized HLA transgenic mouse strain DR4 has functioning cellular and humoral immunity, but the murine class II MHC locus has been replaced with a functional form of human MHC II allele DRB1*0401, a common allele among human populations and a representative MHC II supertype. DR4 mice develop antidrug antibodies as a result of human DRB1*0401 restriction of T cell epitopes.

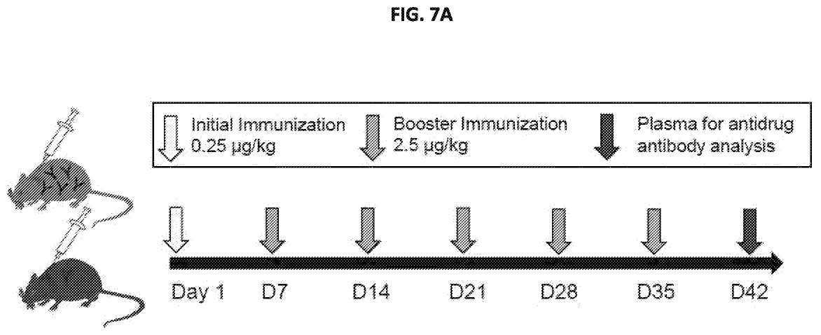

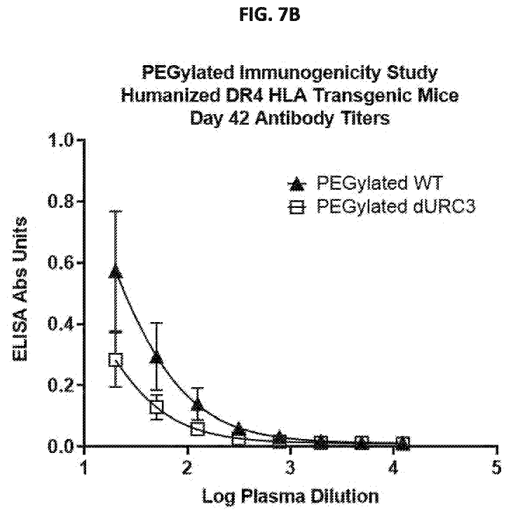

DR4 mice were subject to initial immunization via intraperitoneal administration on Day 1 with 0.25 μg/kg PEGylated dURC3 or PEGylated wild type chimeric pig-baboon uricase and booster immunizations via intraperitoneal administration on Days 7, 14, 21, 28, and 35 with 2.5 μg/kg PEGylated dURC3 and PEGylated wild type chimeric pig-baboon uricase, respectively, with 10 mice in each test drug (i.e., PEGylated uricase) group. Mice were dosed at about 1/500 of the human equivalent dose by allometric scaling to minimize drug interference in the measurement of antidrug antibodies. Mouse plasma was collected on Day 42 for antidrug antibody titer analysis by ELISA (FIG. 7A).

FIG. 7B shows the curves representing the relationship between the antidrug antibody titer determined by ELISA absorbance and plasma dilution on a log scale, using the Day 42 plasma collected from the humanized DR4 HLA transgenic mice immunized with the PEGylated dURC3 or the PEGylated wild type counterpart. Higher ELISA absorbance at larger plasma dilutions is indicative of a stronger antidrug antibody response. One summary measure of overall response is area under the plasma dose-response curve (AUC), where larger AUC indicates a stronger antidrug immune response. Table 5 shows the antidrug antibody titer AUC. The results demonstrate that PEGylated dURC3 was less immunogenic than PEGylated wild type chimeric pig-baboon uricase in DR4 mice despite low dosing levels required by the study design.

TABLE 5
Day 42 plasma antidrug antibody titer AUC of humanized
DR4 HLA transgenic mice immunized with PEGylated dURC3
or PEGylated wild type chimeric pig-baboon uricase

| | PEGylated dURC3 | PEGylated wild type |
|---|---|---|
| Day 42 plasma antidrug | 0.15 | 0.33 |
| antibody titer AUC | | |

### Example 3—Creation and Characterization of Non-PEGylated Deimmunized Arthrobacter globiformis Uricase Variants, Aspergillus flavus Uricase Variants, and Candida utilis Uricase Variants

This example describes the development of highly engineered, deimmunized non-PEGylated variants of the wild type Arthrobacter globiformis uricase of SEQ ID NO:6, the wild type Aspergillus flavus uricase of SEQ ID NO:7, and the wild type Candida utilis uricase of SEQ ID NO:8 using the same computationally-driven protein engineering platform as used in Example 1. Variants of each uricase exhibited activity and/or expression levels comparable to or, better than, those of the corresponding wild type uricase, while having reduced immunogenic potential effected by mutagenic silencing of constituent T cell epitopes.

### Methods

### 1. Uricase Halo Assay

Uricase halo assay similar to that described in Example 1 was performed in this example. Briefly, E. coli transformed with the expression vector pET-26b(+) containing the cDNA sequence encoding a wild type uricase or a uricase variant was inoculated onto LB agar plates containing uric acid. Bacterial colonies expressing active uricase converted the suspended uric acid into water soluble allantoin, such that a clear zone (halo) around target bacteria indicates production of an active enzyme. The diameter of each clear zone was recorded as a semi-quantitative measure of enzyme expression and activity. The variants selected based on halo size were subject to soluble enzyme activity assay and expression assay described below.

### 2. Soluble Enzyme Activity Assay

Small volume cultures of E. coli transformed with the expression vector pET-26b(+) containing the cDNA sequence encoding a wild type uricase or a uricase variant were grown and induced in deep 96-well plates. The expression vector pET-26b(+) encodes a His tag containing 6 histidine residues at the C-terminus of the inserted uricase gene. Induced E. coli cells were pelleted by centrifugation. Cell pellets were resuspended and lysed in BUGBUSTER® HT Protein Extraction Reagent (MilliporeSigma, MA, USA). Cell debris were pelleted by centrifugation, and the soluble supernatant was collected and transferred to a UV transparent 96-well plate, where the soluble supernatant was combined with a uric acid substrate solution (0.1 M boric acid, 0.12 mM uric acid, pH 9.0), and absorbance at 293 nm was measured as a function of time. Uricase activity was measured as the slope of the absorbance at 293 nm vs time curve, with steeper negative slopes indicative of higher activity. As such, the slopes were quantified as raw activity measures in arbitrary units.

### 3. Expression Assay

The expression assay is based on measuring total activity in the soluble supernatant following lysis of E. coli cell pellets with BUGBUSTER® HT Protein Extraction Reagent, as described above, and separately measuring activity of immobilized uricase enzyme (on a 96-well Ni-NTA HisSorb plate (Qiagen)) from the soluble supernatant (described below), as the wild type uricase or its variant produced in E. coli contained a His tag at the C-terminus. In theory the HisSorb immobilized surface is saturated with enzyme, and thus dividing total activity by this saturated immobilized activity yields a relative expression level.

To measure the activity of immobilized uricase enzyme from the soluble supernatant, small volume cultures of E. coli transformed with the expression vector pET-26b(+) containing the cDNA sequence encoding a wild type uricase or a uricase variant were grown and induced in deep 96-well plates. The induced E. coli cells were pelleted by centrifugation. Cell pellets were resuspended and lysed in BUGBUSTER® HT Protein Extraction Reagent. Cell debris were pelleted by centrifugation. The soluble supernatant was collected and transferred to a 96-well Ni-NTA HisSorb plate (Qiagen) bearing immobilized metal ion affinity ligands on the bottom of the wells. The HisSorb plate was incubated for one hour to bind the His-tagged uricase enzymes to the wells, followed by the removal of the supernatant from the wells and washing of the wells five times with a phosphate buffer. Thereafter, a uric acid substrate solution (0.1 M boric acid, 0.12 mM uric acid, pH 9.0) was added to the HisSorb plate, and absorbance at 293 nm was measured as a function of time. Uricase activity was measured as the slope of the absorbance at 293 nm vs time curve, with steeper negative slopes indicative of higher activity. Relative expression level was calculated as the activity of the total soluble enzyme fraction divided by the activity of the HisSorb-bound enzyme fraction.

### Results

1. Creation and Characterization of Non-PEGylated Deimmunized Arthrobacter globiformis Uricase Variants

Non-PEGylated deimmunized Arthrobacter globiformis uricase variants were created by introducing additional amino acid substitutions into the R49S variant Arthrobacter globiformis uricase of SEQ ID NO:6 disclosed in Nyborg et al., PLOS One, 2016, DOI: 10.1371/journal.pone.0167935. For ease of description, the R49S variant Arthrobacter globiformis uricase of SEQ ID NO:6 is referred to as the wild type Arthrobacter globiformis uricase herein, as it serves as the enzyme being mutated to arrive at the variants. The monomeric form of the wild type Arthrobacter globiformis uricase comprises the amino acid sequence of SEQ ID NO:6, where methionine is residue #1. The specified mutations in the Arthrobacter globiformis uricase variants of this example are indexed to methionine as residue #1, as in SEQ ID NO:6. In this example, the wild type Arthrobacter globiformis uricase and each of its variants were expressed in E. coli transformed with the expression vector pET-26b(+) containing the cDNA sequence encoding the wild type Arthrobacter globiformis uricase or its variant. The expression vector pET-26b(+) encodes a His tag containing 6 histidine residues at the C-terminus of the inserted uricase gene.

(wild type Arthrobacter globiformis uricase)
SEQ ID NO: 6
MTATAETSTG TKVVLGQNQY GKAEVRLVKV TRNTARHEIQ DLNVTSQLSG DFEAAHTAGD
NAHVVATDTQ KNTVYAFARD GFATTEEFLL RLGKHFTEGF DWVTGGRWAA QQFFWDRIND
HDHAFSRNKS EVRTAVLEIS GSEQAIVAGI EGLTVLKSTG SEFHGFPRDK YTTLQETTDR
ILATDVSARW RYNTVEVDFD AVYASVRGLL LKAFAETHSL ALQQTMYEMG RAVIETHPEI
DEIKMSLPNK HHFLVDLQPF GQDNPNEVFY AADRPYGLIE ATIQREGSRA D

### 1.1. Deimmunized Library Design

Deimmunized library design was carried out as described in Example 1, with the selection of a 17-site library with 663,552 members. The library members were predicted by the design algorithms to have reduced MHC-II binding potential as compared to the wild type Arthrobacter globiformis uricase. The variants of this library design had the designed amino acids summarized in Table 6. The amino acid positions are indexed to methionine as residue #1, as in SEQ ID NO:6.

TABLE 6
Designed amino acids in Arthrobacter globiformis uricase
variants according to the selected deimmunized library design

| Target | Wild Type (WT) | Variant Amino |
|---|---|---|
| Site | Amino Acid | Acid |
| 33 | N | EN |
| 35 | A | AD |
| 82 | F | FV |
| 83 | A | AG |
| 89 | L | GL |
| 113 | F | FH |
| 145 | A | AHT |
| 148 | A | AS |
| 195 | V | DEV |
| 203 | Y | HY |
| 204 | A | ADG |
| 213 | A | AG |
| 220 | L | HKL |
| 243 | I | IV |
| 244 | K | KS |
| 250 | K | KN |
| 270 | Y | HY |

1.2. Identification, Activity and Expression Levels of Functional Arthrobacter globiformis Uricase Variants

Upon selection of the library design described above, functional Arthrobacter globiformis uricase variants were identified via (i) a high-throughput halo-based assay to select active variants, and (ii) purification and detailed analysis of the activity and expression levels of the variants as compared to the wild type Arthrobacter globiformis uricase. The details of the halo-based assay and the activity and expression level assays are described above in the “Methods” section.

Table 7 shows 185 functional Arthrobacter globiformis uricase variants identified. With the genes of the variants sequenced, Table 7 shows each variant's SEQ ID NO and mutations relative to the wild type Arthrobacter globiformis uricase of SEQ ID NO:6, as well as the activity and expression level of each variant. Besides the mutations according to the library design, i.e., designed mutations, some of the variants comprised “off-amino acid mutation(s),” i.e., non-designed amino acid(s) at target site(s), and/or “off-position amino acid mutation(s),” i.e., non-designed amino acid(s) at non-target site(s). Data for the wild type Arthrobacter globiformis uricase are also included in Table 7 for comparison.

TABLE 7
Exemplary functional Arthrobacter globiformis uricase variants

| SEQ ID NO | | | | | | |
|---|---|---|---|---|---|---|
| for wild type | WT or Code | | | | | |
| (WT) or | Name for | | | | | |
| variant | variant | | | | | |
| Arthrobacter | Arthrobacter | | Off-amino | Off-position | | |
| globiformis | globiformis | Designed | acid | amino acid | | |
| uricase | uricase | mutations | mutations | mutations | Expression | Activity |
| 6 | WT | N/A | N/A | N/A | 0.7 | −33 |
| 12. | MedU_DF5 | F113H/A145T/V195E/ | | G152S | n/a | n/a |
| | | A213G/I243V/K244S | | | | |
| 13. | MedU_DF6 | A83G/A145H/A148S/ | F82L/I243M | | n/a | n/a |
| | | A204G/L220H/K244S/ | | | | |
| | | Y270H | | | | |
| 14. | MedU_DT3 | N33E/F82V/A145T/ | | A183S | n/a | n/a |
| | | V195D/A213G/L220K/ | | | | |
| | | I243V/K244S/Y270H | | | | |
| 15. | MedU_LT1 | A35D/F82V/A213G/ | | | n/a | n/a |
| | | L220K/I243V/K244S | | | | |
| 16. | MedU_QT3 | F82V/A145H/A148S/ | | | n/a | n/a |
| | | V195D/A204D/A213G/ | | | | |
| | | I243V/K244S | | | | |
| 17. | MedU_RT2 | A35D/A83G/V195D/ | | T194A | n/a | n/a |
| | | A213G/L220K | | | | |
| 18. | MedU_RT4 | A35D/A145H/A148S/ | | E6K/E24Q/R180C/ | n/a | n/a |
| | | V195D/A204G/I243V/ | | L182V/T184A/ | | |
| | | K250N/Y270H | | S187F/A188G/ | | |
| | | | | L257M/Q258H | | |
| 19. | MedU_SF4 | F113H/A148S/V195D/ | | V147I | n/a | n/a |
| | | A204G/L220K/K244S/ | | | | |
| | | K250N/Y270H | | | | |
| 20. | MedU_SF5 | A35D/A83G/A145H/ | | V25A | n/a | n/a |
| | | L220K/K244S | | | | |
| 21. | MedU_SF7 | N33E/A83G/A148S/ | | H237P/H251P | n/a | n/a |
| | | V195D/A204D/L220K/ | | | | |
| | | I243V/K250N/Y270H | | | | |
| 22. | MedU_SF8 | F82V/A83G/V195E/ | A145D/L220N | | n/a | n/a |
| | | Y203H/A204G/A213G/ | | | | |
| | | I243V | | | | |
| 23. | MedU_ST1 | A83G/A145T/A148S/ | | | n/a | n/a |
| | | V195D/A204G | | | | |
| 24. | MedU_ST2 | N33E/A83G/A145H/ | | | n/a | n/a |
| | | A148S/V195E/L220H/ | | | | |
| | | I243V/K250N | | | | |
| 25. | MedU_TF4 | F82V/A83G/F113H/ | | | n/a | n/a |
| | | V195E/K244S/Y270H | | | | |
| 26. | MedU_TT1 | A35D/V195D/A204D/ | | | n/a | n/a |
| | | A213G/L220H/I243V | | | | |
| 27. | MedU_aF6 | A83G/F113H/A145T/ | | | n/a | n/a |
| | | A148S/V195D/L220H/ | | | | |
| | | I243V | | | | |
| 28. | MedU_aF7 | N33E/A83G/A148S/ | | A124T/V132I | n/a | n/a |
| | | V195D/A204D/L220K/ | | | | |
| | | I243V/Y270H | | | | |
| 29. | MedU_aT1 | A35D/A145T/A148S/ | | | n/a | n/a |
| | | V195E/A204G/A213G/ | | | | |
| | | L220H/I243V/K250N | | | | |
| 30. | MedU_bT1 | A83G/F113H/A145T/ | | | n/a | n/a |
| | | V195E/A204D/L220K | | | | |
| 31. | MedU_bT2 1 | N33E/A35D/A83G/ | | | n/a | n/a |
| | | A145T/A148S/V195E/ | | | | |
| | | A204D/L220K/K250N/ | | | | |
| | | Y270H | | | | |
| 32. | MedU_eT1 | L89G/A148S/A204D/ | N33D/A145D | | n/a | n/a |
| | | A213G/L220H | | | | |
| 33. | MedU_eT2 | N33E/A145T/A148S/ | | L257M/Q258H | n/a | n/a |
| | | V195D/A204D/L220H/ | | | | |
| | | Y270H | | | | |
| 34. | MedU_fF1 | F113H/A145T/V195E/ | | | n/a | n/a |
| | | A204G/A213G/L220H/ | | | | |
| | | I243V | | | | |
| 35. | MedU_fF2 | V195E/A213G/K250N/ | | | n/a | n/a |
| | | Y270H | | | | |
| 36. | MedU_gT2 | N33E/A35D/A204G/ | | | n/a | n/a |
| | | A213G/L220H/K244S/ | | | | |
| | | K250N | | | | |
| 37. | MedU_hF4 | N33E/A145H/A213G/ | | | n/a | n/a |
| | | L220K/I243V/K250N/ | | | | |
| | | Y270H | | | | |
| 38. | MedU_hT2 | N33E/V195E/A204G/ | | | n/a | n/a |
| | | L220K/I243V | | | | |
| 39. | MedU_iT2 | N33E/A35D/F113H/ | | | n/a | n/a |
| | | A145T/V195D/L220K/ | | | | |
| | | I243V/K244S | | | | |
| 40. | MedU_jT1 | N33E/A83G/A145T/ | | | n/a | n/a |
| | | A148S/V195E/A213G/ | | | | |
| | | I243V/K244S | | | | |
| 41. | MedU_jT2 | A35D/A83G/F113H/ | | | n/a | n/a |
| | | A145H/V195E/L220H/ | | | | |
| | | I243V | | | | |
| 42. | MedU_jT4 | A145T/A148S/V195E/ | | | n/a | n/a |
| | | A213G/L220H/I243V/ | | | | |
| | | K244S | | | | |
| 43. | MedU_jT7 | A83G/A148S/V195E/ | | P265L | n/a | n/a |
| | | A204D/L220K/K244S/ | | | | |
| | | Y270H | | | | |
| 44. | MedU_jT9 | A35D/F82V/A145T/ | | | n/a | n/a |
| | | A148S/V195E/A204G/ | | | | |
| | | K244S | | | | |
| 45. | MedU_kF13 | A83G/A204D/A213G/ | | P248Q | n/a | n/a |
| | | L220K/I243V | | | | |
| 46. | MedU_kF14 | N33E/A35D/V195D/ | | | n/a | n/a |
| | | L220H/I243V/K244S/ | | | | |
| | | K250N/Y270H | | | | |
| 47. | MedU_kF17 | A83G/A145H/A204G/ | | | n/a | n/a |
| | | L220H/I243V/K250N/ | | | | |
| | | Y270H | | | | |
| 48. | MedU_kT1 | N33E/A35D/F82V/ | | | n/a | n/a |
| | | F113H/A145T/A148S/ | | | | |
| | | V195D | | | | |
| 49. | MedU_kT11 | A145H/Y203H/ | | | n/a | n/a |
| | | I243V/K244S | | | | |
| 50. | MedU_kT12 | A83G/A148S/V195E/ | | | n/a | n/a |
| | | A204G/L220H/I243V/ | | | | |
| | | K244S | | | | |
| 51. | MedU_kT6 | A145H/A204D/A213G/ | | | n/a | n/a |
| | | L220K/I243V | | | | |
| 52. | MedU_kT7 | A145H/A148S/V195D/ | | | n/a | n/a |
| | | A204D/L220H/K244S/ | | | | |
| | | K250N | | | | |
| 53. | MedU_kT9 | N33E/A35D/F113H/ | | | n/a | n/a |
| | | A148S/V195D/A204G/ | | | | |
| | | A213G/L220H/K244S | | | | |
| 54. | MedU_mF4 | F82V/A145T/V195E/ | N33D/L220Q | S46I | n/a | n/a |
| | | Y203H/A204G/Y270H | | | | |
| 55. | MedU_mT1 | A35D/A83G/V195D/ | | | n/a | n/a |
| | | A213G/L220H | | | | |
| 56. | MedU_mT2 | N33E/A35D/A83G/ | | | n/a | n/a |
| | | A145H/A148S/V195E/ | | | | |
| | | A213G/L220K/I243V/ | | | | |
| | | K244S | | | | |
| 57. | MedU_oF11 | N33E/A35D/F82V/ | | | n/a | n/a |
| | | A145T/A148S/A204G/ | | | | |
| | | L220H/I243V/K250N/ | | | | |
| | | Y270H | | | | |
| 58. | MedU_oF13 | A35D/A83G/F113H/ | | | n/a | n/a |
| | | A145H/A148S/V195E/ | | | | |
| | | A204G/L220H/I243V/ | | | | |
| | | Y270H | | | | |
| 59. | MedU_oT1 | A35D/A83G/A148S/ | | H63R | n/a | n/a |
| | | V195D/A204D/L220H | | | | |
| 60. | MedU_oT3 | A35D/A145T/A148S/ | | | n/a | n/a |
| | | Y203H/L220K | | | | |
| 61. | MedU_oT4 | A35D/F82V/A148S/ | | | n/a | n/a |
| | | A204G/I243V/K244S | | | | |
| 62. | MedU_oT5 | A83G/F113H/A145T/ | | E6K/S8N/E24Q/ | n/a | n/a |
| | | V195D/A204G/A213G/ | | T73P/S142F/ | | |
| | | Y270H | | L156V/S161F/ | | |
| | | | | T178P/R180C/ | | |
| | | | | L182V/S187F/ | | |
| | | | | A188G/L257M | | |
| 63. | MedU_pF8 | N33E/A145H/A148S/ | | | n/a | n/a |
| | | A204G/L220H/K250N | | | | |
| 64. | MedU_pF9 | N33E/A35D/A83G/ | | | n/a | n/a |
| | | F113H/A145T/A148S/ | | | | |
| | | V195E/A204G/I243V/ | | | | |
| | | K244S/K250N/Y270H | | | | |
| 65. | MedU_pT1 | N33E/A204D/L220H/ | | | n/a | n/a |
| | | K244S/K250N/Y270H | | | | |
| 66. | MedU_gT3 | N33E/F82V/A83G/ | | | n/a | n/a |
| | | A145T/A213G/L220K/ | | | | |
| | | Y270H | | | | |
| 67. | MedU_1A1 | N33E/A35D/F82V/ | | | 9.1 | −6.4 |
| | | A145H/V195D/A213G/ | | | | |
| | | I243V/K244S | | | | |
| 68. | MedU_1A10 | A35D/F113H/A145T/ | | | 48.8 | −3.3 |
| | | A148S/V195E/L220K/ | | | | |
| | | K244S/K250N/Y270H | | | | |
| 69. | MedU_1A11 | N33E/V195D/A204G/ | | | 14.3 | −10 |
| | | A213G/L220K/I243V/ | | | | |
| | | K244S | | | | |
| 70. | MedU_1A2 | F82V/A83G/A148S/ | A213V | | 2.8 | −11.6 |
| | | V195D/L220K/I243V/ | | | | |
| | | K250N | | | | |
| 71. | MedU_1A3 | A35D/F82V/A83G/ | | | 22.8 | −4.7 |
| | | A145T/V195D/L220K/ | | | | |
| | | I243V/K244S/K250N/ | | | | |
| | | Y270H | | | | |
| 72. | MedU_1A4 | F82V/A145T/V195E/ | | | 6.2 | −7.4 |
| | | A204G/L220H/I243V/ | | | | |
| | | Y270H | | | | |
| 73. | MedU_1A8 | N33E/A35D/A83G/ | | | 10.1 | −3.6 |
| | | V195E/L220H/K244S/ | | | | |
| | | K250N/Y270H | | | | |
| 74. | MedU_1A9 | N33E/A35D/F82V/ | | | 2.4 | −4.1 |
| | | F113H/A148S/A204G/ | | | | |
| | | L220K/K244S | | | | |
| 75. | MedU_1B3 | N33E/F113H/A145T/ | | | 1.8 | −3 |
| | | A148S/V195D/A204D/ | | | | |
| | | A213G/L220K/I243V/ | | | | |
| | | K244S/Y270H | | | | |
| 76. | MedU_1B4 | A35D/F82V/A83G/ | | | −0.5 | −0.5 |
| | | L220H/I243V/K250N | | | | |
| 77. | MedU_1B7 | A35D/A83G/A145T/ | | | 7.4 | −16.5 |
| | | A148S/A204G/A213G/ | | | | |
| | | I243V/K244S | | | | |
| 78. | MedU_1B8 | N33E/A35D/F113H/ | | | 0.9 | −4.6 |
| | | A145H/A148S/V195D/ | | | | |
| | | I243V/K250N/Y270H | | | | |
| 79. | MedU_1C12 | A35D/F82V/A83G/ | K244N | | 8.6 | −5.3 |
| | | A145T/A148S/V195D/ | | | | |
| | | A213G/L220K/I243V/ | | | | |
| | | Y270H | | | | |
| 80. | MedU_1C2 | A145T/V195E/I243V/ | | | 21.4 | −5.4 |
| | | K244S/K250N/Y270H | | | | |
| 81. | MedU_1C5 | N33E/A35D/A145H/ | | | 8.7 | −66.3 |
| | | A204G/A213G/L220H/ | | | | |
| | | Y270H | | | | |
| 82. | MedU_1C7 | F82V/A83G/V195D/ | | | 5.0 | −6.3 |
| | | K250N/Y270H | | | | |
| 83. | MedU_1C8 | F82V/A145H/A148S/ | | | −0.4 | −4.1 |
| | | A213G/L220H/I243V/ | | | | |
| | | K250N | | | | |
| 84. | MedU_1D12 | N33E/A35D/F82V/ | | | −2.9 | −3.7 |
| | | F113H/A204G/A213G | | | | |
| 85. | MedU_1D4 | N33E/F82V/A83G/ | | I150V | −1.2 | −3 |
| | | A145T/A148S/V195E/ | | | | |
| | | A204D/L220K/I243V/ | | | | |
| | | K250N | | | | |
| 86. | MedU_1D5 | N33E/F82V/A83G/ | | | 9.9 | −6.6 |
| | | V195E/A204G/A213G/ | | | | |
| | | Y270H | | | | |
| 87. | MedU_1D6 | A35D/A145T/A148S/ | | A271V/T282I | 3.2 | −3.2 |
| | | Y203H/A213G/I243V/ | | | | |
| | | K244S/Y270H | | | | |
| 88. | MedU_1D7 | F82V/A204D/ | | A110V | 0.4 | −1.7 |
| | | L220K/Y270H | | | | |
| 89. | MedU_1D8 | A83G/A148S/ | | S246G | 3.5 | −6.5 |
| | | I243V/Y270H | | | | |
| 90. | MedU_1E1 | A83G/A145T/A148S/ | | | 11.7 | −9.6 |
| | | A204G/I243V/K250N | | | | |
| 91. | MedU_1E10 | F113H/A145T/A148S/ | | | −1.2 | −4.6 |
| | | A204D/A213G/L220H/ | | | | |
| | | I243V/K244S/K250N/ | | | | |
| | | Y270H | | | | |
| 92. | MedU_1E12 | F82V/A83G/V195E/ | F113Y | | 12.5 | −14.8 |
| | | A204D/L220H | | | | |
| 93. | MedU_1E4 | N33E/F82V/A83G/ | | | −1.4 | −3.7 |
| | | F113H/A145T/A148S/ | | | | |
| | | V195E/A204D/L220H/ | | | | |
| | | I243V/K250N | | | | |
| 94. | MedU_1E7 | A35D/A83G/A145T/ | | R189H | 11.9 | −5.4 |
| | | A213G/I243V/K250N | | | | |
| 95. | MedU_1F1 | A35D/A83G/L89G/ | A204E | | 9.0 | −16.4 |
| | | V195E/Y203H/A213G/ | | | | |
| | | L220H/I243V/K244S/ | | | | |
| | | K250N | | | | |
| 96. | MedU_1F10 | N33E/A145T/A148S/ | | | 13.9 | −13.1 |
| | | V195D/A204D/A213G/ | | | | |
| | | K244S/K250N/Y270H | | | | |
| 97. | MedU_1F11 | A35D/F82V/A145T/ | | A66V | −1.0 | −4.1 |
| | | A213G/L220K/I243V | | | | |
| 98. | MedU_1F12 | A35D/F113H/A145T/ | | | 0.2 | −10.3 |
| | | V195E/A204D/A213G/ | | | | |
| | | L220K/K250N | | | | |
| 99. | MedU_1F2 | F82V/A145T/A148S/ | | | 8.2 | −5.8 |
| | | V195D/A213G/I243V/ | | | | |
| | | Y270H | | | | |
| 100. | MedU_1F3 | A35D/F82V/A83G/ | | | 14.8 | −3.1 |
| | | A148S/V195D/K244S/ | | | | |
| | | K250N/Y270H | | | | |
| 101. | MedU_1F4 | N33E/A35D/A83G/ | | | 0.9 | −4.1 |
| | | F113H/A145T/A148S/ | | | | |
| | | V195D/A204G/A213G/ | | | | |
| | | L220K | | | | |
| 102. | MedU_1F5 | A35D/F82V/V195D/ | | T178A | 7.3 | −3.6 |
| | | A213G/I243V/K244S | | | | |
| 103. | MedU_1F6 | N33E/A145T/Y203H/ | | | −1.0 | 1.5 |
| | | A204G/I243V/K250N | | | | |
| 104. | MedU_1F7 | N33E/A35D/F113H/ | | | 3.2 | −2.9 |
| | | V195E/L220H/K244S/ | | | | |
| | | K250N | | | | |
| 105. | MedU_1F8 | A83G/A148S/V195D/ | | | 9.4 | −7.4 |
| | | A204D/A213G/I243V/ | | | | |
| | | K250N | | | | |
| 106. | MedU_1F9 | F82V/F113H/A145T/ | | | 14.3 | −3.4 |
| | | A148S/A204G/L220K/ | | | | |
| | | I243V/K244S | | | | |
| 107. | MedU_1G11 | N33E/A83G/A148S/ | | | 2.6 | −6.7 |
| | | V195D/A204D/L220K/ | | | | |
| | | I243V/K244S/Y270H | | | | |
| 108. | MedU_1G2 | N33E/A35D/A148S/ | | | −1.6 | −3.7 |
| | | Y203H/A204G/I243V/ | | | | |
| | | K244S | | | | |
| 109. | MedU_1G3 | A145H/A148S/V195E/ | | | 9.3 | −15.1 |
| | | A213G/I243V/K244S/ | | | | |
| | | K250N | | | | |
| 110. | MedU_1G5 | N33E/A83G/A145T/ | | V28A | −2.9 | 9.7 |
| | | A213G/I243V/K244S | | | | |
| 111. | MedU_1G7 | F82V/F113H/A145H/ | | E98K | 2.2 | −3.6 |
| | | A148S/A213G | | | | |
| 112. | MedU_1H4 | N33E/A83G/F113H/ | | | −1.8 | 0.2 |
| | | L220H/I243V/K244S/ | | | | |
| | | K250N | | | | |
| 113. | MedU_1H6 | N33E/A35D/F113H/ | | | 2.3 | 4 |
| | | A145T/A148S/V195D/ | | | | |
| | | A204D/L220K/I243V/ | | | | |
| | | K244S/Y270H | | | | |
| 114. | MedU_1H7 | F113H/A145T/A148S/ | | | −4.3 | −2.5 |
| | | V195E/Y203H/L220K | | | | |
| 115. | MedU_1H8 | N33E/V195E/A204G/ | | | 6.4 | −7.5 |
| | | L220H | | | | |
| 116. | MedU_1H9 | F82V/A83G/A148S/ | | | 7.7 | −3.8 |
| | | V195E/A204G/A213G/ | | | | |
| | | K244S/K250N/Y270H | | | | |
| 117. | MedU_5A1 | N33E/A145H/V195D/ | | A290T | 38.7 | −6.5 |
| | | A204D/I243V/K250N/ | | | | |
| | | Y270H | | | | |
| 118. | MedU_5A10 | N33E/A35D/F82V/ | | | 0.3 | 0.4 |
| | | A83G/A145T/A204D/ | | | | |
| | | A213G/L220H/K250N | | | | |
| 119. | MedU_5A11 | N33E/A83G/A145T/ | | | 27.9 | −6.1 |
| | | A148S/V195E/L220H/ | | | | |
| | | K250N/Y270H | | | | |
| 120. | MedU_5A12 | A35D/F113H/A145T/ | | H63Y | −0.2 | 6.2 |
| | | A148S/V195E/A204G/ | | | | |
| | | L220K/I243V/K244S/ | | | | |
| | | K250N | | | | |
| 121. | MedU_5A2 | N33E/A35D/A83G/ | | | 17.5 | −4.5 |
| | | F113H/A145H/L220K/ | | | | |
| | | I243V/K244S/Y270H | | | | |
| 122. | MedU_5A4 | A83G/F113H/A145T/ | | | 6.0 | −7.9 |
| | | A204G/Y270H | | | | |
| 123. | MedU_5A5 | N33E/A35D/A145H/ | | | 14.1 | −11.9 |
| | | A148S/V195E/A204D/ | | | | |
| | | L220H/I243V | | | | |
| 124. | MedU_5A6 | A35D/F82V/A83G/ | | | 0.5 | −21.6 |
| | | A145H/A148S/V195E/ | | | | |
| | | A213G/L220K | | | | |
| 125. | MedU_5A7 | N33E/A83G/F113H/ | | | −16.1 | −0.9 |
| | | A145T/A148S/V195D/ | | | | |
| | | A204G/L220K/I243V | | | | |
| 126. | MedU_5A8 | N33E/A83G/V195E/ | A145V | | 13.0 | −6.5 |
| | | L220K/K250N/Y270H | | | | |
| 127. | MedU_5A9 | A35D/A83G/F113H/ | | | 13.9 | −5 |
| | | V195E/A204G/L220K/ | | | | |
| | | I243V/K244S | | | | |
| 128. | MedU_5B1 | N33E/F113H/A145H/ | | | 15.2 | −4.2 |
| | | A148S/V195E/A204D/ | | | | |
| | | A213G/I243V | | | | |
| 129. | MedU_5B10 | V195E/A204G/I243V/ | | | 15.0 | −7.3 |
| | | K244S | | | | |
| 130. | MedU_5B12 | N33E/A83G/V195D/ | | | 8.0 | −16.5 |
| | | A213G/L220K | | | | |
| 131. | MedU_5B2 | N33E/F113H/A145T/ | | | 6.2 | −3.2 |
| | | I243V/K250N | | | | |
| 132. | MedU_5B4 | A35D/A83G/A145H/ | | | 2.3 | −7.6 |
| | | A213G/L220K/K244S/ | | | | |
| | | K250N/Y270H | | | | |
| 133. | MedU_5B6 | F113H/A145T/V195D/ | | H237Y | 0.4 | −5.6 |
| | | A204G/A213G | | | | |
| 134. | MedU_5B8 | A35D/A83G/A145T/ | | | 2.5 | −12.7 |
| | | A148S/V195E/A204D/ | | | | |
| | | A213G/L220H/I243V/ | | | | |
| | | K244S | | | | |
| 135. | MedU_5B9 | A35D/F82V/F113H/ | | | 8.1 | −3.6 |
| | | A145H/V195E/A213G/ | | | | |
| | | I243V/K244S | | | | |
| 136. | MedU_5C10 | A35D/F82V/A83G/ | | | 2.5 | −10.9 |
| | | A148S/A213G/L220H/ | | | | |
| | | K244S | | | | |
| 137. | MedU_5C11 | A83G/V195D/A204D/ | | | 6.2 | −6.6 |
| | | L220K/I243V/K250N | | | | |
| 138. | MedU_5C12 | A83G/F113H/V195D/ | | | 3.0 | −4 |
| | | A204D/L220H/I243V | | | | |
| 139. | MedU_5C2 | N33E/F82V/A83G/ | | | 6.1 | −9.9 |
| | | A145H/A148S/V195E/ | | | | |
| | | A204D/A213G/K244S/ | | | | |
| | | Y270H | | | | |
| 140. | MedU_5C3 | N33E/A83G/A145H/ | | | −3.0 | 3.5 |
| | | V195E/Y203H/L220H | | | | |
| 141. | MedU_5C4 | A83G/A145H/A148S/ | | | 7.1 | −8.4 |
| | | V195E/A213G/L220K/ | | | | |
| | | K250N | | | | |
| 142. | MedU_5C5 | N33E/F82V/A145H/ | | | 4.4 | −4.8 |
| | | A148S/A204D/I243V/ | | | | |
| | | K250N/Y270H | | | | |
| 143. | MedU_5C6 | N33E/A35D/V195D/ | | | −79.1 | −3.3 |
| | | A204D/L220K/I243V/ | | | | |
| | | K250N/Y270H | | | | |
| 144. | MedU_5C9 | A35D/A145H/A148S/ | | | 11.5 | −7.6 |
| | | V195D/A204D/A213G/ | | | | |
| | | K244S/K250N | | | | |
| 145. | MedU_5D11 | F113H/A145H/A204G/ | | G16S | −0.3 | −3.3 |
| | | I243V/K250N/Y270H | | | | |
| 146. | MedU_5D12 | A145H/A148S/V195D/ | | A290V | 27.7 | −5.9 |
| | | A204D/L220K/I243V/ | | | | |
| | | K244S/K250N | | | | |
| 147. | MedU_5D2 | N33E/A83G/A148S/ | | R231C | 0.0 | 1.1 |
| | | V195D/L220H/I243V/ | | | | |
| | | K244S | | | | |
| 148. | MedU_5D3 | F113H/A145T/A148S/ | | | 2.2 | −3.3 |
| | | A204D/A213G/I243V/ | | | | |
| | | K250N/Y270H | | | | |
| 149. | MedU_5D4 | A83G/L220K/K244S/ | | | 13.3 | −3.4 |
| | | K250N/Y270H | | | | |
| 150. | MedU_5D6 | N33E/A35D/F113H/ | | | −65.3 | −2.2 |
| | | A145H/A213G/K244S | | | | |
| 151. | MedU_5D7 | N33E/A35D/F82V/ | | | 2.7 | 3.5 |
| | | A83G/F113H/V195D/ | | | | |
| | | A204D/L220K | | | | |
| 152. | MedU_5D8 | F113H/A145T/A148S/ | | E53K | −2.2 | −6.2 |
| | | V195D/A204G/ | | | | |
| | | L220K/I243V/K250N | | | | |
| 153. | MedU_5D9 | F82V/A83G/F113H/ | | | 3.3 | −4.2 |
| | | A145H/V195E/A213G | | | | |
| 154. | MedU_5E10 | A35D/F113H/A145T/ | | | 0.9 | −6 |
| | | V195E/A204D/L220H/ | | | | |
| | | I243V/K244S/Y270H | | | | |
| 155. | MedU_5E11 | F82V/A83G/A148S/ | | | 0.0 | 0.4 |
| | | V195D/A204G/ | | | | |
| | | L220H/I243V/Y270H | | | | |
| 156. | MedU_5E12 | N33E/A35D/A83G/ | I243M | E6K | 0.8 | 6.2 |
| | | V195E/Y203H/L220K | | | | |
| 157. | MedU_5E2 | N33E/A35D/A83G/ | | | 23.6 | −3.3 |
| | | F113H/A145H/A148S/ | | | | |
| | | I243V/K244S | | | | |
| 158. | MedU_5E3 | N33E/F82V/A83G/ | | | 3.7 | −2.6 |
| | | F113H/A145T/V195E/ | | | | |
| | | A204G/L220K/I243V | | | | |
| 159. | MedU_5E4 | N33E/A145H/A148S/ | | | 6.0 | −2.6 |
| | | V195D/A213G/L220H/ | | | | |
| | | I243V/K244S/K250N | | | | |
| 160. | MedU_5E5 | A35D/A83G/A145T/ | | R231C | 0.5 | −2.7 |
| | | A148S/V195E/ | | | | |
| | | A204G/I243V/K244S/ | | | | |
| | | K250N/Y270H | | | | |
| 161. | MedU_5E6 | A35D/A83G/F113H/ | | | 3.6 | −2.3 |
| | | A145T/A204D/ | | | | |
| | | A213G/L220K/ | | | | |
| | | I243V/K244S | | | | |
| 162. | MedU_5E7 | A35D/F82V/A83G/ | | | −1.5 | −1.7 |
| | | F113H/V195D/ | | | | |
| | | A213G/K244S | | | | |
| 163. | MedU_5E8 | N33E/A83G/A148S/ | | | 4.9 | −12 |
| | | A204G/L220H/K244S | | | | |
| 164. | MedU_5E9 | N33E/A35D/A83G/ | | | 7.9 | −10 |
| | | A145T/A148S/ | | | | |
| | | V195D/A204D/A213G/ | | | | |
| | | L220K/K244S/Y270H | | | | |
| 165. | MedU_5F1 | N33E/A35D/F82V/ | | S49Y | 1.8 | −3.2 |
| | | A145H/V195E/A204D/ | | | | |
| | | L220K/K244S/K250N | | | | |
| 166. | MedU_5F10 | F113H/A145H/A213G/ | | | 4.3 | −5.5 |
| | | K250N | | | | |
| 167. | MedU_5F11 | N33E/A35D/A83G/ | | | 5.2 | −3.5 |
| | | A145T/A148S/ | | | | |
| | | A204D/A213G/L220K/ | | | | |
| | | K244S/K250N | | | | |
| 168. | MedU_5F2 | N33E/A35D/Y203H/ | | | −1.4 | −3.4 |
| | | A204G/L220K/Y270H | | | | |
| 169. | MedU_5F3 | A35D/A83G/A148S/ | | A183V/A188V | 1.2 | −3.6 |
| | | A204D/L220K/I243V | | | | |
| 170. | MedU_5F4 | A83G/A145T/A148S/ | | | 5.1 | −9.8 |
| | | V195D/A204G/ | | | | |
| | | K244S/K250N | | | | |
| 171. | MedU_5F5 | N33E/A35D/F82V/ | | | −0.2 | −0.4 |
| | | A83G/F113H/A145T/ | | | | |
| | | A148S/V195E/ | | | | |
| | | A204D/I243V/Y270H | | | | |
| 172. | MedU_5F6 | A83G/F113H/A145T/ | | | 12.1 | −8.1 |
| | | A148S/V195D/ | | | | |
| | | A204G/L220K | | | | |
| 173. | MedU_5F7 | A35D/F82V/A83G/ | | | 2.7 | 0.3 |
| | | F113H/A145T/L220H/ | | | | |
| | | K244S | | | | |
| 174. | MedU_5F8 | N33E/A35D/A83G/ | | | −1.5 | −2.9 |
| | | F113H/A145H/V195E/ | | | | |
| | | A204G/L220H/Y270H | | | | |
| 175. | MedU_5F9 | A148S/V195D/A204D/ | | | −1.9 | −1.3 |
| | | L220H/K244S/ | | | | |
| | | K250N/Y270H | | | | |
| 176. | MedU_5G1 | A83G/F113H/A145T/ | A213V | | 1.8 | −2.6 |
| | | V195D/A204D/ | | | | |
| | | I243V/K250N/Y270H | | | | |
| 177. | MedU_5G10 | N33E/V195E/L220K/ | | | 14.7 | −6.3 |
| | | I243V/K244S | | | | |
| 178. | MedU_5G11 | F82V/A145H/A148S/ | | | 2.2 | −2.9 |
| | | V195D/A204D/ | | | | |
| | | L220H/K250N/Y270H | | | | |
| 179. | MedU_5G12 | N33E/F82V/A83G/ | A148G/I243M/ | | 1.6 | −8.2 |
| | | V195E/Y203H/A213G/ | K244N | | | |
| | | L220K/Y270H | | | | |
| 180. | MedU_5G2 | A83G/A145T/V195D/ | | | 1.7 | 1.3 |
| | | A204D/A213G/ | | | | |
| | | L220K/K244S/K250N | | | | |
| 181. | MedU_5G3 | N33E/A35D/A83G/ | | | 9.3 | −6.4 |
| | | A145T/V195E/A204D/ | | | | |
| | | I243V/K244S/Y270H | | | | |
| 182. | MedU_5G4 | A35D/A83G/F113H/ | | | 8.6 | −3.2 |
| | | A148S/A213G/I243V | | | | |
| 183. | MedU_5G5 | F113H/A145T/A204G/ | | A76T | 1.8 | −3.1 |
| | | L220K/I243V/K244S | | | | |
| 184. | MedU_5G6 | N33E/A35D/A83G/ | K244N | | −0.7 | 0.9 |
| | | F113H/A148S/V195D/ | | | | |
| | | A204G/I243V/Y270H | | | | |
| 185. | MedU_5G7 | N33E/A35D/A83G/ | | | 1.8 | −3.7 |
| | | A148S/Y203H/L220K/ | | | | |
| | | K244S | | | | |
| 186. | MedU_5G8 | A83G/F113H/A145H/ | | | −3.0 | −3.4 |
| | | A148S/V195E/A204D/ | | | | |
| | | L220H/I243V/K250N | | | | |
| 187. | MedU_5G9 | N33E/F82V/A83G/ | | | 0.9 | 0.1 |
| | | V195E/A204G/L220K/ | | | | |
| | | K244S/K250N/Y270H | | | | |
| 188. | MedU_5H1 | N33E/A145H/A148S/ | | | −0.3 | −3.1 |
| | | V195D/A204G/ | | | | |
| | | A213G/L220H/I243V/ | | | | |
| | | K250N/Y270H | | | | |
| 189. | MedU_5H10 | A35D/A83G/F113H/ | | E98K | 1.0 | −0.9 |
| | | A145H/A148S/V195D/ | | | | |
| | | L220H/I243V/K250N | | | | |
| 190. | MedU_5H3 | N33E/A83G/F113H/ | K244N | | −1.2 | −2.9 |
| | | A148S/A204G/ | | | | |
| | | L220H/K250N | | | | |
| 191. | MedU_5H4 | N33E/A35D/A204D/ | | A272T | 5.8 | −12.3 |
| | | I243V/K250N/Y270H | | | | |
| 192. | MedU_5H5 | N33E/A35D/F82V/ | | | −1.4 | −4 |
| | | F113H/A145H/A213G/ | | | | |
| | | I243V/Y270H | | | | |
| 193. | MedU_5H6 | A35D/A213G/L220H/ | | | −1.4 | 4.7 |
| | | I243V/K244S/ | | | | |
| | | K250N/Y270H | | | | |
| 194. | MedU_5H7 | A83G/A145T/A148S/ | | A272V | 4.3 | −11.7 |
| | | A204D/A213G/ | | | | |
| | | L220K/I243V/K250N | | | | |
| 195. | MedU_5H8 | A35D/F82V/A83G/ | | | 1.0 | −6 |
| | | A145T/V195D/A213G/ | | | | |
| | | I243V/K250N/Y270H | | | | |
| 196. | MedU_5H9 | A35D/A83G/F113H/ | | | 3.0 | −5 |
| | | A145T/A148S/ | | | | |
| | | A204D/A213G/L220K/ | | | | |
| | | I243V/K250N/Y270H | | | | |

1.3. Further Characterization of Select Arthrobacter globiformis Uricase Variants

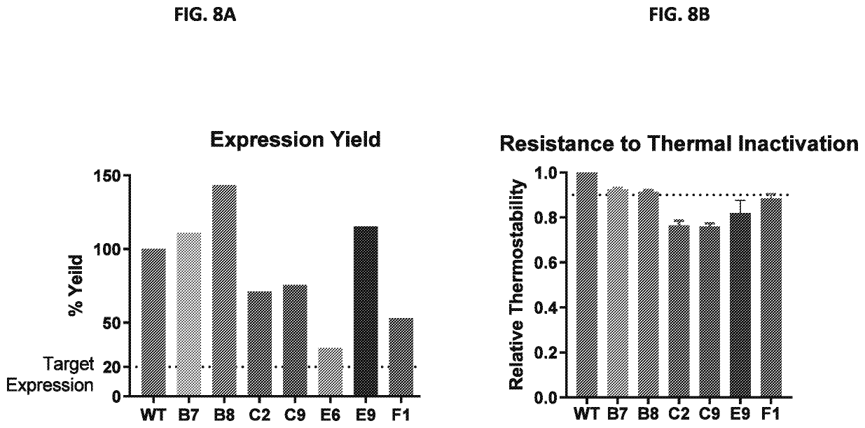

Select Arthrobacter globiformis uricase variants MedU_1B7, MedU_5B8, MedU_1C2, MedU_5C9, MedU_5E6, MedU_5E9, and MedU_IF1 were subject to further characterization by comparing their expression yields, Michaelis-Menten kinetics parameters, and thermostability measured by temperatures at which 50% of normal activity was lost determined by heat stress activity assay, with those of the wild type Arthrobacter globiformis uricase. The expression yields were determined as described above in the “Methods” section. The Michaelis-Menten kinetics parameters k_{cat} and K_{M}, and temperatures at which 50% of normal activity was lost (i.e., 50% thermal inactivation temperatures, referred to as TI50 (° C.)) measured by heat stress activity assay were likewise determined using the methods described in Example 1. The SEQ ID NOs and amino acid sequences of the select variants are shown in Table 8A. As shown in FIG. 8A, all of the variants exceeded the target yield of 20% of the wild type yield, with variants MedU_1B7 (abbreviated as B7 in the figure), MedU_5B8 (abbreviated as B8 in the figure) and MedU_5E9 (abbreviated as E9 in the figure) exhibiting even higher yields than the wild type Arthrobacter globiformis uricase (WT). Michaelis-Menten kinetic analysis data shown in Table 8B reveals that all of the variants exhibited higher uricase activity than the wild type enzyme measured by k_{cat}. FIG. 8B and Table 8C show that all the variants retained stability above 37° C.

TABLE 8A
SEQ ID NOs and amino acid sequences of select Arthrobacter globiformis
uricase variants

| | SEQ ID | |
|---|---|---|
| Uricase Enzyme | NO | Amino acid sequence |
| MedU_1B7 | 77 | MTATAETSTGTKVVLGQNQYGKAEVRLVKVTRNTDRHEIQD |
| | | LNVTSQLSGDFEAAHTAGDNAHVVATDTQKNTVYAFARDGF |
| | | GTTEEFLLRLGKHFTEGFDWVTGGRWAAQQFFWDRINDHDH |
| | | AFSRNKSEVRTAVLEISGSEQTIVSGIEGLTVLKSTGSEFHGFPR |
| | | DKYTTLQETTDRILATDVSARWRYNTVEVDFDAVYGSVRGL |
| | | LLKGFAETHSLALQQTMYEMGRAVIETHPEIDEVSMSLPNKH |
| | | HFLVDLQPFGQDNPNEVFYAADRPYGLIEATIQREGSRAD |
| MedU_5B8 | 134 | MTATAETSTGTKVVLGQNQYGKAEVRLVKVTRNTDRHEIQD |
| | | LNVTSQLSGDFEAAHTAGDNAHVVATDTQKNTVYAFARDGF |
| | | GTTEEFLLRLGKHFTEGFDWVTGGRWAAQQFFWDRINDHDH |
| | | AFSRNKSEVRTAVLEISGSEQTIVSGIEGLTVLKSTGSEFHGFPR |
| | | DKYTTLQETTDRILATDVSARWRYNTEEVDFDAVYDSVRGLL |
| | | LKGFAETHSHALQQTMYEMGRAVIETHPEIDEVSMSLPNKHH |
| | | FLVDLQPFGQDNPNEVFYAADRPYGLIEATIQREGSRAD |
| MedU_1C2 | 80 | MTATAETSTGTKVVLGQNQYGKAEVRLVKVTRNTARHEIQD |
| | | LNVTSQLSGDFEAAHTAGDNAHVVATDTQKNTVYAFARDGF |
| | | ATTEEFLLRLGKHFTEGFDWVTGGRWAAQQFFWDRINDHDH |
| | | AFSRNKSEVRTAVLEISGSEQTIVAGIEGLTVLKSTGSEFHGFP |
| | | RDKYTTLQETTDRILATDVSARWRYNTEEVDFDAVYASVRG |
| | | LLLKAFAETHSLALQQTMYEMGRAVIETHPEIDEVSMSLPNN |
| | | HHFLVDLQPFGQDNPNEVFHAADRPYGLIEATIQREGSRAD |
| MedU_5C9 | 144 | MTATAETSTGTKVVLGQNQYGKAEVRLVKVTRNTDRHEIQD |
| | | LNVTSQLSGDFEAAHTAGDNAHVVATDTQKNTVYAFARDGF |
| | | ATTEEFLLRLGKHFTEGFDWVTGGRWAAQQFFWDRINDHDH |
| | | AFSRNKSEVRTAVLEISGSEQHIVSGIEGLTVLKSTGSEFHGFP |
| | | RDKYTTLQETTDRILATDVSARWRYNTDEVDFDAVYDSVRG |
| | | LLLKGFAETHSLALQQTMYEMGRAVIETHPEIDEISMSLPNNH |
| | | HFLVDLQPFGQDNPNEVFYAADRPYGLIEATIQREGSRAD |
| MedU_5E6 | 161 | MTATAETSTGTKVVLGQNQYGKAEVRLVKVTRNTDRHEIQD |
| | | LNVTSQLSGDFEAAHTAGDNAHVVATDTQKNTVYAFARDGF |
| | | GTTEEFLLRLGKHFTEGFDWVTGGRWAAQQHFWDRINDHDH |
| | | AFSRNKSEVRTAVLEISGSEQTIVAGIEGLTVLKSTGSEFHGFP |
| | | RDKYTTLQETTDRILATDVSARWRYNTVEVDFDAVYDSVRG |
| | | LLLKGFAETHSKALQQTMYEMGRAVIETHPEIDEVSMSLPNK |
| | | HHFLVDLQPFGQDNPNEVFYAADRPYGLIEATIQREGSRAD |
| MedU_5E9 | 164 | MTATAETSTGTKVVLGQNQYGKAEVRLVKVTRETDRHEIQD |
| | | LNVTSQLSGDFEAAHTAGDNAHVVATDTQKNTVYAFARDGF |
| | | GTTEEFLLRLGKHFTEGFDWVTGGRWAAQQFFWDRINDHDH |
| | | AFSRNKSEVRTAVLEISGSEQTIVSGIEGLTVLKSTGSEFHGFPR |
| | | DKYTTLQETTDRILATDVSARWRYNTDEVDFDAVYDSVRGL |
| | | LLKGFAETHSKALQQTMYEMGRAVIETHPEIDEISMSLPNKHH |
| | | FLVDLQPFGQDNPNEVFHAADRPYGLIEATIQREGSRAD |
| MedU_1F1 | 95 | MTATAETSTGTKVVLGQNQYGKAEVRLVKVTRNTDRHEIQD |
| | | LNVTSQLSGDFEAAHTAGDNAHVVATDTQKNTVYAFARDGF |
| | | GTTEEFGLRLGKHFTEGFDWVTGGRWAAQQFFWDRINDHDH |
| | | AFSRNKSEVRTAVLEISGSEQAIVAGIEGLTVLKSTGSEFHGFP |
| | | RDKYTTLQETTDRILATDVSARWRYNTEEVDFDAVHESVRGL |
| | | LLKGFAETHSHALQQTMYEMGRAVIETHPEIDEVSMSLPNNH |
| | | HFLVDLQPFGQDNPNEVFYAADRPYGLIEATIQREGSRAD |

TABLE 8B
Kinetic parameters of wild type Arthrobacter
globiformis uricase (WT) and select variants

| Uricase Enzyme | k_{cat} (s⁻¹) | K_{M} (μM) |
|---|---|---|
| MedU_5E9 | 6.0 ± 0.4 | 800 ± 100 |
| MedU_5B8 | 3.9 ± 0.2 | 390 ± 80 |
| MedU_1B7 | 3.8 ± 0.2 | 440 ± 90 |
| MedU_1C2 | 3.4 ± 0.2 | 500 ± 90 |
| MedU_1F1 | 2.5 ± 0.1 | 370 ± 60 |
| MedU_5C9 | 2.4 ± 0.1 | 340 ± 80 |
| WT | 2.3 ± 0.2 | 270 ± 80 |

TABLE 8C
Thermal inactivation property of wild type Arthrobacter globiformis
uricase (WT) and select variants measured by TI50 (° C.)

| Uricase Enzyme | TI50 (° C.) |
|---|---|
| WT | 58 ± 1.0 |
| MedU_1B7 | 53 ± 0.9 |
| MedU_5B8 | 53 ± 0.9 |
| MedU_1F1 | 51 ± 0.9 |
| MedU_5E9 | 47 ± 0.8 |
| MedU_1C2 | 44 ± 0.8 |
| MedU_5C9 | 44 ± 0.8 |

2. Creation and Characterization of Non-PEGylated Deimmunized Aspergillus flavus Uricase Variants

The monomeric form of the wild type Aspergillus flavus uricase comprises the amino acid sequence of SEQ ID NO:7, where serine is residue #1. The specified mutations in the Aspergillus flavus uricase variants of this example are indexed to serine as residue #1, as in SEQ ID NO:7. However, in this example, the wild type Aspergillus flavus uricase and its variants expressed in E. coli possessed a methionine (preceding the serine) as residue #1, with the serine as residue #2, due to the presence of an Ndel restriction site encoding methionine in-frame with the wild type and the variant Aspergillus flavus uricase genes in the expression vector pET-26b(+). The expression vector pET-26b(+) encodes a His tag containing 6 histidine residues at the C-terminus of the inserted uricase gene.

(wild type Aspergillus flavus uricase)
SEQ ID NO: 7
SAVKAARYGK DNVRVYKVHK DEKTGVQTVY EMTVCVLLEG EIETSYTKAD NSVIVATDSI
KNTIYITAKQ NPVTPPELFG SILGTHFIEK YNHIHAAHVN IVCHRWTRMD IDGKPHPHSF
IRDSEEKRNV QVDVVEGKGI DIKSSLSGLT VLKSTNSQFW GFLRDEYTTL KETWDRILST
DVDATWQWKN FSGLQEVRSH VPKFDATWAT AREVTLKTFA EDNSASVQAT MYKMAEQILA
RQQLIETVEY SLPNKHYFEI DLSWHKGLQN TGKNAEVFAP QSDPNGLIKC TVGRSSLKSK
L

### 2.1. Deimmunized Library Design

Deimmunized library design was carried out as described in Example 1, with the selection of a 19-site library with 524,288 members. The library members were predicted by the design algorithms to have reduced MHC-II binding potential as compared to the wild type Aspergillus flavus uricase. The variants of this library design had the designed amino acids summarized in Table 9. The amino acid positions are indexed to serine as residue #1, as in SEQ ID NO:7.

TABLE 9
Designed amino acids in Aspergillus flavus uricase variants
according to the selected deimmunized library design

| Target | Wild Type (WT) | Variant Amino |
|---|---|---|
| Site | Amino Acid | Acid |
| 33 | T | TV |
| 41 | E | EN |
| 53 | V | NV |
| 56 | A | AT |
| 65 | Y | HY |
| 79 | F | FW |
| 82 | I | EI |
| 85 | T | DT |
| 96 | A | AK |
| 98 | H | HN |
| 100 | N | DN |
| 147 | S | DS |
| 148 | G | DG |
| 160 | W | EW |
| 209 | A | AN |
| 210 | T | KT |
| 229 | A | AN |
| 235 | A | AG |
| 240 | A | AD |

2.2. Identification and Activity of Functional Aspergillus flavus Uricase Variants

Upon selection of the library design described above, functional Aspergillus flavus uricase variants were identified via (i) a high-throughput halo-based assay to select active variants, and (ii) purification and detailed analysis of the activity of the variants. The details of the halo-based assay and the activity assay are described above in the “Methods” section.

Table 10 shows 70 functional Aspergillus flavus uricase variants identified. With the genes of the variants sequenced, Table 10 shows each variant's SEQ ID NO and mutations relative to the wild type Aspergillus flavus uricase of SEQ ID NO:7, as well as the activity of each uricase variant. Besides the mutations according to the library design, i.e., designed mutations, some of the variants comprised “off-amino acid mutation(s),” i.e., non-designed amino acid(s) at target site(s), and/or “off-position amino acid mutation(s),” i.e., non-designed amino acid(s) at non-target site(s).

TABLE 10
Exemplary functional Aspergillus flavus uricase variants

| SEQ ID NO | | | | | |
|---|---|---|---|---|---|
| for variant | | | Off amino | Off position | |
| Aspergillus flavus | | | acid | amino acid | |
| uricase | Code Name | Designed mutations | mutations | mutations | Activity |
| 197. | RasU_A1 | T33V/E41N/A96K/ | | | −79.9 |
| | | G148D/A209N/ | | | |
| | | T210K/A229N | | | |
| 198. | RasU_A10 | E41N/F79W/G148D/ | | | −81.7 |
| | | A209N/A229N/ | | | |
| | | A235G/A240D | | | |
| 199. | RasU_A11 | E41N/F79W/T85D/ | | | −66.4 |
| | | A96K/G148D/ | | | |
| | | T210K/A240D | | | |
| 200. | RasU_A12 | E41N/V53N/I82E/ | | | −71.7 |
| | | A96K/N100D/G148D/ | | | |
| | | A229N/A240D | | | |
| 201. | RasU_A2 | E41N/F79W/A96K/ | | | −57.1 |
| | | N100D/S147D/ | | | |
| | | T210K/A240D | | | |
| 202. | RasU_A4 | V53N/F79W/A96K/ | | M231I | −60.7 |
| | | H98N/G148D/ | | | |
| | | W160E/A229N/A240D | | | |
| 203. | RasU_A5 | T33V/E41N/V53N/ | | | −67 |
| | | A96K/H98N/N100D/ | | | |
| | | G148D/T210K/ | | | |
| | | A229N/A240D | | | |
| 204. | RasU_A6 | V53N/T85D/H98N/ | | | −79.8 |
| | | N100D/S147D/ | | | |
| | | G148D/A229N/A240D | | | |
| 205. | RasU_A7 | E41N/Y65H/A96K/ | W160L | | −14 |
| | | H98N/N100D/A240D | | | |
| 206. | RasU_A8 | Y65H/F79W/T85D/ | | | −65.8 |
| | | A96K/N100D/G148D | | | |
| 207. | RasU_B10 | V53N/Y65H/N100D/ | | | −43.8 |
| | | G148D/A209N/T210K | | | |
| 208. | RasU_B11 | E41N/V53N/F79W/ | | | −71.3 |
| | | N100D/S147D | | | |
| 209. | RasU_B2 | H98N/N100D/G148D/ | | | −60.2 |
| | | W160E/A229N/A240D | | | |
| 210. | RasU_B3 | A96K/S147D/W160E/ | | S192R | −51.7 |
| | | A235G | | | |
| 211. | RasU_B4 | E41N/Y65H/A96K/ | | | −48.9 |
| | | H98N/N100D/T210K/ | | | |
| | | A229N/A235G/A240D | | | |
| 212. | RasU_B5 | T85D/A96K/S147D/ | | | −85.3 |
| | | G148D/A209N/A235G | | | |
| 213. | RasU_B6 | E41N/A56T/I82E/ | | | −49.9 |
| | | A96K/N100D/G148D/ | | | |
| | | A229N/A240D | | | |
| 214. | RasU_B8 | T33V/T85D/A96K/ | | | −57.7 |
| | | N100D/A240D | | | |
| 215. | RasU_B9 | T33V/A96K/S147D/ | | | −81.5 |
| | | T210K/A229N/A240D | | | |
| 216. | RasU_C10 | E41N/V53N/T85D/ | | | −66.7 |
| | | G148D/A209N/A235G | | | |
| 217. | RasU_C11 | I82E/A96K/N100D/ | | | −69.55 |
| | | A209N/T210K | | | |
| 218. | RasU_C12 | T33V/V53N/N100D/ | | | −76.9 |
| | | S147D/A229N | | | |
| 219. | RasU_C3 | V53N/F79W/A96K/ | | | −32.9 |
| | | H98N/N100D/G148D/ | | | |
| | | W160E/A209N/A229N | | | |
| 220. | RasU_C4 | E41N/A56T/Y65H/ | | | −81.2 |
| | | A96K/N100D/A229N | | | |
| 221. | RasU_C5 | E41N/V53N/S147D/ | | | −50.7 |
| | | G148D/W160E/ | | | |
| | | A209N/A229N/A240D | | | |
| 222. | RasU_C6 | T33V/E41N/F79W/ | | | −6.7 |
| | | A96K/G148D/T210K/ | | | |
| | | A235G/A240D | | | |
| 223. | RasU_C7 | T33V/F79W/A96K/ | | | −58.5 |
| | | H98N/N100D/S147D/ | | | |
| | | A209N/A229N/A240D | | | |
| 224. | RasU_C8 | V53N/F79W/A96K/ | | | −80.8 |
| | | N100D/G148D/A209N/ | | | |
| | | A229N/A240D | | | |
| 225. | RasU_C9 | E41N/V53N/A96K/ | | | −46.7 |
| | | S147D/W160E/A240D | | | |
| 226. | RasU_D1 | F79W/A96K/H98N/ | | | −46.6 |
| | | N100D/S147D/W160E/ | | | |
| | | A209N/A229N/A240D | | | |
| 227. | RasU_D10 | V53N/T85D/A96K/ | | | −91.8 |
| | | S147D/G148D/A209N | | | |
| 228. | RasU_D11 | A56T/F79W/A96K/ | | | −91.6 |
| | | H98N/S147D/G148D/ | | | |
| | | A209N/A229N/A240D | | | |
| 229. | RasU_D12 | E41N/I82E/A96K/ | | | −85.7 |
| | | N100D/S147D/T210K/ | | | |
| | | A229N/A240D | | | |
| 230. | RasU_D2 | V53N/Y65H/A96K/ | | | −71.4 |
| | | H98N/N100D/S147D/ | | | |
| | | A229N | | | |
| 231. | RasU_D3 | T33V/V53N/I82E/ | | | −44.1 |
| | | A96K/N100D/A209N/ | | | |
| | | A229N | | | |
| 232. | RasU_D4 | T33V/V53N/T85D/ | | | −6.2 |
| | | A96K/S147D/T210K/ | | | |
| | | A229N/A235G | | | |
| 233. | RasU_D5 | H98N/G148D/T210K/ | | | −82.7 |
| | | A229N | | | |
| 234. | RasU_D6 | F79W/T85D/T210K/ | | | −83.4 |
| | | A229N/A235G | | | |
| 235. | RasU_D7 | E41N/F79W/A96K/ | | | −80.1 |
| | | N100D/S147D/G148D/ | | | |
| | | A209N/A229N | | | |
| 236. | RasU_D8 | E41N/V53N/I82E/ | | | −81.5 |
| | | A96K/N100D/S147D/ | | | |
| | | A229N/A240D | | | |
| 237. | RasU_D9 | Y65H/F79W/A96K/ | | | −84.2 |
| | | N100D/G148D/ | | | |
| | | A229N/A235G/A240D | | | |
| 238. | RasU_E1 | E41N/T85D/A96K/ | | | −62.4 |
| | | G148D/W160E/A209N/ | | | |
| | | A229N/A240D | | | |
| 239. | RasU_E10 | E41N/V53N/F79W/ | | | −71.8 |
| | | A96K/G148D/A235G | | | |
| 240. | RasU_E11 | T33V/E41N/V53N/ | | | −3.5 |
| | | T85D/A96K/H98N/ | | | |
| | | N100D/G148D/T210K/ | | | |
| | | A229N | | | |
| 241. | RasU_E12 | E41N/A56T/A96K/ | | | −83.3 |
| | | S147D/G148D/A229N/ | | | |
| | | A235G/A240D | | | |
| 242. | RasU_E2 | E41N/A56T/Y65H/ | | | −3 |
| | | I82E/A96K/A209N/ | | | |
| | | T210K/A229N/A240D | | | |
| 243. | RasU_E3 | T85D/H98N/N100D/ | | | −11 |
| | | G148D/T210K/A235G/ | | | |
| | | A240D | | | |
| 244. | RasU_E4 | V53N/A56T/T85D/ | | | −48.5 |
| | | A96K/N100D/G148D/ | | | |
| | | A209N/A229N/A235G | | | |
| 245. | RasU_E5 | A56T/F79W/N100D/ | E41D | W174C | −3.8 |
| | | S147D/W160E/T210K/ | | | |
| | | A235G | | | |
| 246. | RasU_E6 | E41N/F79W/T85D/ | | A184V | −7.5 |
| | | A96K/W160E/T210K/ | | | |
| | | A229N/A240D | | | |
| 247. | RasU_E8 | A56T/Y65H/H98N/ | | | −30.3 |
| | | G148D/W160E/T210K/ | | | |
| | | A229N | | | |
| 248. | RasU_E9 | T33V/Y65H/A96K/ | | | −5.9 |
| | | G148D/W160E/A240D | | | |
| 249. | RasU_F1 | E41N/V53N/A56T/ | | | −74.3 |
| | | N100D/G148D/A209N/ | | | |
| | | T210K/A229N/A240D | | | |
| 250. | RasU_F10 | E41N/Y65H/A96K/ | | | −28.3 |
| | | G148D/W160E/A209N/ | | | |
| | | A229N/A235G | | | |
| 251. | RasU_F11 | I82E/S147D/W160E/ | | | −26.9 |
| | | A209N/T210K | | | |
| 252. | RasU_F12 | A56T/Y65H/F79W/ | | | −13 |
| | | T85D/A96K/N100D/ | | | |
| | | S147D/A209N/A229N | | | |
| 253. | RasU_F2 | E41N/F79W/I82E/ | | | −33.5 |
| | | H98N/T210K/A229N/ | | | |
| | | A235G | | | |
| 254. | RasU_F3 | E41N/A56T/F79W/ | | | −51.9 |
| | | I82E/T85D/A96K/ | | | |
| | | A229N | | | |
| 255. | RasU_F4 | V53N/Y65H/N100D/ | | | −13.4 |
| | | A209N/A235G/A240D | | | |
| 256. | RasU_F5 | E41N/V53N/A96K/ | | | −42.2 |
| | | H98N/N100D/W160E/ | | | |
| | | T210K/A229N/A240D | | | |
| 257. | RasU_F6 | T33V/F79W/A96K/ | | | −61.4 |
| | | H98N/N100D/S147D/ | | | |
| | | A229N/A240D | | | |
| 258. | RasU_F7 | E41N/V53N/F79W/ | | | −22 |
| | | A96K/N100D/S147D/ | | | |
| | | W160E/A209N/A229N | | | |
| 259. | RasU_F8 | V53N/A56T/G148D/ | | | −42.3 |
| | | W160E/A209N/A229N | | | |
| 260. | RasU_F9 | T33V/V53N/A96K/ | | | −30.9 |
| | | N100D/S147D/A209N/ | | | |
| | | T210K/A229N/A235G/ | | | |
| | | A240D | | | |
| 261. | RasU_G1 | E41N/Y65H/A96K/ | | | −67.9 |
| | | N100D/A209N | | | |
| 262. | RasU_G2 | I82E/A96K/N100D/ | | | −118.8 |
| | | S147D/G148D/A240D | | | |
| 263. | RasU_G3 | E41N/A56T/Y65H/ | T33M | | −73.7 |
| | | F79W/T85D/A229N/ | | | |
| | | A235G | | | |
| 264. | RasU_G4 | F79W/H98N/N100D/ | | | −58.7 |
| | | G148D/W160E/A209N/ | | | |
| | | A240D | | | |
| 265. | RasU_G5 | Y65H/A96K/N100D/ | | | −46.8 |
| | | W160E/A229N/A240D | | | |
| 266. | RasU_G6 | Y65H/T85D/N100D/ | | | −103.7 |
| | | S147D/A229N/A240D | | | |

2.3. Further Characterization of Select Aspergillus flavus Uricase Variants

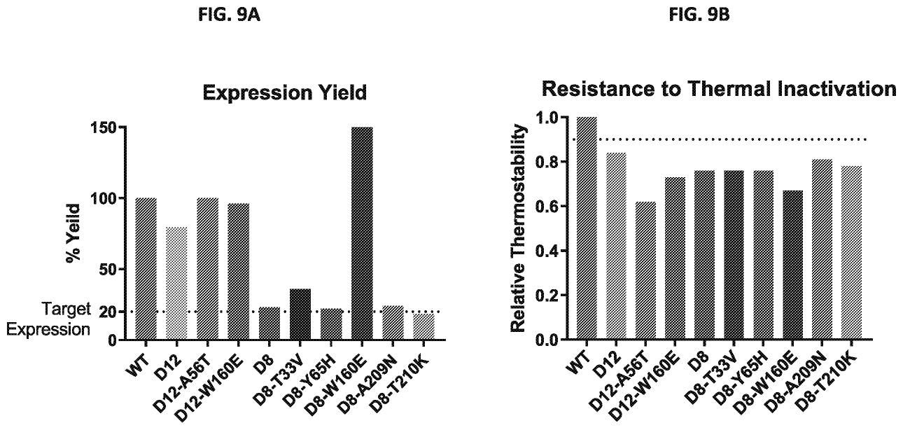

Select Aspergillus flavus uricase variants RasU_D12, as well as its subvariants RasU_D12-A56T, and RasU_D12-W160E; and RasU_D8, as well as its subvariants RasU_D8-T33V, RasU_D8-Y65H, RasU_D8-W160E, RasU_D8-A209N, and RasU_D8-T210K were subject to further characterization by comparing their expression yields, Michaelis-Menten kinetics parameters, and thermostability measured by temperatures at which 50% of normal activity was lost determined by heat stress activity assay, with those of the wild type Aspergillus flavus uricase. As the names of the subvariants indicated, the subvariants of RasU_D12 were constructed by introducing an additional amino acid substitution A56T or W160E into the RasU_D12 variant, while the subvariants of RasU_D8 were constructed by introducing an additional amino acid substitution T33V, Y65H, W160E, A209N, or T210K into the RasU_D8 variant. Each of the additional amino acid substitutions was to address residual T cell epitopes in the RasU_D8 or RasU_D12 variant. The expression yields were determined as described above in the “Methods” section. The determination of the Michaelis-Menten kinetics parameters k_{cat} and K_{M}, and the heat stress activity assay were likewise performed using the methods described in Example 1. The SEQ ID NOs, mutations to the wild type Aspergillus flavus uricase of SEQ ID NO:7, and amino acid sequences of the select variants are shown in Table 11A. As shown in FIG. 9A, all of the variants except RasU_D8-T210K exceeded the target yield of 20% of the wild type yield. The expression yield of RasU_D8-T210K was only slightly below 20% of the wild type yield. Importantly, compared to the wild type Aspergillus flavus uricase (WT), variants RasU_D12-A56T and RasU_D12-W160E each had a similar yield and variant RasU_D8-W160E had an even higher yield. Michaelis-Menten kinetic analysis data shown in Table 11B reveals that overall, the variants exhibited WT-like uricase activity measured by k_{cat}. FIG. 9B shows that all the variants also possessed WT-like thermostability.

TABLE 11A
SEQ ID NOs, mutations to wild type Aspergillus flavus uricase of SEQ ID NO: 7, and
amino acid sequences of select Aspergillus flavus uricase variants

| | | Mutations to wild | |
|---|---|---|---|
| | SEQ | type Aspergillus flavus | |
| | ID | uricase of | |
| Uricase Enzyme | NO | SEQ ID NO: 7 | Amino acid sequence |
| RasU_D12 | 229 | E41N/I82E/A96K/N100D/ | SAVKAARYGKDNVRVYKVHKDEKTG |
| | | S147D/T210K/A229N/ | VQTVYEMTVCVLLEGNIETSYTKADNS |
| | | A240D | VIVATDSIKNTIYITAKQNPVTPPELFGS |
| | | | ELGTHFIEKYNHIHKAHVDIVCHRWTR |
| | | | MDIDGKPHPHSFIRDSEEKRNVQVDVV |
| | | | EGKGIDIKSSLDGLTVLKSTNSQFWGFL |
| | | | RDEYTTLKETWDRILSTDVDATWQWK |
| | | | NFSGLQEVRSHVPKFDATWAKAREVT |
| | | | LKTFAEDNSASVQNTMYKMAEQILDR |
| | | | QQLIETVEYSLPNKHYFEIDLSWHKGL |
| | | | QNTGKNAEVFAPQSDPNGLIKCTVGRS |
| | | | SLKSKL |
| RasU_D12-A56T | 267 | E41N/A56T/I82E/A96K/ | SAVKAARYGKDNVRVYKVHKDEKTG |
| | | N100D/S147D/T210K/ | VQTVYEMTVCVLLEGNIETSYTKADNS |
| | | A229N/A240D | VIVTTDSIKNTIYITAKQNPVTPPELFGS |
| | | | ELGTHFIEKYNHIHKAHVDIVCHRWTR |
| | | | MDIDGKPHPHSFIRDSEEKRNVQVDVV |
| | | | EGKGIDIKSSLDGLTVLKSTNSQFWGFL |
| | | | RDEYTTLKETWDRILSTDVDATWQWK |
| | | | NFSGLQEVRSHVPKFDATWAKAREVT |
| | | | LKTFAEDNSASVQNTMYKMAEQILDR |
| | | | QQLIETVEYSLPNKHYFEIDLSWHKGL |
| | | | QNTGKNAEVFAPQSDPNGLIKCTVGRS |
| | | | SLKSKL |
| RasU_D12-W160E | 268 | E41N/I82E/A96K/N100D/ | SAVKAARYGKDNVRVYKVHKDEKTG |
| | | S147D/W160E/T210K/ | VQTVYEMTVCVLLEGNIETSYTKADNS |
| | | A229N/A240D | VIVATDSIKNTIYITAKQNPVTPPELFGS |
| | | | ELGTHFIEKYNHIHKAHVDIVCHRWTR |
| | | | MDIDGKPHPHSFIRDSEEKRNVQVDVV |
| | | | EGKGIDIKSSLDGLTVLKSTNSQFEGFL |
| | | | RDEYTTLKETWDRILSTDVDATWQWK |
| | | | NFSGLQEVRSHVPKFDATWAKAREVT |
| | | | LKTFAEDNSASVQNTMYKMAEQILDR |
| | | | QQLIETVEYSLPNKHYFEIDLSWHKGL |
| | | | QNTGKNAEVFAPQSDPNGLIKCTVGRS |
| | | | SLKSKL |
| RasU D8 | 236 | E41N/V53N/I82E/A96K/ | SAVKAARYGKDNVRVYKVHKDEKTG |
| | | N100D/S147D/A229N/ | VQTVYEMTVCVLLEGNIETSYTKADNS |
| | | A240D | NIVATDSIKNTIYITAKQNPVTPPELFGS |
| | | | ELGTHFIEKYNHIHKAHVDIVCHRWTR |
| | | | MDIDGKPHPHSFIRDSEEKRNVQVDVV |
| | | | EGKGIDIKSSLDGLTVLKSTNSQFWGFL |
| | | | RDEYTTLKETWDRILSTDVDATWQWK |
| | | | NFSGLQEVRSHVPKFDATWATAREVT |
| | | | LKTFAEDNSASVQNTMYKMAEQILDR |
| | | | QQLIETVEYSLPNKHYFEIDLSWHKGL |
| | | | QNTGKNAEVFAPQSDPNGLIKCTVGRS |
| | | | SLKSKL |
| RasU_D8-T33V | 269 | T33V/E41N/V53N/I82E/ | SAVKAARYGKDNVRVYKVHKDEKTG |
| | | A96K/N100D/S147D/ | VQTVYEMVVCVLLEGNIETSYTKADN |
| | | A229N/A240D | SNIVATDSIKNTIYITAKQNPVTPPELFG |
| | | | SELGTHFIEKYNHIHKAHVDIVCHRWT |
| | | | RMDIDGKPHPHSFIRDSEEKRNVQVDV |
| | | | VEGKGIDIKSSLDGLTVLKSTNSQFWG |
| | | | FLRDEYTTLKETWDRILSTDVDATWQ |
| | | | WKNFSGLQEVRSHVPKFDATWATARE |
| | | | VTLKTFAEDNSASVQNTMYKMAEQIL |
| | | | DRQQLIETVEYSLPNKHYFEIDLSWHK |
| | | | GLQNTGKNAEVFAPQSDPNGLIKCTVG |
| | | | RSSLKSKL |
| RasU_D8-Y65H | 270 | E41N/V53N/Y65H/I82E/ | SAVKAARYGKDNVRVYKVHKDEKTG |
| | | A96K/N100D/S147D/ | VQTVYEMTVCVLLEGNIETSYTKADNS |
| | | A229N/A240D | NIVATDSIKNTIHITAKQNPVTPPELFGS |
| | | | ELGTHFIEKYNHIHKAHVDIVCHRWTR |
| | | | MDIDGKPHPHSFIRDSEEKRNVQVDVV |
| | | | EGKGIDIKSSLDGLTVLKSTNSQFWGFL |
| | | | RDEYTTLKETWDRILSTDVDATWQWK |
| | | | NFSGLQEVRSHVPKFDATWATAREVT |
| | | | LKTFAEDNSASVQNTMYKMAEQILDR |
| | | | QQLIETVEYSLPNKHYFEIDLSWHKGL |
| | | | QNTGKNAEVFAPQSDPNGLIKCTVGRS |
| | | | SLKSKL |
| RasU_D8-W160E | 271 | E41N/V53N/I82E/A96K/ | SAVKAARYGKDNVRVYKVHKDEKTG |
| | | N100D/S147D/W160E/ | VQTVYEMTVCVLLEGNIETSYTKADNS |
| | | A229N/A240D | NIVATDSIKNTIYITAKQNPVTPPELFGS |
| | | | ELGTHFIEKYNHIHKAHVDIVCHRWTR |
| | | | MDIDGKPHPHSFIRDSEEKRNVQVDVV |
| | | | EGKGIDIKSSLDGLTVLKSTNSQFEGFL |
| | | | RDEYTTLKETWDRILSTDVDATWQWK |
| | | | NFSGLQEVRSHVPKFDATWATAREVT |
| | | | LKTFAEDNSASVQNTMYKMAEQILDR |
| | | | QQLIETVEYSLPNKHYFEIDLSWHKGL |
| | | | QNTGKNAEVFAPQSDPNGLIKCTVGRS |
| | | | SLKSKL |
| RasU_D8-A209N | 272 | E41N/V53N/I82E/A96K/ | SAVKAARYGKDNVRVYKVHKDEKTG |
| | | N100D/S147D/A209N/ | VQTVYEMTVCVLLEGNIETSYTKADNS |
| | | A229N/A240D | NIVATDSIKNTIYITAKQNPVTPPELFGS |
| | | | ELGTHFIEKYNHIHKAHVDIVCHRWTR |
| | | | MDIDGKPHPHSFIRDSEEKRNVQVDVV |
| | | | EGKGIDIKSSLDGLTVLKSTNSQFWGFL |
| | | | RDEYTTLKETWDRILSTDVDATWQWK |
| | | | NFSGLQEVRSHVPKFDATWNTAREVT |
| | | | LKTFAEDNSASVQNTMYKMAEQILDR |
| | | | QQLIETVEYSLPNKHYFEIDLSWHKGL |
| | | | QNTGKNAEVFAPQSDPNGLIKCTVGRS |
| | | | SLKSKL |
| RasU_D8-T210K | 273 | E41N/V53N/I82E/A96K/ | SAVKAARYGKDNVRVYKVHKDEKTG |
| | | N100D/S147D/T210K/ | VQTVYEMTVCVLLEGNIETSYTKADNS |
| | | A229N/A240D | NIVATDSIKNTIYITAKQNPVTPPELFGS |
| | | | ELGTHFIEKYNHIHKAHVDIVCHRWTR |
| | | | MDIDGKPHPHSFIRDSEEKRNVQVDVV |
| | | | EGKGIDIKSSLDGLTVLKSTNSQFWGFL |
| | | | RDEYTTLKETWDRILSTDVDATWQWK |
| | | | NFSGLQEVRSHVPKFDATWAKAREVT |
| | | | LKTFAEDNSASVQNTMYKMAEQILDR |
| | | | QQLIETVEYSLPNKHYFEIDLSWHKGL |
| | | | QNTGKNAEVFAPQSDPNGLIKCTVGRS |
| | | | SLKSKL |

TABLE 11B
Kinetic parameters of wild type Aspergillus
flavus uricase (WT) and select variants

| Uricase Enzyme | k_{cat} (s⁻¹) | K_{M} (μM) |
|---|---|---|
| WT | 1.23 ± 0.04 | 290 ± 30 |
| RasU_D12 | 1.3 ± 0.1 | 400 ± 100 |
| RasU_D12-A56T | 1.4 ± 0.3 | 1500 ± 600 |
| RasU_D12-W160E | 1.7 ± 0.5 | 1700 ± 1000 |
| RasU_D8 | 0.8 ± 0.1 | 900 ± 200 |
| RasU_D8-T33V | 1.6 ± 0.5 | 3000 ± 1000 |
| RasU_D8-Y65H | 1.7 ± 0.5 | 3000 ± 1000 |
| RasU_D8-W160E | 1.2 ± 0.2 | 600 ± 200 |
| RasU_D8-A209N | 1.1 ± 0.1 | 1100 ± 300 |
| RasU_D8-T210K | 2.0 ± 0.7 | 3000 ± 2000 |

3. Creation and Characterization of Non-PEGylated Deimmunized Candida utilis Uricase Variants

The monomeric form of the wild type Candida utilis uricase comprises the amino acid sequence of SEQ ID NO:8, where methionine is residue #1. The specified mutations in the Candida utilis uricase variants of this example are indexed to methionine as residue #1, as in SEQ ID NO:8. In this example, the wild type Candida utilis uricase and each of its variants were expressed in E. coli transformed with the expression vector pET-26b(+) containing the cDNA sequence encoding the wild type Candida utilis uricase or its variant. The expression vector pET-26b(+) encodes a His tag containing 6 histidine residues at the C-terminus of the inserted uricase gene.

(wild type Candida utilis uricase)
SEQ ID NO: 8
MSTTLSSSTY GKDNVKFLKV KKDPQNPKKQ EVMEATVTCL LEGGFDTSYT EADNSSIVPT
DTVKNTILVL AKTTEIWPIE RFAAKLATHF VEKYSHVSGV SVKIVQDRWV KYAVDGKPHD
HSFIHEGGEK RITDLYYKRS GDYKLSSAIK DLTVLKSTGS MFYGYNKCDF TTLQPTTDRI
LSTDVDATWV WDNKKIGTVY DIAKAADKGI FDNVYNQARE ITLTTFALEN SPSVQATMFN
MATQILEKAC SVYSVSYALP NKHYFLIDLK WKGLENDNEL FYPSPHPNGL IKCTVVRKEK
TKL

### 3.1. Deimmunized Library Design

Deimmunized library design was carried out as described in Example 1, with the selection of an 18-site library with 884,736 members. The library members were predicted by the design algorithms to have reduced MHC-II binding potential as compared to the wild type Candida utilis uricase. The variants of this library design had the designed amino acids summarized in Table 12. The amino acid positions are indexed to methionine as residue #1, as in SEQ ID NO:8.

TABLE 12
Designed amino acids in Candida utilis uricase variants
according to the selected deimmunized library design

| Target | Wild Type (WT) | Variant Amino |
|---|---|---|
| Site | Amino Acid | Acid |
| 70 | L | ELT |
| 82 | F | FWY |
| 83 | A | AG |
| 87 | A | AG |
| 95 | S | DS |
| 98 | S | DS |
| 144 | K | DK |
| 146 | S | ES |
| 148 | A | AK |
| 161 | M | EM |
| 221 | I | IT |
| 224 | T | KT |
| 228 | L | ELN |
| 236 | A | AN |
| 242 | A | AG |
| 253 | Y | DY |
| 258 | A | AE |
| 266 | L | EL |

3.2. Identification, Activity and Expression Levels of Functional Candida utilis Uricase Variants

Upon selection of the library design described above, functional Candida utilis uricase variants were identified via (i) a high-throughput halo-based assay to select active variants, and (ii) purification and detailed analysis of the activity and expression levels of the variants as compared to the wild type Candida utilis uricase. The details of the halo-based assay and the activity and expression level assays are described above in the “Methods” section.

Table 13 shows 68 functional Candida utilis uricase variants identified. With the genes of the variants sequenced, Table 13 shows each variant's SEQ ID NO and mutations relative to the wild type Candida utilis uricase of SEQ ID NO:8, as well as the activity and expression level of each variant. Besides the mutations according to the library design, i.e., designed mutations, some of the variants comprised “off-amino acid mutation(s),” i.e., non-designed amino acid(s) at target site(s), and/or “off-position amino acid mutation(s),” i.e., non-designed amino acid(s) at non-target site(s). Data for the wild type Candida utilis uricase are also included in Table 13 for comparison.

TABLE 13
Exemplary functional Candida utilis uricase variants

| SEQ ID NO | | | | | | |
|---|---|---|---|---|---|---|
| for wild type | WT or Code | | | | | |
| (WT) or | Name for | | | | | |
| variant | variant | | | | | |
| Candida | Candida | | Off amino | Off position | | |
| utilis | utilis | Designed | acid | amino acid | | |
| uricase | uricase | mutations | mutations | mutations | Expression | Activity |
| 8 | WT | N/A | N/A | N/A | 3.1 | −97 |
| 274. | SelU_A1 | F82Y/S98D/M161E/ | | | 0.9 | −94.4 |
| | | L228E/A236N | | | | |
| 275. | SelU_A10 | F82Y/A87G/S95D/ | K144N | | 0.4 | −36.6 |
| | | S98D/A148K/M161E/ | | | | |
| | | L266E | | | | |
| 276. | SelU_A11 | K144D/M161E/I221T/ | | | 2.8 | −96.3 |
| | | T224K/A236N/A242G | | | | |
| 277. | SelU_A12 | S95D/A148K/M161E/ | A83V | | 2.7 | −103.5 |
| | | T224K/A236N/A242G | | | | |
| 278. | SelU_A3 | L70E/A148K/A236N/ | | | 1.7 | −67.6 |
| | | A258E | | | | |
| 279. | SelU_A4 | A87G/S95D/A148K/ | A236T | K195R | 2.5 | −64.3 |
| | | M161E/L228E/A242G/ | | | | |
| | | Y253D/A258E | | | | |
| 280. | SelU_A6 | L70T/S95D/K144D/ | | G127S | 2.3 | −48.4 |
| | | A148K/M161E | | | | |
| 281. | SelU_A7 | F82Y/S95D/S146E/ | | | 1.8 | −89.6 |
| | | M161E/A236N/A258E | | | | |
| 282. | SelU_A8 | L70T/F82Y/S95D/ | | | 4.6 | −75.5 |
| | | T224K/L228E/A236N | | | | |
| 283. | SelU_A9 | L70T/A87G/T224K/ | | | 2.6 | −91.4 |
| | | L228E/A236N/A258E | | | | |
| 284. | SelU_B1 | A83G/A258E/L266E | | V97I | 1.2 | −93.9 |
| 285. | SelU_B10 | S95D/S146E/A148K/ | | | 3.7 | −51.1 |
| | | M161E/I221T/T224K/ | | | | |
| | | L228N/A236N/ | | | | |
| | | A258E/L266E | | | | |
| 286. | SelU_B11 | A83G/A148K/M161E/ | | D178G | 5.9 | −35.9 |
| | | L228N/A236N/ | | | | |
| | | A242G/Y253D | | | | |
| 287. | SelU_B4 | F82Y/K144D/L228E/ | | T237S | 1.4 | −82.8 |
| | | A258E/L266E | | | | |
| 288. | SelU_B7 | L70T/F82Y/S95D/ | | G44S | 4.1 | −42.8 |
| | | A148K/M161E/L228N/ | | | | |
| | | A236N/L266E | | | | |
| 289. | SelU_B8 | A87G/S95D/K144D/ | | | 2.9 | 9.1 |
| | | M161E/T224K/ | | | | |
| | | L228N/L266E | | | | |
| 290. | SelU_B9 | L70T/S95D/A148K/ | T224N/L228K | | 3.0 | −55 |
| | | M161E/A236N/Y253D/ | | | | |
| | | A258E/L266E | | | | |
| 291. | SelU_C1 | L70T/S95D/S98D/ | | | 2.3 | −58.8 |
| | | I221T/T224K/L228N/ | | | | |
| | | A242G/Y253D | | | | |
| 292. | SelU_C11 | F82Y/S98D/K144D/ | L228K | | 4.5 | −48.8 |
| | | M161E/Y253D/L266E | | | | |
| 293. | SelU_C12 | L70T/S95D/S98D/ | | | 3.7 | −52.6 |
| | | A148K/M161E/T224K/ | | | | |
| | | A236N/A242G/ | | | | |
| | | A258E/L266E | | | | |
| 294. | SelU_C2 | F82Y/A83G/S98D/ | | | 0.7 | −48.2 |
| | | L228N/A242G/L266E | | | | |
| 295. | SelU_C3 | F82Y/S98D/A148K/ | | T222I | 2.4 | −44.3 |
| | | M161E/T224K/L228N | | | | |
| 296. | SelU_C4 | A83G/S95D/S98D/ | | | 1.0 | −44.4 |
| | | S146E/M161E/L228N/ | | | | |
| | | A236N/A242G/L266E | | | | |
| 297. | SelU_C5 | F82W/S95D/T224K/ | | P232T | 1.4 | −81.5 |
| | | Y253D/A258E | | | | |
| 298. | SelU_C6 | L70T/S98D/T224K/ | F82C/K144N | | 3.9 | −86.4 |
| | | A236N/A242G/L266E | | | | |
| 299. | SelU_C8 | L70E/S95D/S98D/ | | | 4.8 | −63.9 |
| | | S146E/L228E/A236N | | | | |
| 300. | SelU_C9 | F82Y/A83G/A87G/ | A236K | G43D | 0.3 | −35.2 |
| | | S95D/A148K/L228E/ | | | | |
| | | A242G/L266E | | | | |
| 301. | SelU_D1 | F82Y/S95D/K144D/ | | | 0.1 | 32.5 |
| | | A148K/M161E/L228E/ | | | | |
| | | Y253D/A258E/L266E | | | | |
| 302. | SelU_D10 | A87G/S95D/S98D/ | | S140C | 1.1 | −58 |
| | | K144D/A148K/T224K/ | | | | |
| | | L228N/A236N/ | | | | |
| | | A258E/L266E | | | | |
| 303. | SelU_D11 | A87G/A148K/M161E/ | L70Q/F82C/ | | 1.3 | −69 |
| | | A242G/Y253D | S98N/S146K/ | | | |
| | | | T224N/L228K | | | |
| 304. | SelU_D12 | L70T/S95D/S98D/ | | P24Q/V63I/ | 3.5 | −70.4 |
| | | L266E | | S231G | | |
| 305. | SelU_D3 | F82Y/A87G/S98D/ | L70M | | 1.3 | −51.4 |
| | | A148K/L228N/A242G/ | | | | |
| | | A258E | | | | |
| 306. | SelU_D5 | A83G/S95D/S98D/ | | Y163C | 2.6 | −53.5 |
| | | A148K/I221T/T224K/ | | | | |
| | | A236N/A258E/L266E | | | | |
| 307. | SelU_D6 | S95D/S146E/L228N/ | | | 1.7 | −74.5 |
| | | A236N/A242G/Y253D/ | | | | |
| | | A258E/L266E | | | | |
| 308. | SelU_D7 | A87G/S95D/S98D/ | | | 4.1 | −54.4 |
| | | L228E/A236N/A242G/ | | | | |
| | | A258E/L266E | | | | |
| 309. | SelU_D8 | L70T/F82Y/S95D/ | | | 3.5 | −61.6 |
| | | S98D/A148K/A242G | | | | |
| 310. | SelU_D9 | F82Y/S95D/S98D/ | | V114I | 4.4 | −53.7 |
| | | S146E/A148K/I221T/ | | | | |
| | | T224K/L228E/A258E/ | | | | |
| | | L266E | | | | |
| 311. | SelU_E11 | S98D/S146E/A148K/ | | | 3.9 | −68.4 |
| | | L228E/A236N/A242G | | | | |
| 312. | SelU_E2 | F82Y/A83G/S95D/ | | | 1.8 | −46.5 |
| | | S98D/M161E/L228N | | | | |
| 313. | SelU_E4 | F82Y/K144D/M161E/ | | | 2.0 | −68.2 |
| | | L228N/A242G/L266E | | | | |
| 314. | SelU_E5 | A87G/A148K/M161E/ | S98N | | 1.3 | −46.9 |
| | | I221T/Y253D/A258E/ | | | | |
| | | L266E | | | | |
| 315. | SelU_E6 | L228E/A236N/A258E | | R81C/T177I | 4.3 | −58.6 |
| 316. | SelU_E7 | F82Y/S95D/A148K/ | | | 8.4 | −39.7 |
| | | M161E/I221T/L228E/ | | | | |
| | | A236N/A242G/ | | | | |
| | | Y253D/A258E | | | | |
| 317. | SelU_E9 | A87G/S95D/A148K/ | | | 3.1 | −50.4 |
| | | M161E/L266E | | | | |
| 318. | SelU_F1 | S95D/A148K/M161E/ | | T73P | 1.3 | −85.3 |
| | | L228E/A236N/ | | | | |
| | | A258E/L266E | | | | |
| 319. | SelU_F10 | F82Y/S95D/A148K/ | | | 4.1 | −62.4 |
| | | L228N/A236N/Y253D | | | | |
| 320. | SelU_F11 | L70E/A87G/S98D/ | | | 0.5 | −53.3 |
| | | I221T/L228N/A236N/ | | | | |
| | | A258E | | | | |
| 321. | SelU_F12 | F82W/A148K/A236N | | | 0.2 | −34.4 |
| 322. | SelU_F3 | L70T/F82Y/S98D/ | T224N/A236E/ | A218V | 2.1 | −67.4 |
| | | M161E/I221T/L228E/ | L266V | | | |
| | | A242G/Y253D/A258E | | | | |
| 323. | SelU_F4 | F82Y/A87G/S95D/ | | | 0.2 | −57.2 |
| | | A148K/I221T/L228E/ | | | | |
| | | A242G | | | | |
| 324. | SelU_F6 | S95D/A148K/I221T/ | | V234I | 7.9 | −33.4 |
| | | T224K/L228N/A242G/ | | | | |
| | | Y253D | | | | |
| 325. | SelU_F7 | A87G/S95D/S98D/ | | | 2.9 | −85 |
| | | A148K/M161E/L228E/ | | | | |
| | | A236N/A258E | | | | |
| 326. | SelU_F8 | F82Y/S95D/S98D/ | | | 4.1 | −62.9 |
| | | A148K/M161E/L228N/ | | | | |
| | | A258E | | | | |
| 327. | SelU_G1 | L70T/F82Y/A87G/ | | G128S | 0.9 | −73 |
| | | S98D/A148K/L228E/ | | | | |
| | | A242G/A258E | | | | |
| 328. | SelU_G11 | L70T/F82Y/S98D/ | | | 0.3 | −35.6 |
| | | A148K/M161E/I221T/ | | | | |
| | | L228E/A236N/ | | | | |
| | | A242G/A258E | | | | |
| 329. | SelU_G12 | L70E/A83G/S95D/ | | G159D | 0.4 | −16.2 |
| | | L228N/A236N | | | | |
| 330. | SelU_G3 | F82Y/S95D/A148K/ | | | 1.7 | −77.1 |
| | | A242G/Y253D/L266E | | | | |
| 331. | SelU_G5 | L70T/F82Y/S95D/ | | | 1.5 | −49.9 |
| | | S98D/S146E/A148K/ | | | | |
| | | M161E/T224K/ | | | | |
| | | Y253D/A258E/L266E | | | | |
| 332. | SelU_G7 | L70E/S146E/A236N/ | | E51D | 0.3 | −63.2 |
| | | L266E | | | | |
| 333. | SelU_G8 | L70T/F82Y/S95D/ | | | 6.0 | −33.1 |
| | | S98D/A148K/M161E/ | | | | |
| | | L228E/A242G/L266E | | | | |
| 334. | SelU_H10 | S98D/S146E/I221T/ | | | 1.7 | −115.3 |
| | | T224K/L228E/A242G/ | | | | |
| | | A258E/L266E | | | | |
| 335. | SelU_H3 | S98D/A148K/M161E/ | | G209C | 1.3 | −59.1 |
| | | L228E/A236N/L266E | | | | |
| 336. | SelU_H4 | F82Y/A83G/S95D/ | | | 0.3 | −49.7 |
| | | S98D/S146E/A148K/ | | | | |
| | | L228E/A236N/A258E/ | | | | |
| | | L266E | | | | |
| 337. | SelU_H5 | L70T/F82Y/S95D/ | | | 0.8 | −42.1 |
| | | A148K/M161E/T224K/ | | | | |
| | | A236N/A242G/L266E | | | | |
| 338. | SelU_H6 | F82Y/S98D/K144D/ | | V63L | −4.5 | −26.3 |
| | | S146E/A148K/L228N/ | | | | |
| | | A236N/A258E/ | | | | |
| | | L266E | | | | |
| 339. | SelU_H7 | L70T/S98D/A148K/ | | | 1.6 | −103.6 |
| | | T224K/A236N/L266E | | | | |
| 340. | SelU_G4 | S95D/A148K/T224K/ | | | 1.9 | −78.3 |
| | | L228N/A242G/Y253D/ | | | | |
| | | L266E | | | | |
| 341. | SelU_H8 | S95D/S98D/S146E/ | | | 1.8 | −105 |
| | | A148K/M161E/I221T/ | | | | |
| | | L228N/Y253D/A258E | | | | |

3.3. Further Characterization of Select Candida utilis Uricase Variants

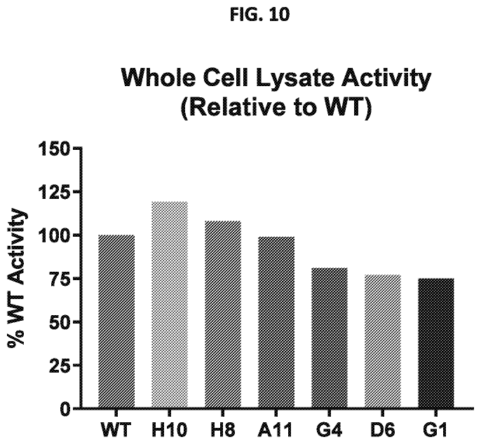

Select Candida utilis uricase variants SelU_A11, SelU_D6, SelU_G1, SelU_G4, SelU_H8, and SelU_H10 were subject to further characterization by comparing their soluble enzyme activity with that of the wild type Candida utilis uricase (WT). Soluble enzyme activity was measured with whole cell lysates of induced E. coli cells, as described above in the “Methods” section. The SEQ ID NOs and amino acid sequences of the select variants are shown in Table 14. As shown in FIG. 10, all of the variants exhibited WT-like activity in solution phase activity testing.

TABLE 14
SEQ ID NOs and amino acid sequences of select Candida utilis uricase variants

| Uricase | SEQ ID | |
|---|---|---|
| Enzyme | NO | Amino acid sequence |
| SelU_A11 | 276 | MSTTLSSSTYGKDNVKFLKVKKDPQNPKKQEVMEATVTCLLEG |
| | | GFDTSYTEADNSSIVPTDTVKNTILVLAKTTEIWPIERFAAKLATH |
| | | FVEKYSHVSGVSVKIVQDRWVKYAVDGKPHDHSFIHEGGEKRIT |
| | | DLYYKRSGDYDLSSAIKDLTVLKSTGSEFYGYNKCDFTTLQPTT |
| | | DRILSTDVDATWVWDNKKIGTVYDIAKAADKGIFDNVYNQARE |
| | | TTLKTFALENSPSVQNTMFNMGTQILEKACSVYSVSYALPNKHY |
| | | FLIDLKWKGLENDNELFYPSPHPNGLIKCTVVRKEKTKL |
| SelU_D6 | 307 | MSTTLSSSTYGKDNVKFLKVKKDPQNPKKQEVMEATVTCLLEG |
| | | GFDTSYTEADNSSIVPTDTVKNTILVLAKTTEIWPIERFAAKLATH |
| | | FVEKYDHVSGVSVKIVQDRWVKYAVDGKPHDHSFIHEGGEKRI |
| | | TDLYYKRSGDYKLESAIKDLTVLKSTGSMFYGYNKCDFTTLQPT |
| | | TDRILSTDVDATWVWDNKKIGTVYDIAKAADKGIFDNVYNQAR |
| | | EITLTTFANENSPSVQNTMFNMGTQILEKACSVDSVSYELPNKHY |
| | | FEIDLKWKGLENDNELFYPSPHPNGLIKCTVVRKEKTKL |
| SelU_G1 | 327 | MSTTLSSSTYGKDNVKFLKVKKDPQNPKKQEVMEATVTCLLEG |
| | | GFDTSYTEADNSSIVPTDTVKNTILVTAKTTEIWPIERYAAKLGT |
| | | HFVEKYSHVDGVSVKIVQDRWVKYAVDGKPHDHSFIHEGSEKRI |
| | | TDLYYKRSGDYKLSSKIKDLTVLKSTGSMFYGYNKCDFTTLQPT |
| | | TDRILSTDVDATWVWDNKKIGTVYDIAKAADKGIFDNVYNQAR |
| | | EITLTTFAEENSPSVQATMFNMGTQILEKACSVYSVSYELPNKHY |
| | | FLIDLKWKGLENDNELFYPSPHPNGLIKCTVVRKEKTKL |
| SelU_G4 | 340 | MSTTLSSSTYGKDNVKFLKVKKDPQNPKKQEVMEATVTCLLEG |
| | | GFDTSYTEADNSSIVPTDTVKNTILVLAKTTEIWPIERFAAKLATH |
| | | FVEKYDHVSGVSVKIVQDRWVKYAVDGKPHDHSFIHEGGEKRI |
| | | TDLYYKRSGDYKLSSKIKDLTVLKSTGSMFYGYNKCDFTTLQPT |
| | | TDRILSTDVDATWVWDNKKIGTVYDIAKAADKGIFDNVYNQAR |
| | | EITLKTFANENSPSVQATMFNMGTQILEKACSVDSVSYALPNKH |
| | | YFEIDLKWKGLENDNELFYPSPHPNGLIKCTVVRKEKTKL |
| SelU_H8 | 341 | MSTTLSSSTYGKDNVKFLKVKKDPQNPKKQEVMEATVTCLLEG |
| | | GFDTSYTEADNSSIVPTDTVKNTILVLAKTTEIWPIERFAAKLATH |
| | | FVEKYDHVDGVSVKIVQDRWVKYAVDGKPHDHSFIHEGGEKRI |
| | | TDLYYKRSGDYKLESKIKDLTVLKSTGSEFYGYNKCDFTTLQPT |
| | | TDRILSTDVDATWVWDNKKIGTVYDIAKAADKGIFDNVYNQAR |
| | | ETTLTTFANENSPSVQATMFNMATQILEKACSVDSVSYELPNKH |
| | | YFLIDLKWKGLENDNELFYPSPHPNGLIKCTVVRKEKTKL |
| SelU_H10 | 334 | MSTTLSSSTYGKDNVKFLKVKKDPQNPKKQEVMEATVTCLLEG |
| | | GFDTSYTEADNSSIVPTDTVKNTILVLAKTTEIWPIERFAAKLATH |
| | | FVEKYSHVDGVSVKIVQDRWVKYAVDGKPHDHSFIHEGGEKRI |
| | | TDLYYKRSGDYKLESAIKDLTVLKSTGSMFYGYNKCDFTTLQPT |
| | | TDRILSTDVDATWVWDNKKIGTVYDIAKAADKGIFDNVYNQAR |
| | | ETTLKTFAEENSPSVQATMFNMGTQILEKACSVYSVSYELPNKH |
| | | YFEIDLKWKGLENDNELFYPSPHPNGLIKCTVVRKEKTKL |

While the described invention has been described with reference to the specific embodiments thereof it should be understood by those skilled in the art that various changes may be made and equivalents may be substituted without departing from the true spirit and scope of the invention. In addition, many modifications may be made to adopt a particular situation, material, composition of matter, process, process step or steps, to the objective spirit and scope of the described invention. All such modifications are intended to be within the scope of the claims appended hereto.

Patents, patent applications, patent application publications, journal articles and protocols referenced herein are incorporated by reference in their entireties, for all purposes.

1. A uricase variant comprising a plurality of amino acid substitutions relative to the chimeric pig-baboon uricase of SEQ ID NO:5, wherein the plurality of amino acid substitutions is selected from the group consisting of (i), (ii), (iii) and (iv):
(i) F75Q; K79E; T83Q; E90H; F96H; I100H; Y105D; V119K; Y127H; F172Y; and L260N;
(ii) F75Q; K79E; T83Q; E90H; F96H; I100H; Y105D; V119K; Y127H; K155N; F172Y; and L260N;
(iii) F75Q; K76N; K79N; T83Q; E90H; F96H; I100E; Y105D; V119K; F172Y; L260N; and I262T; and
(iv) F75Q; K76N; K79N; T83Q; E90H; F96H; I100E; Y105D; V119K; K155N; F172Y; L260N; and I262T.

2. The uricase variant of claim 1, comprising the following amino acid substitutions relative to the chimeric pig-baboon uricase of SEQ ID NO:5: (i) F75Q; K79E; T83Q; E90H; F96H; I100H; Y105D; V119K; Y127H; F172Y; and L260N.

3. The uricase variant of claim 2, comprising an amino acid sequence of SEQ ID NO:1.

4. The uricase variant of claim 1, comprising the following amino acid substitutions relative to the chimeric pig-baboon uricase of SEQ ID NO:5: (ii) F75Q; K79E; T83Q; E90H; F96H; I100H; Y105D; V119K; Y127H; K155N; F172Y; and L260N.

5. The uricase variant of claim 4, comprising an amino acid sequence of SEQ ID NO:2.

6. The uricase variant of claim 1, comprising the following amino acid substitutions relative to the chimeric pig-baboon uricase of SEQ ID NO:5: (iii) F75Q; K76N; K79N; T83Q; E90H; F96H; I100E; Y105D; V119K; F172Y; L260N; and I262T.

7. The uricase variant of claim 6, comprising an amino acid sequence of SEQ ID NO:3.

8. The uricase variant of claim 1, comprising the following amino acid substitutions relative to the chimeric pig-baboon uricase of SEQ ID NO:5: (iv) F75Q; K76N; K79N; T83Q; E90H; F96H; I100E; Y105D; V119K; K155N; F172Y; L260N; and I262T.

9. The uricase variant of claim 8, comprising an amino acid sequence of SEQ ID NO:4.

10. A uricase variant comprising an amino acid sequence selected from SEQ ID NOs: 197-273.

11. The uricase variant of claim 10, comprising the amino acid sequence of SEQ ID NO: 229.

12. The uricase variant of claim 10, comprising the amino acid sequence of SEQ ID NO: 236.

13. The uricase variant of claim 10, comprising the amino acid sequence of SEQ ID NO: 267.

14. The uricase variant of claim 10, comprising the amino acid sequence of SEQ ID NO: 268.

15. The uricase variant of claim 10, comprising the amino acid sequence of SEQ ID NO: 269.

16. The uricase variant of claim 10, comprising the amino acid sequence of SEQ ID NO: 270.

17. The uricase variant of claim 10, comprising the amino acid sequence of SEQ ID NO: 271.

18. The uricase variant of claim 10, comprising the amino acid sequence of SEQ ID NO: 272.

19. The uricase variant of claim 10, comprising the amino acid sequence of SEQ ID NO: 273.

20. The uricase variant of any one of claims 1-19, further comprising an N-terminal methionine residue.

21. A uricase variant comprising an amino acid sequence selected from SEQ ID NOs: 12-196.

22. The uricase variant of claim 21, comprising the amino acid sequence of SEQ ID NO: 77.

23. The uricase variant of claim 21, comprising the amino acid sequence of SEQ ID NO: 134.

24. The uricase variant of claim 21, comprising the amino acid sequence of SEQ ID NO: 80.

25. The uricase variant of claim 21, comprising the amino acid sequence of SEQ ID NO: 144.

26. The uricase variant of claim 21, comprising the amino acid sequence of SEQ ID NO: 161.

27. The uricase variant of claim 21, comprising the amino acid sequence of SEQ ID NO: 164.

28. The uricase variant of claim 21, comprising the amino acid sequence of SEQ ID NO: 95.

29. A uricase variant comprising an amino acid sequence selected from SEQ ID NOs: 274-341.

30. The uricase variant of claim 29, comprising the amino acid sequence of SEQ ID NO: 276.

31. The uricase variant of claim 29, comprising the amino acid sequence of SEQ ID NO: 307.

32. The uricase variant of claim 29, comprising the amino acid sequence of SEQ ID NO: 327.

33. The uricase variant of claim 29, comprising the amino acid sequence of SEQ ID NO: 340.

34. The uricase variant of claim 29, comprising the amino acid sequence of SEQ ID NO: 341.

35. The uricase variant of claim 29, comprising the amino acid sequence of SEQ ID NO: 334.

36. The uricase variant of any one of claims 21-35, wherein the uricase variant does not include the N-terminal methionine residue.

37. The uricase variant of any one of claims 1-36, wherein the uricase variant is isolated.

38. The uricase variant of any one of claims 1-37, wherein the uricase variant is a monomer.

39. The uricase variant of any one of claims 1-37, wherein the uricase variant is a homodimer.

40. The uricase variant of any one of claims 1-37, wherein the uricase variant is a homotetramer.

41. The uricase variant of any one of claims 1-40, wherein the uricase variant is non-PEGylated.

42. The uricase variant of any one of claims 1-40, wherein the uricase variant is PEGylated.

43. The uricase variant of any one of claims 1-42, further comprising a purification tag at the C-terminus.

44. The uricase variant of any one of claims 1-43, further comprising a purification tag at the N-terminus.

45. The uricase variant of claim 43 or 44, wherein the purification tag is a polyhistidine tag.

46. An isolated nucleic acid encoding the uricase variant of any one of claims 1-45.

47. A nucleic acid vector comprising the nucleic acid of claim 46.

48. A host cell comprising the nucleic acid vector of claim 47.

49. A pharmaceutical composition comprising the uricase variant of any one of claims 1-45, and a pharmaceutically acceptable carrier.

50. A method of reducing elevated uric acid levels in a subject in need thereof, comprising administering to the subject an effective amount of the uricase variant of any one of claims 1-45, or the pharmaceutical composition of claim 49.

51. The method of claim 50, wherein the uric acid levels are reduced in the plasma of the subject.

52. The method of claim 50 or 51, wherein the subject is a gout patient.

53. The method of claim 52, wherein the subject is a refractory gout patient.

54. The method of any one of claims 50-53, wherein the subject has been diagnosed with tumor lysis syndrome.

55. A method of treating gout in a subject in need thereof, comprising administering to the subject an effective amount of the uricase variant of any one of claims 1-45, or the pharmaceutical composition of claim 49.

56. The method of claim 55, wherein the gout is refractory gout.

57. A method of treating tumor lysis syndrome in a subject in need thereof, comprising administering to the subject an effective amount of the uricase variant of any one of claims 1-45, or the pharmaceutical composition of claim 49.

58. The method of any one of claims 50-57, wherein the subject is a human patient.

59. The method of claim 58, wherein the human patient is an adult human patient.

60. The method of any one of claims 50-59, wherein the uricase variant or the pharmaceutical composition is administered parenterally.

61. The method of claim 60, wherein the uricase variant or the pharmaceutical composition is administered intravenously.

62. The method of claim 60, wherein the uricase variant or the pharmaceutical composition is administered subcutaneously.

63. A method of recombinantly producing the uricase variant of any one of claims 1-45, comprising:
(i) culturing a host cell comprising a nucleic acid vector comprising a nucleic acid sequence encoding the uricase variant of any one of claims 1-45, wherein the nucleic acid sequence is operatively linked to a heterologous promoter under conditions to allow for expression of the nucleic acid sequence encoding the uricase variant and recombinant production of the uricase variant by the host cell; and
(ii) isolating the recombinantly produced uricase variant.


SEQUENCE LISTING (341 sequences)

SEQ ID NO: 1 - protein, 298 aa, synthetic construct
TYKKNDEVEFVRTGYGKDMIKVLHIQRDGKYHSIKEVATTVQLTLSSKKDYLHGDNSDVIPTDTIKNTVNVLAKQKGIESIEQFAVTICHHFLSSHKHVHRAQVDVEEVPWKRFEKNGKKHVHAFIHTPTGTHFCEVEQIRNGPPVIHSGIKDLKVLKTTQSGFEGFIKDQYTTLPEVKDRCFATQVYCKWRYHQGRDVDFEATWDTVRSIVLQKFAGPYDKGEYSPSVQKTLYDIQVLTLGQVPEIEDMEISLPNIHYNNIDMSKMGLINKEEVLLPLDNPYGKITGTVKRKLSSRL

SEQ ID NO: 2 - protein, 298 aa, synthetic construct
TYKKNDEVEFVRTGYGKDMIKVLHIQRDGKYHSIKEVATTVQLTLSSKKDYLHGDNSDVIPTDTIKNTVNVLAKQKGIESIEQFAVTICHHFLSSHKHVHRAQVDVEEVPWKRFEKNGKKHVHAFIHTPTGTHFCEVEQIRNGPPVIHSGIKDLNVLKTTQSGFEGFIKDQYTTLPEVKDRCFATQVYCKWRYHQGRDVDFEATWDTVRSIVLQKFAGPYDKGEYSPSVQKTLYDIQVLTLGQVPEIEDMEISLPNIHYNNIDMSKMGLINKEEVLLPLDNPYGKITGTVKRKLSSRL

SEQ ID NO: 3 - protein, 298 aa, synthetic construct
TYKKNDEVEFVRTGYGKDMIKVLHIQRDGKYHSIKEVATTVQLTLSSKKDYLHGDNSDVIPTDTIKNTVNVLAKQNGINSIEQFAVTICHHFLSSHKHVERAQVDVEEVPWKRFEKNGKKHVHAFIYTPTGTHFCEVEQIRNGPPVIHSGIKDLKVLKTTQSGFEGFIKDQYTTLPEVKDRCFATQVYCKWRYHQGRDVDFEATWDTVRSIVLQKFAGPYDKGEYSPSVQKTLYDIQVLTLGQVPEIEDMEISLPNIHYNNTDMSKMGLINKEEVLLPLDNPYGKITGTVKRKLSSRL

SEQ ID NO: 4 - protein, 298 aa, synthetic construct
TYKKNDEVEFVRTGYGKDMIKVLHIQRDGKYHSIKEVATTVQLTLSSKKDYLHGDNSDVIPTDTIKNTVNVLAKQNGINSIEQFAVTICHHFLSSHKHVERAQVDVEEVPWKRFEKNGKKHVHAFIYTPTGTHFCEVEQIRNGPPVIHSGIKDLNVLKTTQSGFEGFIKDQYTTLPEVKDRCFATQVYCKWRYHQGRDVDFEATWDTVRSIVLQKFAGPYDKGEYSPSVQKTLYDIQVLTLGQVPEIEDMEISLPNIHYNNTDMSKMGLINKEEVLLPLDNPYGKITGTVKRKLSSRL

SEQ ID NO: 5 - protein, 298 aa, synthetic construct
TYKKNDEVEFVRTGYGKDMIKVLHIQRDGKYHSIKEVATTVQLTLSSKKDYLHGDNSDVIPTDTIKNTVNVLAKFKGIKSIETFAVTICEHFLSSFKHVIRAQVYVEEVPWKRFEKNGVKHVHAFIYTPTGTHFCEVEQIRNGPPVIHSGIKDLKVLKTTQSGFEGFIKDQFTTLPEVKDRCFATQVYCKWRYHQGRDVDFEATWDTVRSIVLQKFAGPYDKGEYSPSVQKTLYDIQVLTLGQVPEIEDMEISLPNIHYLNIDMSKMGLINKEEVLLPLDNPYGKITGTVKRKLSSRL

SEQ ID NO: 6 - protein, 291 aa, Arthrobacter globiformis
MTATAETSTGTKVVLGQNQYGKAEVRLVKVTRNTARHEIQDLNVTSQLSGDFEAAHTAGDNAHVVATDTQKNTVYAFARDGFATTEEFLLRLGKHFTEGFDWVTGGRWAAQQFFWDRINDHDHAFSRNKSEVRTAVLEISGSEQAIVAGIEGLTVLKSTGSEFHGFPRDKYTTLQETTDRILATDVSARWRYNTVEVDFDAVYASVRGLLLKAFAETHSLALQQTMYEMGRAVIETHPEIDEIKMSLPNKHHFLVDLQPFGQDNPNEVFYAADRPYGLIEATIQREGSRAD

SEQ ID NO: 7 - protein, 301 aa, Aspergillus flavus
SAVKAARYGKDNVRVYKVHKDEKTGVQTVYEMTVCVLLEGEIETSYTKADNSVIVATDSIKNTIYITAKQNPVTPPELFGSILGTHFIEKYNHIHAAHVNIVCHRWTRMDIDGKPHPHSFIRDSEEKRNVQVDVVEGKGIDIKSSLSGLTVLKSTNSQFWGFLRDEYTTLKETWDRILSTDVDATWQWKNFSGLQEVRSHVPKFDATWATAREVTLKTFAEDNSASVQATMYKMAEQILARQQLIETVEYSLPNKHYFEIDLSWHKGLQNTGKNAEVFAPQSDPNGLIKCTVGRSSLKSKL

SEQ ID NO: 8 - protein, 303 aa, Candida utilis
MSTTLSSSTYGKDNVKFLKVKKDPQNPKKQEVMEATVTCLLEGGFDTSYTEADNSSIVPTDTVKNTILVLAKTTEIWPIERFAAKLATHFVEKYSHVSGVSVKIVQDRWVKYAVDGKPHDHSFIHEGGEKRITDLYYKRSGDYKLSSAIKDLTVLKSTGSMFYGYNKCDFTTLQPTTDRILSTDVDATWVWDNKKIGTVYDIAKAADKGIFDNVYNQAREITLTTFALENSPSVQATMFNMATQILEKACSVYSVSYALPNKHYFLIDLKWKGLENDNELFYPSPHPNGLIKCTVVRKEKTKL

SEQ ID NO: 9 - protein, 6 aa, synthetic construct
HQHQHQ

SEQ ID NO: 10 - protein, 12 aa, synthetic construct
HNHNHNHNHNHN

SEQ ID NO: 11 - protein, 19 aa, synthetic construct
KDHLIHNVHKEEHAHAHNK

SEQ ID NO: 12 - protein, 291 aa, synthetic construct
MTATAETSTGTKVVLGQNQYGKAEVRLVKVTRNTARHEIQDLNVTSQLSGDFEAAHTAGDNAHVVATDTQKNTVYAFARDGFATTEEFLLRLGKHFTEGFDWVTGGRWAAQQHFWDRINDHDHAFSRNKSEVRTAVLEISGSEQTIVAGIESLTVLKSTGSEFHGFPRDKYTTLQETTDRILATDVSARWRYNTEEVDFDAVYASVRGLLLKGFAETHSLALQQTMYEMGRAVIETHPEIDEVSMSLPNKHHFLVDLQPFGQDNPNEVFYAADRPYGLIEATIQREGSRAD

SEQ ID NO: 13 - protein, 291 aa, synthetic construct
MTATAETSTGTKVVLGQNQYGKAEVRLVKVTRNTARHEIQDLNVTSQLSGDFEAAHTAGDNAHVVATDTQKNTVYAFARDGLGTTEEFLLRLGKHFTEGFDWVTGGRWAAQQFFWDRINDHDHAFSRNKSEVRTAVLEISGSEQHIVSGIEGLTVLKSTGSEFHGFPRDKYTTLQETTDRILATDVSARWRYNTVEVDFDAVYGSVRGLLLKAFAETHSHALQQTMYEMGRAVIETHPEIDEMSMSLPNKHHFLVDLQPFGQDNPNEVFHAADRPYGLIEATIQREGSRAD

SEQ ID NO: 14 - protein, 291 aa, synthetic construct
MTATAETSTGTKVVLGQNQYGKAEVRLVKVTRETARHEIQDLNVTSQLSGDFEAAHTAGDNAHVVATDTQKNTVYAFARDGVATTEEFLLRLGKHFTEGFDWVTGGRWAAQQFFWDRINDHDHAFSRNKSEVRTAVLEISGSEQTIVAGIEGLTVLKSTGSEFHGFPRDKYTTLQETTDRILSTDVSARWRYNTDEVDFDAVYASVRGLLLKGFAETHSKALQQTMYEMGRAVIETHPEIDEVSMSLPNKHHFLVDLQPFGQDNPNEVFHAADRPYGLIEATIQREGSRAD

SEQ ID NO: 15 - protein, 291 aa, synthetic construct
MTATAETSTGTKVVLGQNQYGKAEVRLVKVTRNTDRHEIQDLNVTSQLSGDFEAAHTAGDNAHVVATDTQKNTVYAFARDGVATTEEFLLRLGKHFTEGFDWVTGGRWAAQQFFWDRINDHDHAFSRNKSEVRTAVLEISGSEQAIVAGIEGLTVLKSTGSEFHGFPRDKYTTLQETTDRILATDVSARWRYNTVEVDFDAVYASVRGLLLKGFAETHSKALQQTMYEMGRAVIETHPEIDEVSMSLPNKHHFLVDLQPFGQDNPNEVFYAADRPYGLIEATIQREGSRAD

SEQ ID NO: 16 - protein, 291 aa, synthetic construct
MTATAETSTGTKVVLGQNQYGKAEVRLVKVTRNTARHEIQDLNVTSQLSGDFEAAHTAGDNAHVVATDTQKNTVYAFARDGVATTEEFLLRLGKHFTEGFDWVTGGRWAAQQFFWDRINDHDHAFSRNKSEVRTAVLEISGSEQHIVSGIEGLTVLKSTGSEFHGFPRDKYTTLQETTDRILATDVSARWRYNTDEVDFDAVYDSVRGLLLKGFAETHSLALQQTMYEMGRAVIETHPEIDEVSMSLPNKHHFLVDLQPFGQDNPNEVFYAADRPYGLIEATIQREGSRAD

SEQ ID NO: 17 - protein, 291 aa, synthetic construct
MTATAETSTGTKVVLGQNQYGKAEVRLVKVTRNTDRHEIQDLNVTSQLSGDFEAAHTAGDNAHVVATDTQKNTVYAFARDGFGTTEEFLLRLGKHFTEGFDWVTGGRWAAQQFFWDRINDHDHAFSRNKSEVRTAVLEISGSEQAIVAGIEGLTVLKSTGSEFHGFPRDKYTTLQETTDRILATDVSARWRYNADEVDFDAVYASVRGLLLKGFAETHSKALQQTMYEMGRAVIETHPEIDEIKMSLPNKHHFLVDLQPFGQDNPNEVFYAADRPYGLIEATIQREGSRAD

SEQ ID NO: 18 - protein, 291 aa, synthetic construct
MTATAKTSTGTKVVLGQNQYGKAQVRLVKVTRNTDRHEIQDLNVTSQLSGDFEAAHTAGDNAHVVATDTQKNTVYAFARDGFATTEEFLLRLGKHFTEGFDWVTGGRWAAQQFFWDRINDHDHAFSRNKSEVRTAVLEISGSEQHIVSGIEGLTVLKSTGSEFHGFPRDKYTTLQETTDCIVAADVFGRWRYNTDEVDFDAVYGSVRGLLLKAFAETHSLALQQTMYEMGRAVIETHPEIDEVKMSLPNNHHFLVDMHPFGQDNPNEVFHAADRPYGLIEATIQREGSRAD

SEQ ID NO: 19 - protein, 291 aa, synthetic construct
MTATAETSTGTKVVLGQNQYGKAEVRLVKVTRNTARHEIQDLNVTSQLSGDFEAAHTAGDNAHVVATDTQKNTVYAFARDGFATTEEFLLRLGKHFTEGFDWVTGGRWAAQQHFWDRINDHDHAFSRNKSEVRTAVLEISGSEQAIISGIEGLTVLKSTGSEFHGFPRDKYTTLQETTDRILATDVSARWRYNTDEVDFDAVYGSVRGLLLKAFAETHSKALQQTMYEMGRAVIETHPEIDEISMSLPNNHHFLVDLQPFGQDNPNEVFHAADRPYGLIEATIQREGSRAD

SEQ ID NO: 20 - protein, 291 aa, synthetic construct
MTATAETSTGTKVVLGQNQYGKAEARLVKVTRNTDRHEIQDLNVTSQLSGDFEAAHTAGDNAHVVATDTQKNTVYAFARDGFGTTEEFLLRLGKHFTEGFDWVTGGRWAAQQFFWDRINDHDHAFSRNKSEVRTAVLEISGSEQHIVAGIEGLTVLKSTGSEFHGFPRDKYTTLQETTDRILATDVSARWRYNTVEVDFDAVYASVRGLLLKAFAETHSKALQQTMYEMGRAVIETHPEIDEISMSLPNKHHFLVDLQPFGQDNPNEVFYAADRPYGLIEATIQREGSRAD

SEQ ID NO: 21 - protein, 291 aa, synthetic construct
MTATAETSTGTKVVLGQNQYGKAEVRLVKVTRETARHEIQDLNVTSQLSGDFEAAHTAGDNAHVVATDTQKNTVYAFARDGFGTTEEFLLRLGKHFTEGFDWVTGGRWAAQQFFWDRINDHDHAFSRNKSEVRTAVLEISGSEQAIVSGIEGLTVLKSTGSEFHGFPRDKYTTLQETTDRILATDVSARWRYNTDEVDFDAVYDSVRGLLLKAFAETHSKALQQTMYEMGRAVIETPPEIDEVKMSLPNNPHFLVDLQPFGQDNPNEVFHAADRPYGLIEATIQREGSRAD

SEQ ID NO: 22 - protein, 291 aa, synthetic construct
MTATAETSTGTKVVLGQNQYGKAEVRLVKVTRNTARHEIQDLNVTSQLSGDFEAAHTAGDNAHVVATDTQKNTVYAFARDGVGTTEEFLLRLGKHFTEGFDWVTGGRWAAQQFFWDRINDHDHAFSRNKSEVRTAVLEISGSEQDIVAGIEGLTVLKSTGSEFHGFPRDKYTTLQETTDRILATDVSARWRYNTEEVDFDAVHGSVRGLLLKGFAETHSNALQQTMYEMGRAVIETHPEIDEVKMSLPNKHHFLVDLQPFGQDNPNEVFYAADRPYGLIEATIQREGSRAD

SEQ ID NO: 23 - protein, 291 aa, synthetic construct
MTATAETSTGTKVVLGQNQYGKAEVRLVKVTRNTARHEIQDLNVTSQLSGDFEAAHTAGDNAHVVATDTQKNTVYAFARDGFGTTEEFLLRLGKHFTEGFDWVTGGRWAAQQFFWDRINDHDHAFSRNKSEVRTAVLEISGSEQTIVSGIEGLTVLKSTGSEFHGFPRDKYTTLQETTDRILATDVSARWRYNTDEVDFDAVYGSVRGLLLKAFAETHSLALQQTMYEMGRAVIETHPEIDEIKMSLPNKHHFLVDLQPFGQDNPNEVFYAADRPYGLIEATIQREGSRAD

SEQ ID NO: 24 - protein, 291 aa, synthetic construct
MTATAETSTGTKVVLGQNQYGKAEVRLVKVTRETARHEIQDLNVTSQLSGDFEAAHTAGDNAHVVATDTQKNTVYAFARDGFGTTEEFLLRLGKHFTEGFDWVTGGRWAAQQFFWDRINDHDHAFSRNKSEVRTAVLEISGSEQHIVSGIEGLTVLKSTGSEFHGFPRDKYTTLQETTDRILATDVSARWRYNTEEVDFDAVYASVRGLLLKAFAETHSHALQQTMYEMGRAVIETHPEIDEVKMSLPNNHHFLVDLQPFGQDNPNEVFYAADRPYGLIEATIQREGSRAD

SEQ ID NO: 25 - protein, 291 aa, synthetic construct
MTATAETSTGTKVVLGQNQYGKAEVRLVKVTRNTARHEIQDLNVTSQLSGDFEAAHTAGDNAHVVATDTQKNTVYAFARDGVGTTEEFLLRLGKHFTEGFDWVTGGRWAAQQHFWDRINDHDHAFSRNKSEVRTAVLEISGSEQAIVAGIEGLTVLKSTGSEFHGFPRDKYTTLQETTDRILATDVSARWRYNTEEVDFDAVYASVRGLLLKAFAETHSLALQQTMYEMGRAVIETHPEIDEISMSLPNKHHFLVDLQPFGQDNPNEVFHAADRPYGLIEATIQREGSRAD

SEQ ID NO: 26 - protein, 291 aa, synthetic construct
MTATAETSTGTKVVLGQNQYGKAEVRLVKVTRNTDRHEIQDLNVTSQLSGDFEAAHTAGDNAHVVATDTQKNTVYAFARDGFATTEEFLLRLGKHFTEGFDWVTGGRWAAQQFFWDRINDHDHAFSRNKSEVRTAVLEISGSEQAIVAGIEGLTVLKSTGSEFHGFPRDKYTTLQETTDRILATDVSARWRYNTDEVDFDAVYDSVRGLLLKGFAETHSHALQQTMYEMGRAVIETHPEIDEVKMSLPNKHHFLVDLQPFGQDNPNEVFYAADRPYGLIEATIQREGSRAD

SEQ ID NO: 27 - protein, 291 aa, synthetic construct
MTATAETSTGTKVVLGQNQYGKAEVRLVKVTRNTARHEIQDLNVTSQLSGDFEAAHTAGDNAHVVATDTQKNTVYAFARDGFGTTEEFLLRLGKHFTEGFDWVTGGRWAAQQHFWDRINDHDHAFSRNKSEVRTAVLEISGSEQTIVSGIEGLTVLKSTGSEFHGFPRDKYTTLQETTDRILATDVSARWRYNTDEVDFDAVYASVRGLLLKAFAETHSHALQQTMYEMGRAVIETHPEIDEVKMSLPNKHHFLVDLQPFGQDNPNEVFYAADRPYGLIEATIQREGSRAD

SEQ ID NO: 28 - protein, 291 aa, synthetic construct
MTATAETSTGTKVVLGQNQYGKAEVRLVKVTRETARHEIQDLNVTSQLSGDFEAAHTAGDNAHVVATDTQKNTVYAFARDGFGTTEEFLLRLGKHFTEGFDWVTGGRWAAQQFFWDRINDHDHTFSRNKSEIRTAVLEISGSEQAIVSGIEGLTVLKSTGSEFHGFPRDKYTTLQETTDRILATDVSARWRYNTDEVDFDAVYDSVRGLLLKAFAETHSKALQQTMYEMGRAVIETHPEIDEVKMSLPNKHHFLVDLQPFGQDNPNEVFHAADRPYGLIEATIQREGSRAD

SEQ ID NO: 29 - protein, 291 aa, synthetic construct
MTATAETSTGTKVVLGQNQYGKAEVRLVKVTRNTDRHEIQDLNVTSQLSGDFEAAHTAGDNAHVVATDTQKNTVYAFARDGFATTEEFLLRLGKHFTEGFDWVTGGRWAAQQFFWDRINDHDHAFSRNKSEVRTAVLEISGSEQTIVSGIEGLTVLKSTGSEFHGFPRDKYTTLQETTDRILATDVSARWRYNTEEVDFDAVYGSVRGLLLKGFAETHSHALQQTMYEMGRAVIETHPEIDEVKMSLPNNHHFLVDLQPFGQDNPNEVFYAADRPYGLIEATIQREGSRAD

SEQ ID NO: 30 - protein, 291 aa, synthetic construct
MTATAETSTGTKVVLGQNQYGKAEVRLVKVTRNTARHEIQDLNVTSQLSGDFEAAHTAGDNAHVVATDTQKNTVYAFARDGFGTTEEFLLRLGKHFTEGFDWVTGGRWAAQQHFWDRINDHDHAFSRNKSEVRTAVLEISGSEQTIVAGIEGLTVLKSTGSEFHGFPRDKYTTLQETTDRILATDVSARWRYNTEEVDFDAVYDSVRGLLLKAFAETHSKALQQTMYEMGRAVIETHPEIDEIKMSLPNKHHFLVDLQPFGQDNPNEVFYAADRPYGLIEATIQREGSRAD

SEQ ID NO: 31 - protein, 291 aa, synthetic construct
MTATAETSTGTKVVLGQNQYGKAEVRLVKVTRETDRHEIQDLNVTSQLSGDFEAAHTAGDNAHVVATDTQKNTVYAFARDGFGTTEEFLLRLGKHFTEGFDWVTGGRWAAQQFFWDRINDHDHAFSRNKSEVRTAVLEISGSEQTIVSGIEGLTVLKSTGSEFHGFPRDKYTTLQETTDRILATDVSARWRYNTEEVDFDAVYDSVRGLLLKAFAETHSKALQQTMYEMGRAVIETHPEIDEIKMSLPNNHHFLVDLQPFGQDNPNEVFHAADRPYGLIEATIQREGSRAD

SEQ ID NO: 32 - protein, 291 aa, synthetic construct
MTATAETSTGTKVVLGQNQYGKAEVRLVKVTRDTARHEIQDLNVTSQLSGDFEAAHTAGDNAHVVATDTQKNTVYAFARDGFATTEEFGLRLGKHFTEGFDWVTGGRWAAQQFFWDRINDHDHAFSRNKSEVRTAVLEISGSEQDIVSGIEGLTVLKSTGSEFHGFPRDKYTTLQETTDRILATDVSARWRYNTVEVDFDAVYDSVRGLLLKGFAETHSHALQQTMYEMGRAVIETHPEIDEIKMSLPNKHHFLVDLQPFGQDNPNEVFYAADRPYGLIEATIQREGSRAD

SEQ ID NO: 33 - protein, 291 aa, synthetic construct
MTATAETSTGTKVVLGQNQYGKAEVRLVKVTRETARHEIQDLNVTSQLSGDFEAAHTAGDNAHVVATDTQKNTVYAFARDGFATTEEFLLRLGKHFTEGFDWVTGGRWAAQQFFWDRINDHDHAFSRNKSEVRTAVLEISGSEQTIVSGIEGLTVLKSTGSEFHGFPRDKYTTLQETTDRILATDVSARWRYNTDEVDFDAVYDSVRGLLLKAFAETHSHALQQTMYEMGRAVIETHPEIDEIKMSLPNKHHFLVDMHPFGQDNPNEVFHAADRPYGLIEATIQREGSRAD

SEQ ID NO: 34 - protein, 291 aa, synthetic construct
MTATAETSTGTKVVLGQNQYGKAEVRLVKVTRNTARHEIQDLNVTSQLSGDFEAAHTAGDNAHVVATDTQKNTVYAFARDGFATTEEFLLRLGKHFTEGFDWVTGGRWAAQQHFWDRINDHDHAFSRNKSEVRTAVLEISGSEQTIVAGIEGLTVLKSTGSEFHGFPRDKYTTLQETTDRILATDVSARWRYNTEEVDFDAVYGSVRGLLLKGFAETHSHALQQTMYEMGRAVIETHPEIDEVKMSLPNKHHFLVDLQPFGQDNPNEVFYAADRPYGLIEATIQREGSRAD

SEQ ID NO: 35 - protein, 291 aa, synthetic construct
MTATAETSTGTKVVLGQNQYGKAEVRLVKVTRNTARHEIQDLNVTSQLSGDFEAAHTAGDNAHVVATDTQKNTVYAFARDGFATTEEFLLRLGKHFTEGFDWVTGGRWAAQQFFWDRINDHDHAFSRNKSEVRTAVLEISGSEQAIVAGIEGLTVLKSTGSEFHGFPRDKYTTLQETTDRILATDVSARWRYNTEEVDFDAVYASVRGLLLKGFAETHSLALQQTMYEMGRAVIETHPEIDEIKMSLPNNHHFLVDLQPFGQDNPNEVFHAADRPYGLIEATIQREGSRAD

SEQ ID NO: 36 - protein, 291 aa, synthetic construct
MTATAETSTGTKVVLGQNQYGKAEVRLVKVTRETDRHEIQDLNVTSQLSGDFEAAHTAGDNAHVVATDTQKNTVYAFARDGFATTEEFLLRLGKHFTEGFDWVTGGRWAAQQFFWDRINDHDHAFSRNKSEVRTAVLEISGSEQAIVAGIEGLTVLKSTGSEFHGFPRDKYTTLQETTDRILATDVSARWRYNTVEVDFDAVYGSVRGLLLKGFAETHSHALQQTMYEMGRAVIETHPEIDEISMSLPNNHHFLVDLQPFGQDNPNEVFYAADRPYGLIEATIQREGSRAD

SEQ ID NO: 37 - protein, 291 aa, synthetic construct
MTATAETSTGTKVVLGQNQYGKAEVRLVKVTRETARHEIQDLNVTSQLSGDFEAAHTAGDNAHVVATDTQKNTVYAFARDGFATTEEFLLRLGKHFTEGFDWVTGGRWAAQQFFWDRINDHDHAFSRNKSEVRTAVLEISGSEQHIVAGIEGLTVLKSTGSEFHGFPRDKYTTLQETTDRILATDVSARWRYNTVEVDFDAVYASVRGLLLKGFAETHSKALQQTMYEMGRAVIETHPEIDEVKMSLPNNHHFLVDLQPFGQDNPNEVFHAADRPYGLIEATIQREGSRAD

SEQ ID NO: 38 - protein, 291 aa, synthetic construct
MTATAETSTGTKVVLGQNQYGKAEVRLVKVTRETARHEIQDLNVTSQLSGDFEAAHTAGDNAHVVATDTQKNTVYAFARDGFATTEEFLLRLGKHFTEGFDWVTGGRWAAQQFFWDRINDHDHAFSRNKSEVRTAVLEISGSEQAIVAGIEGLTVLKSTGSEFHGFPRDKYTTLQETTDRILATDVSARWRYNTEEVDFDAVYGSVRGLLLKAFAETHSKALQQTMYEMGRAVIETHPEIDEVKMSLPNKHHFLVDLQPFGQDNPNEVFYAADRPYGLIEATIQREGSRAD

SEQ ID NO: 39 - protein, 291 aa, synthetic construct
MTATAETSTGTKVVLGQNQYGKAEVRLVKVTRETDRHEIQDLNVTSQLSGDFEAAHTAGDNAHVVATDTQKNTVYAFARDGFATTEEFLLRLGKHFTEGFDWVTGGRWAAQQHFWDRINDHDHAFSRNKSEVRTAVLEISGSEQTIVAGIEGLTVLKSTGSEFHGFPRDKYTTLQETTDRILATDVSARWRYNTDEVDFDAVYASVRGLLLKAFAETHSKALQQTMYEMGRAVIETHPEIDEVSMSLPNKHHFLVDLQPFGQDNPNEVFYAADRPYGLIEATIQREGSRAD

SEQ ID NO: 40 - protein, 291 aa, synthetic construct
MTATAETSTGTKVVLGQNQYGKAEVRLVKVTRETARHEIQDLNVTSQLSGDFEAAHTAGDNAHVVATDTQKNTVYAFARDGFGTTEEFLLRLGKHFTEGFDWVTGGRWAAQQFFWDRINDHDHAFSRNKSEVRTAVLEISGSEQTIVSGIEGLTVLKSTGSEFHGFPRDKYTTLQETTDRILATDVSARWRYNTEEVDFDAVYASVRGLLLKGFAETHSLALQQTMYEMGRAVIETHPEIDEVSMSLPNKHHFLVDLQPFGQDNPNEVFYAADRPYGLIEATIQREGSRAD

SEQ ID NO: 41 - protein, 291 aa, synthetic construct
MTATAETSTGTKVVLGQNQYGKAEVRLVKVTRNTDRHEIQDLNVTSQLSGDFEAAHTAGDNAHVVATDTQKNTVYAFARDGFGTTEEFLLRLGKHFTEGFDWVTGGRWAAQQHFWDRINDHDHAFSRNKSEVRTAVLEISGSEQHIVAGIEGLTVLKSTGSEFHGFPRDKYTTLQETTDRILATDVSARWRYNTEEVDFDAVYASVRGLLLKAFAETHSHALQQTMYEMGRAVIETHPEIDEVKMSLPNKHHFLVDLQPFGQDNPNEVFYAADRPYGLIEATIQREGSRAD

SEQ ID NO: 42 - protein, 291 aa, synthetic construct
MTATAETSTGTKVVLGQNQYGKAEVRLVKVTRNTARHEIQDLNVTSQLSGDFEAAHTAGDNAHVVATDTQKNTVYAFARDGFATTEEFLLRLGKHFTEGFDWVTGGRWAAQQFFWDRINDHDHAFSRNKSEVRTAVLEISGSEQTIVSGIEGLTVLKSTGSEFHGFPRDKYTTLQETTDRILATDVSARWRYNTEEVDFDAVYASVRGLLLKGFAETHSHALQQTMYEMGRAVIETHPEIDEVSMSLPNKHHFLVDLQPFGQDNPNEVFYAADRPYGLIEATIQREGSRAD

SEQ ID NO: 43 - protein, 291 aa, synthetic construct
MTATAETSTGTKVVLGQNQYGKAEVRLVKVTRNTARHEIQDLNVTSQLSGDFEAAHTAGDNAHVVATDTQKNTVYAFARDGFGTTEEFLLRLGKHFTEGFDWVTGGRWAAQQFFWDRINDHDHAFSRNKSEVRTAVLEISGSEQAIVSGIEGLTVLKSTGSEFHGFPRDKYTTLQETTDRILATDVSARWRYNTEEVDFDAVYDSVRGLLLKAFAETHSKALQQTMYEMGRAVIETHPEIDEISMSLPNKHHFLVDLQPFGQDNLNEVFHAADRPYGLIEATIQREGSRAD

SEQ ID NO: 44 - protein, 291 aa, synthetic construct
MTATAETSTGTKVVLGQNQYGKAEVRLVKVTRNTDRHEIQDLNVTSQLSGDFEAAHTAGDNAHVVATDTQKNTVYAFARDGVATTEEFLLRLGKHFTEGFDWVTGGRWAAQQFFWDRINDHDHAFSRNKSEVRTAVLEISGSEQTIVSGIEGLTVLKSTGSEFHGFPRDKYTTLQETTDRILATDVSARWRYNTEEVDFDAVYGSVRGLLLKAFAETHSLALQQTMYEMGRAVIETHPEIDEISMSLPNKHHFLVDLQPFGQDNPNEVFYAADRPYGLIEATIQREGSRAD

SEQ ID NO: 45 - protein, 291 aa, synthetic construct
MTATAETSTGTKVVLGQNQYGKAEVRLVKVTRNTARHEIQDLNVTSQLSGDFEAAHTAGDNAHVVATDTQKNTVYAFARDGFGTTEEFLLRLGKHFTEGFDWVTGGRWAAQQFFWDRINDHDHAFSRNKSEVRTAVLEISGSEQAIVAGIEGLTVLKSTGSEFHGFPRDKYTTLQETTDRILATDVSARWRYNTVEVDFDAVYDSVRGLLLKGFAETHSKALQQTMYEMGRAVIETHPEIDEVKMSLQNKHHFLVDLQPFGQDNPNEVFYAADRPYGLIEATIQREGSRAD

SEQ ID NO: 46 - protein, 291 aa, synthetic construct
MTATAETSTGTKVVLGQNQYGKAEVRLVKVTRETDRHEIQDLNVTSQLSGDFEAAHTAGDNAHVVATDTQKNTVYAFARDGFATTEEFLLRLGKHFTEGFDWVTGGRWAAQQFFWDRINDHDHAFSRNKSEVRTAVLEISGSEQAIVAGIEGLTVLKSTGSEFHGFPRDKYTTLQETTDRILATDVSARWRYNTDEVDFDAVYASVRGLLLKAFAETHSHALQQTMYEMGRAVIETHPEIDEVSMSLPNNHHFLVDLQPFGQDNPNEVFHAADRPYGLIEATIQREGSRAD

SEQ ID NO: 47 - protein, 291 aa, synthetic construct
MTATAETSTGTKVVLGQNQYGKAEVRLVKVTRNTARHEIQDLNVTSQLSGDFEAAHTAGDNAHVVATDTQKNTVYAFARDGFGTTEEFLLRLGKHFTEGFDWVTGGRWAAQQFFWDRINDHDHAFSRNKSEVRTAVLEISGSEQHIVAGIEGLTVLKSTGSEFHGFPRDKYTTLQETTDRILATDVSARWRYNTVEVDFDAVYGSVRGLLLKAFAETHSHALQQTMYEMGRAVIETHPEIDEVKMSLPNNHHFLVDLQPFGQDNPNEVFHAADRPYGLIEATIQREGSRAD

SEQ ID NO: 48 - protein, 291 aa, synthetic construct
MTATAETSTGTKVVLGQNQYGKAEVRLVKVTRETDRHEIQDLNVTSQLSGDFEAAHTAGDNAHVVATDTQKNTVYAFARDGVATTEEFLLRLGKHFTEGFDWVTGGRWAAQQHFWDRINDHDHAFSRNKSEVRTAVLEISGSEQTIVSGIEGLTVLKSTGSEFHGFPRDKYTTLQETTDRILATDVSARWRYNTDEVDFDAVYASVRGLLLKAFAETHSLALQQTMYEMGRAVIETHPEIDEIKMSLPNKHHFLVDLQPFGQDNPNEVFYAADRPYGLIEATIQREGSRAD

SEQ ID NO: 49 - protein, 291 aa, synthetic construct
MTATAETSTGTKVVLGQNQYGKAEVRLVKVTRNTARHEIQDLNVTSQLSGDFEAAHTAGDNAHVVATDTQKNTVYAFARDGFATTEEFLLRLGKHFTEGFDWVTGGRWAAQQFFWDRINDHDHAFSRNKSEVRTAVLEISGSEQHIVAGIEGLTVLKSTGSEFHGFPRDKYTTLQETTDRILATDVSARWRYNTVEVDFDAVHASVRGLLLKAFAETHSLALQQTMYEMGRAVIETHPEIDEVSMSLPNKHHFLVDLQPFGQDNPNEVFYAADRPYGLIEATIQREGSRAD

SEQ ID NO: 50 - protein, 291 aa, synthetic construct
MTATAETSTGTKVVLGQNQYGKAEVRLVKVTRNTARHEIQDLNVTSQLSGDFEAAHTAGDNAHVVATDTQKNTVYAFARDGFGTTEEFLLRLGKHFTEGFDWVTGGRWAAQQFFWDRINDHDHAFSRNKSEVRTAVLEISGSEQAIVSGIEGLTVLKSTGSEFHGFPRDKYTTLQETTDRILATDVSARWRYNTEEVDFDAVYGSVRGLLLKAFAETHSHALQQTMYEMGRAVIETHPEIDEVSMSLPNKHHFLVDLQPFGQDNPNEVFYAADRPYGLIEATIQREGSRAD

SEQ ID NO: 51 - protein, 291 aa, synthetic construct
MTATAETSTGTKVVLGQNQYGKAEVRLVKVTRNTARHEIQDLNVTSQLSGDFEAAHTAGDNAHVVATDTQKNTVYAFARDGFATTEEFLLRLGKHFTEGFDWVTGGRWAAQQFFWDRINDHDHAFSRNKSEVRTAVLEISGSEQHIVAGIEGLTVLKSTGSEFHGFPRDKYTTLQETTDRILATDVSARWRYNTVEVDFDAVYDSVRGLLLKGFAETHSKALQQTMYEMGRAVIETHPEIDEVKMSLPNKHHFLVDLQPFGQDNPNEVFYAADRPYGLIEATIQREGSRAD

SEQ ID NO: 52 - protein, 291 aa, synthetic construct
MTATAETSTGTKVVLGQNQYGKAEVRLVKVTRNTARHEIQDLNVTSQLSGDFEAAHTAGDNAHVVATDTQKNTVYAFARDGFATTEEFLLRLGKHFTEGFDWVTGGRWAAQQFFWDRINDHDHAFSRNKSEVRTAVLEISGSEQHIVSGIEGLTVLKSTGSEFHGFPRDKYTTLQETTDRILATDVSARWRYNTDEVDFDAVYDSVRGLLLKAFAETHSHALQQTMYEMGRAVIETHPEIDEISMSLPNNHHFLVDLQPFGQDNPNEVFYAADRPYGLIEATIQREGSRAD

SEQ ID NO: 53 - protein, 291 aa, synthetic construct
MTATAETSTGTKVVLGQNQYGKAEVRLVKVTRETDRHEIQDLNVTSQLSGDFEAAHTAGDNAHVVATDTQKNTVYAFARDGFATTEEFLLRLGKHFTEGFDWVTGGRWAAQQHFWDRINDHDHAFSRNKSEVRTAVLEISGSEQAIVSGIEGLTVLKSTGSEFHGFPRDKYTTLQETTDRILATDVSARWRYNTDEVDFDAVYGSVRGLLLKGFAETHSHALQQTMYEMGRAVIETHPEIDEISMSLPNKHHFLVDLQPFGQDNPNEVFYAADRPYGLIEATIQREGSRAD

SEQ ID NO: 54 - protein, 291 aa, synthetic construct
MTATAETSTGTKVVLGQNQYGKAEVRLVKVTRDTARHEIQDLNVTIQLSGDFEAAHTAGDNAHVVATDTQKNTVYAFARDGVATTEEFLLRLGKHFTEGFDWVTGGRWAAQQFFWDRINDHDHAFSRNKSEVRTAVLEISGSEQTIVAGIEGLTVLKSTGSEFHGFPRDKYTTLQETTDRILATDVSARWRYNTEEVDFDAVHGSVRGLLLKAFAETHSQALQQTMYEMGRAVIETHPEIDEIKMSLPNKHHFLVDLQPFGQDNPNEVFHAADRPYGLIEATIQREGSRAD

SEQ ID NO: 55 - protein, 291 aa, synthetic construct
MTATAETSTGTKVVLGQNQYGKAEVRLVKVTRNTDRHEIQDLNVTSQLSGDFEAAHTAGDNAHVVATDTQKNTVYAFARDGFGTTEEFLLRLGKHFTEGFDWVTGGRWAAQQFFWDRINDHDHAFSRNKSEVRTAVLEISGSEQAIVAGIEGLTVLKSTGSEFHGFPRDKYTTLQETTDRILATDVSARWRYNTDEVDFDAVYASVRGLLLKGFAETHSHALQQTMYEMGRAVIETHPEIDEIKMSLPNKHHFLVDLQPFGQDNPNEVFYAADRPYGLIEATIQREGSRAD

SEQ ID NO: 56 - protein, 291 aa, synthetic construct
MTATAETSTGTKVVLGQNQYGKAEVRLVKVTRETDRHEIQDLNVTSQLSGDFEAAHTAGDNAHVVATDTQKNTVYAFARDGFGTTEEFLLRLGKHFTEGFDWVTGGRWAAQQFFWDRINDHDHAFSRNKSEVRTAVLEISGSEQHIVSGIEGLTVLKSTGSEFHGFPRDKYTTLQETTDRILATDVSARWRYNTEEVDFDAVYASVRGLLLKGFAETHSKALQQTMYEMGRAVIETHPEIDEVSMSLPNKHHFLVDLQPFGQDNPNEVFYAADRPYGLIEATIQREGSRAD

SEQ ID NO: 57 - protein, 291 aa, synthetic construct
MTATAETSTGTKVVLGQNQYGKAEVRLVKVTRETDRHEIQDLNVTSQLSGDFEAAHTAGDNAHVVATDTQKNTVYAFARDGVATTEEFLLRLGKHFTEGFDWVTGGRWAAQQFFWDRINDHDHAFSRNKSEVRTAVLEISGSEQTIVSGIEGLTVLKSTGSEFHGFPRDKYTTLQETTDRILATDVSARWRYNTVEVDFDAVYGSVRGLLLKAFAETHSHALQQTMYEMGRAVIETHPEIDEVKMSLPNNHHFLVDLQPFGQDNPNEVFHAADRPYGLIEATIQREGSRAD

SEQ ID NO: 58 - protein, 291 aa, synthetic construct
MTATAETSTGTKVVLGQNQYGKAEVRLVKVTRNTDRHEIQDLNVTSQLSGDFEAAHTAGDNAHVVATDTQKNTVYAFARDGFGTTEEFLLRLGKHFTEGFDWVTGGRWAAQQHFWDRINDHDHAFSRNKSEVRTAVLEISGSEQHIVSGIEGLTVLKSTGSEFHGFPRDKYTTLQETTDRILATDVSARWRYNTEEVDFDAVYGSVRGLLLKAFAETHSHALQQTMYEMGRAVIETHPEIDEVKMSLPNKHHFLVDLQPFGQDNPNEVFHAADRPYGLIEATIQREGSRAD

SEQ ID NO: 59 - protein, 291 aa, synthetic construct
MTATAETSTGTKVVLGQNQYGKAEVRLVKVTRNTDRHEIQDLNVTSQLSGDFEAAHTAGDNARVVATDTQKNTVYAFARDGFGTTEEFLLRLGKHFTEGFDWVTGGRWAAQQFFWDRINDHDHAFSRNKSEVRTAVLEISGSEQAIVSGIEGLTVLKSTGSEFHGFPRDKYTTLQETTDRILATDVSARWRYNTDEVDFDAVYDSVRGLLLKAFAETHSHALQQTMYEMGRAVIETHPEIDEIKMSLPNKHHFLVDLQPFGQDNPNEVFYAADRPYGLIEATIQREGSRAD

SEQ ID NO: 60 - protein, 291 aa, synthetic construct
MTATAETSTGTKVVLGQNQYGKAEVRLVKVTRNTDRHEIQDLNVTSQLSGDFEAAHTAGDNAHVVATDTQKNTVYAFARDGFATTEEFLLRLGKHFTEGFDWVTGGRWAAQQFFWDRINDHDHAFSRNKSEVRTAVLEISGSEQTIVSGIEGLTVLKSTGSEFHGFPRDKYTTLQETTDRILATDVSARWRYNTVEVDFDAVHASVRGLLLKAFAETHSKALQQTMYEMGRAVIETHPEIDEIKMSLPNKHHFLVDLQPFGQDNPNEVFYAADRPYGLIEATIQREGSRAD

SEQ ID NO: 61 - protein, 291 aa, synthetic construct
MTATAETSTGTKVVLGQNQYGKAEVRLVKVTRNTDRHEIQDLNVTSQLSGDFEAAHTAGDNAHVVATDTQKNTVYAFARDGVATTEEFLLRLGKHFTEGFDWVTGGRWAAQQFFWDRINDHDHAFSRNKSEVRTAVLEISGSEQAIVSGIEGLTVLKSTGSEFHGFPRDKYTTLQETTDRILATDVSARWRYNTVEVDFDAVYGSVRGLLLKAFAETHSLALQQTMYEMGRAVIETHPEIDEVSMSLPNKHHFLVDLQPFGQDNPNEVFYAADRPYGLIEATIQREGSRAD

SEQ ID NO: 62 - protein, 291 aa, synthetic construct
MTATAKTNTGTKVVLGQNQYGKAQVRLVKVTRNTARHEIQDLNVTSQLSGDFEAAHTAGDNAHVVATDTQKNPVYAFARDGFGTTEEFLLRLGKHFTEGFDWVTGGRWAAQQHFWDRINDHDHAFSRNKSEVRTAVLEISGFEQTIVAGIEGLTVVKSTGFEFHGFPRDKYTTLQETPDCIVATDVFGRWRYNTDEVDFDAVYGSVRGLLLKGFAETHSLALQQTMYEMGRAVIETHPEIDEIKMSLPNKHHFLVDMQPFGQDNPNEVFHAADRPYGLIEATIQREGSRAD

SEQ ID NO: 63 - protein, 291 aa, synthetic construct
MTATAETSTGTKVVLGQNQYGKAEVRLVKVTRETARHEIQDLNVTSQLSGDFEAAHTAGDNAHVVATDTQKNTVYAFARDGFATTEEFLLRLGKHFTEGFDWVTGGRWAAQQFFWDRINDHDHAFSRNKSEVRTAVLEISGSEQHIVSGIEGLTVLKSTGSEFHGFPRDKYTTLQETTDRILATDVSARWRYNTVEVDFDAVYGSVRGLLLKAFAETHSHALQQTMYEMGRAVIETHPEIDEIKMSLPNNHHFLVDLQPFGQDNPNEVFYAADRPYGLIEATIQREGSRAD

SEQ ID NO: 64 - protein, 291 aa, synthetic construct
MTATAETSTGTKVVLGQNQYGKAEVRLVKVTRETDRHEIQDLNVTSQLSGDFEAAHTAGDNAHVVATDTQKNTVYAFARDGFGTTEEFLLRLGKHFTEGFDWVTGGRWAAQQHFWDRINDHDHAFSRNKSEVRTAVLEISGSEQTIVSGIEGLTVLKSTGSEFHGFPRDKYTTLQETTDRILATDVSARWRYNTEEVDFDAVYGSVRGLLLKAFAETHSLALQQTMYEMGRAVIETHPEIDEVSMSLPNNHHFLVDLQPFGQDNPNEVFHAADRPYGLIEATIQREGSRAD

SEQ ID NO: 65 - protein, 291 aa, synthetic construct
MTATAETSTGTKVVLGQNQYGKAEVRLVKVTRETARHEIQDLNVTSQLSGDFEAAHTAGDNAHVVATDTQKNTVYAFARDGFATTEEFLLRLGKHFTEGFDWVTGGRWAAQQFFWDRINDHDHAFSRNKSEVRTAVLEISGSEQAIVAGIEGLTVLKSTGSEFHGFPRDKYTTLQETTDRILATDVSARWRYNTVEVDFDAVYDSVRGLLLKAFAETHSHALQQTMYEMGRAVIETHPEIDEISMSLPNNHHFLVDLQPFGQDNPNEVFHAADRPYGLIEATIQREGSRAD

SEQ ID NO: 66 - protein, 291 aa, synthetic construct
MTATAETSTGTKVVLGQNQYGKAEVRLVKVTRETARHEIQDLNVTSQLSGDFEAAHTAGDNAHVVATDTQKNTVYAFARDGVGTTEEFLLRLGKHFTEGFDWVTGGRWAAQQFFWDRINDHDHAFSRNKSEVRTAVLEISGSEQTIVAGIEGLTVLKSTGSEFHGFPRDKYTTLQETTDRILATDVSARWRYNTVEVDFDAVYASVRGLLLKGFAETHSKALQQTMYEMGRAVIETHPEIDEIKMSLPNKHHFLVDLQPFGQDNPNEVFHAADRPYGLIEATIQREGSRAD

SEQ ID NO: 67 - protein, 291 aa, synthetic construct
MTATAETSTGTKVVLGQNQYGKAEVRLVKVTRETDRHEIQDLNVTSQLSGDFEAAHTAGDNAHVVATDTQKNTVYAFARDGVATTEEFLLRLGKHFTEGFDWVTGGRWAAQQFFWDRINDHDHAFSRNKSEVRTAVLEISGSEQHIVAGIEGLTVLKSTGSEFHGFPRDKYTTLQETTDRILATDVSARWRYNTDEVDFDAVYASVRGLLLKGFAETHSLALQQTMYEMGRAVIETHPEIDEVSMSLPNKHHFLVDLQPFGQDNPNEVFYAADRPYGLIEATIQREGSRAD

SEQ ID NO: 68 - protein, 291 aa, synthetic construct
MTATAETSTGTKVVLGQNQYGKAEVRLVKVTRNTDRHEIQDLNVTSQLSGDFEAAHTAGDNAHVVATDTQKNTVYAFARDGFATTEEFLLRLGKHFTEGFDWVTGGRWAAQQHFWDRINDHDHAFSRNKSEVRTAVLEISGSEQTIVSGIEGLTVLKSTGSEFHGFPRDKYTTLQETTDRILATDVSARWRYNTEEVDFDAVYASVRGLLLKAFAETHSKALQQTMYEMGRAVIETHPEIDEISMSLPNNHHFLVDLQPFGQDNPNEVFHAADRPYGLIEATIQREGSRAD

SEQ ID NO: 69 - protein, 291 aa, synthetic construct
MTATAETSTGTKVVLGQNQYGKAEVRLVKVTRETARHEIQDLNVTSQLSGDFEAAHTAGDNAHVVATDTQKNTVYAFARDGFATTEEFLLRLGKHFTEGFDWVTGGRWAAQQFFWDRINDHDHAFSRNKSEVRTAVLEISGSEQAIVAGIEGLTVLKSTGSEFHGFPRDKYTTLQETTDRILATDVSARWRYNTDEVDFDAVYGSVRGLLLKGFAETHSKALQQTMYEMGRAVIETHPEIDEVSMSLPNKHHFLVDLQPFGQDNPNEVFYAADRPYGLIEATIQREGSRAD

SEQ ID NO: 70 - protein, 291 aa, synthetic construct
MTATAETSTGTKVVLGQNQYGKAEVRLVKVTRNTARHEIQDLNVTSQLSGDFEAAHTAGDNAHVVATDTQKNTVYAFARDGVGTTEEFLLRLGKHFTEGFDWVTGGRWAAQQFFWDRINDHDHAFSRNKSEVRTAVLEISGSEQAIVSGIEGLTVLKSTGSEFHGFPRDKYTTLQETTDRILATDVSARWRYNTDEVDFDAVYASVRGLLLKVFAETHSKALQQTMYEMGRAVIETHPEIDEVKMSLPNNHHFLVDLQPFGQDNPNEVFYAADRPYGLIEATIQREGSRAD

SEQ ID NO: 71 - protein, 291 aa, synthetic construct
MTATAETSTGTKVVLGQNQYGKAEVRLVKVTRNTDRHEIQDLNVTSQLSGDFEAAHTAGDNAHVVATDTQKNTVYAFARDGVGTTEEFLLRLGKHFTEGFDWVTGGRWAAQQFFWDRINDHDHAFSRNKSEVRTAVLEISGSEQTIVAGIEGLTVLKSTGSEFHGFPRDKYTTLQETTDRILATDVSARWRYNTDEVDFDAVYASVRGLLLKAFAETHSKALQQTMYEMGRAVIETHPEIDEVSMSLPNNHHFLVDLQPFGQDNPNEVFHAADRPYGLIEATIQREGSRAD

SEQ ID NO: 72 - protein, 291 aa, synthetic construct
MTATAETSTGTKVVLGQNQYGKAEVRLVKVTRNTARHEIQDLNVTSQLSGDFEAAHTAGDNAHVVATDTQKNTVYAFARDGVATTEEFLLRLGKHFTEGFDWVTGGRWAAQQFFWDRINDHDHAFSRNKSEVRTAVLEISGSEQTIVAGIEGLTVLKSTGSEFHGFPRDKYTTLQETTDRILATDVSARWRYNTEEVDFDAVYGSVRGLLLKAFAETHSHALQQTMYEMGRAVIETHPEIDEVKMSLPNKHHFLVDLQPFGQDNPNEVFHAADRPYGLIEATIQREGSRAD

SEQ ID NO: 73 - protein, 291 aa, synthetic construct
MTATAETSTGTKVVLGQNQYGKAEVRLVKVTRETDRHEIQDLNVTSQLSGDFEAAHTAGDNAHVVATDTQKNTVYAFARDGFGTTEEFLLRLGKHFTEGFDWVTGGRWAAQQFFWDRINDHDHAFSRNKSEVRTAVLEISGSEQAIVAGIEGLTVLKSTGSEFHGFPRDKYTTLQETTDRILATDVSARWRYNTEEVDFDAVYASVRGLLLKAFAETHSHALQQTMYEMGRAVIETHPEIDEISMSLPNNHHFLVDLQPFGQDNPNEVFHAADRPYGLIEATIQREGSRAD

SEQ ID NO: 74 - protein, 291 aa, synthetic construct
MTATAETSTGTKVVLGQNQYGKAEVRLVKVTRETDRHEIQDLNVTSQLSGDFEAAHTAGDNAHVVATDTQKNTVYAFARDGVATTEEFLLRLGKHFTEGFDWVTGGRWAAQQHFWDRINDHDHAFSRNKSEVRTAVLEISGSEQAIVSGIEGLTVLKSTGSEFHGFPRDKYTTLQETTDRILATDVSARWRYNTVEVDFDAVYGSVRGLLLKAFAETHSKALQQTMYEMGRAVIETHPEIDEISMSLPNKHHFLVDLQPFGQDNPNEVFYAADRPYGLIEATIQREGSRAD

SEQ ID NO: 75 - protein, 291 aa, synthetic construct
MTATAETSTGTKVVLGQNQYGKAEVRLVKVTRETARHEIQDLNVTSQLSGDFEAAHTAGDNAHVVATDTQKNTVYAFARDGFATTEEFLLRLGKHFTEGFDWVTGGRWAAQQHFWDRINDHDHAFSRNKSEVRTAVLEISGSEQTIVSGIEGLTVLKSTGSEFHGFPRDKYTTLQETTDRILATDVSARWRYNTDEVDFDAVYDSVRGLLLKGFAETHSKALQQTMYEMGRAVIETHPEIDEVSMSLPNKHHFLVDLQPFGQDNPNEVFHAADRPYGLIEATIQREGSRAD

SEQ ID NO: 76 - protein, 291 aa, synthetic construct
MTATAETSTGTKVVLGQNQYGKAEVRLVKVTRNTDRHEIQDLNVTSQLSGDFEAAHTAGDNAHVVATDTQKNTVYAFARDGVGTTEEFLLRLGKHFTEGFDWVTGGRWAAQQFFWDRINDHDHAFSRNKSEVRTAVLEISGSEQAIVAGIEGLTVLKSTGSEFHGFPRDKYTTLQETTDRILATDVSARWRYNTVEVDFDAVYASVRGLLLKAFAETHSHALQQTMYEMGRAVIETHPEIDEVKMSLPNNHHFLVDLQPFGQDNPNEVFYAADRPYGLIEATIQREGSRAD

SEQ ID NO: 77 - protein, 291 aa, synthetic construct
MTATAETSTGTKVVLGQNQYGKAEVRLVKVTRNTDRHEIQDLNVTSQLSGDFEAAHTAGDNAHVVATDTQKNTVYAFARDGFGTTEEFLLRLGKHFTEGFDWVTGGRWAAQQFFWDRINDHDHAFSRNKSEVRTAVLEISGSEQTIVSGIEGLTVLKSTGSEFHGFPRDKYTTLQETTDRILATDVSARWRYNTVEVDFDAVYGSVRGLLLKGFAETHSLALQQTMYEMGRAVIETHPEIDEVSMSLPNKHHFLVDLQPFGQDNPNEVFYAADRPYGLIEATIQREGSRAD

SEQ ID NO: 78 - protein, 291 aa, synthetic construct
MTATAETSTGTKVVLGQNQYGKAEVRLVKVTRETDRHEIQDLNVTSQLSGDFEAAHTAGDNAHVVATDTQKNTVYAFARDGFATTEEFLLRLGKHFTEGFDWVTGGRWAAQQHFWDRINDHDHAFSRNKSEVRTAVLEISGSEQHIVSGIEGLTVLKSTGSEFHGFPRDKYTTLQETTDRILATDVSARWRYNTDEVDFDAVYASVRGLLLKAFAETHSLALQQTMYEMGRAVIETHPEIDEVKMSLPNNHHFLVDLQPFGQDNPNEVFHAADRPYGLIEATIQREGSRAD

SEQ ID NO: 79 - protein, 291 aa, synthetic construct
MTATAETSTGTKVVLGQNQYGKAEVRLVKVTRNTDRHEIQDLNVTSQLSGDFEAAHTAGDNAHVVATDTQKNTVYAFARDGVGTTEEFLLRLGKHFTEGFDWVTGGRWAAQQFFWDRINDHDHAFSRNKSEVRTAVLEISGSEQTIVSGIEGLTVLKSTGSEFHGFPRDKYTTLQETTDRILATDVSARWRYNTDEVDFDAVYASVRGLLLKGFAETHSKALQQTMYEMGRAVIETHPEIDEVNMSLPNKHHFLVDLQPFGQDNPNEVFHAADRPYGLIEATIQREGSRAD

SEQ ID NO: 80 - protein, 291 aa, synthetic construct
MTATAETSTGTKVVLGQNQYGKAEVRLVKVTRNTARHEIQDLNVTSQLSGDFEAAHTAGDNAHVVATDTQKNTVYAFARDGFATTEEFLLRLGKHFTEGFDWVTGGRWAAQQFFWDRINDHDHAFSRNKSEVRTAVLEISGSEQTIVAGIEGLTVLKSTGSEFHGFPRDKYTTLQETTDRILATDVSARWRYNTEEVDFDAVYASVRGLLLKAFAETHSLALQQTMYEMGRAVIETHPEIDEVSMSLPNNHHFLVDLQPFGQDNPNEVFHAADRPYGLIEATIQREGSRAD

SEQ ID NO: 81 - protein, 291 aa, synthetic construct
MTATAETSTGTKVVLGQNQYGKAEVRLVKVTRETDRHEIQDLNVTSQLSGDFEAAHTAGDNAHVVATDTQKNTVYAFARDGFATTEEFLLRLGKHFTEGFDWVTGGRWAAQQFFWDRINDHDHAFSRNKSEVRTAVLEISGSEQHIVAGIEGLTVLKSTGSEFHGFPRDKYTTLQETTDRILATDVSARWRYNTVEVDFDAVYGSVRGLLLKGFAETHSHALQQTMYEMGRAVIETHPEIDEIKMSLPNKHHFLVDLQPFGQDNPNEVFHAADRPYGLIEATIQREGSRAD

SEQ ID NO: 82 - protein, 291 aa, synthetic construct
MTATAETSTGTKVVLGQNQYGKAEVRLVKVTRNTARHEIQDLNVTSQLSGDFEAAHTAGDNAHVVATDTQKNTVYAFARDGVGTTEEFLLRLGKHFTEGFDWVTGGRWAAQQFFWDRINDHDHAFSRNKSEVRTAVLEISGSEQAIVAGIEGLTVLKSTGSEFHGFPRDKYTTLQETTDRILATDVSARWRYNTDEVDFDAVYASVRGLLLKAFAETHSLALQQTMYEMGRAVIETHPEIDEIKMSLPNNHHFLVDLQPFGQDNPNEVFHAADRPYGLIEATIQREGSRAD

SEQ ID NO: 83 - protein, 291 aa, synthetic construct
MTATAETSTGTKVVLGQNQYGKAEVRLVKVTRNTARHEIQDLNVTSQLSGDFEAAHTAGDNAHVVATDTQKNTVYAFARDGVATTEEFLLRLGKHFTEGFDWVTGGRWAAQQFFWDRINDHDHAFSRNKSEVRTAVLEISGSEQHIVSGIEGLTVLKSTGSEFHGFPRDKYTTLQETTDRILATDVSARWRYNTVEVDFDAVYASVRGLLLKGFAETHSHALQQTMYEMGRAVIETHPEIDEVKMSLPNNHHFLVDLQPFGQDNPNEVFYAADRPYGLIEATIQREGSRAD

SEQ ID NO: 84 - protein, 291 aa, synthetic construct
MTATAETSTGTKVVLGQNQYGKAEVRLVKVTRETDRHEIQDLNVTSQLSGDFEAAHTAGDNAHVVATDTQKNTVYAFARDGVATTEEFLLRLGKHFTEGFDWVTGGRWAAQQHFWDRINDHDHAFSRNKSEVRTAVLEISGSEQAIVAGIEGLTVLKSTGSEFHGFPRDKYTTLQETTDRILATDVSARWRYNTVEVDFDAVYGSVRGLLLKGFAETHSLALQQTMYEMGRAVIETHPEIDEIKMSLPNKHHFLVDLQPFGQDNPNEVFYAADRPYGLIEATIQREGSRAD

SEQ ID NO: 85 - protein, 291 aa, synthetic construct
MTATAETSTGTKVVLGQNQYGKAEVRLVKVTRETARHEIQDLNVTSQLSGDFEAAHTAGDNAHVVATDTQKNTVYAFARDGVGTTEEFLLRLGKHFTEGFDWVTGGRWAAQQFFWDRINDHDHAFSRNKSEVRTAVLEISGSEQTIVSGVEGLTVLKSTGSEFHGFPRDKYTTLQETTDRILATDVSARWRYNTEEVDFDAVYDSVRGLLLKAFAETHSKALQQTMYEMGRAVIETHPEIDEVKMSLPNNHHFLVDLQPFGQDNPNEVFYAADRPYGLIEATIQREGSRAD

SEQ ID NO: 86 - protein, 291 aa, synthetic construct
MTATAETSTGTKVVLGQNQYGKAEVRLVKVTRETARHEIQDLNVTSQLSGDFEAAHTAGDNAHVVATDTQKNTVYAFARDGVGTTEEFLLRLGKHFTEGFDWVTGGRWAAQQFFWDRINDHDHAFSRNKSEVRTAVLEISGSEQAIVAGIEGLTVLKSTGSEFHGFPRDKYTTLQETTDRILATDVSARWRYNTEEVDFDAVYGSVRGLLLKGFAETHSLALQQTMYEMGRAVIETHPEIDEIKMSLPNKHHFLVDLQPFGQDNPNEVFHAADRPYGLIEATIQREGSRAD

SEQ ID NO: 87 - protein, 291 aa, synthetic construct
MTATAETSTGTKVVLGQNQYGKAEVRLVKVTRNTDRHEIQDLNVTSQLSGDFEAAHTAGDNAHVVATDTQKNTVYAFARDGFATTEEFLLRLGKHFTEGFDWVTGGRWAAQQFFWDRINDHDHAFSRNKSEVRTAVLEISGSEQTIVSGIEGLTVLKSTGSEFHGFPRDKYTTLQETTDRILATDVSARWRYNTVEVDFDAVHASVRGLLLKGFAETHSLALQQTMYEMGRAVIETHPEIDEVSMSLPNKHHFLVDLQPFGQDNPNEVFHVADRPYGLIEAIIQREGSRAD

SEQ ID NO: 88 - protein, 291 aa, synthetic construct
MTATAETSTGTKVVLGQNQYGKAEVRLVKVTRNTARHEIQDLNVTSQLSGDFEAAHTAGDNAHVVATDTQKNTVYAFARDGVATTEEFLLRLGKHFTEGFDWVTGGRWAVQQFFWDRINDHDHAFSRNKSEVRTAVLEISGSEQAIVAGIEGLTVLKSTGSEFHGFPRDKYTTLQETTDRILATDVSARWRYNTVEVDFDAVYDSVRGLLLKAFAETHSKALQQTMYEMGRAVIETHPEIDEIKMSLPNKHHFLVDLQPFGQDNPNEVFHAADRPYGLIEATIQREGSRAD

SEQ ID NO: 89 - protein, 291 aa, synthetic construct
MTATAETSTGTKVVLGQNQYGKAEVRLVKVTRNTARHEIQDLNVTSQLSGDFEAAHTAGDNAHVVATDTQKNTVYAFARDGFGTTEEFLLRLGKHFTEGFDWVTGGRWAAQQFFWDRINDHDHAFSRNKSEVRTAVLEISGSEQAIVSGIEGLTVLKSTGSEFHGFPRDKYTTLQETTDRILATDVSARWRYNTVEVDFDAVYASVRGLLLKAFAETHSLALQQTMYEMGRAVIETHPEIDEVKMGLPNKHHFLVDLQPFGQDNPNEVFHAADRPYGLIEATIQREGSRAD

SEQ ID NO: 90 - protein, 291 aa, synthetic construct
MTATAETSTGTKVVLGQNQYGKAEVRLVKVTRNTARHEIQDLNVTSQLSGDFEAAHTAGDNAHVVATDTQKNTVYAFARDGFGTTEEFLLRLGKHFTEGFDWVTGGRWAAQQFFWDRINDHDHAFSRNKSEVRTAVLEISGSEQTIVSGIEGLTVLKSTGSEFHGFPRDKYTTLQETTDRILATDVSARWRYNTVEVDFDAVYGSVRGLLLKAFAETHSLALQQTMYEMGRAVIETHPEIDEVKMSLPNNHHFLVDLQPFGQDNPNEVFYAADRPYGLIEATIQREGSRAD

SEQ ID NO: 91 - protein, 291 aa, synthetic construct
MTATAETSTGTKVVLGQNQYGKAEVRLVKVTRNTARHEIQDLNVTSQLSGDFEAAHTAGDNAHVVATDTQKNTVYAFARDGFATTEEFLLRLGKHFTEGFDWVTGGRWAAQQHFWDRINDHDHAFSRNKSEVRTAVLEISGSEQTIVSGIEGLTVLKSTGSEFHGFPRDKYTTLQETTDRILATDVSARWRYNTVEVDFDAVYDSVRGLLLKGFAETHSHALQQTMYEMGRAVIETHPEIDEVSMSLPNNHHFLVDLQPFGQDNPNEVFHAADRPYGLIEATIQREGSRAD

SEQ ID NO: 92 - protein, 291 aa, synthetic construct
MTATAETSTGTKVVLGQNQYGKAEVRLVKVTRNTARHEIQDLNVTSQLSGDFEAAHTAGDNAHVVATDTQKNTVYAFARDGVGTTEEFLLRLGKHFTEGFDWVTGGRWAAQQYFWDRINDHDHAFSRNKSEVRTAVLEISGSEQAIVAGIEGLTVLKSTGSEFHGFPRDKYTTLQETTDRILATDVSARWRYNTEEVDFDAVYDSVRGLLLKAFAETHSHALQQTMYEMGRAVIETHPEIDEIKMSLPNKHHFLVDLQPFGQDNPNEVFYAADRPYGLIEATIQREGSRAD

SEQ ID NO: 93 - protein, 291 aa, synthetic construct
MTATAETSTGTKVVLGQNQYGKAEVRLVKVTRETARHEIQDLNVTSQLSGDFEAAHTAGDNAHVVATDTQKNTVYAFARDGVGTTEEFLLRLGKHFTEGFDWVTGGRWAAQQHFWDRINDHDHAFSRNKSEVRTAVLEISGSEQTIVSGIEGLTVLKSTGSEFHGFPRDKYTTLQETTDRILATDVSARWRYNTEEVDFDAVYDSVRGLLLKAFAETHSHALQQTMYEMGRAVIETHPEIDEVKMSLPNNHHFLVDLQPFGQDNPNEVFYAADRPYGLIEATIQREGSRAD

SEQ ID NO: 94 - protein, 291 aa, synthetic construct
MTATAETSTGTKVVLGQNQYGKAEVRLVKVTRNTDRHEIQDLNVTSQLSGDFEAAHTAGDNAHVVATDTQKNTVYAFARDGFGTTEEFLLRLGKHFTEGFDWVTGGRWAAQQFFWDRINDHDHAFSRNKSEVRTAVLEISGSEQTIVAGIEGLTVLKSTGSEFHGFPRDKYTTLQETTDRILATDVSAHWRYNTVEVDFDAVYASVRGLLLKGFAETHSLALQQTMYEMGRAVIETHPEIDEVKMSLPNNHHFLVDLQPFGQDNPNEVFYAADRPYGLIEATIQREGSRAD

SEQ ID NO: 95 - protein, 291 aa, synthetic construct
MTATAETSTGTKVVLGQNQYGKAEVRLVKVTRNTDRHEIQDLNVTSQLSGDFEAAHTAGDNAHVVATDTQKNTVYAFARDGFGTTEEFGLRLGKHFTEGFDWVTGGRWAAQQFFWDRINDHDHAFSRNKSEVRTAVLEISGSEQAIVAGIEGLTVLKSTGSEFHGFPRDKYTTLQETTDRILATDVSARWRYNTEEVDFDAVHESVRGLLLKGFAETHSHALQQTMYEMGRAVIETHPEIDEVSMSLPNNHHFLVDLQPFGQDNPNEVFYAADRPYGLIEATIQREGSRAD

SEQ ID NO: 96 - protein, 291 aa, synthetic construct
MTATAETSTGTKVVLGQNQYGKAEVRLVKVTRETARHEIQDLNVTSQLSGDFEAAHTAGDNAHVVATDTQKNTVYAFARDGFATTEEFLLRLGKHFTEGFDWVTGGRWAAQQFFWDRINDHDHAFSRNKSEVRTAVLEISGSEQTIVSGIEGLTVLKSTGSEFHGFPRDKYTTLQETTDRILATDVSARWRYNTDEVDFDAVYDSVRGLLLKGFAETHSLALQQTMYEMGRAVIETHPEIDEISMSLPNNHHFLVDLQPFGQDNPNEVFHAADRPYGLIEATIQREGSRAD

SEQ ID NO: 97 - protein, 291 aa, synthetic construct
MTATAETSTGTKVVLGQNQYGKAEVRLVKVTRNTDRHEIQDLNVTSQLSGDFEAAHTAGDNAHVVVTDTQKNTVYAFARDGVATTEEFLLRLGKHFTEGFDWVTGGRWAAQQFFWDRINDHDHAFSRNKSEVRTAVLEISGSEQTIVAGIEGLTVLKSTGSEFHGFPRDKYTTLQETTDRILATDVSARWRYNTVEVDFDAVYASVRGLLLKGFAETHSKALQQTMYEMGRAVIETHPEIDEVKMSLPNKHHFLVDLQPFGQDNPNEVFYAADRPYGLIEATIQREGSRAD

SEQ ID NO: 98 - protein, 291 aa, synthetic construct
MTATAETSTGTKVVLGQNQYGKAEVRLVKVTRNTDRHEIQDLNVTSQLSGDFEAAHTAGDNAHVVATDTQKNTVYAFARDGFATTEEFLLRLGKHFTEGFDWVTGGRWAAQQHFWDRINDHDHAFSRNKSEVRTAVLEISGSEQTIVAGIEGLTVLKSTGSEFHGFPRDKYTTLQETTDRILATDVSARWRYNTEEVDFDAVYDSVRGLLLKGFAETHSKALQQTMYEMGRAVIETHPEIDEIKMSLPNNHHFLVDLQPFGQDNPNEVFYAADRPYGLIEATIQREGSRAD

SEQ ID NO: 99 - protein, 291 aa, synthetic construct
MTATAETSTGTKVVLGQNQYGKAEVRLVKVTRNTARHEIQDLNVTSQLSGDFEAAHTAGDNAHVVATDTQKNTVYAFARDGVATTEEFLLRLGKHFTEGFDWVTGGRWAAQQFFWDRINDHDHAFSRNKSEVRTAVLEISGSEQTIVSGIEGLTVLKSTGSEFHGFPRDKYTTLQETTDRILATDVSARWRYNTDEVDFDAVYASVRGLLLKGFAETHSLALQQTMYEMGRAVIETHPEIDEVKMSLPNKHHFLVDLQPFGQDNPNEVFHAADRPYGLIEATIQREGSRAD

SEQ ID NO: 100 - protein, 291 aa, synthetic construct
MTATAETSTGTKVVLGQNQYGKAEVRLVKVTRNTDRHEIQDLNVTSQLSGDFEAAHTAGDNAHVVATDTQKNTVYAFARDGVGTTEEFLLRLGKHFTEGFDWVTGGRWAAQQFFWDRINDHDHAFSRNKSEVRTAVLEISGSEQAIVSGIEGLTVLKSTGSEFHGFPRDKYTTLQETTDRILATDVSARWRYNTDEVDFDAVYASVRGLLLKAFAETHSLALQQTMYEMGRAVIETHPEIDEISMSLPNNHHFLVDLQPFGQDNPNEVFHAADRPYGLIEATIQREGSRAD

SEQ ID NO: 101 - protein, 291 aa, synthetic construct
MTATAETSTGTKVVLGQNQYGKAEVRLVKVTRETDRHEIQDLNVTSQLSGDFEAAHTAGDNAHVVATDTQKNTVYAFARDGFGTTEEFLLRLGKHFTEGFDWVTGGRWAAQQHFWDRINDHDHAFSRNKSEVRTAVLEISGSEQTIVSGIEGLTVLKSTGSEFHGFPRDKYTTLQETTDRILATDVSARWRYNTDEVDFDAVYGSVRGLLLKGFAETHSKALQQTMYEMGRAVIETHPEIDEIKMSLPNKHHFLVDLQPFGQDNPNEVFYAADRPYGLIEATIQREGSRAD

SEQ ID NO: 102 - protein, 291 aa, synthetic construct
MTATAETSTGTKVVLGQNQYGKAEVRLVKVTRNTDRHEIQDLNVTSQLSGDFEAAHTAGDNAHVVATDTQKNTVYAFARDGVATTEEFLLRLGKHFTEGFDWVTGGRWAAQQFFWDRINDHDHAFSRNKSEVRTAVLEISGSEQAIVAGIEGLTVLKSTGSEFHGFPRDKYTTLQETADRILATDVSARWRYNTDEVDFDAVYASVRGLLLKGFAETHSLALQQTMYEMGRAVIETHPEIDEVSMSLPNKHHFLVDLQPFGQDNPNEVFYAADRPYGLIEATIQREGSRAD

SEQ ID NO: 103 - protein, 291 aa, synthetic construct
MTATAETSTGTKVVLGQNQYGKAEVRLVKVTRETARHEIQDLNVTSQLSGDFEAAHTAGDNAHVVATDTQKNTVYAFARDGFATTEEFLLRLGKHFTEGFDWVTGGRWAAQQFFWDRINDHDHAFSRNKSEVRTAVLEISGSEQTIVAGIEGLTVLKSTGSEFHGFPRDKYTTLQETTDRILATDVSARWRYNTVEVDFDAVHGSVRGLLLKAFAETHSLALQQTMYEMGRAVIETHPEIDEVKMSLPNNHHFLVDLQPFGQDNPNEVFYAADRPYGLIEATIQREGSRAD

SEQ ID NO: 104 - protein, 291 aa, synthetic construct
MTATAETSTGTKVVLGQNQYGKAEVRLVKVTRETDRHEIQDLNVTSQLSGDFEAAHTAGDNAHVVATDTQKNTVYAFARDGFATTEEFLLRLGKHFTEGFDWVTGGRWAAQQHFWDRINDHDHAFSRNKSEVRTAVLEISGSEQAIVAGIEGLTVLKSTGSEFHGFPRDKYTTLQETTDRILATDVSARWRYNTEEVDFDAVYASVRGLLLKAFAETHSHALQQTMYEMGRAVIETHPEIDEISMSLPNNHHFLVDLQPFGQDNPNEVFYAADRPYGLIEATIQREGSRAD

SEQ ID NO: 105 - protein, 291 aa, synthetic construct
MTATAETSTGTKVVLGQNQYGKAEVRLVKVTRNTARHEIQDLNVTSQLSGDFEAAHTAGDNAHVVATDTQKNTVYAFARDGFGTTEEFLLRLGKHFTEGFDWVTGGRWAAQQFFWDRINDHDHAFSRNKSEVRTAVLEISGSEQAIVSGIEGLTVLKSTGSEFHGFPRDKYTTLQETTDRILATDVSARWRYNTDEVDFDAVYDSVRGLLLKGFAETHSLALQQTMYEMGRAVIETHPEIDEVKMSLPNNHHFLVDLQPFGQDNPNEVFYAADRPYGLIEATIQREGSRAD

SEQ ID NO: 106 - protein, 291 aa, synthetic construct
MTATAETSTGTKVVLGQNQYGKAEVRLVKVTRNTARHEIQDLNVTSQLSGDFEAAHTAGDNAHVVATDTQKNTVYAFARDGVATTEEFLLRLGKHFTEGFDWVTGGRWAAQQHFWDRINDHDHAFSRNKSEVRTAVLEISGSEQTIVSGIEGLTVLKSTGSEFHGFPRDKYTTLQETTDRILATDVSARWRYNTVEVDFDAVYGSVRGLLLKAFAETHSKALQQTMYEMGRAVIETHPEIDEVSMSLPNKHHFLVDLQPFGQDNPNEVFYAADRPYGLIEATIQREGSRAD

SEQ ID NO: 107 - protein, 291 aa, synthetic construct
MTATAETSTGTKVVLGQNQYGKAEVRLVKVTRETARHEIQDLNVTSQLSGDFEAAHTAGDNAHVVATDTQKNTVYAFARDGFGTTEEFLLRLGKHFTEGFDWVTGGRWAAQQFFWDRINDHDHAFSRNKSEVRTAVLEISGSEQAIVSGIEGLTVLKSTGSEFHGFPRDKYTTLQETTDRILATDVSARWRYNTDEVDFDAVYDSVRGLLLKAFAETHSKALQQTMYEMGRAVIETHPEIDEVSMSLPNKHHFLVDLQPFGQDNPNEVFHAADRPYGLIEATIQREGSRAD

SEQ ID NO: 108 - protein, 291 aa, synthetic construct
MTATAETSTGTKVVLGQNQYGKAEVRLVKVTRETDRHEIQDLNVTSQLSGDFEAAHTAGDNAHVVATDTQKNTVYAFARDGFATTEEFLLRLGKHFTEGFDWVTGGRWAAQQFFWDRINDHDHAFSRNKSEVRTAVLEISGSEQAIVSGIEGLTVLKSTGSEFHGFPRDKYTTLQETTDRILATDVSARWRYNTVEVDFDAVHGSVRGLLLKAFAETHSLALQQTMYEMGRAVIETHPEIDEVSMSLPNKHHFLVDLQPFGQDNPNEVFYAADRPYGLIEATIQREGSRAD

SEQ ID NO: 109 - protein, 291 aa, synthetic construct
MTATAETSTGTKVVLGQNQYGKAEVRLVKVTRNTARHEIQDLNVTSQLSGDFEAAHTAGDNAHVVATDTQKNTVYAFARDGFATTEEFLLRLGKHFTEGFDWVTGGRWAAQQFFWDRINDHDHAFSRNKSEVRTAVLEISGSEQHIVSGIEGLTVLKSTGSEFHGFPRDKYTTLQETTDRILATDVSARWRYNTEEVDFDAVYASVRGLLLKGFAETHSLALQQTMYEMGRAVIETHPEIDEVSMSLPNNHHFLVDLQPFGQDNPNEVFYAADRPYGLIEATIQREGSRAD

SEQ ID NO: 110 - protein, 291 aa, synthetic construct
MTATAETSTGTKVVLGQNQYGKAEVRLAKVTRETARHEIQDLNVTSQLSGDFEAAHTAGDNAHVVATDTQKNTVYAFARDGFGTTEEFLLRLGKHFTEGFDWVTGGRWAAQQFFWDRINDHDHAFSRNKSEVRTAVLEISGSEQTIVAGIEGLTVLKSTGSEFHGFPRDKYTTLQETTDRILATDVSARWRYNTVEVDFDAVYASVRGLLLKGFAETHSLALQQTMYEMGRAVIETHPEIDEVSMSLPNKHHFLVDLQPFGQDNPNEVFYAADRPYGLIEATIQREGSRAD

SEQ ID NO: 111 - protein, 291 aa, synthetic construct
MTATAETSTGTKVVLGQNQYGKAEVRLVKVTRNTARHEIQDLNVTSQLSGDFEAAHTAGDNAHVVATDTQKNTVYAFARDGVATTEEFLLRLGKHFTKGFDWVTGGRWAAQQHFWDRINDHDHAFSRNKSEVRTAVLEISGSEQHIVSGIEGLTVLKSTGSEFHGFPRDKYTTLQETTDRILATDVSARWRYNTVEVDFDAVYASVRGLLLKGFAETHSLALQQTMYEMGRAVIETHPEIDEIKMSLPNKHHFLVDLQPFGQDNPNEVFYAADRPYGLIEATIQREGSRAD

SEQ ID NO: 112 - protein, 291 aa, synthetic construct
MTATAETSTGTKVVLGQNQYGKAEVRLVKVTRETARHEIQDLNVTSQLSGDFEAAHTAGDNAHVVATDTQKNTVYAFARDGFGTTEEFLLRLGKHFTEGFDWVTGGRWAAQQHFWDRINDHDHAFSRNKSEVRTAVLEISGSEQAIVAGIEGLTVLKSTGSEFHGFPRDKYTTLQETTDRILATDVSARWRYNTVEVDFDAVYASVRGLLLKAFAETHSHALQQTMYEMGRAVIETHPEIDEVSMSLPNNHHFLVDLQPFGQDNPNEVFYAADRPYGLIEATIQREGSRAD

SEQ ID NO: 113 - protein, 291 aa, synthetic construct
MTATAETSTGTKVVLGQNQYGKAEVRLVKVTRETDRHEIQDLNVTSQLSGDFEAAHTAGDNAHVVATDTQKNTVYAFARDGFATTEEFLLRLGKHFTEGFDWVTGGRWAAQQHFWDRINDHDHAFSRNKSEVRTAVLEISGSEQTIVSGIEGLTVLKSTGSEFHGFPRDKYTTLQETTDRILATDVSARWRYNTDEVDFDAVYDSVRGLLLKAFAETHSKALQQTMYEMGRAVIETHPEIDEVSMSLPNKHHFLVDLQPFGQDNPNEVFHAADRPYGLIEATIQREGSRAD

SEQ ID NO: 114 - protein, 291 aa, synthetic construct
MTATAETSTGTKVVLGQNQYGKAEVRLVKVTRNTARHEIQDLNVTSQLSGDFEAAHTAGDNAHVVATDTQKNTVYAFARDGFATTEEFLLRLGKHFTEGFDWVTGGRWAAQQHFWDRINDHDHAFSRNKSEVRTAVLEISGSEQTIVSGIEGLTVLKSTGSEFHGFPRDKYTTLQETTDRILATDVSARWRYNTEEVDFDAVHASVRGLLLKAFAETHSKALQQTMYEMGRAVIETHPEIDEIKMSLPNKHHFLVDLQPFGQDNPNEVFYAADRPYGLIEATIQREGSRAD

SEQ ID NO: 115 - protein, 291 aa, synthetic construct
MTATAETSTGTKVVLGQNQYGKAEVRLVKVTRETARHEIQDLNVTSQLSGDFEAAHTAGDNAHVVATDTQKNTVYAFARDGFATTEEFLLRLGKHFTEGFDWVTGGRWAAQQFFWDRINDHDHAFSRNKSEVRTAVLEISGSEQAIVAGIEGLTVLKSTGSEFHGFPRDKYTTLQETTDRILATDVSARWRYNTEEVDFDAVYGSVRGLLLKAFAETHSHALQQTMYEMGRAVIETHPEIDEIKMSLPNKHHFLVDLQPFGQDNPNEVFYAADRPYGLIEATIQREGSRAD

SEQ ID NO: 116 - protein, 291 aa, synthetic construct
MTATAETSTGTKVVLGQNQYGKAEVRLVKVTRNTARHEIQDLNVTSQLSGDFEAAHTAGDNAHVVATDTQKNTVYAFARDGVGTTEEFLLRLGKHFTEGFDWVTGGRWAAQQFFWDRINDHDHAFSRNKSEVRTAVLEISGSEQAIVSGIEGLTVLKSTGSEFHGFPRDKYTTLQETTDRILATDVSARWRYNTEEVDFDAVYGSVRGLLLKGFAETHSLALQQTMYEMGRAVIETHPEIDEISMSLPNNHHFLVDLQPFGQDNPNEVFHAADRPYGLIEATIQREGSRAD

SEQ ID NO: 117 - protein, 291 aa, synthetic construct
MTATAETSTGTKVVLGQNQYGKAEVRLVKVTRETARHEIQDLNVTSQLSGDFEAAHTAGDNAHVVATDTQKNTVYAFARDGFATTEEFLLRLGKHFTEGFDWVTGGRWAAQQFFWDRINDHDHAFSRNKSEVRTAVLEISGSEQHIVAGIEGLTVLKSTGSEFHGFPRDKYTTLQETTDRILATDVSARWRYNTDEVDFDAVYDSVRGLLLKAFAETHSLALQQTMYEMGRAVIETHPEIDEVKMSLPNNHHFLVDLQPFGQDNPNEVFHAADRPYGLIEATIQREGSRTD

SEQ ID NO: 118 - protein, 291 aa, synthetic construct
MTATAETSTGTKVVLGQNQYGKAEVRLVKVTRETDRHEIQDLNVTSQLSGDFEAAHTAGDNAHVVATDTQKNTVYAFARDGVGTTEEFLLRLGKHFTEGFDWVTGGRWAAQQFFWDRINDHDHAFSRNKSEVRTAVLEISGSEQTIVAGIEGLTVLKSTGSEFHGFPRDKYTTLQETTDRILATDVSARWRYNTVEVDFDAVYDSVRGLLLKGFAETHSHALQQTMYEMGRAVIETHPEIDEIKMSLPNNHHFLVDLQPFGQDNPNEVFYAADRPYGLIEATIQREGSRAD

SEQ ID NO: 119 - protein, 291 aa, synthetic construct
MTATAETSTGTKVVLGQNQYGKAEVRLVKVTRETARHEIQDLNVTSQLSGDFEAAHTAGDNAHVVATDTQKNTVYAFARDGFGTTEEFLLRLGKHFTEGFDWVTGGRWAAQQFFWDRINDHDHAFSRNKSEVRTAVLEISGSEQTIVSGIEGLTVLKSTGSEFHGFPRDKYTTLQETTDRILATDVSARWRYNTEEVDFDAVYASVRGLLLKAFAETHSHALQQTMYEMGRAVIETHPEIDEIKMSLPNNHHFLVDLQPFGQDNPNEVFHAADRPYGLIEATIQREGSRAD

SEQ ID NO: 120 - protein, 291 aa, synthetic construct
MTATAETSTGTKVVLGQNQYGKAEVRLVKVTRNTDRHEIQDLNVTSQLSGDFEAAHTAGDNAYVVATDTQKNTVYAFARDGFATTEEFLLRLGKHFTEGFDWVTGGRWAAQQHFWDRINDHDHAFSRNKSEVRTAVLEISGSEQTIVSGIEGLTVLKSTGSEFHGFPRDKYTTLQETTDRILATDVSARWRYNTEEVDFDAVYGSVRGLLLKAFAETHSKALQQTMYEMGRAVIETHPEIDEVSMSLPNNHHFLVDLQPFGQDNPNEVFYAADRPYGLIEATIQREGSRAD

SEQ ID NO: 121 - protein, 291 aa, synthetic construct
MTATAETSTGTKVVLGQNQYGKAEVRLVKVTRETDRHEIQDLNVTSQLSGDFEAAHTAGDNAHVVATDTQKNTVYAFARDGFGTTEEFLLRLGKHFTEGFDWVTGGRWAAQQHFWDRINDHDHAFSRNKSEVRTAVLEISGSEQHIVAGIEGLTVLKSTGSEFHGFPRDKYTTLQETTDRILATDVSARWRYNTVEVDFDAVYASVRGLLLKAFAETHSKALQQTMYEMGRAVIETHPEIDEVSMSLPNKHHFLVDLQPFGQDNPNEVFHAADRPYGLIEATIQREGSRAD

SEQ ID NO: 122 - protein, 291 aa, synthetic construct
MTATAETSTGTKVVLGQNQYGKAEVRLVKVTRNTARHEIQDLNVTSQLSGDFEAAHTAGDNAHVVATDTQKNTVYAFARDGFGTTEEFLLRLGKHFTEGFDWVTGGRWAAQQHFWDRINDHDHAFSRNKSEVRTAVLEISGSEQTIVAGIEGLTVLKSTGSEFHGFPRDKYTTLQETTDRILATDVSARWRYNTVEVDFDAVYGSVRGLLLKAFAETHSLALQQTMYEMGRAVIETHPEIDEIKMSLPNKHHFLVDLQPFGQDNPNEVFHAADRPYGLIEATIQREGSRAD

SEQ ID NO: 123 - protein, 291 aa, synthetic construct
MTATAETSTGTKVVLGQNQYGKAEVRLVKVTRETDRHEIQDLNVTSQLSGDFEAAHTAGDNAHVVATDTQKNTVYAFARDGFATTEEFLLRLGKHFTEGFDWVTGGRWAAQQFFWDRINDHDHAFSRNKSEVRTAVLEISGSEQHIVSGIEGLTVLKSTGSEFHGFPRDKYTTLQETTDRILATDVSARWRYNTEEVDFDAVYDSVRGLLLKAFAETHSHALQQTMYEMGRAVIETHPEIDEVKMSLPNKHHFLVDLQPFGQDNPNEVFYAADRPYGLIEATIQREGSRAD

SEQ ID NO: 124 - protein, 291 aa, synthetic construct
MTATAETSTGTKVVLGQNQYGKAEVRLVKVTRNTDRHEIQDLNVTSQLSGDFEAAHTAGDNAHVVATDTQKNTVYAFARDGVGTTEEFLLRLGKHFTEGFDWVTGGRWAAQQFFWDRINDHDHAFSRNKSEVRTAVLEISGSEQHIVSGIEGLTVLKSTGSEFHGFPRDKYTTLQETTDRILATDVSARWRYNTEEVDFDAVYASVRGLLLKGFAETHSKALQQTMYEMGRAVIETHPEIDEIKMSLPNKHHFLVDLQPFGQDNPNEVFYAADRPYGLIEATIQREGSRAD

SEQ ID NO: 125 - protein, 291 aa, synthetic construct
MTATAETSTGTKVVLGQNQYGKAEVRLVKVTRETARHEIQDLNVTSQLSGDFEAAHTAGDNAHVVATDTQKNTVYAFARDGFGTTEEFLLRLGKHFTEGFDWVTGGRWAAQQHFWDRINDHDHAFSRNKSEVRTAVLEISGSEQTIVSGIEGLTVLKSTGSEFHGFPRDKYTTLQETTDRILATDVSARWRYNTDEVDFDAVYGSVRGLLLKAFAETHSKALQQTMYEMGRAVIETHPEIDEVKMSLPNKHHFLVDLQPFGQDNPNEVFYAADRPYGLIEATIQREGSRAD

SEQ ID NO: 126 - protein, 291 aa, synthetic construct
MTATAETSTGTKVVLGQNQYGKAEVRLVKVTRETARHEIQDLNVTSQLSGDFEAAHTAGDNAHVVATDTQKNTVYAFARDGFGTTEEFLLRLGKHFTEGFDWVTGGRWAAQQFFWDRINDHDHAFSRNKSEVRTAVLEISGSEQVIVAGIEGLTVLKSTGSEFHGFPRDKYTTLQETTDRILATDVSARWRYNTEEVDFDAVYASVRGLLLKAFAETHSKALQQTMYEMGRAVIETHPEIDEIKMSLPNNHHFLVDLQPFGQDNPNEVFHAADRPYGLIEATIQREGSRAD

SEQ ID NO: 127 - protein, 291 aa, synthetic construct
MTATAETSTGTKVVLGQNQYGKAEVRLVKVTRNTDRHEIQDLNVTSQLSGDFEAAHTAGDNAHVVATDTQKNTVYAFARDGFGTTEEFLLRLGKHFTEGFDWVTGGRWAAQQHFWDRINDHDHAFSRNKSEVRTAVLEISGSEQAIVAGIEGLTVLKSTGSEFHGFPRDKYTTLQETTDRILATDVSARWRYNTEEVDFDAVYGSVRGLLLKAFAETHSKALQQTMYEMGRAVIETHPEIDEVSMSLPNKHHFLVDLQPFGQDNPNEVFYAADRPYGLIEATIQREGSRAD

SEQ ID NO: 128 - protein, 291 aa, synthetic construct
MTATAETSTGTKVVLGQNQYGKAEVRLVKVTRETARHEIQDLNVTSQLSGDFEAAHTAGDNAHVVATDTQKNTVYAFARDGFATTEEFLLRLGKHFTEGFDWVTGGRWAAQQHFWDRINDHDHAFSRNKSEVRTAVLEISGSEQHIVSGIEGLTVLKSTGSEFHGFPRDKYTTLQETTDRILATDVSARWRYNTEEVDFDAVYDSVRGLLLKGFAETHSLALQQTMYEMGRAVIETHPEIDEVKMSLPNKHHFLVDLQPFGQDNPNEVFYAADRPYGLIEATIQREGSRAD

SEQ ID NO: 129 - protein, 291 aa, synthetic construct
MTATAETSTGTKVVLGQNQYGKAEVRLVKVTRNTARHEIQDLNVTSQLSGDFEAAHTAGDNAHVVATDTQKNTVYAFARDGFATTEEFLLRLGKHFTEGFDWVTGGRWAAQQFFWDRINDHDHAFSRNKSEVRTAVLEISGSEQAIVAGIEGLTVLKSTGSEFHGFPRDKYTTLQETTDRILATDVSARWRYNTEEVDFDAVYGSVRGLLLKAFAETHSLALQQTMYEMGRAVIETHPEIDEVSMSLPNKHHFLVDLQPFGQDNPNEVFYAADRPYGLIEATIQREGSRAD

SEQ ID NO: 130 - protein, 291 aa, synthetic construct
MTATAETSTGTKVVLGQNQYGKAEVRLVKVTRETARHEIQDLNVTSQLSGDFEAAHTAGDNAHVVATDTQKNTVYAFARDGFGTTEEFLLRLGKHFTEGFDWVTGGRWAAQQFFWDRINDHDHAFSRNKSEVRTAVLEISGSEQAIVAGIEGLTVLKSTGSEFHGFPRDKYTTLQETTDRILATDVSARWRYNTDEVDFDAVYASVRGLLLKGFAETHSKALQQTMYEMGRAVIETHPEIDEIKMSLPNKHHFLVDLQPFGQDNPNEVFYAADRPYGLIEATIQREGSRAD

SEQ ID NO: 131 - protein, 291 aa, synthetic construct
MTATAETSTGTKVVLGQNQYGKAEVRLVKVTRETARHEIQDLNVTSQLSGDFEAAHTAGDNAHVVATDTQKNTVYAFARDGFATTEEFLLRLGKHFTEGFDWVTGGRWAAQQHFWDRINDHDHAFSRNKSEVRTAVLEISGSEQTIVAGIEGLTVLKSTGSEFHGFPRDKYTTLQETTDRILATDVSARWRYNTVEVDFDAVYASVRGLLLKAFAETHSLALQQTMYEMGRAVIETHPEIDEVKMSLPNNHHFLVDLQPFGQDNPNEVFYAADRPYGLIEATIQREGSRAD

SEQ ID NO: 132 - protein, 291 aa, synthetic construct
MTATAETSTGTKVVLGQNQYGKAEVRLVKVTRNTDRHEIQDLNVTSQLSGDFEAAHTAGDNAHVVATDTQKNTVYAFARDGFGTTEEFLLRLGKHFTEGFDWVTGGRWAAQQFFWDRINDHDHAFSRNKSEVRTAVLEISGSEQHIVAGIEGLTVLKSTGSEFHGFPRDKYTTLQETTDRILATDVSARWRYNTVEVDFDAVYASVRGLLLKGFAETHSKALQQTMYEMGRAVIETHPEIDEISMSLPNNHHFLVDLQPFGQDNPNEVFHAADRPYGLIEATIQREGSRAD

SEQ ID NO: 133 - protein, 291 aa, synthetic construct
MTATAETSTGTKVVLGQNQYGKAEVRLVKVTRNTARHEIQDLNVTSQLSGDFEAAHTAGDNAHVVATDTQKNTVYAFARDGFATTEEFLLRLGKHFTEGFDWVTGGRWAAQQHFWDRINDHDHAFSRNKSEVRTAVLEISGSEQTIVAGIEGLTVLKSTGSEFHGFPRDKYTTLQETTDRILATDVSARWRYNTDEVDFDAVYGSVRGLLLKGFAETHSLALQQTMYEMGRAVIETYPEIDEIKMSLPNKHHFLVDLQPFGQDNPNEVFYAADRPYGLIEATIQREGSRAD

SEQ ID NO: 134 - protein, 291 aa, synthetic construct
MTATAETSTGTKVVLGQNQYGKAEVRLVKVTRNTDRHEIQDLNVTSQLSGDFEAAHTAGDNAHVVATDTQKNTVYAFARDGFGTTEEFLLRLGKHFTEGFDWVTGGRWAAQQFFWDRINDHDHAFSRNKSEVRTAVLEISGSEQTIVSGIEGLTVLKSTGSEFHGFPRDKYTTLQETTDRILATDVSARWRYNTEEVDFDAVYDSVRGLLLKGFAETHSHALQQTMYEMGRAVIETHPEIDEVSMSLPNKHHFLVDLQPFGQDNPNEVFYAADRPYGLIEATIQREGSRAD

SEQ ID NO: 135 - protein, 291 aa, synthetic construct
MTATAETSTGTKVVLGQNQYGKAEVRLVKVTRNTDRHEIQDLNVTSQLSGDFEAAHTAGDNAHVVATDTQKNTVYAFARDGVATTEEFLLRLGKHFTEGFDWVTGGRWAAQQHFWDRINDHDHAFSRNKSEVRTAVLEISGSEQHIVAGIEGLTVLKSTGSEFHGFPRDKYTTLQETTDRILATDVSARWRYNTEEVDFDAVYASVRGLLLKGFAETHSLALQQTMYEMGRAVIETHPEIDEVSMSLPNKHHFLVDLQPFGQDNPNEVFYAADRPYGLIEATIQREGSRAD

SEQ ID NO: 136 - protein, 291 aa, synthetic construct
MTATAETSTGTKVVLGQNQYGKAEVRLVKVTRNTDRHEIQDLNVTSQLSGDFEAAHTAGDNAHVVATDTQKNTVYAFARDGVGTTEEFLLRLGKHFTEGFDWVTGGRWAAQQFFWDRINDHDHAFSRNKSEVRTAVLEISGSEQAIVSGIEGLTVLKSTGSEFHGFPRDKYTTLQETTDRILATDVSARWRYNTVEVDFDAVYASVRGLLLKGFAETHSHALQQTMYEMGRAVIETHPEIDEISMSLPNKHHFLVDLQPFGQDNPNEVFYAADRPYGLIEATIQREGSRAD

SEQ ID NO: 137 - protein, 291 aa, synthetic construct
MTATAETSTGTKVVLGQNQYGKAEVRLVKVTRNTARHEIQDLNVTSQLSGDFEAAHTAGDNAHVVATDTQKNTVYAFARDGFGTTEEFLLRLGKHFTEGFDWVTGGRWAAQQFFWDRINDHDHAFSRNKSEVRTAVLEISGSEQAIVAGIEGLTVLKSTGSEFHGFPRDKYTTLQETTDRILATDVSARWRYNTDEVDFDAVYDSVRGLLLKAFAETHSKALQQTMYEMGRAVIETHPEIDEVKMSLPNNHHFLVDLQPFGQDNPNEVFYAADRPYGLIEATIQREGSRAD

SEQ ID NO: 138 - protein, 291 aa, synthetic construct
MTATAETSTGTKVVLGQNQYGKAEVRLVKVTRNTARHEIQDLNVTSQLSGDFEAAHTAGDNAHVVATDTQKNTVYAFARDGFGTTEEFLLRLGKHFTEGFDWVTGGRWAAQQHFWDRINDHDHAFSRNKSEVRTAVLEISGSEQAIVAGIEGLTVLKSTGSEFHGFPRDKYTTLQETTDRILATDVSARWRYNTDEVDFDAVYDSVRGLLLKAFAETHSHALQQTMYEMGRAVIETHPEIDEVKMSLPNKHHFLVDLQPFGQDNPNEVFYAADRPYGLIEATIQREGSRAD

SEQ ID NO: 139 - protein, 291 aa, synthetic construct
MTATAETSTGTKVVLGQNQYGKAEVRLVKVTRETARHEIQDLNVTSQLSGDFEAAHTAGDNAHVVATDTQKNTVYAFARDGVGTTEEFLLRLGKHFTEGFDWVTGGRWAAQQFFWDRINDHDHAFSRNKSEVRTAVLEISGSEQHIVSGIEGLTVLKSTGSEFHGFPRDKYTTLQETTDRILATDVSARWRYNTEEVDFDAVYDSVRGLLLKGFAETHSLALQQTMYEMGRAVIETHPEIDEISMSLPNKHHFLVDLQPFGQDNPNEVFHAADRPYGLIEATIQREGSRAD

SEQ ID NO: 140 - protein, 291 aa, synthetic construct
MTATAETSTGTKVVLGQNQYGKAEVRLVKVTRETARHEIQDLNVTSQLSGDFEAAHTAGDNAHVVATDTQKNTVYAFARDGFGTTEEFLLRLGKHFTEGFDWVTGGRWAAQQFFWDRINDHDHAFSRNKSEVRTAVLEISGSEQHIVAGIEGLTVLKSTGSEFHGFPRDKYTTLQETTDRILATDVSARWRYNTEEVDFDAVHASVRGLLLKAFAETHSHALQQTMYEMGRAVIETHPEIDEIKMSLPNKHHFLVDLQPFGQDNPNEVFYAADRPYGLIEATIQREGSRAD

SEQ ID NO: 141 - protein, 291 aa, synthetic construct
MTATAETSTGTKVVLGQNQYGKAEVRLVKVTRNTARHEIQDLNVTSQLSGDFEAAHTAGDNAHVVATDTQKNTVYAFARDGFGTTEEFLLRLGKHFTEGFDWVTGGRWAAQQFFWDRINDHDHAFSRNKSEVRTAVLEISGSEQHIVSGIEGLTVLKSTGSEFHGFPRDKYTTLQETTDRILATDVSARWRYNTEEVDFDAVYASVRGLLLKGFAETHSKALQQTMYEMGRAVIETHPEIDEIKMSLPNNHHFLVDLQPFGQDNPNEVFYAADRPYGLIEATIQREGSRAD

SEQ ID NO: 142 - protein, 291 aa, synthetic construct
MTATAETSTGTKVVLGQNQYGKAEVRLVKVTRETARHEIQDLNVTSQLSGDFEAAHTAGDNAHVVATDTQKNTVYAFARDGVATTEEFLLRLGKHFTEGFDWVTGGRWAAQQFFWDRINDHDHAFSRNKSEVRTAVLEISGSEQHIVSGIEGLTVLKSTGSEFHGFPRDKYTTLQETTDRILATDVSARWRYNTVEVDFDAVYDSVRGLLLKAFAETHSLALQQTMYEMGRAVIETHPEIDEVKMSLPNNHHFLVDLQPFGQDNPNEVFHAADRPYGLIEATIQREGSRAD

SEQ ID NO: 143 - protein, 291 aa, synthetic construct
MTATAETSTGTKVVLGQNQYGKAEVRLVKVTRETDRHEIQDLNVTSQLSGDFEAAHTAGDNAHVVATDTQKNTVYAFARDGFATTEEFLLRLGKHFTEGFDWVTGGRWAAQQFFWDRINDHDHAFSRNKSEVRTAVLEISGSEQAIVAGIEGLTVLKSTGSEFHGFPRDKYTTLQETTDRILATDVSARWRYNTDEVDFDAVYDSVRGLLLKAFAETHSKALQQTMYEMGRAVIETHPEIDEVKMSLPNNHHFLVDLQPFGQDNPNEVFHAADRPYGLIEATIQREGSRAD

SEQ ID NO: 144 - protein, 291 aa, synthetic construct
MTATAETSTGTKVVLGQNQYGKAEVRLVKVTRNTDRHEIQDLNVTSQLSGDFEAAHTAGDNAHVVATDTQKNTVYAFARDGFATTEEFLLRLGKHFTEGFDWVTGGRWAAQQFFWDRINDHDHAFSRNKSEVRTAVLEISGSEQHIVSGIEGLTVLKSTGSEFHGFPRDKYTTLQETTDRILATDVSARWRYNTDEVDFDAVYDSVRGLLLKGFAETHSLALQQTMYEMGRAVIETHPEIDEISMSLPNNHHFLVDLQPFGQDNPNEVFYAADRPYGLIEATIQREGSRAD

SEQ ID NO: 145 - protein, 291 aa, synthetic construct
MTATAETSTGTKVVLSQNQYGKAEVRLVKVTRNTARHEIQDLNVTSQLSGDFEAAHTAGDNAHVVATDTQKNTVYAFARDGFATTEEFLLRLGKHFTEGFDWVTGGRWAAQQHFWDRINDHDHAFSRNKSEVRTAVLEISGSEQHIVAGIEGLTVLKSTGSEFHGFPRDKYTTLQETTDRILATDVSARWRYNTVEVDFDAVYGSVRGLLLKAFAETHSLALQQTMYEMGRAVIETHPEIDEVKMSLPNNHHFLVDLQPFGQDNPNEVFHAADRPYGLIEATIQREGSRAD

SEQ ID NO: 146 - protein, 291 aa, synthetic construct
MTATAETSTGTKVVLGQNQYGKAEVRLVKVTRNTARHEIQDLNVTSQLSGDFEAAHTAGDNAHVVATDTQKNTVYAFARDGFATTEEFLLRLGKHFTEGFDWVTGGRWAAQQFFWDRINDHDHAFSRNKSEVRTAVLEISGSEQHIVSGIEGLTVLKSTGSEFHGFPRDKYTTLQETTDRILATDVSARWRYNTDEVDFDAVYDSVRGLLLKAFAETHSKALQQTMYEMGRAVIETHPEIDEVSMSLPNNHHFLVDLQPFGQDNPNEVFYAADRPYGLIEATIQREGSRVD

SEQ ID NO: 147 - protein, 291 aa, synthetic construct
MTATAETSTGTKVVLGQNQYGKAEVRLVKVTRETARHEIQDLNVTSQLSGDFEAAHTAGDNAHVVATDTQKNTVYAFARDGFGTTEEFLLRLGKHFTEGFDWVTGGRWAAQQFFWDRINDHDHAFSRNKSEVRTAVLEISGSEQAIVSGIEGLTVLKSTGSEFHGFPRDKYTTLQETTDRILATDVSARWRYNTDEVDFDAVYASVRGLLLKAFAETHSHALQQTMYEMGCAVIETHPEIDEVSMSLPNKHHFLVDLQPFGQDNPNEVFYAADRPYGLIEATIQREGSRAD

SEQ ID NO: 148 - protein, 291 aa, synthetic construct
MTATAETSTGTKVVLGQNQYGKAEVRLVKVTRNTARHEIQDLNVTSQLSGDFEAAHTAGDNAHVVATDTQKNTVYAFARDGFATTEEFLLRLGKHFTEGFDWVTGGRWAAQQHFWDRINDHDHAFSRNKSEVRTAVLEISGSEQTIVSGIEGLTVLKSTGSEFHGFPRDKYTTLQETTDRILATDVSARWRYNTVEVDFDAVYDSVRGLLLKGFAETHSLALQQTMYEMGRAVIETHPEIDEVKMSLPNNHHFLVDLQPFGQDNPNEVFHAADRPYGLIEATIQREGSRAD

SEQ ID NO: 149 - protein, 291 aa, synthetic construct
MTATAETSTGTKVVLGQNQYGKAEVRLVKVTRNTARHEIQDLNVTSQLSGDFEAAHTAGDNAHVVATDTQKNTVYAFARDGFGTTEEFLLRLGKHFTEGFDWVTGGRWAAQQFFWDRINDHDHAFSRNKSEVRTAVLEISGSEQAIVAGIEGLTVLKSTGSEFHGFPRDKYTTLQETTDRILATDVSARWRYNTVEVDFDAVYASVRGLLLKAFAETHSKALQQTMYEMGRAVIETHPEIDEISMSLPNNHHFLVDLQPFGQDNPNEVFHAADRPYGLIEATIQREGSRAD

SEQ ID NO: 150 - protein, 291 aa, synthetic construct
MTATAETSTGTKVVLGQNQYGKAEVRLVKVTRETDRHEIQDLNVTSQLSGDFEAAHTAGDNAHVVATDTQKNTVYAFARDGFATTEEFLLRLGKHFTEGFDWVTGGRWAAQQHFWDRINDHDHAFSRNKSEVRTAVLEISGSEQHIVAGIEGLTVLKSTGSEFHGFPRDKYTTLQETTDRILATDVSARWRYNTVEVDFDAVYASVRGLLLKGFAETHSLALQQTMYEMGRAVIETHPEIDEISMSLPNKHHFLVDLQPFGQDNPNEVFYAADRPYGLIEATIQREGSRAD

SEQ ID NO: 151 - protein, 291 aa, synthetic construct
MTATAETSTGTKVVLGQNQYGKAEVRLVKVTRETDRHEIQDLNVTSQLSGDFEAAHTAGDNAHVVATDTQKNTVYAFARDGVGTTEEFLLRLGKHFTEGFDWVTGGRWAAQQHFWDRINDHDHAFSRNKSEVRTAVLEISGSEQAIVAGIEGLTVLKSTGSEFHGFPRDKYTTLQETTDRILATDVSARWRYNTDEVDFDAVYDSVRGLLLKAFAETHSKALQQTMYEMGRAVIETHPEIDEIKMSLPNKHHFLVDLQPFGQDNPNEVFYAADRPYGLIEATIQREGSRAD

SEQ ID NO: 152 - protein, 291 aa, synthetic construct
MTATAETSTGTKVVLGQNQYGKAEVRLVKVTRNTARHEIQDLNVTSQLSGDFKAAHTAGDNAHVVATDTQKNTVYAFARDGFATTEEFLLRLGKHFTEGFDWVTGGRWAAQQHFWDRINDHDHAFSRNKSEVRTAVLEISGSEQTIVSGIEGLTVLKSTGSEFHGFPRDKYTTLQETTDRILATDVSARWRYNTDEVDFDAVYGSVRGLLLKAFAETHSKALQQTMYEMGRAVIETHPEIDEVKMSLPNNHHFLVDLQPFGQDNPNEVFYAADRPYGLIEATIQREGSRAD

SEQ ID NO: 153 - protein, 291 aa, synthetic construct
MTATAETSTGTKVVLGQNQYGKAEVRLVKVTRNTARHEIQDLNVTSQLSGDFEAAHTAGDNAHVVATDTQKNTVYAFARDGVGTTEEFLLRLGKHFTEGFDWVTGGRWAAQQHFWDRINDHDHAFSRNKSEVRTAVLEISGSEQHIVAGIEGLTVLKSTGSEFHGFPRDKYTTLQETTDRILATDVSARWRYNTEEVDFDAVYASVRGLLLKGFAETHSLALQQTMYEMGRAVIETHPEIDEIKMSLPNKHHFLVDLQPFGQDNPNEVFYAADRPYGLIEATIQREGSRAD

SEQ ID NO: 154 - protein, 291 aa, synthetic construct
MTATAETSTGTKVVLGQNQYGKAEVRLVKVTRNTDRHEIQDLNVTSQLSGDFEAAHTAGDNAHVVATDTQKNTVYAFARDGFATTEEFLLRLGKHFTEGFDWVTGGRWAAQQHFWDRINDHDHAFSRNKSEVRTAVLEISGSEQTIVAGIEGLTVLKSTGSEFHGFPRDKYTTLQETTDRILATDVSARWRYNTEEVDFDAVYDSVRGLLLKAFAETHSHALQQTMYEMGRAVIETHPEIDEVSMSLPNKHHFLVDLQPFGQDNPNEVFHAADRPYGLIEATIQREGSRAD

SEQ ID NO: 155 - protein, 291 aa, synthetic construct
MTATAETSTGTKVVLGQNQYGKAEVRLVKVTRNTARHEIQDLNVTSQLSGDFEAAHTAGDNAHVVATDTQKNTVYAFARDGVGTTEEFLLRLGKHFTEGFDWVTGGRWAAQQFFWDRINDHDHAFSRNKSEVRTAVLEISGSEQAIVSGIEGLTVLKSTGSEFHGFPRDKYTTLQETTDRILATDVSARWRYNTDEVDFDAVYGSVRGLLLKAFAETHSHALQQTMYEMGRAVIETHPEIDEVKMSLPNKHHFLVDLQPFGQDNPNEVFHAADRPYGLIEATIQREGSRAD

SEQ ID NO: 156 - protein, 291 aa, synthetic construct
MTATAKTSTGTKVVLGQNQYGKAEVRLVKVTRETDRHEIQDLNVTSQLSGDFEAAHTAGDNAHVVATDTQKNTVYAFARDGFGTTEEFLLRLGKHFTEGFDWVTGGRWAAQQFFWDRINDHDHAFSRNKSEVRTAVLEISGSEQAIVAGIEGLTVLKSTGSEFHGFPRDKYTTLQETTDRILATDVSARWRYNTEEVDFDAVHASVRGLLLKAFAETHSKALQQTMYEMGRAVIETHPEIDEMKMSLPNKHHFLVDLQPFGQDNPNEVFYAADRPYGLIEATIQREGSRAD

SEQ ID NO: 157 - protein, 291 aa, synthetic construct
MTATAETSTGTKVVLGQNQYGKAEVRLVKVTRETDRHEIQDLNVTSQLSGDFEAAHTAGDNAHVVATDTQKNTVYAFARDGFGTTEEFLLRLGKHFTEGFDWVTGGRWAAQQHFWDRINDHDHAFSRNKSEVRTAVLEISGSEQHIVSGIEGLTVLKSTGSEFHGFPRDKYTTLQETTDRILATDVSARWRYNTVEVDFDAVYASVRGLLLKAFAETHSLALQQTMYEMGRAVIETHPEIDEVSMSLPNKHHFLVDLQPFGQDNPNEVFYAADRPYGLIEATIQREGSRAD

SEQ ID NO: 158 - protein, 291 aa, synthetic construct
MTATAETSTGTKVVLGQNQYGKAEVRLVKVTRETARHEIQDLNVTSQLSGDFEAAHTAGDNAHVVATDTQKNTVYAFARDGVGTTEEFLLRLGKHFTEGFDWVTGGRWAAQQHFWDRINDHDHAFSRNKSEVRTAVLEISGSEQTIVAGIEGLTVLKSTGSEFHGFPRDKYTTLQETTDRILATDVSARWRYNTEEVDFDAVYGSVRGLLLKAFAETHSKALQQTMYEMGRAVIETHPEIDEVKMSLPNKHHFLVDLQPFGQDNPNEVFYAADRPYGLIEATIQREGSRAD

SEQ ID NO: 159 - protein, 291 aa, synthetic construct
MTATAETSTGTKVVLGQNQYGKAEVRLVKVTRETARHEIQDLNVTSQLSGDFEAAHTAGDNAHVVATDTQKNTVYAFARDGFATTEEFLLRLGKHFTEGFDWVTGGRWAAQQFFWDRINDHDHAFSRNKSEVRTAVLEISGSEQHIVSGIEGLTVLKSTGSEFHGFPRDKYTTLQETTDRILATDVSARWRYNTDEVDFDAVYASVRGLLLKGFAETHSHALQQTMYEMGRAVIETHPEIDEVSMSLPNNHHFLVDLQPFGQDNPNEVFYAADRPYGLIEATIQREGSRAD

SEQ ID NO: 160 - protein, 291 aa, synthetic construct
MTATAETSTGTKVVLGQNQYGKAEVRLVKVTRNTDRHEIQDLNVTSQLSGDFEAAHTAGDNAHVVATDTQKNTVYAFARDGFGTTEEFLLRLGKHFTEGFDWVTGGRWAAQQFFWDRINDHDHAFSRNKSEVRTAVLEISGSEQTIVSGIEGLTVLKSTGSEFHGFPRDKYTTLQETTDRILATDVSARWRYNTEEVDFDAVYGSVRGLLLKAFAETHSLALQQTMYEMGCAVIETHPEIDEVSMSLPNNHHFLVDLQPFGQDNPNEVFHAADRPYGLIEATIQREGSRAD

SEQ ID NO: 161 - protein, 291 aa, synthetic construct
MTATAETSTGTKVVLGQNQYGKAEVRLVKVTRNTDRHEIQDLNVTSQLSGDFEAAHTAGDNAHVVATDTQKNTVYAFARDGFGTTEEFLLRLGKHFTEGFDWVTGGRWAAQQHFWDRINDHDHAFSRNKSEVRTAVLEISGSEQTIVAGIEGLTVLKSTGSEFHGFPRDKYTTLQETTDRILATDVSARWRYNTVEVDFDAVYDSVRGLLLKGFAETHSKALQQTMYEMGRAVIETHPEIDEVSMSLPNKHHFLVDLQPFGQDNPNEVFYAADRPYGLIEATIQREGSRAD

SEQ ID NO: 162 - protein, 291 aa, synthetic construct
MTATAETSTGTKVVLGQNQYGKAEVRLVKVTRNTDRHEIQDLNVTSQLSGDFEAAHTAGDNAHVVATDTQKNTVYAFARDGVGTTEEFLLRLGKHFTEGFDWVTGGRWAAQQHFWDRINDHDHAFSRNKSEVRTAVLEISGSEQAIVAGIEGLTVLKSTGSEFHGFPRDKYTTLQETTDRILATDVSARWRYNTDEVDFDAVYASVRGLLLKGFAETHSLALQQTMYEMGRAVIETHPEIDEISMSLPNKHHFLVDLQPFGQDNPNEVFYAADRPYGLIEATIQREGSRAD

SEQ ID NO: 163 - protein, 291 aa, synthetic construct
MTATAETSTGTKVVLGQNQYGKAEVRLVKVTRETARHEIQDLNVTSQLSGDFEAAHTAGDNAHVVATDTQKNTVYAFARDGFGTTEEFLLRLGKHFTEGFDWVTGGRWAAQQFFWDRINDHDHAFSRNKSEVRTAVLEISGSEQAIVSGIEGLTVLKSTGSEFHGFPRDKYTTLQETTDRILATDVSARWRYNTVEVDFDAVYGSVRGLLLKAFAETHSHALQQTMYEMGRAVIETHPEIDEISMSLPNKHHFLVDLQPFGQDNPNEVFYAADRPYGLIEATIQREGSRAD

SEQ ID NO: 164 - protein, 291 aa, synthetic construct
MTATAETSTGTKVVLGQNQYGKAEVRLVKVTRETDRHEIQDLNVTSQLSGDFEAAHTAGDNAHVVATDTQKNTVYAFARDGFGTTEEFLLRLGKHFTEGFDWVTGGRWAAQQFFWDRINDHDHAFSRNKSEVRTAVLEISGSEQTIVSGIEGLTVLKSTGSEFHGFPRDKYTTLQETTDRILATDVSARWRYNTDEVDFDAVYDSVRGLLLKGFAETHSKALQQTMYEMGRAVIETHPEIDEISMSLPNKHHFLVDLQPFGQDNPNEVFHAADRPYGLIEATIQREGSRAD

SEQ ID NO: 165 - protein, 291 aa, synthetic construct
MTATAETSTGTKVVLGQNQYGKAEVRLVKVTRETDRHEIQDLNVTSQLYGDFEAAHTAGDNAHVVATDTQKNTVYAFARDGVATTEEFLLRLGKHFTEGFDWVTGGRWAAQQFFWDRINDHDHAFSRNKSEVRTAVLEISGSEQHIVAGIEGLTVLKSTGSEFHGFPRDKYTTLQETTDRILATDVSARWRYNTEEVDFDAVYDSVRGLLLKAFAETHSKALQQTMYEMGRAVIETHPEIDEISMSLPNNHHFLVDLQPFGQDNPNEVFYAADRPYGLIEATIQREGSRAD

SEQ ID NO: 166 - protein, 291 aa, synthetic construct
MTATAETSTGTKVVLGQNQYGKAEVRLVKVTRNTARHEIQDLNVTSQLSGDFEAAHTAGDNAHVVATDTQKNTVYAFARDGFATTEEFLLRLGKHFTEGFDWVTGGRWAAQQHFWDRINDHDHAFSRNKSEVRTAVLEISGSEQHIVAGIEGLTVLKSTGSEFHGFPRDKYTTLQETTDRILATDVSARWRYNTVEVDFDAVYASVRGLLLKGFAETHSLALQQTMYEMGRAVIETHPEIDEIKMSLPNNHHFLVDLQPFGQDNPNEVFYAADRPYGLIEATIQREGSRAD

SEQ ID NO: 167 - protein, 291 aa, synthetic construct
MTATAETSTGTKVVLGQNQYGKAEVRLVKVTRETDRHEIQDLNVTSQLSGDFEAAHTAGDNAHVVATDTQKNTVYAFARDGFGTTEEFLLRLGKHFTEGFDWVTGGRWAAQQFFWDRINDHDHAFSRNKSEVRTAVLEISGSEQTIVSGIEGLTVLKSTGSEFHGFPRDKYTTLQETTDRILATDVSARWRYNTVEVDFDAVYDSVRGLLLKGFAETHSKALQQTMYEMGRAVIETHPEIDEISMSLPNNHHFLVDLQPFGQDNPNEVFYAADRPYGLIEATIQREGSRAD

SEQ ID NO: 168 - protein, 291 aa, synthetic construct
MTATAETSTGTKVVLGQNQYGKAEVRLVKVTRETDRHEIQDLNVTSQLSGDFEAAHTAGDNAHVVATDTQKNTVYAFARDGFATTEEFLLRLGKHFTEGFDWVTGGRWAAQQFFWDRINDHDHAFSRNKSEVRTAVLEISGSEQAIVAGIEGLTVLKSTGSEFHGFPRDKYTTLQETTDRILATDVSARWRYNTVEVDFDAVHGSVRGLLLKAFAETHSKALQQTMYEMGRAVIETHPEIDEIKMSLPNKHHFLVDLQPFGQDNPNEVFHAADRPYGLIEATIQREGSRAD

SEQ ID NO: 169 - protein, 291 aa, synthetic construct
MTATAETSTGTKVVLGQNQYGKAEVRLVKVTRNTDRHEIQDLNVTSQLSGDFEAAHTAGDNAHVVATDTQKNTVYAFARDGFGTTEEFLLRLGKHFTEGFDWVTGGRWAAQQFFWDRINDHDHAFSRNKSEVRTAVLEISGSEQAIVSGIEGLTVLKSTGSEFHGFPRDKYTTLQETTDRILVTDVSVRWRYNTVEVDFDAVYDSVRGLLLKAFAETHSKALQQTMYEMGRAVIETHPEIDEVKMSLPNKHHFLVDLQPFGQDNPNEVFYAADRPYGLIEATIQREGSRAD

SEQ ID NO: 170 - protein, 291 aa, synthetic construct
MTATAETSTGTKVVLGQNQYGKAEVRLVKVTRNTARHEIQDLNVTSQLSGDFEAAHTAGDNAHVVATDTQKNTVYAFARDGFGTTEEFLLRLGKHFTEGFDWVTGGRWAAQQFFWDRINDHDHAFSRNKSEVRTAVLEISGSEQTIVSGIEGLTVLKSTGSEFHGFPRDKYTTLQETTDRILATDVSARWRYNTDEVDFDAVYGSVRGLLLKAFAETHSLALQQTMYEMGRAVIETHPEIDEISMSLPNNHHFLVDLQPFGQDNPNEVFYAADRPYGLIEATIQREGSRAD

SEQ ID NO: 171 - protein, 291 aa, synthetic construct
MTATAETSTGTKVVLGQNQYGKAEVRLVKVTRETDRHEIQDLNVTSQLSGDFEAAHTAGDNAHVVATDTQKNTVYAFARDGVGTTEEFLLRLGKHFTEGFDWVTGGRWAAQQHFWDRINDHDHAFSRNKSEVRTAVLEISGSEQTIVSGIEGLTVLKSTGSEFHGFPRDKYTTLQETTDRILATDVSARWRYNTEEVDFDAVYDSVRGLLLKAFAETHSLALQQTMYEMGRAVIETHPEIDEVKMSLPNKHHFLVDLQPFGQDNPNEVFHAADRPYGLIEATIQREGSRAD

SEQ ID NO: 172 - protein, 291 aa, synthetic construct
MTATAETSTGTKVVLGQNQYGKAEVRLVKVTRNTARHEIQDLNVTSQLSGDFEAAHTAGDNAHVVATDTQKNTVYAFARDGFGTTEEFLLRLGKHFTEGFDWVTGGRWAAQQHFWDRINDHDHAFSRNKSEVRTAVLEISGSEQTIVSGIEGLTVLKSTGSEFHGFPRDKYTTLQETTDRILATDVSARWRYNTDEVDFDAVYGSVRGLLLKAFAETHSKALQQTMYEMGRAVIETHPEIDEIKMSLPNKHHFLVDLQPFGQDNPNEVFYAADRPYGLIEATIQREGSRAD

SEQ ID NO: 173 - protein, 291 aa, synthetic construct
MTATAETSTGTKVVLGQNQYGKAEVRLVKVTRNTDRHEIQDLNVTSQLSGDFEAAHTAGDNAHVVATDTQKNTVYAFARDGVGTTEEFLLRLGKHFTEGFDWVTGGRWAAQQHFWDRINDHDHAFSRNKSEVRTAVLEISGSEQTIVAGIEGLTVLKSTGSEFHGFPRDKYTTLQETTDRILATDVSARWRYNTVEVDFDAVYASVRGLLLKAFAETHSHALQQTMYEMGRAVIETHPEIDEISMSLPNKHHFLVDLQPFGQDNPNEVFYAADRPYGLIEATIQREGSRAD

SEQ ID NO: 174 - protein, 291 aa, synthetic construct
MTATAETSTGTKVVLGQNQYGKAEVRLVKVTRETDRHEIQDLNVTSQLSGDFEAAHTAGDNAHVVATDTQKNTVYAFARDGFGTTEEFLLRLGKHFTEGFDWVTGGRWAAQQHFWDRINDHDHAFSRNKSEVRTAVLEISGSEQHIVAGIEGLTVLKSTGSEFHGFPRDKYTTLQETTDRILATDVSARWRYNTEEVDFDAVYGSVRGLLLKAFAETHSHALQQTMYEMGRAVIETHPEIDEIKMSLPNKHHFLVDLQPFGQDNPNEVFHAADRPYGLIEATIQREGSRAD

SEQ ID NO: 175 - protein, 291 aa, synthetic construct
MTATAETSTGTKVVLGQNQYGKAEVRLVKVTRNTARHEIQDLNVTSQLSGDFEAAHTAGDNAHVVATDTQKNTVYAFARDGFATTEEFLLRLGKHFTEGFDWVTGGRWAAQQFFWDRINDHDHAFSRNKSEVRTAVLEISGSEQAIVSGIEGLTVLKSTGSEFHGFPRDKYTTLQETTDRILATDVSARWRYNTDEVDFDAVYDSVRGLLLKAFAETHSHALQQTMYEMGRAVIETHPEIDEISMSLPNNHHFLVDLQPFGQDNPNEVFHAADRPYGLIEATIQREGSRAD

SEQ ID NO: 176 - protein, 291 aa, synthetic construct
MTATAETSTGTKVVLGQNQYGKAEVRLVKVTRNTARHEIQDLNVTSQLSGDFEAAHTAGDNAHVVATDTQKNTVYAFARDGFGTTEEFLLRLGKHFTEGFDWVTGGRWAAQQHFWDRINDHDHAFSRNKSEVRTAVLEISGSEQTIVAGIEGLTVLKSTGSEFHGFPRDKYTTLQETTDRILATDVSARWRYNTDEVDFDAVYDSVRGLLLKVFAETHSLALQQTMYEMGRAVIETHPEIDEVKMSLPNNHHFLVDLQPFGQDNPNEVFHAADRPYGLIEATIQREGSRAD

SEQ ID NO: 177 - protein, 291 aa, synthetic construct
MTATAETSTGTKVVLGQNQYGKAEVRLVKVTRETARHEIQDLNVTSQLSGDFEAAHTAGDNAHVVATDTQKNTVYAFARDGFATTEEFLLRLGKHFTEGFDWVTGGRWAAQQFFWDRINDHDHAFSRNKSEVRTAVLEISGSEQAIVAGIEGLTVLKSTGSEFHGFPRDKYTTLQETTDRILATDVSARWRYNTEEVDFDAVYASVRGLLLKAFAETHSKALQQTMYEMGRAVIETHPEIDEVSMSLPNKHHFLVDLQPFGQDNPNEVFYAADRPYGLIEATIQREGSRAD

SEQ ID NO: 178 - protein, 291 aa, synthetic construct
MTATAETSTGTKVVLGQNQYGKAEVRLVKVTRNTARHEIQDLNVTSQLSGDFEAAHTAGDNAHVVATDTQKNTVYAFARDGVATTEEFLLRLGKHFTEGFDWVTGGRWAAQQFFWDRINDHDHAFSRNKSEVRTAVLEISGSEQHIVSGIEGLTVLKSTGSEFHGFPRDKYTTLQETTDRILATDVSARWRYNTDEVDFDAVYDSVRGLLLKAFAETHSHALQQTMYEMGRAVIETHPEIDEIKMSLPNNHHFLVDLQPFGQDNPNEVFHAADRPYGLIEATIQREGSRAD

SEQ ID NO: 179 - protein, 291 aa, synthetic construct
MTATAETSTGTKVVLGQNQYGKAEVRLVKVTRETARHEIQDLNVTSQLSGDFEAAHTAGDNAHVVATDTQKNTVYAFARDGVGTTEEFLLRLGKHFTEGFDWVTGGRWAAQQFFWDRINDHDHAFSRNKSEVRTAVLEISGSEQAIVGGIEGLTVLKSTGSEFHGFPRDKYTTLQETTDRILATDVSARWRYNTEEVDFDAVHASVRGLLLKGFAETHSKALQQTMYEMGRAVIETHPEIDEMNMSLPNKHHFLVDLQPFGQDNPNEVFHAADRPYGLIEATIQREGSRAD

SEQ ID NO: 180 - protein, 291 aa, synthetic construct
MTATAETSTGTKVVLGQNQYGKAEVRLVKVTRNTARHEIQDLNVTSQLSGDFEAAHTAGDNAHVVATDTQKNTVYAFARDGFGTTEEFLLRLGKHFTEGFDWVTGGRWAAQQFFWDRINDHDHAFSRNKSEVRTAVLEISGSEQTIVAGIEGLTVLKSTGSEFHGFPRDKYTTLQETTDRILATDVSARWRYNTDEVDFDAVYDSVRGLLLKGFAETHSKALQQTMYEMGRAVIETHPEIDEISMSLPNNHHFLVDLQPFGQDNPNEVFYAADRPYGLIEATIQREGSRAD

SEQ ID NO: 181 - protein, 291 aa, synthetic construct
MTATAETSTGTKVVLGQNQYGKAEVRLVKVTRETDRHEIQDLNVTSQLSGDFEAAHTAGDNAHVVATDTQKNTVYAFARDGFGTTEEFLLRLGKHFTEGFDWVTGGRWAAQQFFWDRINDHDHAFSRNKSEVRTAVLEISGSEQTIVAGIEGLTVLKSTGSEFHGFPRDKYTTLQETTDRILATDVSARWRYNTEEVDFDAVYDSVRGLLLKAFAETHSLALQQTMYEMGRAVIETHPEIDEVSMSLPNKHHFLVDLQPFGQDNPNEVFHAADRPYGLIEATIQREGSRAD

SEQ ID NO: 182 - protein, 291 aa, synthetic construct
MTATAETSTGTKVVLGQNQYGKAEVRLVKVTRNTDRHEIQDLNVTSQLSGDFEAAHTAGDNAHVVATDTQKNTVYAFARDGFGTTEEFLLRLGKHFTEGFDWVTGGRWAAQQHFWDRINDHDHAFSRNKSEVRTAVLEISGSEQAIVSGIEGLTVLKSTGSEFHGFPRDKYTTLQETTDRILATDVSARWRYNTVEVDFDAVYASVRGLLLKGFAETHSLALQQTMYEMGRAVIETHPEIDEVKMSLPNKHHFLVDLQPFGQDNPNEVFYAADRPYGLIEATIQREGSRAD

SEQ ID NO: 183 - protein, 291 aa, synthetic construct
MTATAETSTGTKVVLGQNQYGKAEVRLVKVTRNTARHEIQDLNVTSQLSGDFEAAHTAGDNAHVVATDTQKNTVYTFARDGFATTEEFLLRLGKHFTEGFDWVTGGRWAAQQHFWDRINDHDHAFSRNKSEVRTAVLEISGSEQTIVAGIEGLTVLKSTGSEFHGFPRDKYTTLQETTDRILATDVSARWRYNTVEVDFDAVYGSVRGLLLKAFAETHSKALQQTMYEMGRAVIETHPEIDEVSMSLPNKHHFLVDLQPFGQDNPNEVFYAADRPYGLIEATIQREGSRAD

SEQ ID NO: 184 - protein, 291 aa, synthetic construct
MTATAETSTGTKVVLGQNQYGKAEVRLVKVTRETDRHEIQDLNVTSQLSGDFEAAHTAGDNAHVVATDTQKNTVYAFARDGFGTTEEFLLRLGKHFTEGFDWVTGGRWAAQQHFWDRINDHDHAFSRNKSEVRTAVLEISGSEQAIVSGIEGLTVLKSTGSEFHGFPRDKYTTLQETTDRILATDVSARWRYNTDEVDFDAVYGSVRGLLLKAFAETHSLALQQTMYEMGRAVIETHPEIDEVNMSLPNKHHFLVDLQPFGQDNPNEVFHAADRPYGLIEATIQREGSRAD

SEQ ID NO: 185 - protein, 291 aa, synthetic construct
MTATAETSTGTKVVLGQNQYGKAEVRLVKVTRETDRHEIQDLNVTSQLSGDFEAAHTAGDNAHVVATDTQKNTVYAFARDGFGTTEEFLLRLGKHFTEGFDWVTGGRWAAQQFFWDRINDHDHAFSRNKSEVRTAVLEISGSEQAIVSGIEGLTVLKSTGSEFHGFPRDKYTTLQETTDRILATDVSARWRYNTVEVDFDAVHASVRGLLLKAFAETHSKALQQTMYEMGRAVIETHPEIDEISMSLPNKHHFLVDLQPFGQDNPNEVFYAADRPYGLIEATIQREGSRAD

SEQ ID NO: 186 - protein, 291 aa, synthetic construct
MTATAETSTGTKVVLGQNQYGKAEVRLVKVTRNTARHEIQDLNVTSQLSGDFEAAHTAGDNAHVVATDTQKNTVYAFARDGFGTTEEFLLRLGKHFTEGFDWVTGGRWAAQQHFWDRINDHDHAFSRNKSEVRTAVLEISGSEQHIVSGIEGLTVLKSTGSEFHGFPRDKYTTLQETTDRILATDVSARWRYNTEEVDFDAVYDSVRGLLLKAFAETHSHALQQTMYEMGRAVIETHPEIDEVKMSLPNNHHFLVDLQPFGQDNPNEVFYAADRPYGLIEATIQREGSRAD

SEQ ID NO: 187 - protein, 291 aa, synthetic construct
MTATAETSTGTKVVLGQNQYGKAEVRLVKVTRETARHEIQDLNVTSQLSGDFEAAHTAGDNAHVVATDTQKNTVYAFARDGVGTTEEFLLRLGKHFTEGFDWVTGGRWAAQQFFWDRINDHDHAFSRNKSEVRTAVLEISGSEQAIVAGIEGLTVLKSTGSEFHGFPRDKYTTLQETTDRILATDVSARWRYNTEEVDFDAVYGSVRGLLLKAFAETHSKALQQTMYEMGRAVIETHPEIDEISMSLPNNHHFLVDLQPFGQDNPNEVFHAADRPYGLIEATIQREGSRAD

SEQ ID NO: 188 - protein, 291 aa, synthetic construct
MTATAETSTGTKVVLGQNQYGKAEVRLVKVTRETARHEIQDLNVTSQLSGDFEAAHTAGDNAHVVATDTQKNTVYAFARDGFATTEEFLLRLGKHFTEGFDWVTGGRWAAQQFFWDRINDHDHAFSRNKSEVRTAVLEISGSEQHIVSGIEGLTVLKSTGSEFHGFPRDKYTTLQETTDRILATDVSARWRYNTDEVDFDAVYGSVRGLLLKGFAETHSHALQQTMYEMGRAVIETHPEIDEVKMSLPNNHHFLVDLQPFGQDNPNEVFHAADRPYGLIEATIQREGSRAD

SEQ ID NO: 189 - protein, 291 aa, synthetic construct
MTATAETSTGTKVVLGQNQYGKAEVRLVKVTRNTDRHEIQDLNVTSQLSGDFEAAHTAGDNAHVVATDTQKNTVYAFARDGFGTTEEFLLRLGKHFTKGFDWVTGGRWAAQQHFWDRINDHDHAFSRNKSEVRTAVLEISGSEQHIVSGIEGLTVLKSTGSEFHGFPRDKYTTLQETTDRILATDVSARWRYNTDEVDFDAVYASVRGLLLKAFAETHSHALQQTMYEMGRAVIETHPEIDEVKMSLPNNHHFLVDLQPFGQDNPNEVFYAADRPYGLIEATIQREGSRAD

SEQ ID NO: 190 - protein, 291 aa, synthetic construct
MTATAETSTGTKVVLGQNQYGKAEVRLVKVTRETARHEIQDLNVTSQLSGDFEAAHTAGDNAHVVATDTQKNTVYAFARDGFGTTEEFLLRLGKHFTEGFDWVTGGRWAAQQHFWDRINDHDHAFSRNKSEVRTAVLEISGSEQAIVSGIEGLTVLKSTGSEFHGFPRDKYTTLQETTDRILATDVSARWRYNTVEVDFDAVYGSVRGLLLKAFAETHSHALQQTMYEMGRAVIETHPEIDEINMSLPNNHHFLVDLQPFGQDNPNEVFYAADRPYGLIEATIQREGSRAD

SEQ ID NO: 191 - protein, 291 aa, synthetic construct
MTATAETSTGTKVVLGQNQYGKAEVRLVKVTRETDRHEIQDLNVTSQLSGDFEAAHTAGDNAHVVATDTQKNTVYAFARDGFATTEEFLLRLGKHFTEGFDWVTGGRWAAQQFFWDRINDHDHAFSRNKSEVRTAVLEISGSEQAIVAGIEGLTVLKSTGSEFHGFPRDKYTTLQETTDRILATDVSARWRYNTVEVDFDAVYDSVRGLLLKAFAETHSLALQQTMYEMGRAVIETHPEIDEVKMSLPNNHHFLVDLQPFGQDNPNEVFHATDRPYGLIEATIQREGSRAD

SEQ ID NO: 192 - protein, 291 aa, synthetic construct
MTATAETSTGTKVVLGQNQYGKAEVRLVKVTRETDRHEIQDLNVTSQLSGDFEAAHTAGDNAHVVATDTQKNTVYAFARDGVATTEEFLLRLGKHFTEGFDWVTGGRWAAQQHFWDRINDHDHAFSRNKSEVRTAVLEISGSEQHIVAGIEGLTVLKSTGSEFHGFPRDKYTTLQETTDRILATDVSARWRYNTVEVDFDAVYASVRGLLLKGFAETHSLALQQTMYEMGRAVIETHPEIDEVKMSLPNKHHFLVDLQPFGQDNPNEVFHAADRPYGLIEATIQREGSRAD

SEQ ID NO: 193 - protein, 291 aa, synthetic construct
MTATAETSTGTKVVLGQNQYGKAEVRLVKVTRNTDRHEIQDLNVTSQLSGDFEAAHTAGDNAHVVATDTQKNTVYAFARDGFATTEEFLLRLGKHFTEGFDWVTGGRWAAQQFFWDRINDHDHAFSRNKSEVRTAVLEISGSEQAIVAGIEGLTVLKSTGSEFHGFPRDKYTTLQETTDRILATDVSARWRYNTVEVDFDAVYASVRGLLLKGFAETHSHALQQTMYEMGRAVIETHPEIDEVSMSLPNNHHFLVDLQPFGQDNPNEVFHAADRPYGLIEATIQREGSRAD

SEQ ID NO: 194 - protein, 291 aa, synthetic construct
MTATAETSTGTKVVLGQNQYGKAEVRLVKVTRNTARHEIQDLNVTSQLSGDFEAAHTAGDNAHVVATDTQKNTVYAFARDGFGTTEEFLLRLGKHFTEGFDWVTGGRWAAQQFFWDRINDHDHAFSRNKSEVRTAVLEISGSEQTIVSGIEGLTVLKSTGSEFHGFPRDKYTTLQETTDRILATDVSARWRYNTVEVDFDAVYDSVRGLLLKGFAETHSKALQQTMYEMGRAVIETHPEIDEVKMSLPNNHHFLVDLQPFGQDNPNEVFYAVDRPYGLIEATIQREGSRAD

SEQ ID NO: 195 - protein, 291 aa, synthetic construct
MTATAETSTGTKVVLGQNQYGKAEVRLVKVTRNTDRHEIQDLNVTSQLSGDFEAAHTAGDNAHVVATDTQKNTVYAFARDGVGTTEEFLLRLGKHFTEGFDWVTGGRWAAQQFFWDRINDHDHAFSRNKSEVRTAVLEISGSEQTIVAGIEGLTVLKSTGSEFHGFPRDKYTTLQETTDRILATDVSARWRYNTDEVDFDAVYASVRGLLLKGFAETHSLALQQTMYEMGRAVIETHPEIDEVKMSLPNNHHFLVDLQPFGQDNPNEVFHAADRPYGLIEATIQREGSRAD

SEQ ID NO: 196 - protein, 291 aa, synthetic construct
MTATAETSTGTKVVLGQNQYGKAEVRLVKVTRNTDRHEIQDLNVTSQLSGDFEAAHTAGDNAHVVATDTQKNTVYAFARDGFGTTEEFLLRLGKHFTEGFDWVTGGRWAAQQHFWDRINDHDHAFSRNKSEVRTAVLEISGSEQTIVSGIEGLTVLKSTGSEFHGFPRDKYTTLQETTDRILATDVSARWRYNTVEVDFDAVYDSVRGLLLKGFAETHSKALQQTMYEMGRAVIETHPEIDEVKMSLPNNHHFLVDLQPFGQDNPNEVFHAADRPYGLIEATIQREGSRAD

SEQ ID NO: 197 - protein, 301 aa, synthetic construct
SAVKAARYGKDNVRVYKVHKDEKTGVQTVYEMVVCVLLEGNIETSYTKADNSVIVATDSIKNTIYITAKQNPVTPPELFGSILGTHFIEKYNHIHKAHVNIVCHRWTRMDIDGKPHPHSFIRDSEEKRNVQVDVVEGKGIDIKSSLSDLTVLKSTNSQFWGFLRDEYTTLKETWDRILSTDVDATWQWKNFSGLQEVRSHVPKFDATWNKAREVTLKTFAEDNSASVQNTMYKMAEQILARQQLIETVEYSLPNKHYFEIDLSWHKGLQNTGKNAEVFAPQSDPNGLIKCTVGRSSLKSKL

SEQ ID NO: 198 - protein, 301 aa, synthetic construct
SAVKAARYGKDNVRVYKVHKDEKTGVQTVYEMTVCVLLEGNIETSYTKADNSVIVATDSIKNTIYITAKQNPVTPPELWGSILGTHFIEKYNHIHAAHVNIVCHRWTRMDIDGKPHPHSFIRDSEEKRNVQVDVVEGKGIDIKSSLSDLTVLKSTNSQFWGFLRDEYTTLKETWDRILSTDVDATWQWKNFSGLQEVRSHVPKFDATWNTAREVTLKTFAEDNSASVQNTMYKMGEQILDRQQLIETVEYSLPNKHYFEIDLSWHKGLQNTGKNAEVFAPQSDPNGLIKCTVGRSSLKSKL

SEQ ID NO: 199 - protein, 301 aa, synthetic construct
SAVKAARYGKDNVRVYKVHKDEKTGVQTVYEMTVCVLLEGNIETSYTKADNSVIVATDSIKNTIYITAKQNPVTPPELWGSILGDHFIEKYNHIHKAHVNIVCHRWTRMDIDGKPHPHSFIRDSEEKRNVQVDVVEGKGIDIKSSLSDLTVLKSTNSQFWGFLRDEYTTLKETWDRILSTDVDATWQWKNFSGLQEVRSHVPKFDATWAKAREVTLKTFAEDNSASVQATMYKMAEQILDRQQLIETVEYSLPNKHYFEIDLSWHKGLQNTGKNAEVFAPQSDPNGLIKCTVGRSSLKSKL

SEQ ID NO: 200 - protein, 301 aa, synthetic construct
SAVKAARYGKDNVRVYKVHKDEKTGVQTVYEMTVCVLLEGNIETSYTKADNSNIVATDSIKNTIYITAKQNPVTPPELFGSELGTHFIEKYNHIHKAHVDIVCHRWTRMDIDGKPHPHSFIRDSEEKRNVQVDVVEGKGIDIKSSLSDLTVLKSTNSQFWGFLRDEYTTLKETWDRILSTDVDATWQWKNFSGLQEVRSHVPKFDATWATAREVTLKTFAEDNSASVQNTMYKMAEQILDRQQLIETVEYSLPNKHYFEIDLSWHKGLQNTGKNAEVFAPQSDPNGLIKCTVGRSSLKSKL

SEQ ID NO: 201 - protein, 301 aa, synthetic construct
SAVKAARYGKDNVRVYKVHKDEKTGVQTVYEMTVCVLLEGNIETSYTKADNSVIVATDSIKNTIYITAKQNPVTPPELWGSILGTHFIEKYNHIHKAHVDIVCHRWTRMDIDGKPHPHSFIRDSEEKRNVQVDVVEGKGIDIKSSLDGLTVLKSTNSQFWGFLRDEYTTLKETWDRILSTDVDATWQWKNFSGLQEVRSHVPKFDATWAKAREVTLKTFAEDNSASVQATMYKMAEQILDRQQLIETVEYSLPNKHYFEIDLSWHKGLQNTGKNAEVFAPQSDPNGLIKCTVGRSSLKSKL

SEQ ID NO: 202 - protein, 301 aa, synthetic construct
SAVKAARYGKDNVRVYKVHKDEKTGVQTVYEMTVCVLLEGEIETSYTKADNSNIVATDSIKNTIYITAKQNPVTPPELWGSILGTHFIEKYNHIHKANVNIVCHRWTRMDIDGKPHPHSFIRDSEEKRNVQVDVVEGKGIDIKSSLSDLTVLKSTNSQFEGFLRDEYTTLKETWDRILSTDVDATWQWKNFSGLQEVRSHVPKFDATWATAREVTLKTFAEDNSASVQNTIYKMAEQILDRQQLIETVEYSLPNKHYFEIDLSWHKGLQNTGKNAEVFAPQSDPNGLIKCTVGRSSLKSKL

SEQ ID NO: 203 - protein, 301 aa, synthetic construct
SAVKAARYGKDNVRVYKVHKDEKTGVQTVYEMVVCVLLEGNIETSYTKADNSNIVATDSIKNTIYITAKQNPVTPPELFGSILGTHFIEKYNHIHKANVDIVCHRWTRMDIDGKPHPHSFIRDSEEKRNVQVDVVEGKGIDIKSSLSDLTVLKSTNSQFWGFLRDEYTTLKETWDRILSTDVDATWQWKNFSGLQEVRSHVPKFDATWAKAREVTLKTFAEDNSASVQNTMYKMAEQILDRQQLIETVEYSLPNKHYFEIDLSWHKGLQNTGKNAEVFAPQSDPNGLIKCTVGRSSLKSKL

SEQ ID NO: 204 - protein, 301 aa, synthetic construct
SAVKAARYGKDNVRVYKVHKDEKTGVQTVYEMTVCVLLEGEIETSYTKADNSNIVATDSIKNTIYITAKQNPVTPPELFGSILGDHFIEKYNHIHAANVDIVCHRWTRMDIDGKPHPHSFIRDSEEKRNVQVDVVEGKGIDIKSSLDDLTVLKSTNSQFWGFLRDEYTTLKETWDRILSTDVDATWQWKNFSGLQEVRSHVPKFDATWATAREVTLKTFAEDNSASVQNTMYKMAEQILDRQQLIETVEYSLPNKHYFEIDLSWHKGLQNTGKNAEVFAPQSDPNGLIKCTVGRSSLKSKL

SEQ ID NO: 205 - protein, 301 aa, synthetic construct
SAVKAARYGKDNVRVYKVHKDEKTGVQTVYEMTVCVLLEGNIETSYTKADNSVIVATDSIKNTIHITAKQNPVTPPELFGSILGTHFIEKYNHIHKANVDIVCHRWTRMDIDGKPHPHSFIRDSEEKRNVQVDVVEGKGIDIKSSLSGLTVLKSTNSQFLGFLRDEYTTLKETWDRILSTDVDATWQWKNFSGLQEVRSHVPKFDATWATAREVTLKTFAEDNSASVQATMYKMAEQILDRQQLIETVEYSLPNKHYFEIDLSWHKGLQNTGKNAEVFAPQSDPNGLIKCTVGRSSLKSKL

SEQ ID NO: 206 - protein, 301 aa, synthetic construct
SAVKAARYGKDNVRVYKVHKDEKTGVQTVYEMTVCVLLEGEIETSYTKADNSVIVATDSIKNTIHITAKQNPVTPPELWGSILGDHFIEKYNHIHKAHVDIVCHRWTRMDIDGKPHPHSFIRDSEEKRNVQVDVVEGKGIDIKSSLSDLTVLKSTNSQFWGFLRDEYTTLKETWDRILSTDVDATWQWKNFSGLQEVRSHVPKFDATWATAREVTLKTFAEDNSASVQATMYKMAEQILARQQLIETVEYSLPNKHYFEIDLSWHKGLQNTGKNAEVFAPQSDPNGLIKCTVGRSSLKSKL

SEQ ID NO: 207 - protein, 301 aa, synthetic construct
SAVKAARYGKDNVRVYKVHKDEKTGVQTVYEMTVCVLLEGEIETSYTKADNSNIVATDSIKNTIHITAKQNPVTPPELFGSILGTHFIEKYNHIHAAHVDIVCHRWTRMDIDGKPHPHSFIRDSEEKRNVQVDVVEGKGIDIKSSLSDLTVLKSTNSQFWGFLRDEYTTLKETWDRILSTDVDATWQWKNFSGLQEVRSHVPKFDATWNKAREVTLKTFAEDNSASVQATMYKMAEQILARQQLIETVEYSLPNKHYFEIDLSWHKGLQNTGKNAEVFAPQSDPNGLIKCTVGRSSLKSKL

SEQ ID NO: 208 - protein, 301 aa, synthetic construct
SAVKAARYGKDNVRVYKVHKDEKTGVQTVYEMTVCVLLEGNIETSYTKADNSNIVATDSIKNTIYITAKQNPVTPPELWGSILGTHFIEKYNHIHAAHVDIVCHRWTRMDIDGKPHPHSFIRDSEEKRNVQVDVVEGKGIDIKSSLDGLTVLKSTNSQFWGFLRDEYTTLKETWDRILSTDVDATWQWKNFSGLQEVRSHVPKFDATWATAREVTLKTFAEDNSASVQATMYKMAEQILARQQLIETVEYSLPNKHYFEIDLSWHKGLQNTGKNAEVFAPQSDPNGLIKCTVGRSSLKSKL

SEQ ID NO: 209 - protein, 301 aa, synthetic construct
SAVKAARYGKDNVRVYKVHKDEKTGVQTVYEMTVCVLLEGEIETSYTKADNSVIVATDSIKNTIYITAKQNPVTPPELFGSILGTHFIEKYNHIHAANVDIVCHRWTRMDIDGKPHPHSFIRDSEEKRNVQVDVVEGKGIDIKSSLSDLTVLKSTNSQFEGFLRDEYTTLKETWDRILSTDVDATWQWKNFSGLQEVRSHVPKFDATWATAREVTLKTFAEDNSASVQNTMYKMAEQILDRQQLIETVEYSLPNKHYFEIDLSWHKGLQNTGKNAEVFAPQSDPNGLIKCTVGRSSLKSKL

SEQ ID NO: 210 - protein, 301 aa, synthetic construct
SAVKAARYGKDNVRVYKVHKDEKTGVQTVYEMTVCVLLEGEIETSYTKADNSVIVATDSIKNTIYITAKQNPVTPPELFGSILGTHFIEKYNHIHKAHVNIVCHRWTRMDIDGKPHPHSFIRDSEEKRNVQVDVVEGKGIDIKSSLDGLTVLKSTNSQFEGFLRDEYTTLKETWDRILSTDVDATWQWKNFRGLQEVRSHVPKFDATWATAREVTLKTFAEDNSASVQATMYKMGEQILARQQLIETVEYSLPNKHYFEIDLSWHKGLQNTGKNAEVFAPQSDPNGLIKCTVGRSSLKSKL

SEQ ID NO: 211 - protein, 301 aa, synthetic construct
SAVKAARYGKDNVRVYKVHKDEKTGVQTVYEMTVCVLLEGNIETSYTKADNSVIVATDSIKNTIHITAKQNPVTPPELFGSILGTHFIEKYNHIHKANVDIVCHRWTRMDIDGKPHPHSFIRDSEEKRNVQVDVVEGKGIDIKSSLSGLTVLKSTNSQFWGFLRDEYTTLKETWDRILSTDVDATWQWKNFSGLQEVRSHVPKFDATWAKAREVTLKTFAEDNSASVQNTMYKMGEQILDRQQLIETVEYSLPNKHYFEIDLSWHKGLQNTGKNAEVFAPQSDPNGLIKCTVGRSSLKSKL

SEQ ID NO: 212 - protein, 301 aa, synthetic construct
SAVKAARYGKDNVRVYKVHKDEKTGVQTVYEMTVCVLLEGEIETSYTKADNSVIVATDSIKNTIYITAKQNPVTPPELFGSILGDHFIEKYNHIHKAHVNIVCHRWTRMDIDGKPHPHSFIRDSEEKRNVQVDVVEGKGIDIKSSLDDLTVLKSTNSQFWGFLRDEYTTLKETWDRILSTDVDATWQWKNFSGLQEVRSHVPKFDATWNTAREVTLKTFAEDNSASVQATMYKMGEQILARQQLIETVEYSLPNKHYFEIDLSWHKGLQNTGKNAEVFAPQSDPNGLIKCTVGRSSLKSKL

SEQ ID NO: 213 - protein, 301 aa, synthetic construct
SAVKAARYGKDNVRVYKVHKDEKTGVQTVYEMTVCVLLEGNIETSYTKADNSVIVTTDSIKNTIYITAKQNPVTPPELFGSELGTHFIEKYNHIHKAHVDIVCHRWTRMDIDGKPHPHSFIRDSEEKRNVQVDVVEGKGIDIKSSLSDLTVLKSTNSQFWGFLRDEYTTLKETWDRILSTDVDATWQWKNFSGLQEVRSHVPKFDATWATAREVTLKTFAEDNSASVQNTMYKMAEQILDRQQLIETVEYSLPNKHYFEIDLSWHKGLQNTGKNAEVFAPQSDPNGLIKCTVGRSSLKSKL

SEQ ID NO: 214 - protein, 301 aa, synthetic construct
SAVKAARYGKDNVRVYKVHKDEKTGVQTVYEMVVCVLLEGEIETSYTKADNSVIVATDSIKNTIYITAKQNPVTPPELFGSILGDHFIEKYNHIHKAHVDIVCHRWTRMDIDGKPHPHSFIRDSEEKRNVQVDVVEGKGIDIKSSLSGLTVLKSTNSQFWGFLRDEYTTLKETWDRILSTDVDATWQWKNFSGLQEVRSHVPKFDATWATAREVTLKTFAEDNSASVQATMYKMAEQILDRQQLIETVEYSLPNKHYFEIDLSWHKGLQNTGKNAEVFAPQSDPNGLIKCTVGRSSLKSKL

SEQ ID NO: 215 - protein, 301 aa, synthetic construct
SAVKAARYGKDNVRVYKVHKDEKTGVQTVYEMVVCVLLEGEIETSYTKADNSVIVATDSIKNTIYITAKQNPVTPPELFGSILGTHFIEKYNHIHKAHVNIVCHRWTRMDIDGKPHPHSFIRDSEEKRNVQVDVVEGKGIDIKSSLDGLTVLKSTNSQFWGFLRDEYTTLKETWDRILSTDVDATWQWKNFSGLQEVRSHVPKFDATWAKAREVTLKTFAEDNSASVQNTMYKMAEQILDRQQLIETVEYSLPNKHYFEIDLSWHKGLQNTGKNAEVFAPQSDPNGLIKCTVGRSSLKSKL

SEQ ID NO: 216 - protein, 301 aa, synthetic construct
SAVKAARYGKDNVRVYKVHKDEKTGVQTVYEMTVCVLLEGNIETSYTKADNSNIVATDSIKNTIYITAKQNPVTPPELFGSILGDHFIEKYNHIHAAHVNIVCHRWTRMDIDGKPHPHSFIRDSEEKRNVQVDVVEGKGIDIKSSLSDLTVLKSTNSQFWGFLRDEYTTLKETWDRILSTDVDATWQWKNFSGLQEVRSHVPKFDATWNTAREVTLKTFAEDNSASVQATMYKMGEQILARQQLIETVEYSLPNKHYFEIDLSWHKGLQNTGKNAEVFAPQSDPNGLIKCTVGRSSLKSKL

SEQ ID NO: 217 - protein, 301 aa, synthetic construct
SAVKAARYGKDNVRVYKVHKDEKTGVQTVYEMTVCVLLEGEIETSYTKADNSVIVATDSIKNTIYITAKQNPVTPPELFGSELGTHFIEKYNHIHKAHVDIVCHRWTRMDIDGKPHPHSFIRDSEEKRNVQVDVVEGKGIDIKSSLSGLTVLKSTNSQFWGFLRDEYTTLKETWDRILSTDVDATWQWKNFSGLQEVRSHVPKFDATWNKAREVTLKTFAEDNSASVQATMYKMAEQILARQQLIETVEYSLPNKHYFEIDLSWHKGLQNTGKNAEVFAPQSDPNGLIKCTVGRSSLKSKL

SEQ ID NO: 218 - protein, 301 aa, synthetic construct
SAVKAARYGKDNVRVYKVHKDEKTGVQTVYEMVVCVLLEGEIETSYTKADNSNIVATDSIKNTIYITAKQNPVTPPELFGSILGTHFIEKYNHIHAAHVDIVCHRWTRMDIDGKPHPHSFIRDSEEKRNVQVDVVEGKGIDIKSSLDGLTVLKSTNSQFWGFLRDEYTTLKETWDRILSTDVDATWQWKNFSGLQEVRSHVPKFDATWATAREVTLKTFAEDNSASVQNTMYKMAEQILARQQLIETVEYSLPNKHYFEIDLSWHKGLQNTGKNAEVFAPQSDPNGLIKCTVGRSSLKSKL

SEQ ID NO: 219 - protein, 301 aa, synthetic construct
SAVKAARYGKDNVRVYKVHKDEKTGVQTVYEMTVCVLLEGEIETSYTKADNSNIVATDSIKNTIYITAKQNPVTPPELWGSILGTHFIEKYNHIHKANVDIVCHRWTRMDIDGKPHPHSFIRDSEEKRNVQVDVVEGKGIDIKSSLSDLTVLKSTNSQFEGFLRDEYTTLKETWDRILSTDVDATWQWKNFSGLQEVRSHVPKFDATWNTAREVTLKTFAEDNSASVQNTMYKMAEQILARQQLIETVEYSLPNKHYFEIDLSWHKGLQNTGKNAEVFAPQSDPNGLIKCTVGRSSLKSKL

SEQ ID NO: 220 - protein, 301 aa, synthetic construct
SAVKAARYGKDNVRVYKVHKDEKTGVQTVYEMTVCVLLEGNIETSYTKADNSVIVTTDSIKNTIHITAKQNPVTPPELFGSILGTHFIEKYNHIHKAHVDIVCHRWTRMDIDGKPHPHSFIRDSEEKRNVQVDVVEGKGIDIKSSLSGLTVLKSTNSQFWGFLRDEYTTLKETWDRILSTDVDATWQWKNFSGLQEVRSHVPKFDATWATAREVTLKTFAEDNSASVQNTMYKMAEQILARQQLIETVEYSLPNKHYFEIDLSWHKGLQNTGKNAEVFAPQSDPNGLIKCTVGRSSLKSKL

SEQ ID NO: 221 - protein, 301 aa, synthetic construct
SAVKAARYGKDNVRVYKVHKDEKTGVQTVYEMTVCVLLEGNIETSYTKADNSNIVATDSIKNTIYITAKQNPVTPPELFGSILGTHFIEKYNHIHAAHVNIVCHRWTRMDIDGKPHPHSFIRDSEEKRNVQVDVVEGKGIDIKSSLDDLTVLKSTNSQFEGFLRDEYTTLKETWDRILSTDVDATWQWKNFSGLQEVRSHVPKFDATWNTAREVTLKTFAEDNSASVQNTMYKMAEQILDRQQLIETVEYSLPNKHYFEIDLSWHKGLQNTGKNAEVFAPQSDPNGLIKCTVGRSSLKSKL

SEQ ID NO: 222 - protein, 301 aa, synthetic construct
SAVKAARYGKDNVRVYKVHKDEKTGVQTVYEMVVCVLLEGNIETSYTKADNSVIVATDSIKNTIYITAKQNPVTPPELWGSILGTHFIEKYNHIHKAHVNIVCHRWTRMDIDGKPHPHSFIRDSEEKRNVQVDVVEGKGIDIKSSLSDLTVLKSTNSQFWGFLRDEYTTLKETWDRILSTDVDATWQWKNFSGLQEVRSHVPKFDATWAKAREVTLKTFAEDNSASVQATMYKMGEQILDRQQLIETVEYSLPNKHYFEIDLSWHKGLQNTGKNAEVFAPQSDPNGLIKCTVGRSSLKSKL

SEQ ID NO: 223 - protein, 301 aa, synthetic construct
SAVKAARYGKDNVRVYKVHKDEKTGVQTVYEMVVCVLLEGEIETSYTKADNSVIVATDSIKNTIYITAKQNPVTPPELWGSILGTHFIEKYNHIHKANVDIVCHRWTRMDIDGKPHPHSFIRDSEEKRNVQVDVVEGKGIDIKSSLDGLTVLKSTNSQFWGFLRDEYTTLKETWDRILSTDVDATWQWKNFSGLQEVRSHVPKFDATWNTAREVTLKTFAEDNSASVQNTMYKMAEQILDRQQLIETVEYSLPNKHYFEIDLSWHKGLQNTGKNAEVFAPQSDPNGLIKCTVGRSSLKSKL

SEQ ID NO: 224 - protein, 301 aa, synthetic construct
SAVKAARYGKDNVRVYKVHKDEKTGVQTVYEMTVCVLLEGEIETSYTKADNSNIVATDSIKNTIYITAKQNPVTPPELWGSILGTHFIEKYNHIHKAHVDIVCHRWTRMDIDGKPHPHSFIRDSEEKRNVQVDVVEGKGIDIKSSLSDLTVLKSTNSQFWGFLRDEYTTLKETWDRILSTDVDATWQWKNFSGLQEVRSHVPKFDATWNTAREVTLKTFAEDNSASVQNTMYKMAEQILDRQQLIETVEYSLPNKHYFEIDLSWHKGLQNTGKNAEVFAPQSDPNGLIKCTVGRSSLKSKL

SEQ ID NO: 225 - protein, 301 aa, synthetic construct
SAVKAARYGKDNVRVYKVHKDEKTGVQTVYEMTVCVLLEGNIETSYTKADNSNIVATDSIKNTIYITAKQNPVTPPELFGSILGTHFIEKYNHIHKAHVNIVCHRWTRMDIDGKPHPHSFIRDSEEKRNVQVDVVEGKGIDIKSSLDGLTVLKSTNSQFEGFLRDEYTTLKETWDRILSTDVDATWQWKNFSGLQEVRSHVPKFDATWATAREVTLKTFAEDNSASVQATMYKMAEQILDRQQLIETVEYSLPNKHYFEIDLSWHKGLQNTGKNAEVFAPQSDPNGLIKCTVGRSSLKSKL

SEQ ID NO: 226 - protein, 301 aa, synthetic construct
SAVKAARYGKDNVRVYKVHKDEKTGVQTVYEMTVCVLLEGEIETSYTKADNSVIVATDSIKNTIYITAKQNPVTPPELWGSILGTHFIEKYNHIHKANVDIVCHRWTRMDIDGKPHPHSFIRDSEEKRNVQVDVVEGKGIDIKSSLDGLTVLKSTNSQFEGFLRDEYTTLKETWDRILSTDVDATWQWKNFSGLQEVRSHVPKFDATWNTAREVTLKTFAEDNSASVQNTMYKMAEQILDRQQLIETVEYSLPNKHYFEIDLSWHKGLQNTGKNAEVFAPQSDPNGLIKCTVGRSSLKSKL

SEQ ID NO: 227 - protein, 301 aa, synthetic construct
SAVKAARYGKDNVRVYKVHKDEKTGVQTVYEMTVCVLLEGEIETSYTKADNSNIVATDSIKNTIYITAKQNPVTPPELFGSILGDHFIEKYNHIHKAHVNIVCHRWTRMDIDGKPHPHSFIRDSEEKRNVQVDVVEGKGIDIKSSLDDLTVLKSTNSQFWGFLRDEYTTLKETWDRILSTDVDATWQWKNFSGLQEVRSHVPKFDATWNTAREVTLKTFAEDNSASVQATMYKMAEQILARQQLIETVEYSLPNKHYFEIDLSWHKGLQNTGKNAEVFAPQSDPNGLIKCTVGRSSLKSKL

SEQ ID NO: 228 - protein, 301 aa, synthetic construct
SAVKAARYGKDNVRVYKVHKDEKTGVQTVYEMTVCVLLEGEIETSYTKADNSVIVTTDSIKNTIYITAKQNPVTPPELWGSILGTHFIEKYNHIHKANVNIVCHRWTRMDIDGKPHPHSFIRDSEEKRNVQVDVVEGKGIDIKSSLDDLTVLKSTNSQFWGFLRDEYTTLKETWDRILSTDVDATWQWKNFSGLQEVRSHVPKFDATWNTAREVTLKTFAEDNSASVQNTMYKMAEQILDRQQLIETVEYSLPNKHYFEIDLSWHKGLQNTGKNAEVFAPQSDPNGLIKCTVGRSSLKSKL

SEQ ID NO: 229 - protein, 301 aa, synthetic construct
SAVKAARYGKDNVRVYKVHKDEKTGVQTVYEMTVCVLLEGNIETSYTKADNSVIVATDSIKNTIYITAKQNPVTPPELFGSELGTHFIEKYNHIHKAHVDIVCHRWTRMDIDGKPHPHSFIRDSEEKRNVQVDVVEGKGIDIKSSLDGLTVLKSTNSQFWGFLRDEYTTLKETWDRILSTDVDATWQWKNFSGLQEVRSHVPKFDATWAKAREVTLKTFAEDNSASVQNTMYKMAEQILDRQQLIETVEYSLPNKHYFEIDLSWHKGLQNTGKNAEVFAPQSDPNGLIKCTVGRSSLKSKL

SEQ ID NO: 230 - protein, 301 aa, synthetic construct
SAVKAARYGKDNVRVYKVHKDEKTGVQTVYEMTVCVLLEGEIETSYTKADNSNIVATDSIKNTIHITAKQNPVTPPELFGSILGTHFIEKYNHIHKANVDIVCHRWTRMDIDGKPHPHSFIRDSEEKRNVQVDVVEGKGIDIKSSLDGLTVLKSTNSQFWGFLRDEYTTLKETWDRILSTDVDATWQWKNFSGLQEVRSHVPKFDATWATAREVTLKTFAEDNSASVQNTMYKMAEQILARQQLIETVEYSLPNKHYFEIDLSWHKGLQNTGKNAEVFAPQSDPNGLIKCTVGRSSLKSKL

SEQ ID NO: 231 - protein, 301 aa, synthetic construct
SAVKAARYGKDNVRVYKVHKDEKTGVQTVYEMVVCVLLEGEIETSYTKADNSNIVATDSIKNTIYITAKQNPVTPPELFGSELGTHFIEKYNHIHKAHVDIVCHRWTRMDIDGKPHPHSFIRDSEEKRNVQVDVVEGKGIDIKSSLSGLTVLKSTNSQFWGFLRDEYTTLKETWDRILSTDVDATWQWKNFSGLQEVRSHVPKFDATWNTAREVTLKTFAEDNSASVQNTMYKMAEQILARQQLIETVEYSLPNKHYFEIDLSWHKGLQNTGKNAEVFAPQSDPNGLIKCTVGRSSLKSKL

SEQ ID NO: 232 - protein, 301 aa, synthetic construct
SAVKAARYGKDNVRVYKVHKDEKTGVQTVYEMVVCVLLEGEIETSYTKADNSNIVATDSIKNTIYITAKQNPVTPPELFGSILGDHFIEKYNHIHKAHVNIVCHRWTRMDIDGKPHPHSFIRDSEEKRNVQVDVVEGKGIDIKSSLDGLTVLKSTNSQFWGFLRDEYTTLKETWDRILSTDVDATWQWKNFSGLQEVRSHVPKFDATWAKAREVTLKTFAEDNSASVQNTMYKMGEQILARQQLIETVEYSLPNKHYFEIDLSWHKGLQNTGKNAEVFAPQSDPNGLIKCTVGRSSLKSKL

SEQ ID NO: 233 - protein, 301 aa, synthetic construct
SAVKAARYGKDNVRVYKVHKDEKTGVQTVYEMTVCVLLEGEIETSYTKADNSVIVATDSIKNTIYITAKQNPVTPPELFGSILGTHFIEKYNHIHAANVNIVCHRWTRMDIDGKPHPHSFIRDSEEKRNVQVDVVEGKGIDIKSSLSDLTVLKSTNSQFWGFLRDEYTTLKETWDRILSTDVDATWQWKNFSGLQEVRSHVPKFDATWAKAREVTLKTFAEDNSASVQNTMYKMAEQILARQQLIETVEYSLPNKHYFEIDLSWHKGLQNTGKNAEVFAPQSDPNGLIKCTVGRSSLKSKL

SEQ ID NO: 234 - protein, 301 aa, synthetic construct
SAVKAARYGKDNVRVYKVHKDEKTGVQTVYEMTVCVLLEGEIETSYTKADNSVIVATDSIKNTIYITAKQNPVTPPELWGSILGDHFIEKYNHIHAAHVNIVCHRWTRMDIDGKPHPHSFIRDSEEKRNVQVDVVEGKGIDIKSSLSGLTVLKSTNSQFWGFLRDEYTTLKETWDRILSTDVDATWQWKNFSGLQEVRSHVPKFDATWAKAREVTLKTFAEDNSASVQNTMYKMGEQILARQQLIETVEYSLPNKHYFEIDLSWHKGLQNTGKNAEVFAPQSDPNGLIKCTVGRSSLKSKL

SEQ ID NO: 235 - protein, 301 aa, synthetic construct
SAVKAARYGKDNVRVYKVHKDEKTGVQTVYEMTVCVLLEGNIETSYTKADNSVIVATDSIKNTIYITAKQNPVTPPELWGSILGTHFIEKYNHIHKAHVDIVCHRWTRMDIDGKPHPHSFIRDSEEKRNVQVDVVEGKGIDIKSSLDDLTVLKSTNSQFWGFLRDEYTTLKETWDRILSTDVDATWQWKNFSGLQEVRSHVPKFDATWNTAREVTLKTFAEDNSASVQNTMYKMAEQILARQQLIETVEYSLPNKHYFEIDLSWHKGLQNTGKNAEVFAPQSDPNGLIKCTVGRSSLKSKL

SEQ ID NO: 236 - protein, 301 aa, synthetic construct
SAVKAARYGKDNVRVYKVHKDEKTGVQTVYEMTVCVLLEGNIETSYTKADNSNIVATDSIKNTIYITAKQNPVTPPELFGSELGTHFIEKYNHIHKAHVDIVCHRWTRMDIDGKPHPHSFIRDSEEKRNVQVDVVEGKGIDIKSSLDGLTVLKSTNSQFWGFLRDEYTTLKETWDRILSTDVDATWQWKNFSGLQEVRSHVPKFDATWATAREVTLKTFAEDNSASVQNTMYKMAEQILDRQQLIETVEYSLPNKHYFEIDLSWHKGLQNTGKNAEVFAPQSDPNGLIKCTVGRSSLKSKL

SEQ ID NO: 237 - protein, 301 aa, synthetic construct
SAVKAARYGKDNVRVYKVHKDEKTGVQTVYEMTVCVLLEGEIETSYTKADNSVIVATDSIKNTIHITAKQNPVTPPELWGSILGTHFIEKYNHIHKAHVDIVCHRWTRMDIDGKPHPHSFIRDSEEKRNVQVDVVEGKGIDIKSSLSDLTVLKSTNSQFWGFLRDEYTTLKETWDRILSTDVDATWQWKNFSGLQEVRSHVPKFDATWATAREVTLKTFAEDNSASVQNTMYKMGEQILDRQQLIETVEYSLPNKHYFEIDLSWHKGLQNTGKNAEVFAPQSDPNGLIKCTVGRSSLKSKL

SEQ ID NO: 238 - protein, 301 aa, synthetic construct
SAVKAARYGKDNVRVYKVHKDEKTGVQTVYEMTVCVLLEGNIETSYTKADNSVIVATDSIKNTIYITAKQNPVTPPELFGSILGDHFIEKYNHIHKAHVNIVCHRWTRMDIDGKPHPHSFIRDSEEKRNVQVDVVEGKGIDIKSSLSDLTVLKSTNSQFEGFLRDEYTTLKETWDRILSTDVDATWQWKNFSGLQEVRSHVPKFDATWNTAREVTLKTFAEDNSASVQNTMYKMAEQILDRQQLIETVEYSLPNKHYFEIDLSWHKGLQNTGKNAEVFAPQSDPNGLIKCTVGRSSLKSKL

SEQ ID NO: 239 - protein, 301 aa, synthetic construct
SAVKAARYGKDNVRVYKVHKDEKTGVQTVYEMTVCVLLEGNIETSYTKADNSNIVATDSIKNTIYITAKQNPVTPPELWGSILGTHFIEKYNHIHKAHVNIVCHRWTRMDIDGKPHPHSFIRDSEEKRNVQVDVVEGKGIDIKSSLSDLTVLKSTNSQFWGFLRDEYTTLKETWDRILSTDVDATWQWKNFSGLQEVRSHVPKFDATWATAREVTLKTFAEDNSASVQATMYKMGEQILARQQLIETVEYSLPNKHYFEIDLSWHKGLQNTGKNAEVFAPQSDPNGLIKCTVGRSSLKSKL

SEQ ID NO: 240 - protein, 301 aa, synthetic construct
SAVKAARYGKDNVRVYKVHKDEKTGVQTVYEMVVCVLLEGNIETSYTKADNSNIVATDSIKNTIYITAKQNPVTPPELFGSILGDHFIEKYNHIHKANVDIVCHRWTRMDIDGKPHPHSFIRDSEEKRNVQVDVVEGKGIDIKSSLSDLTVLKSTNSQFWGFLRDEYTTLKETWDRILSTDVDATWQWKNFSGLQEVRSHVPKFDATWAKAREVTLKTFAEDNSASVQNTMYKMAEQILARQQLIETVEYSLPNKHYFEIDLSWHKGLQNTGKNAEVFAPQSDPNGLIKCTVGRSSLKSKL

SEQ ID NO: 241 - protein, 301 aa, synthetic construct
SAVKAARYGKDNVRVYKVHKDEKTGVQTVYEMTVCVLLEGNIETSYTKADNSVIVTTDSIKNTIYITAKQNPVTPPELFGSILGTHFIEKYNHIHKAHVNIVCHRWTRMDIDGKPHPHSFIRDSEEKRNVQVDVVEGKGIDIKSSLDDLTVLKSTNSQFWGFLRDEYTTLKETWDRILSTDVDATWQWKNFSGLQEVRSHVPKFDATWATAREVTLKTFAEDNSASVQNTMYKMGEQILDRQQLIETVEYSLPNKHYFEIDLSWHKGLQNTGKNAEVFAPQSDPNGLIKCTVGRSSLKSKL

SEQ ID NO: 242 - protein, 301 aa, synthetic construct
SAVKAARYGKDNVRVYKVHKDEKTGVQTVYEMTVCVLLEGNIETSYTKADNSVIVTTDSIKNTIHITAKQNPVTPPELFGSELGTHFIEKYNHIHKAHVNIVCHRWTRMDIDGKPHPHSFIRDSEEKRNVQVDVVEGKGIDIKSSLSGLTVLKSTNSQFWGFLRDEYTTLKETWDRILSTDVDATWQWKNFSGLQEVRSHVPKFDATWNKAREVTLKTFAEDNSASVQNTMYKMAEQILDRQQLIETVEYSLPNKHYFEIDLSWHKGLQNTGKNAEVFAPQSDPNGLIKCTVGRSSLKSKL

SEQ ID NO: 243 - protein, 301 aa, synthetic construct
SAVKAARYGKDNVRVYKVHKDEKTGVQTVYEMTVCVLLEGEIETSYTKADNSVIVATDSIKNTIYITAKQNPVTPPELFGSILGDHFIEKYNHIHAANVDIVCHRWTRMDIDGKPHPHSFIRDSEEKRNVQVDVVEGKGIDIKSSLSDLTVLKSTNSQFWGFLRDEYTTLKETWDRILSTDVDATWQWKNFSGLQEVRSHVPKFDATWAKAREVTLKTFAEDNSASVQATMYKMGEQILDRQQLIETVEYSLPNKHYFEIDLSWHKGLQNTGKNAEVFAPQSDPNGLIKCTVGRSSLKSKL

SEQ ID NO: 244 - protein, 301 aa, synthetic construct
SAVKAARYGKDNVRVYKVHKDEKTGVQTVYEMTVCVLLEGEIETSYTKADNSNIVTTDSIKNTIYITAKQNPVTPPELFGSILGDHFIEKYNHIHKAHVDIVCHRWTRMDIDGKPHPHSFIRDSEEKRNVQVDVVEGKGIDIKSSLSDLTVLKSTNSQFWGFLRDEYTTLKETWDRILSTDVDATWQWKNFSGLQEVRSHVPKFDATWNTAREVTLKTFAEDNSASVQNTMYKMGEQILARQQLIETVEYSLPNKHYFEIDLSWHKGLQNTGKNAEVFAPQSDPNGLIKCTVGRSSLKSKL

SEQ ID NO: 245 - protein, 301 aa, synthetic construct
SAVKAARYGKDNVRVYKVHKDEKTGVQTVYEMTVCVLLEGDIETSYTKADNSVIVTTDSIKNTIYITAKQNPVTPPELWGSILGTHFIEKYNHIHAAHVDIVCHRWTRMDIDGKPHPHSFIRDSEEKRNVQVDVVEGKGIDIKSSLDGLTVLKSTNSQFEGFLRDEYTTLKETCDRILSTDVDATWQWKNFSGLQEVRSHVPKFDATWAKAREVTLKTFAEDNSASVQATMYKMGEQILARQQLIETVEYSLPNKHYFEIDLSWHKGLQNTGKNAEVFAPQSDPNGLIKCTVGRSSLKSKL

SEQ ID NO: 246 - protein, 301 aa, synthetic construct
SAVKAARYGKDNVRVYKVHKDEKTGVQTVYEMTVCVLLEGNIETSYTKADNSVIVATDSIKNTIYITAKQNPVTPPELWGSILGDHFIEKYNHIHKAHVNIVCHRWTRMDIDGKPHPHSFIRDSEEKRNVQVDVVEGKGIDIKSSLSGLTVLKSTNSQFEGFLRDEYTTLKETWDRILSTDVDVTWQWKNFSGLQEVRSHVPKFDATWAKAREVTLKTFAEDNSASVQNTMYKMAEQILDRQQLIETVEYSLPNKHYFEIDLSWHKGLQNTGKNAEVFAPQSDPNGLIKCTVGRSSLKSKL

SEQ ID NO: 247 - protein, 301 aa, synthetic construct
SAVKAARYGKDNVRVYKVHKDEKTGVQTVYEMTVCVLLEGEIETSYTKADNSVIVTTDSIKNTIHITAKQNPVTPPELFGSILGTHFIEKYNHIHAANVNIVCHRWTRMDIDGKPHPHSFIRDSEEKRNVQVDVVEGKGIDIKSSLSDLTVLKSTNSQFEGFLRDEYTTLKETWDRILSTDVDATWQWKNFSGLQEVRSHVPKFDATWAKAREVTLKTFAEDNSASVQNTMYKMAEQILARQQLIETVEYSLPNKHYFEIDLSWHKGLQNTGKNAEVFAPQSDPNGLIKCTVGRSSLKSKL

SEQ ID NO: 248 - protein, 301 aa, synthetic construct
SAVKAARYGKDNVRVYKVHKDEKTGVQTVYEMVVCVLLEGEIETSYTKADNSVIVATDSIKNTIHITAKQNPVTPPELFGSILGTHFIEKYNHIHKAHVNIVCHRWTRMDIDGKPHPHSFIRDSEEKRNVQVDVVEGKGIDIKSSLSDLTVLKSTNSQFEGFLRDEYTTLKETWDRILSTDVDATWQWKNFSGLQEVRSHVPKFDATWATAREVTLKTFAEDNSASVQATMYKMAEQILDRQQLIETVEYSLPNKHYFEIDLSWHKGLQNTGKNAEVFAPQSDPNGLIKCTVGRSSLKSKL

SEQ ID NO: 249 - protein, 301 aa, synthetic construct
SAVKAARYGKDNVRVYKVHKDEKTGVQTVYEMTVCVLLEGNIETSYTKADNSNIVTTDSIKNTIYITAKQNPVTPPELFGSILGTHFIEKYNHIHAAHVDIVCHRWTRMDIDGKPHPHSFIRDSEEKRNVQVDVVEGKGIDIKSSLSDLTVLKSTNSQFWGFLRDEYTTLKETWDRILSTDVDATWQWKNFSGLQEVRSHVPKFDATWNKAREVTLKTFAEDNSASVQNTMYKMAEQILDRQQLIETVEYSLPNKHYFEIDLSWHKGLQNTGKNAEVFAPQSDPNGLIKCTVGRSSLKSKL

SEQ ID NO: 250 - protein, 301 aa, synthetic construct
SAVKAARYGKDNVRVYKVHKDEKTGVQTVYEMTVCVLLEGNIETSYTKADNSVIVATDSIKNTIHITAKQNPVTPPELFGSILGTHFIEKYNHIHKAHVNIVCHRWTRMDIDGKPHPHSFIRDSEEKRNVQVDVVEGKGIDIKSSLSDLTVLKSTNSQFEGFLRDEYTTLKETWDRILSTDVDATWQWKNFSGLQEVRSHVPKFDATWNTAREVTLKTFAEDNSASVQNTMYKMGEQILARQQLIETVEYSLPNKHYFEIDLSWHKGLQNTGKNAEVFAPQSDPNGLIKCTVGRSSLKSKL

SEQ ID NO: 251 - protein, 301 aa, synthetic construct
SAVKAARYGKDNVRVYKVHKDEKTGVQTVYEMTVCVLLEGEIETSYTKADNSVIVATDSIKNTIYITAKQNPVTPPELFGSELGTHFIEKYNHIHAAHVNIVCHRWTRMDIDGKPHPHSFIRDSEEKRNVQVDVVEGKGIDIKSSLDGLTVLKSTNSQFEGFLRDEYTTLKETWDRILSTDVDATWQWKNFSGLQEVRSHVPKFDATWNKAREVTLKTFAEDNSASVQATMYKMAEQILARQQLIETVEYSLPNKHYFEIDLSWHKGLQNTGKNAEVFAPQSDPNGLIKCTVGRSSLKSKL

SEQ ID NO: 252 - protein, 301 aa, synthetic construct
SAVKAARYGKDNVRVYKVHKDEKTGVQTVYEMTVCVLLEGEIETSYTKADNSVIVTTDSIKNTIHITAKQNPVTPPELWGSILGDHFIEKYNHIHKAHVDIVCHRWTRMDIDGKPHPHSFIRDSEEKRNVQVDVVEGKGIDIKSSLDGLTVLKSTNSQFWGFLRDEYTTLKETWDRILSTDVDATWQWKNFSGLQEVRSHVPKFDATWNTAREVTLKTFAEDNSASVQNTMYKMAEQILARQQLIETVEYSLPNKHYFEIDLSWHKGLQNTGKNAEVFAPQSDPNGLIKCTVGRSSLKSKL

SEQ ID NO: 253 - protein, 301 aa, synthetic construct
SAVKAARYGKDNVRVYKVHKDEKTGVQTVYEMTVCVLLEGNIETSYTKADNSVIVATDSIKNTIYITAKQNPVTPPELWGSELGTHFIEKYNHIHAANVNIVCHRWTRMDIDGKPHPHSFIRDSEEKRNVQVDVVEGKGIDIKSSLSGLTVLKSTNSQFWGFLRDEYTTLKETWDRILSTDVDATWQWKNFSGLQEVRSHVPKFDATWAKAREVTLKTFAEDNSASVQNTMYKMGEQILARQQLIETVEYSLPNKHYFEIDLSWHKGLQNTGKNAEVFAPQSDPNGLIKCTVGRSSLKSKL

SEQ ID NO: 254 - protein, 301 aa, synthetic construct
SAVKAARYGKDNVRVYKVHKDEKTGVQTVYEMTVCVLLEGNIETSYTKADNSVIVTTDSIKNTIYITAKQNPVTPPELWGSELGDHFIEKYNHIHKAHVNIVCHRWTRMDIDGKPHPHSFIRDSEEKRNVQVDVVEGKGIDIKSSLSGLTVLKSTNSQFWGFLRDEYTTLKETWDRILSTDVDATWQWKNFSGLQEVRSHVPKFDATWATAREVTLKTFAEDNSASVQNTMYKMAEQILARQQLIETVEYSLPNKHYFEIDLSWHKGLQNTGKNAEVFAPQSDPNGLIKCTVGRSSLKSKL

SEQ ID NO: 255 - protein, 301 aa, synthetic construct
SAVKAARYGKDNVRVYKVHKDEKTGVQTVYEMTVCVLLEGEIETSYTKADNSNIVATDSIKNTIHITAKQNPVTPPELFGSILGTHFIEKYNHIHAAHVDIVCHRWTRMDIDGKPHPHSFIRDSEEKRNVQVDVVEGKGIDIKSSLSGLTVLKSTNSQFWGFLRDEYTTLKETWDRILSTDVDATWQWKNFSGLQEVRSHVPKFDATWNTAREVTLKTFAEDNSASVQATMYKMGEQILDRQQLIETVEYSLPNKHYFEIDLSWHKGLQNTGKNAEVFAPQSDPNGLIKCTVGRSSLKSKL

SEQ ID NO: 256 - protein, 301 aa, synthetic construct
SAVKAARYGKDNVRVYKVHKDEKTGVQTVYEMTVCVLLEGNIETSYTKADNSNIVATDSIKNTIYITAKQNPVTPPELFGSILGTHFIEKYNHIHKANVDIVCHRWTRMDIDGKPHPHSFIRDSEEKRNVQVDVVEGKGIDIKSSLSGLTVLKSTNSQFEGFLRDEYTTLKETWDRILSTDVDATWQWKNFSGLQEVRSHVPKFDATWAKAREVTLKTFAEDNSASVQNTMYKMAEQILDRQQLIETVEYSLPNKHYFEIDLSWHKGLQNTGKNAEVFAPQSDPNGLIKCTVGRSSLKSKL

SEQ ID NO: 257 - protein, 301 aa, synthetic construct
SAVKAARYGKDNVRVYKVHKDEKTGVQTVYEMVVCVLLEGEIETSYTKADNSVIVATDSIKNTIYITAKQNPVTPPELWGSILGTHFIEKYNHIHKANVDIVCHRWTRMDIDGKPHPHSFIRDSEEKRNVQVDVVEGKGIDIKSSLDGLTVLKSTNSQFWGFLRDEYTTLKETWDRILSTDVDATWQWKNFSGLQEVRSHVPKFDATWATAREVTLKTFAEDNSASVQNTMYKMAEQILDRQQLIETVEYSLPNKHYFEIDLSWHKGLQNTGKNAEVFAPQSDPNGLIKCTVGRSSLKSKL

SEQ ID NO: 258 - protein, 301 aa, synthetic construct
SAVKAARYGKDNVRVYKVHKDEKTGVQTVYEMTVCVLLEGNIETSYTKADNSNIVATDSIKNTIYITAKQNPVTPPELWGSILGTHFIEKYNHIHKAHVDIVCHRWTRMDIDGKPHPHSFIRDSEEKRNVQVDVVEGKGIDIKSSLDGLTVLKSTNSQFEGFLRDEYTTLKETWDRILSTDVDATWQWKNFSGLQEVRSHVPKFDATWNTAREVTLKTFAEDNSASVQNTMYKMAEQILARQQLIETVEYSLPNKHYFEIDLSWHKGLQNTGKNAEVFAPQSDPNGLIKCTVGRSSLKSKL

SEQ ID NO: 259 - protein, 301 aa, synthetic construct
SAVKAARYGKDNVRVYKVHKDEKTGVQTVYEMTVCVLLEGEIETSYTKADNSNIVTTDSIKNTIYITAKQNPVTPPELFGSILGTHFIEKYNHIHAAHVNIVCHRWTRMDIDGKPHPHSFIRDSEEKRNVQVDVVEGKGIDIKSSLSDLTVLKSTNSQFEGFLRDEYTTLKETWDRILSTDVDATWQWKNFSGLQEVRSHVPKFDATWNTAREVTLKTFAEDNSASVQNTMYKMAEQILARQQLIETVEYSLPNKHYFEIDLSWHKGLQNTGKNAEVFAPQSDPNGLIKCTVGRSSLKSKL

SEQ ID NO: 260 - protein, 301 aa, synthetic construct
SAVKAARYGKDNVRVYKVHKDEKTGVQTVYEMVVCVLLEGEIETSYTKADNSNIVATDSIKNTIYITAKQNPVTPPELFGSILGTHFIEKYNHIHKAHVDIVCHRWTRMDIDGKPHPHSFIRDSEEKRNVQVDVVEGKGIDIKSSLDGLTVLKSTNSQFWGFLRDEYTTLKETWDRILSTDVDATWQWKNFSGLQEVRSHVPKFDATWNKAREVTLKTFAEDNSASVQNTMYKMGEQILDRQQLIETVEYSLPNKHYFEIDLSWHKGLQNTGKNAEVFAPQSDPNGLIKCTVGRSSLKSKL

SEQ ID NO: 261 - protein, 301 aa, synthetic construct
SAVKAARYGKDNVRVYKVHKDEKTGVQTVYEMTVCVLLEGNIETSYTKADNSVIVATDSIKNTIHITAKQNPVTPPELFGSILGTHFIEKYNHIHKAHVDIVCHRWTRMDIDGKPHPHSFIRDSEEKRNVQVDVVEGKGIDIKSSLSGLTVLKSTNSQFWGFLRDEYTTLKETWDRILSTDVDATWQWKNFSGLQEVRSHVPKFDATWNTAREVTLKTFAEDNSASVQATMYKMAEQILARQQLIETVEYSLPNKHYFEIDLSWHKGLQNTGKNAEVFAPQSDPNGLIKCTVGRSSLKSKL

SEQ ID NO: 262 - protein, 301 aa, synthetic construct
SAVKAARYGKDNVRVYKVHKDEKTGVQTVYEMTVCVLLEGEIETSYTKADNSVIVATDSIKNTIYITAKQNPVTPPELFGSELGTHFIEKYNHIHKAHVDIVCHRWTRMDIDGKPHPHSFIRDSEEKRNVQVDVVEGKGIDIKSSLDDLTVLKSTNSQFWGFLRDEYTTLKETWDRILSTDVDATWQWKNFSGLQEVRSHVPKFDATWATAREVTLKTFAEDNSASVQATMYKMAEQILDRQQLIETVEYSLPNKHYFEIDLSWHKGLQNTGKNAEVFAPQSDPNGLIKCTVGRSSLKSKL

SEQ ID NO: 263 - protein, 301 aa, synthetic construct
SAVKAARYGKDNVRVYKVHKDEKTGVQTVYEMMVCVLLEGNIETSYTKADNSVIVTTDSIKNTIHITAKQNPVTPPELWGSILGDHFIEKYNHIHAAHVNIVCHRWTRMDIDGKPHPHSFIRDSEEKRNVQVDVVEGKGIDIKSSLSGLTVLKSTNSQFWGFLRDEYTTLKETWDRILSTDVDATWQWKNFSGLQEVRSHVPKFDATWATAREVTLKTFAEDNSASVQNTMYKMGEQILARQQLIETVEYSLPNKHYFEIDLSWHKGLQNTGKNAEVFAPQSDPNGLIKCTVGRSSLKSKL

SEQ ID NO: 264 - protein, 301 aa, synthetic construct
SAVKAARYGKDNVRVYKVHKDEKTGVQTVYEMTVCVLLEGEIETSYTKADNSVIVATDSIKNTIYITAKQNPVTPPELWGSILGTHFIEKYNHIHAANVDIVCHRWTRMDIDGKPHPHSFIRDSEEKRNVQVDVVEGKGIDIKSSLSDLTVLKSTNSQFEGFLRDEYTTLKETWDRILSTDVDATWQWKNFSGLQEVRSHVPKFDATWNTAREVTLKTFAEDNSASVQATMYKMAEQILDRQQLIETVEYSLPNKHYFEIDLSWHKGLQNTGKNAEVFAPQSDPNGLIKCTVGRSSLKSKL

SEQ ID NO: 265 - protein, 301 aa, synthetic construct
SAVKAARYGKDNVRVYKVHKDEKTGVQTVYEMTVCVLLEGEIETSYTKADNSVIVATDSIKNTIHITAKQNPVTPPELFGSILGTHFIEKYNHIHKAHVDIVCHRWTRMDIDGKPHPHSFIRDSEEKRNVQVDVVEGKGIDIKSSLSGLTVLKSTNSQFEGFLRDEYTTLKETWDRILSTDVDATWQWKNFSGLQEVRSHVPKFDATWATAREVTLKTFAEDNSASVQNTMYKMAEQILDRQQLIETVEYSLPNKHYFEIDLSWHKGLQNTGKNAEVFAPQSDPNGLIKCTVGRSSLKSKL

SEQ ID NO: 266 - protein, 301 aa, synthetic construct
SAVKAARYGKDNVRVYKVHKDEKTGVQTVYEMTVCVLLEGEIETSYTKADNSVIVATDSIKNTIHITAKQNPVTPPELFGSILGDHFIEKYNHIHAAHVDIVCHRWTRMDIDGKPHPHSFIRDSEEKRNVQVDVVEGKGIDIKSSLDGLTVLKSTNSQFWGFLRDEYTTLKETWDRILSTDVDATWQWKNFSGLQEVRSHVPKFDATWATAREVTLKTFAEDNSASVQNTMYKMAEQILDRQQLIETVEYSLPNKHYFEIDLSWHKGLQNTGKNAEVFAPQSDPNGLIKCTVGRSSLKSKL

SEQ ID NO: 267 - protein, 301 aa, synthetic construct
SAVKAARYGKDNVRVYKVHKDEKTGVQTVYEMTVCVLLEGNIETSYTKADNSVIVTTDSIKNTIYITAKQNPVTPPELFGSELGTHFIEKYNHIHKAHVDIVCHRWTRMDIDGKPHPHSFIRDSEEKRNVQVDVVEGKGIDIKSSLDGLTVLKSTNSQFWGFLRDEYTTLKETWDRILSTDVDATWQWKNFSGLQEVRSHVPKFDATWAKAREVTLKTFAEDNSASVQNTMYKMAEQILDRQQLIETVEYSLPNKHYFEIDLSWHKGLQNTGKNAEVFAPQSDPNGLIKCTVGRSSLKSKL

SEQ ID NO: 268 - protein, 301 aa, synthetic construct
SAVKAARYGKDNVRVYKVHKDEKTGVQTVYEMTVCVLLEGNIETSYTKADNSVIVATDSIKNTIYITAKQNPVTPPELFGSELGTHFIEKYNHIHKAHVDIVCHRWTRMDIDGKPHPHSFIRDSEEKRNVQVDVVEGKGIDIKSSLDGLTVLKSTNSQFEGFLRDEYTTLKETWDRILSTDVDATWQWKNFSGLQEVRSHVPKFDATWAKAREVTLKTFAEDNSASVQNTMYKMAEQILDRQQLIETVEYSLPNKHYFEIDLSWHKGLQNTGKNAEVFAPQSDPNGLIKCTVGRSSLKSKL

SEQ ID NO: 269 - protein, 301 aa, synthetic construct
SAVKAARYGKDNVRVYKVHKDEKTGVQTVYEMVVCVLLEGNIETSYTKADNSNIVATDSIKNTIYITAKQNPVTPPELFGSELGTHFIEKYNHIHKAHVDIVCHRWTRMDIDGKPHPHSFIRDSEEKRNVQVDVVEGKGIDIKSSLDGLTVLKSTNSQFWGFLRDEYTTLKETWDRILSTDVDATWQWKNFSGLQEVRSHVPKFDATWATAREVTLKTFAEDNSASVQNTMYKMAEQILDRQQLIETVEYSLPNKHYFEIDLSWHKGLQNTGKNAEVFAPQSDPNGLIKCTVGRSSLKSKL

SEQ ID NO: 270 - protein, 301 aa, synthetic construct
SAVKAARYGKDNVRVYKVHKDEKTGVQTVYEMTVCVLLEGNIETSYTKADNSNIVATDSIKNTIHITAKQNPVTPPELFGSELGTHFIEKYNHIHKAHVDIVCHRWTRMDIDGKPHPHSFIRDSEEKRNVQVDVVEGKGIDIKSSLDGLTVLKSTNSQFWGFLRDEYTTLKETWDRILSTDVDATWQWKNFSGLQEVRSHVPKFDATWATAREVTLKTFAEDNSASVQNTMYKMAEQILDRQQLIETVEYSLPNKHYFEIDLSWHKGLQNTGKNAEVFAPQSDPNGLIKCTVGRSSLKSKL

SEQ ID NO: 271 - protein, 301 aa, synthetic construct
SAVKAARYGKDNVRVYKVHKDEKTGVQTVYEMTVCVLLEGNIETSYTKADNSNIVATDSIKNTIYITAKQNPVTPPELFGSELGTHFIEKYNHIHKAHVDIVCHRWTRMDIDGKPHPHSFIRDSEEKRNVQVDVVEGKGIDIKSSLDGLTVLKSTNSQFEGFLRDEYTTLKETWDRILSTDVDATWQWKNFSGLQEVRSHVPKFDATWATAREVTLKTFAEDNSASVQNTMYKMAEQILDRQQLIETVEYSLPNKHYFEIDLSWHKGLQNTGKNAEVFAPQSDPNGLIKCTVGRSSLKSKL

SEQ ID NO: 272 - protein, 301 aa, synthetic construct
SAVKAARYGKDNVRVYKVHKDEKTGVQTVYEMTVCVLLEGNIETSYTKADNSNIVATDSIKNTIYITAKQNPVTPPELFGSELGTHFIEKYNHIHKAHVDIVCHRWTRMDIDGKPHPHSFIRDSEEKRNVQVDVVEGKGIDIKSSLDGLTVLKSTNSQFWGFLRDEYTTLKETWDRILSTDVDATWQWKNFSGLQEVRSHVPKFDATWNTAREVTLKTFAEDNSASVQNTMYKMAEQILDRQQLIETVEYSLPNKHYFEIDLSWHKGLQNTGKNAEVFAPQSDPNGLIKCTVGRSSLKSKL

SEQ ID NO: 273 - protein, 301 aa, synthetic construct
SAVKAARYGKDNVRVYKVHKDEKTGVQTVYEMTVCVLLEGNIETSYTKADNSNIVATDSIKNTIYITAKQNPVTPPELFGSELGTHFIEKYNHIHKAHVDIVCHRWTRMDIDGKPHPHSFIRDSEEKRNVQVDVVEGKGIDIKSSLDGLTVLKSTNSQFWGFLRDEYTTLKETWDRILSTDVDATWQWKNFSGLQEVRSHVPKFDATWAKAREVTLKTFAEDNSASVQNTMYKMAEQILDRQQLIETVEYSLPNKHYFEIDLSWHKGLQNTGKNAEVFAPQSDPNGLIKCTVGRSSLKSKL

SEQ ID NO: 274 - protein, 303 aa, synthetic construct
MSTTLSSSTYGKDNVKFLKVKKDPQNPKKQEVMEATVTCLLEGGFDTSYTEADNSSIVPTDTVKNTILVLAKTTEIWPIERYAAKLATHFVEKYSHVDGVSVKIVQDRWVKYAVDGKPHDHSFIHEGGEKRITDLYYKRSGDYKLSSAIKDLTVLKSTGSEFYGYNKCDFTTLQPTTDRILSTDVDATWVWDNKKIGTVYDIAKAADKGIFDNVYNQAREITLTTFAEENSPSVQNTMFNMATQILEKACSVYSVSYALPNKHYFLIDLKWKGLENDNELFYPSPHPNGLIKCTVVRKEKTKL

SEQ ID NO: 275 - protein, 303 aa, synthetic construct
MSTTLSSSTYGKDNVKFLKVKKDPQNPKKQEVMEATVTCLLEGGFDTSYTEADNSSIVPTDTVKNTILVLAKTTEIWPIERYAAKLGTHFVEKYDHVDGVSVKIVQDRWVKYAVDGKPHDHSFIHEGGEKRITDLYYKRSGDYNLSSKIKDLTVLKSTGSEFYGYNKCDFTTLQPTTDRILSTDVDATWVWDNKKIGTVYDIAKAADKGIFDNVYNQAREITLTTFALENSPSVQATMFNMATQILEKACSVYSVSYALPNKHYFEIDLKWKGLENDNELFYPSPHPNGLIKCTVVRKEKTKL

SEQ ID NO: 276 - protein, 303 aa, synthetic construct
MSTTLSSSTYGKDNVKFLKVKKDPQNPKKQEVMEATVTCLLEGGFDTSYTEADNSSIVPTDTVKNTILVLAKTTEIWPIERFAAKLATHFVEKYSHVSGVSVKIVQDRWVKYAVDGKPHDHSFIHEGGEKRITDLYYKRSGDYDLSSAIKDLTVLKSTGSEFYGYNKCDFTTLQPTTDRILSTDVDATWVWDNKKIGTVYDIAKAADKGIFDNVYNQARETTLKTFALENSPSVQNTMFNMGTQILEKACSVYSVSYALPNKHYFLIDLKWKGLENDNELFYPSPHPNGLIKCTVVRKEKTKL

SEQ ID NO: 277 - protein, 303 aa, synthetic construct
MSTTLSSSTYGKDNVKFLKVKKDPQNPKKQEVMEATVTCLLEGGFDTSYTEADNSSIVPTDTVKNTILVLAKTTEIWPIERFVAKLATHFVEKYDHVSGVSVKIVQDRWVKYAVDGKPHDHSFIHEGGEKRITDLYYKRSGDYKLSSKIKDLTVLKSTGSEFYGYNKCDFTTLQPTTDRILSTDVDATWVWDNKKIGTVYDIAKAADKGIFDNVYNQAREITLKTFALENSPSVQNTMFNMGTQILEKACSVYSVSYALPNKHYFLIDLKWKGLENDNELFYPSPHPNGLIKCTVVRKEKTKL

SEQ ID NO: 278 - protein, 303 aa, synthetic construct
MSTTLSSSTYGKDNVKFLKVKKDPQNPKKQEVMEATVTCLLEGGFDTSYTEADNSSIVPTDTVKNTILVEAKTTEIWPIERFAAKLATHFVEKYSHVSGVSVKIVQDRWVKYAVDGKPHDHSFIHEGGEKRITDLYYKRSGDYKLSSKIKDLTVLKSTGSMFYGYNKCDFTTLQPTTDRILSTDVDATWVWDNKKIGTVYDIAKAADKGIFDNVYNQAREITLTTFALENSPSVQNTMFNMATQILEKACSVYSVSYELPNKHYFLIDLKWKGLENDNELFYPSPHPNGLIKCTVVRKEKTKL

SEQ ID NO: 279 - protein, 303 aa, synthetic construct
MSTTLSSSTYGKDNVKFLKVKKDPQNPKKQEVMEATVTCLLEGGFDTSYTEADNSSIVPTDTVKNTILVLAKTTEIWPIERFAAKLGTHFVEKYDHVSGVSVKIVQDRWVKYAVDGKPHDHSFIHEGGEKRITDLYYKRSGDYKLSSKIKDLTVLKSTGSEFYGYNKCDFTTLQPTTDRILSTDVDATWVWDNKRIGTVYDIAKAADKGIFDNVYNQAREITLTTFAEENSPSVQTTMFNMGTQILEKACSVDSVSYELPNKHYFLIDLKWKGLENDNELFYPSPHPNGLIKCTVVRKEKTKL

SEQ ID NO: 280 - protein, 303 aa, synthetic construct
MSTTLSSSTYGKDNVKFLKVKKDPQNPKKQEVMEATVTCLLEGGFDTSYTEADNSSIVPTDTVKNTILVTAKTTEIWPIERFAAKLATHFVEKYDHVSGVSVKIVQDRWVKYAVDGKPHDHSFIHESGEKRITDLYYKRSGDYDLSSKIKDLTVLKSTGSEFYGYNKCDFTTLQPTTDRILSTDVDATWVWDNKKIGTVYDIAKAADKGIFDNVYNQAREITLTTFALENSPSVQATMFNMATQILEKACSVYSVSYALPNKHYFLIDLKWKGLENDNELFYPSPHPNGLIKCTVVRKEKTKL

SEQ ID NO: 281 - protein, 303 aa, synthetic construct
MSTTLSSSTYGKDNVKFLKVKKDPQNPKKQEVMEATVTCLLEGGFDTSYTEADNSSIVPTDTVKNTILVLAKTTEIWPIERYAAKLATHFVEKYDHVSGVSVKIVQDRWVKYAVDGKPHDHSFIHEGGEKRITDLYYKRSGDYKLESAIKDLTVLKSTGSEFYGYNKCDFTTLQPTTDRILSTDVDATWVWDNKKIGTVYDIAKAADKGIFDNVYNQAREITLTTFALENSPSVQNTMFNMATQILEKACSVYSVSYELPNKHYFLIDLKWKGLENDNELFYPSPHPNGLIKCTVVRKEKTKL

SEQ ID NO: 282 - protein, 303 aa, synthetic construct
MSTTLSSSTYGKDNVKFLKVKKDPQNPKKQEVMEATVTCLLEGGFDTSYTEADNSSIVPTDTVKNTILVTAKTTEIWPIERYAAKLATHFVEKYDHVSGVSVKIVQDRWVKYAVDGKPHDHSFIHEGGEKRITDLYYKRSGDYKLSSAIKDLTVLKSTGSMFYGYNKCDFTTLQPTTDRILSTDVDATWVWDNKKIGTVYDIAKAADKGIFDNVYNQAREITLKTFAEENSPSVQNTMFNMATQILEKACSVYSVSYALPNKHYFLIDLKWKGLENDNELFYPSPHPNGLIKCTVVRKEKTKL

SEQ ID NO: 283 - protein, 303 aa, synthetic construct
MSTTLSSSTYGKDNVKFLKVKKDPQNPKKQEVMEATVTCLLEGGFDTSYTEADNSSIVPTDTVKNTILVTAKTTEIWPIERFAAKLGTHFVEKYSHVSGVSVKIVQDRWVKYAVDGKPHDHSFIHEGGEKRITDLYYKRSGDYKLSSAIKDLTVLKSTGSMFYGYNKCDFTTLQPTTDRILSTDVDATWVWDNKKIGTVYDIAKAADKGIFDNVYNQAREITLKTFAEENSPSVQNTMFNMATQILEKACSVYSVSYELPNKHYFLIDLKWKGLENDNELFYPSPHPNGLIKCTVVRKEKTKL

SEQ ID NO: 284 - protein, 303 aa, synthetic construct
MSTTLSSSTYGKDNVKFLKVKKDPQNPKKQEVMEATVTCLLEGGFDTSYTEADNSSIVPTDTVKNTILVLAKTTEIWPIERFGAKLATHFVEKYSHISGVSVKIVQDRWVKYAVDGKPHDHSFIHEGGEKRITDLYYKRSGDYKLSSAIKDLTVLKSTGSMFYGYNKCDFTTLQPTTDRILSTDVDATWVWDNKKIGTVYDIAKAADKGIFDNVYNQAREITLTTFALENSPSVQATMFNMATQILEKACSVYSVSYELPNKHYFEIDLKWKGLENDNELFYPSPHPNGLIKCTVVRKEKTKL

SEQ ID NO: 285 - protein, 303 aa, synthetic construct
MSTTLSSSTYGKDNVKFLKVKKDPQNPKKQEVMEATVTCLLEGGFDTSYTEADNSSIVPTDTVKNTILVLAKTTEIWPIERFAAKLATHFVEKYDHVSGVSVKIVQDRWVKYAVDGKPHDHSFIHEGGEKRITDLYYKRSGDYKLESKIKDLTVLKSTGSEFYGYNKCDFTTLQPTTDRILSTDVDATWVWDNKKIGTVYDIAKAADKGIFDNVYNQARETTLKTFANENSPSVQNTMFNMATQILEKACSVYSVSYELPNKHYFEIDLKWKGLENDNELFYPSPHPNGLIKCTVVRKEKTKL

SEQ ID NO: 286 - protein, 303 aa, synthetic construct
MSTTLSSSTYGKDNVKFLKVKKDPQNPKKQEVMEATVTCLLEGGFDTSYTEADNSSIVPTDTVKNTILVLAKTTEIWPIERFGAKLATHFVEKYSHVSGVSVKIVQDRWVKYAVDGKPHDHSFIHEGGEKRITDLYYKRSGDYKLSSKIKDLTVLKSTGSEFYGYNKCDFTTLQPTTGRILSTDVDATWVWDNKKIGTVYDIAKAADKGIFDNVYNQAREITLTTFANENSPSVQNTMFNMGTQILEKACSVDSVSYALPNKHYFLIDLKWKGLENDNELFYPSPHPNGLIKCTVVRKEKTKL

SEQ ID NO: 287 - protein, 303 aa, synthetic construct
MSTTLSSSTYGKDNVKFLKVKKDPQNPKKQEVMEATVTCLLEGGFDTSYTEADNSSIVPTDTVKNTILVLAKTTEIWPIERYAAKLATHFVEKYSHVSGVSVKIVQDRWVKYAVDGKPHDHSFIHEGGEKRITDLYYKRSGDYDLSSAIKDLTVLKSTGSMFYGYNKCDFTTLQPTTDRILSTDVDATWVWDNKKIGTVYDIAKAADKGIFDNVYNQAREITLTTFAEENSPSVQASMFNMATQILEKACSVYSVSYELPNKHYFEIDLKWKGLENDNELFYPSPHPNGLIKCTVVRKEKTKL

SEQ ID NO: 288 - protein, 303 aa, synthetic construct
MSTTLSSSTYGKDNVKFLKVKKDPQNPKKQEVMEATVTCLLEGSFDTSYTEADNSSIVPTDTVKNTILVTAKTTEIWPIERYAAKLATHFVEKYDHVSGVSVKIVQDRWVKYAVDGKPHDHSFIHEGGEKRITDLYYKRSGDYKLSSKIKDLTVLKSTGSEFYGYNKCDFTTLQPTTDRILSTDVDATWVWDNKKIGTVYDIAKAADKGIFDNVYNQAREITLTTFANENSPSVQNTMFNMATQILEKACSVYSVSYALPNKHYFEIDLKWKGLENDNELFYPSPHPNGLIKCTVVRKEKTKL

SEQ ID NO: 289 - protein, 303 aa, synthetic construct
MSTTLSSSTYGKDNVKFLKVKKDPQNPKKQEVMEATVTCLLEGGFDTSYTEADNSSIVPTDTVKNTILVLAKTTEIWPIERFAAKLGTHFVEKYDHVSGVSVKIVQDRWVKYAVDGKPHDHSFIHEGGEKRITDLYYKRSGDYDLSSAIKDLTVLKSTGSEFYGYNKCDFTTLQPTTDRILSTDVDATWVWDNKKIGTVYDIAKAADKGIFDNVYNQAREITLKTFANENSPSVQATMFNMATQILEKACSVYSVSYALPNKHYFEIDLKWKGLENDNELFYPSPHPNGLIKCTVVRKEKTKL

SEQ ID NO: 290 - protein, 303 aa, synthetic construct
MSTTLSSSTYGKDNVKFLKVKKDPQNPKKQEVMEATVTCLLEGGFDTSYTEADNSSIVPTDTVKNTILVTAKTTEIWPIERFAAKLATHFVEKYDHVSGVSVKIVQDRWVKYAVDGKPHDHSFIHEGGEKRITDLYYKRSGDYKLSSKIKDLTVLKSTGSEFYGYNKCDFTTLQPTTDRILSTDVDATWVWDNKKIGTVYDIAKAADKGIFDNVYNQAREITLNTFAKENSPSVQNTMFNMATQILEKACSVDSVSYELPNKHYFEIDLKWKGLENDNELFYPSPHPNGLIKCTVVRKEKTKL

SEQ ID NO: 291 - protein, 303 aa, synthetic construct
MSTTLSSSTYGKDNVKFLKVKKDPQNPKKQEVMEATVTCLLEGGFDTSYTEADNSSIVPTDTVKNTILVTAKTTEIWPIERFAAKLATHFVEKYDHVDGVSVKIVQDRWVKYAVDGKPHDHSFIHEGGEKRITDLYYKRSGDYKLSSAIKDLTVLKSTGSMFYGYNKCDFTTLQPTTDRILSTDVDATWVWDNKKIGTVYDIAKAADKGIFDNVYNQARETTLKTFANENSPSVQATMFNMGTQILEKACSVDSVSYALPNKHYFLIDLKWKGLENDNELFYPSPHPNGLIKCTVVRKEKTKL

SEQ ID NO: 292 - protein, 303 aa, synthetic construct
MSTTLSSSTYGKDNVKFLKVKKDPQNPKKQEVMEATVTCLLEGGFDTSYTEADNSSIVPTDTVKNTILVLAKTTEIWPIERYAAKLATHFVEKYSHVDGVSVKIVQDRWVKYAVDGKPHDHSFIHEGGEKRITDLYYKRSGDYDLSSAIKDLTVLKSTGSEFYGYNKCDFTTLQPTTDRILSTDVDATWVWDNKKIGTVYDIAKAADKGIFDNVYNQAREITLTTFAKENSPSVQATMFNMATQILEKACSVDSVSYALPNKHYFEIDLKWKGLENDNELFYPSPHPNGLIKCTVVRKEKTKL

SEQ ID NO: 293 - protein, 303 aa, synthetic construct
MSTTLSSSTYGKDNVKFLKVKKDPQNPKKQEVMEATVTCLLEGGFDTSYTEADNSSIVPTDTVKNTILVTAKTTEIWPIERFAAKLATHFVEKYDHVDGVSVKIVQDRWVKYAVDGKPHDHSFIHEGGEKRITDLYYKRSGDYKLSSKIKDLTVLKSTGSEFYGYNKCDFTTLQPTTDRILSTDVDATWVWDNKKIGTVYDIAKAADKGIFDNVYNQAREITLKTFALENSPSVQNTMFNMGTQILEKACSVYSVSYELPNKHYFEIDLKWKGLENDNELFYPSPHPNGLIKCTVVRKEKTKL

SEQ ID NO: 294 - protein, 303 aa, synthetic construct
MSTTLSSSTYGKDNVKFLKVKKDPQNPKKQEVMEATVTCLLEGGFDTSYTEADNSSIVPTDTVKNTILVLAKTTEIWPIERYGAKLATHFVEKYSHVDGVSVKIVQDRWVKYAVDGKPHDHSFIHEGGEKRITDLYYKRSGDYKLSSAIKDLTVLKSTGSMFYGYNKCDFTTLQPTTDRILSTDVDATWVWDNKKIGTVYDIAKAADKGIFDNVYNQAREITLTTFANENSPSVQATMFNMGTQILEKACSVYSVSYALPNKHYFEIDLKWKGLENDNELFYPSPHPNGLIKCTVVRKEKTKL

SEQ ID NO: 295 - protein, 303 aa, synthetic construct
MSTTLSSSTYGKDNVKFLKVKKDPQNPKKQEVMEATVTCLLEGGFDTSYTEADNSSIVPTDTVKNTILVLAKTTEIWPIERYAAKLATHFVEKYSHVDGVSVKIVQDRWVKYAVDGKPHDHSFIHEGGEKRITDLYYKRSGDYKLSSKIKDLTVLKSTGSEFYGYNKCDFTTLQPTTDRILSTDVDATWVWDNKKIGTVYDIAKAADKGIFDNVYNQAREIILKTFANENSPSVQATMFNMATQILEKACSVYSVSYALPNKHYFLIDLKWKGLENDNELFYPSPHPNGLIKCTVVRKEKTKL

SEQ ID NO: 296 - protein, 303 aa, synthetic construct
MSTTLSSSTYGKDNVKFLKVKKDPQNPKKQEVMEATVTCLLEGGFDTSYTEADNSSIVPTDTVKNTILVLAKTTEIWPIERFGAKLATHFVEKYDHVDGVSVKIVQDRWVKYAVDGKPHDHSFIHEGGEKRITDLYYKRSGDYKLESAIKDLTVLKSTGSEFYGYNKCDFTTLQPTTDRILSTDVDATWVWDNKKIGTVYDIAKAADKGIFDNVYNQAREITLTTFANENSPSVQNTMFNMGTQILEKACSVYSVSYALPNKHYFEIDLKWKGLENDNELFYPSPHPNGLIKCTVVRKEKTKL

SEQ ID NO: 297 - protein, 303 aa, synthetic construct
MSTTLSSSTYGKDNVKFLKVKKDPQNPKKQEVMEATVTCLLEGGFDTSYTEADNSSIVPTDTVKNTILVLAKTTEIWPIERWAAKLATHFVEKYDHVSGVSVKIVQDRWVKYAVDGKPHDHSFIHEGGEKRITDLYYKRSGDYKLSSAIKDLTVLKSTGSMFYGYNKCDFTTLQPTTDRILSTDVDATWVWDNKKIGTVYDIAKAADKGIFDNVYNQAREITLKTFALENSTSVQATMFNMATQILEKACSVDSVSYELPNKHYFLIDLKWKGLENDNELFYPSPHPNGLIKCTVVRKEKTKL

SEQ ID NO: 298 - protein, 303 aa, synthetic construct
MSTTLSSSTYGKDNVKFLKVKKDPQNPKKQEVMEATVTCLLEGGFDTSYTEADNSSIVPTDTVKNTILVTAKTTEIWPIERCAAKLATHFVEKYSHVDGVSVKIVQDRWVKYAVDGKPHDHSFIHEGGEKRITDLYYKRSGDYNLSSAIKDLTVLKSTGSMFYGYNKCDFTTLQPTTDRILSTDVDATWVWDNKKIGTVYDIAKAADKGIFDNVYNQAREITLKTFALENSPSVQNTMFNMGTQILEKACSVYSVSYALPNKHYFEIDLKWKGLENDNELFYPSPHPNGLIKCTVVRKEKTKL

SEQ ID NO: 299 - protein, 303 aa, synthetic construct
MSTTLSSSTYGKDNVKFLKVKKDPQNPKKQEVMEATVTCLLEGGFDTSYTEADNSSIVPTDTVKNTILVEAKTTEIWPIERFAAKLATHFVEKYDHVDGVSVKIVQDRWVKYAVDGKPHDHSFIHEGGEKRITDLYYKRSGDYKLESAIKDLTVLKSTGSMFYGYNKCDFTTLQPTTDRILSTDVDATWVWDNKKIGTVYDIAKAADKGIFDNVYNQAREITLTTFAEENSPSVQNTMFNMATQILEKACSVYSVSYALPNKHYFLIDLKWKGLENDNELFYPSPHPNGLIKCTVVRKEKTKL

SEQ ID NO: 300 - protein, 303 aa, synthetic construct
MSTTLSSSTYGKDNVKFLKVKKDPQNPKKQEVMEATVTCLLEDGFDTSYTEADNSSIVPTDTVKNTILVLAKTTEIWPIERYGAKLGTHFVEKYDHVSGVSVKIVQDRWVKYAVDGKPHDHSFIHEGGEKRITDLYYKRSGDYKLSSKIKDLTVLKSTGSMFYGYNKCDFTTLQPTTDRILSTDVDATWVWDNKKIGTVYDIAKAADKGIFDNVYNQAREITLTTFAEENSPSVQKTMFNMGTQILEKACSVYSVSYALPNKHYFEIDLKWKGLENDNELFYPSPHPNGLIKCTVVRKEKTKL

SEQ ID NO: 301 - protein, 303 aa, synthetic construct
MSTTLSSSTYGKDNVKFLKVKKDPQNPKKQEVMEATVTCLLEGGFDTSYTEADNSSIVPTDTVKNTILVLAKTTEIWPIERYAAKLATHFVEKYDHVSGVSVKIVQDRWVKYAVDGKPHDHSFIHEGGEKRITDLYYKRSGDYDLSSKIKDLTVLKSTGSEFYGYNKCDFTTLQPTTDRILSTDVDATWVWDNKKIGTVYDIAKAADKGIFDNVYNQAREITLTTFAEENSPSVQATMFNMATQILEKACSVDSVSYELPNKHYFEIDLKWKGLENDNELFYPSPHPNGLIKCTVVRKEKTKL

SEQ ID NO: 302 - protein, 303 aa, synthetic construct
MSTTLSSSTYGKDNVKFLKVKKDPQNPKKQEVMEATVTCLLEGGFDTSYTEADNSSIVPTDTVKNTILVLAKTTEIWPIERFAAKLGTHFVEKYDHVDGVSVKIVQDRWVKYAVDGKPHDHSFIHEGGEKRITDLYYKRCGDYDLSSKIKDLTVLKSTGSMFYGYNKCDFTTLQPTTDRILSTDVDATWVWDNKKIGTVYDIAKAADKGIFDNVYNQAREITLKTFANENSPSVQNTMFNMATQILEKACSVYSVSYELPNKHYFEIDLKWKGLENDNELFYPSPHPNGLIKCTVVRKEKTKL

SEQ ID NO: 303 - protein, 303 aa, synthetic construct
MSTTLSSSTYGKDNVKFLKVKKDPQNPKKQEVMEATVTCLLEGGFDTSYTEADNSSIVPTDTVKNTILVQAKTTEIWPIERCAAKLGTHFVEKYSHVNGVSVKIVQDRWVKYAVDGKPHDHSFIHEGGEKRITDLYYKRSGDYKLKSKIKDLTVLKSTGSEFYGYNKCDFTTLQPTTDRILSTDVDATWVWDNKKIGTVYDIAKAADKGIFDNVYNQAREITLNTFAKENSPSVQATMFNMGTQILEKACSVDSVSYALPNKHYFLIDLKWKGLENDNELFYPSPHPNGLIKCTVVRKEKTKL

SEQ ID NO: 304 - protein, 303 aa, synthetic construct
MSTTLSSSTYGKDNVKFLKVKKDQQNPKKQEVMEATVTCLLEGGFDTSYTEADNSSIVPTDTIKNTILVTAKTTEIWPIERFAAKLATHFVEKYDHVDGVSVKIVQDRWVKYAVDGKPHDHSFIHEGGEKRITDLYYKRSGDYKLSSAIKDLTVLKSTGSMFYGYNKCDFTTLQPTTDRILSTDVDATWVWDNKKIGTVYDIAKAADKGIFDNVYNQAREITLTTFALENGPSVQATMFNMATQILEKACSVYSVSYALPNKHYFEIDLKWKGLENDNELFYPSPHPNGLIKCTVVRKEKTKL

SEQ ID NO: 305 - protein, 303 aa, synthetic construct
MSTTLSSSTYGKDNVKFLKVKKDPQNPKKQEVMEATVTCLLEGGFDTSYTEADNSSIVPTDTVKNTILVMAKTTEIWPIERYAAKLGTHFVEKYSHVDGVSVKIVQDRWVKYAVDGKPHDHSFIHEGGEKRITDLYYKRSGDYKLSSKIKDLTVLKSTGSMFYGYNKCDFTTLQPTTDRILSTDVDATWVWDNKKIGTVYDIAKAADKGIFDNVYNQAREITLTTFANENSPSVQATMFNMGTQILEKACSVYSVSYELPNKHYFLIDLKWKGLENDNELFYPSPHPNGLIKCTVVRKEKTKL

SEQ ID NO: 306 - protein, 303 aa, synthetic construct
MSTTLSSSTYGKDNVKFLKVKKDPQNPKKQEVMEATVTCLLEGGFDTSYTEADNSSIVPTDTVKNTILVLAKTTEIWPIERFGAKLATHFVEKYDHVDGVSVKIVQDRWVKYAVDGKPHDHSFIHEGGEKRITDLYYKRSGDYKLSSKIKDLTVLKSTGSMFCGYNKCDFTTLQPTTDRILSTDVDATWVWDNKKIGTVYDIAKAADKGIFDNVYNQARETTLKTFALENSPSVQNTMFNMATQILEKACSVYSVSYELPNKHYFEIDLKWKGLENDNELFYPSPHPNGLIKCTVVRKEKTKL

SEQ ID NO: 307 - protein, 303 aa, synthetic construct
MSTTLSSSTYGKDNVKFLKVKKDPQNPKKQEVMEATVTCLLEGGFDTSYTEADNSSIVPTDTVKNTILVLAKTTEIWPIERFAAKLATHFVEKYDHVSGVSVKIVQDRWVKYAVDGKPHDHSFIHEGGEKRITDLYYKRSGDYKLESAIKDLTVLKSTGSMFYGYNKCDFTTLQPTTDRILSTDVDATWVWDNKKIGTVYDIAKAADKGIFDNVYNQAREITLTTFANENSPSVQNTMFNMGTQILEKACSVDSVSYELPNKHYFEIDLKWKGLENDNELFYPSPHPNGLIKCTVVRKEKTKL

SEQ ID NO: 308 - protein, 303 aa, synthetic construct
MSTTLSSSTYGKDNVKFLKVKKDPQNPKKQEVMEATVTCLLEGGFDTSYTEADNSSIVPTDTVKNTILVLAKTTEIWPIERFAAKLGTHFVEKYDHVDGVSVKIVQDRWVKYAVDGKPHDHSFIHEGGEKRITDLYYKRSGDYKLSSAIKDLTVLKSTGSMFYGYNKCDFTTLQPTTDRILSTDVDATWVWDNKKIGTVYDIAKAADKGIFDNVYNQAREITLTTFAEENSPSVQNTMFNMGTQILEKACSVYSVSYELPNKHYFEIDLKWKGLENDNELFYPSPHPNGLIKCTVVRKEKTKL

SEQ ID NO: 309 - protein, 303 aa, synthetic construct
MSTTLSSSTYGKDNVKFLKVKKDPQNPKKQEVMEATVTCLLEGGFDTSYTEADNSSIVPTDTVKNTILVTAKTTEIWPIERYAAKLATHFVEKYDHVDGVSVKIVQDRWVKYAVDGKPHDHSFIHEGGEKRITDLYYKRSGDYKLSSKIKDLTVLKSTGSMFYGYNKCDFTTLQPTTDRILSTDVDATWVWDNKKIGTVYDIAKAADKGIFDNVYNQAREITLTTFALENSPSVQATMFNMGTQILEKACSVYSVSYALPNKHYFLIDLKWKGLENDNELFYPSPHPNGLIKCTVVRKEKTKL

SEQ ID NO: 310 - protein, 303 aa, synthetic construct
MSTTLSSSTYGKDNVKFLKVKKDPQNPKKQEVMEATVTCLLEGGFDTSYTEADNSSIVPTDTVKNTILVLAKTTEIWPIERYAAKLATHFVEKYDHVDGVSVKIVQDRWVKYAIDGKPHDHSFIHEGGEKRITDLYYKRSGDYKLESKIKDLTVLKSTGSMFYGYNKCDFTTLQPTTDRILSTDVDATWVWDNKKIGTVYDIAKAADKGIFDNVYNQARETTLKTFAEENSPSVQATMFNMATQILEKACSVYSVSYELPNKHYFEIDLKWKGLENDNELFYPSPHPNGLIKCTVVRKEKTKL

SEQ ID NO: 311 - protein, 303 aa, synthetic construct
MSTTLSSSTYGKDNVKFLKVKKDPQNPKKQEVMEATVTCLLEGGFDTSYTEADNSSIVPTDTVKNTILVLAKTTEIWPIERFAAKLATHFVEKYSHVDGVSVKIVQDRWVKYAVDGKPHDHSFIHEGGEKRITDLYYKRSGDYKLESKIKDLTVLKSTGSMFYGYNKCDFTTLQPTTDRILSTDVDATWVWDNKKIGTVYDIAKAADKGIFDNVYNQAREITLTTFAEENSPSVQNTMFNMGTQILEKACSVYSVSYALPNKHYFLIDLKWKGLENDNELFYPSPHPNGLIKCTVVRKEKTKL

SEQ ID NO: 312 - protein, 303 aa, synthetic construct
MSTTLSSSTYGKDNVKFLKVKKDPQNPKKQEVMEATVTCLLEGGFDTSYTEADNSSIVPTDTVKNTILVLAKTTEIWPIERYGAKLATHFVEKYDHVDGVSVKIVQDRWVKYAVDGKPHDHSFIHEGGEKRITDLYYKRSGDYKLSSAIKDLTVLKSTGSEFYGYNKCDFTTLQPTTDRILSTDVDATWVWDNKKIGTVYDIAKAADKGIFDNVYNQAREITLTTFANENSPSVQATMFNMATQILEKACSVYSVSYALPNKHYFLIDLKWKGLENDNELFYPSPHPNGLIKCTVVRKEKTKL

SEQ ID NO: 313 - protein, 303 aa, synthetic construct
MSTTLSSSTYGKDNVKFLKVKKDPQNPKKQEVMEATVTCLLEGGFDTSYTEADNSSIVPTDTVKNTILVLAKTTEIWPIERYAAKLATHFVEKYSHVSGVSVKIVQDRWVKYAVDGKPHDHSFIHEGGEKRITDLYYKRSGDYDLSSAIKDLTVLKSTGSEFYGYNKCDFTTLQPTTDRILSTDVDATWVWDNKKIGTVYDIAKAADKGIFDNVYNQAREITLTTFANENSPSVQATMFNMGTQILEKACSVYSVSYALPNKHYFEIDLKWKGLENDNELFYPSPHPNGLIKCTVVRKEKTKL

SEQ ID NO: 314 - protein, 303 aa, synthetic construct
MSTTLSSSTYGKDNVKFLKVKKDPQNPKKQEVMEATVTCLLEGGFDTSYTEADNSSIVPTDTVKNTILVLAKTTEIWPIERFAAKLGTHFVEKYSHVNGVSVKIVQDRWVKYAVDGKPHDHSFIHEGGEKRITDLYYKRSGDYKLSSKIKDLTVLKSTGSEFYGYNKCDFTTLQPTTDRILSTDVDATWVWDNKKIGTVYDIAKAADKGIFDNVYNQARETTLTTFALENSPSVQATMFNMATQILEKACSVDSVSYELPNKHYFEIDLKWKGLENDNELFYPSPHPNGLIKCTVVRKEKTKL

SEQ ID NO: 315 - protein, 303 aa, synthetic construct
MSTTLSSSTYGKDNVKFLKVKKDPQNPKKQEVMEATVTCLLEGGFDTSYTEADNSSIVPTDTVKNTILVLAKTTEIWPIECFAAKLATHFVEKYSHVSGVSVKIVQDRWVKYAVDGKPHDHSFIHEGGEKRITDLYYKRSGDYKLSSAIKDLTVLKSTGSMFYGYNKCDFTTLQPTIDRILSTDVDATWVWDNKKIGTVYDIAKAADKGIFDNVYNQAREITLTTFAEENSPSVQNTMFNMATQILEKACSVYSVSYELPNKHYFLIDLKWKGLENDNELFYPSPHPNGLIKCTVVRKEKTKL

SEQ ID NO: 316 - protein, 303 aa, synthetic construct
MSTTLSSSTYGKDNVKFLKVKKDPQNPKKQEVMEATVTCLLEGGFDTSYTEADNSSIVPTDTVKNTILVLAKTTEIWPIERYAAKLATHFVEKYDHVSGVSVKIVQDRWVKYAVDGKPHDHSFIHEGGEKRITDLYYKRSGDYKLSSKIKDLTVLKSTGSEFYGYNKCDFTTLQPTTDRILSTDVDATWVWDNKKIGTVYDIAKAADKGIFDNVYNQARETTLTTFAEENSPSVQNTMFNMGTQILEKACSVDSVSYELPNKHYFLIDLKWKGLENDNELFYPSPHPNGLIKCTVVRKEKTKL

SEQ ID NO: 317 - protein, 303 aa, synthetic construct
MSTTLSSSTYGKDNVKFLKVKKDPQNPKKQEVMEATVTCLLEGGFDTSYTEADNSSIVPTDTVKNTILVLAKTTEIWPIERFAAKLGTHFVEKYDHVSGVSVKIVQDRWVKYAVDGKPHDHSFIHEGGEKRITDLYYKRSGDYKLSSKIKDLTVLKSTGSEFYGYNKCDFTTLQPTTDRILSTDVDATWVWDNKKIGTVYDIAKAADKGIFDNVYNQAREITLTTFALENSPSVQATMFNMATQILEKACSVYSVSYALPNKHYFEIDLKWKGLENDNELFYPSPHPNGLIKCTVVRKEKTKL

SEQ ID NO: 318 - protein, 303 aa, synthetic construct
MSTTLSSSTYGKDNVKFLKVKKDPQNPKKQEVMEATVTCLLEGGFDTSYTEADNSSIVPTDTVKNTILVLAKPTEIWPIERFAAKLATHFVEKYDHVSGVSVKIVQDRWVKYAVDGKPHDHSFIHEGGEKRITDLYYKRSGDYKLSSKIKDLTVLKSTGSEFYGYNKCDFTTLQPTTDRILSTDVDATWVWDNKKIGTVYDIAKAADKGIFDNVYNQAREITLTTFAEENSPSVQNTMFNMATQILEKACSVYSVSYELPNKHYFEIDLKWKGLENDNELFYPSPHPNGLIKCTVVRKEKTKL

SEQ ID NO: 319 - protein, 303 aa, synthetic construct
MSTTLSSSTYGKDNVKFLKVKKDPQNPKKQEVMEATVTCLLEGGFDTSYTEADNSSIVPTDTVKNTILVLAKTTEIWPIERYAAKLATHFVEKYDHVSGVSVKIVQDRWVKYAVDGKPHDHSFIHEGGEKRITDLYYKRSGDYKLSSKIKDLTVLKSTGSMFYGYNKCDFTTLQPTTDRILSTDVDATWVWDNKKIGTVYDIAKAADKGIFDNVYNQAREITLTTFANENSPSVQNTMFNMATQILEKACSVDSVSYALPNKHYFLIDLKWKGLENDNELFYPSPHPNGLIKCTVVRKEKTKL

SEQ ID NO: 320 - protein, 303 aa, synthetic construct
MSTTLSSSTYGKDNVKFLKVKKDPQNPKKQEVMEATVTCLLEGGFDTSYTEADNSSIVPTDTVKNTILVEAKTTEIWPIERFAAKLGTHFVEKYSHVDGVSVKIVQDRWVKYAVDGKPHDHSFIHEGGEKRITDLYYKRSGDYKLSSAIKDLTVLKSTGSMFYGYNKCDFTTLQPTTDRILSTDVDATWVWDNKKIGTVYDIAKAADKGIFDNVYNQARETTLTTFANENSPSVQNTMFNMATQILEKACSVYSVSYELPNKHYFLIDLKWKGLENDNELFYPSPHPNGLIKCTVVRKEKTKL

SEQ ID NO: 321 - protein, 303 aa, synthetic construct
MSTTLSSSTYGKDNVKFLKVKKDPQNPKKQEVMEATVTCLLEGGFDTSYTEADNSSIVPTDTVKNTILVLAKTTEIWPIERWAAKLATHFVEKYSHVSGVSVKIVQDRWVKYAVDGKPHDHSFIHEGGEKRITDLYYKRSGDYKLSSKIKDLTVLKSTGSMFYGYNKCDFTTLQPTTDRILSTDVDATWVWDNKKIGTVYDIAKAADKGIFDNVYNQAREITLTTFALENSPSVQNTMFNMATQILEKACSVYSVSYALPNKHYFLIDLKWKGLENDNELFYPSPHPNGLIKCTVVRKEKTKL

SEQ ID NO: 322 - protein, 303 aa, synthetic construct
MSTTLSSSTYGKDNVKFLKVKKDPQNPKKQEVMEATVTCLLEGGFDTSYTEADNSSIVPTDTVKNTILVTAKTTEIWPIERYAAKLATHFVEKYSHVDGVSVKIVQDRWVKYAVDGKPHDHSFIHEGGEKRITDLYYKRSGDYKLSSAIKDLTVLKSTGSEFYGYNKCDFTTLQPTTDRILSTDVDATWVWDNKKIGTVYDIAKAADKGIFDNVYNQVRETTLNTFAEENSPSVQETMFNMGTQILEKACSVDSVSYELPNKHYFVIDLKWKGLENDNELFYPSPHPNGLIKCTVVRKEKTKL

SEQ ID NO: 323 - protein, 303 aa, synthetic construct
MSTTLSSSTYGKDNVKFLKVKKDPQNPKKQEVMEATVTCLLEGGFDTSYTEADNSSIVPTDTVKNTILVLAKTTEIWPIERYAAKLGTHFVEKYDHVSGVSVKIVQDRWVKYAVDGKPHDHSFIHEGGEKRITDLYYKRSGDYKLSSKIKDLTVLKSTGSMFYGYNKCDFTTLQPTTDRILSTDVDATWVWDNKKIGTVYDIAKAADKGIFDNVYNQARETTLTTFAEENSPSVQATMFNMGTQILEKACSVYSVSYALPNKHYFLIDLKWKGLENDNELFYPSPHPNGLIKCTVVRKEKTKL

SEQ ID NO: 324 - protein, 303 aa, synthetic construct
MSTTLSSSTYGKDNVKFLKVKKDPQNPKKQEVMEATVTCLLEGGFDTSYTEADNSSIVPTDTVKNTILVLAKTTEIWPIERFAAKLATHFVEKYDHVSGVSVKIVQDRWVKYAVDGKPHDHSFIHEGGEKRITDLYYKRSGDYKLSSKIKDLTVLKSTGSMFYGYNKCDFTTLQPTTDRILSTDVDATWVWDNKKIGTVYDIAKAADKGIFDNVYNQARETTLKTFANENSPSIQATMFNMGTQILEKACSVDSVSYALPNKHYFLIDLKWKGLENDNELFYPSPHPNGLIKCTVVRKEKTKL

SEQ ID NO: 325 - protein, 303 aa, synthetic construct
MSTTLSSSTYGKDNVKFLKVKKDPQNPKKQEVMEATVTCLLEGGFDTSYTEADNSSIVPTDTVKNTILVLAKTTEIWPIERFAAKLGTHFVEKYDHVDGVSVKIVQDRWVKYAVDGKPHDHSFIHEGGEKRITDLYYKRSGDYKLSSKIKDLTVLKSTGSEFYGYNKCDFTTLQPTTDRILSTDVDATWVWDNKKIGTVYDIAKAADKGIFDNVYNQAREITLTTFAEENSPSVQNTMFNMATQILEKACSVYSVSYELPNKHYFLIDLKWKGLENDNELFYPSPHPNGLIKCTVVRKEKTKL

SEQ ID NO: 326 - protein, 303 aa, synthetic construct
MSTTLSSSTYGKDNVKFLKVKKDPQNPKKQEVMEATVTCLLEGGFDTSYTEADNSSIVPTDTVKNTILVLAKTTEIWPIERYAAKLATHFVEKYDHVDGVSVKIVQDRWVKYAVDGKPHDHSFIHEGGEKRITDLYYKRSGDYKLSSKIKDLTVLKSTGSEFYGYNKCDFTTLQPTTDRILSTDVDATWVWDNKKIGTVYDIAKAADKGIFDNVYNQAREITLTTFANENSPSVQATMFNMATQILEKACSVYSVSYELPNKHYFLIDLKWKGLENDNELFYPSPHPNGLIKCTVVRKEKTKL

SEQ ID NO: 327 - protein, 303 aa, synthetic construct
MSTTLSSSTYGKDNVKFLKVKKDPQNPKKQEVMEATVTCLLEGGFDTSYTEADNSSIVPTDTVKNTILVTAKTTEIWPIERYAAKLGTHFVEKYSHVDGVSVKIVQDRWVKYAVDGKPHDHSFIHEGSEKRITDLYYKRSGDYKLSSKIKDLTVLKSTGSMFYGYNKCDFTTLQPTTDRILSTDVDATWVWDNKKIGTVYDIAKAADKGIFDNVYNQAREITLTTFAEENSPSVQATMFNMGTQILEKACSVYSVSYELPNKHYFLIDLKWKGLENDNELFYPSPHPNGLIKCTVVRKEKTKL

SEQ ID NO: 328 - protein, 303 aa, synthetic construct
MSTTLSSSTYGKDNVKFLKVKKDPQNPKKQEVMEATVTCLLEGGFDTSYTEADNSSIVPTDTVKNTILVTAKTTEIWPIERYAAKLATHFVEKYSHVDGVSVKIVQDRWVKYAVDGKPHDHSFIHEGGEKRITDLYYKRSGDYKLSSKIKDLTVLKSTGSEFYGYNKCDFTTLQPTTDRILSTDVDATWVWDNKKIGTVYDIAKAADKGIFDNVYNQARETTLTTFAEENSPSVQNTMFNMGTQILEKACSVYSVSYELPNKHYFLIDLKWKGLENDNELFYPSPHPNGLIKCTVVRKEKTKL

SEQ ID NO: 329 - protein, 303 aa, synthetic construct
MSTTLSSSTYGKDNVKFLKVKKDPQNPKKQEVMEATVTCLLEGGFDTSYTEADNSSIVPTDTVKNTILVEAKTTEIWPIERFGAKLATHFVEKYDHVSGVSVKIVQDRWVKYAVDGKPHDHSFIHEGGEKRITDLYYKRSGDYKLSSAIKDLTVLKSTDSMFYGYNKCDFTTLQPTTDRILSTDVDATWVWDNKKIGTVYDIAKAADKGIFDNVYNQAREITLTTFANENSPSVQNTMFNMATQILEKACSVYSVSYALPNKHYFLIDLKWKGLENDNELFYPSPHPNGLIKCTVVRKEKTKL

SEQ ID NO: 330 - protein, 303 aa, synthetic construct
MSTTLSSSTYGKDNVKFLKVKKDPQNPKKQEVMEATVTCLLEGGFDTSYTEADNSSIVPTDTVKNTILVLAKTTEIWPIERYAAKLATHFVEKYDHVSGVSVKIVQDRWVKYAVDGKPHDHSFIHEGGEKRITDLYYKRSGDYKLSSKIKDLTVLKSTGSMFYGYNKCDFTTLQPTTDRILSTDVDATWVWDNKKIGTVYDIAKAADKGIFDNVYNQAREITLTTFALENSPSVQATMFNMGTQILEKACSVDSVSYALPNKHYFEIDLKWKGLENDNELFYPSPHPNGLIKCTVVRKEKTKL

SEQ ID NO: 331 - protein, 303 aa, synthetic construct
MSTTLSSSTYGKDNVKFLKVKKDPQNPKKQEVMEATVTCLLEGGFDTSYTEADNSSIVPTDTVKNTILVTAKTTEIWPIERYAAKLATHFVEKYDHVDGVSVKIVQDRWVKYAVDGKPHDHSFIHEGGEKRITDLYYKRSGDYKLESKIKDLTVLKSTGSEFYGYNKCDFTTLQPTTDRILSTDVDATWVWDNKKIGTVYDIAKAADKGIFDNVYNQAREITLKTFALENSPSVQATMFNMATQILEKACSVDSVSYELPNKHYFEIDLKWKGLENDNELFYPSPHPNGLIKCTVVRKEKTKL

SEQ ID NO: 332 - protein, 303 aa, synthetic construct
MSTTLSSSTYGKDNVKFLKVKKDPQNPKKQEVMEATVTCLLEGGFDTSYTDADNSSIVPTDTVKNTILVEAKTTEIWPIERFAAKLATHFVEKYSHVSGVSVKIVQDRWVKYAVDGKPHDHSFIHEGGEKRITDLYYKRSGDYKLESAIKDLTVLKSTGSMFYGYNKCDFTTLQPTTDRILSTDVDATWVWDNKKIGTVYDIAKAADKGIFDNVYNQAREITLTTFALENSPSVQNTMFNMATQILEKACSVYSVSYALPNKHYFEIDLKWKGLENDNELFYPSPHPNGLIKCTVVRKEKTKL

SEQ ID NO: 333 - protein, 303 aa, synthetic construct
MSTTLSSSTYGKDNVKFLKVKKDPQNPKKQEVMEATVTCLLEGGFDTSYTEADNSSIVPTDTVKNTILVTAKTTEIWPIERYAAKLATHFVEKYDHVDGVSVKIVQDRWVKYAVDGKPHDHSFIHEGGEKRITDLYYKRSGDYKLSSKIKDLTVLKSTGSEFYGYNKCDFTTLQPTTDRILSTDVDATWVWDNKKIGTVYDIAKAADKGIFDNVYNQAREITLTTFAEENSPSVQATMFNMGTQILEKACSVYSVSYALPNKHYFEIDLKWKGLENDNELFYPSPHPNGLIKCTVVRKEKTKL

SEQ ID NO: 334 - protein, 303 aa, synthetic construct
MSTTLSSSTYGKDNVKFLKVKKDPQNPKKQEVMEATVTCLLEGGFDTSYTEADNSSIVPTDTVKNTILVLAKTTEIWPIERFAAKLATHFVEKYSHVDGVSVKIVQDRWVKYAVDGKPHDHSFIHEGGEKRITDLYYKRSGDYKLESAIKDLTVLKSTGSMFYGYNKCDFTTLQPTTDRILSTDVDATWVWDNKKIGTVYDIAKAADKGIFDNVYNQARETTLKTFAEENSPSVQATMFNMGTQILEKACSVYSVSYELPNKHYFEIDLKWKGLENDNELFYPSPHPNGLIKCTVVRKEKTKL

SEQ ID NO: 335 - protein, 303 aa, synthetic construct
MSTTLSSSTYGKDNVKFLKVKKDPQNPKKQEVMEATVTCLLEGGFDTSYTEADNSSIVPTDTVKNTILVLAKTTEIWPIERFAAKLATHFVEKYSHVDGVSVKIVQDRWVKYAVDGKPHDHSFIHEGGEKRITDLYYKRSGDYKLSSKIKDLTVLKSTGSEFYGYNKCDFTTLQPTTDRILSTDVDATWVWDNKKIGTVYDIAKAADKCIFDNVYNQAREITLTTFAEENSPSVQNTMFNMATQILEKACSVYSVSYALPNKHYFEIDLKWKGLENDNELFYPSPHPNGLIKCTVVRKEKTKL

SEQ ID NO: 336 - protein, 303 aa, synthetic construct
MSTTLSSSTYGKDNVKFLKVKKDPQNPKKQEVMEATVTCLLEGGFDTSYTEADNSSIVPTDTVKNTILVLAKTTEIWPIERYGAKLATHFVEKYDHVDGVSVKIVQDRWVKYAVDGKPHDHSFIHEGGEKRITDLYYKRSGDYKLESKIKDLTVLKSTGSMFYGYNKCDFTTLQPTTDRILSTDVDATWVWDNKKIGTVYDIAKAADKGIFDNVYNQAREITLTTFAEENSPSVQNTMFNMATQILEKACSVYSVSYELPNKHYFEIDLKWKGLENDNELFYPSPHPNGLIKCTVVRKEKTKL

SEQ ID NO: 337 - protein, 303 aa, synthetic construct
MSTTLSSSTYGKDNVKFLKVKKDPQNPKKQEVMEATVTCLLEGGFDTSYTEADNSSIVPTDTVKNTILVTAKTTEIWPIERYAAKLATHFVEKYDHVSGVSVKIVQDRWVKYAVDGKPHDHSFIHEGGEKRITDLYYKRSGDYKLSSKIKDLTVLKSTGSEFYGYNKCDFTTLQPTTDRILSTDVDATWVWDNKKIGTVYDIAKAADKGIFDNVYNQAREITLKTFALENSPSVQNTMFNMGTQILEKACSVYSVSYALPNKHYFEIDLKWKGLENDNELFYPSPHPNGLIKCTVVRKEKTKL

SEQ ID NO: 338 - protein, 303 aa, synthetic construct
MSTTLSSSTYGKDNVKFLKVKKDPQNPKKQEVMEATVTCLLEGGFDTSYTEADNSSIVPTDTLKNTILVLAKTTEIWPIERYAAKLATHFVEKYSHVDGVSVKIVQDRWVKYAVDGKPHDHSFIHEGGEKRITDLYYKRSGDYDLESKIKDLTVLKSTGSMFYGYNKCDFTTLQPTTDRILSTDVDATWVWDNKKIGTVYDIAKAADKGIFDNVYNQAREITLTTFANENSPSVQNTMFNMATQILEKACSVYSVSYELPNKHYFEIDLKWKGLENDNELFYPSPHPNGLIKCTVVRKEKTKL

SEQ ID NO: 339 - protein, 303 aa, synthetic construct
MSTTLSSSTYGKDNVKFLKVKKDPQNPKKQEVMEATVTCLLEGGFDTSYTEADNSSIVPTDTVKNTILVTAKTTEIWPIERFAAKLATHFVEKYSHVDGVSVKIVQDRWVKYAVDGKPHDHSFIHEGGEKRITDLYYKRSGDYKLSSKIKDLTVLKSTGSMFYGYNKCDFTTLQPTTDRILSTDVDATWVWDNKKIGTVYDIAKAADKGIFDNVYNQAREITLKTFALENSPSVQNTMFNMATQILEKACSVYSVSYALPNKHYFEIDLKWKGLENDNELFYPSPHPNGLIKCTVVRKEKTKL

SEQ ID NO: 340 - protein, 303 aa, synthetic construct
MSTTLSSSTYGKDNVKFLKVKKDPQNPKKQEVMEATVTCLLEGGFDTSYTEADNSSIVPTDTVKNTILVLAKTTEIWPIERFAAKLATHFVEKYDHVSGVSVKIVQDRWVKYAVDGKPHDHSFIHEGGEKRITDLYYKRSGDYKLSSKIKDLTVLKSTGSMFYGYNKCDFTTLQPTTDRILSTDVDATWVWDNKKIGTVYDIAKAADKGIFDNVYNQAREITLKTFANENSPSVQATMFNMGTQILEKACSVDSVSYALPNKHYFEIDLKWKGLENDNELFYPSPHPNGLIKCTVVRKEKTKL

SEQ ID NO: 341 - protein, 303 aa, synthetic construct
MSTTLSSSTYGKDNVKFLKVKKDPQNPKKQEVMEATVTCLLEGGFDTSYTEADNSSIVPTDTVKNTILVLAKTTEIWPIERFAAKLATHFVEKYDHVDGVSVKIVQDRWVKYAVDGKPHDHSFIHEGGEKRITDLYYKRSGDYKLESKIKDLTVLKSTGSEFYGYNKCDFTTLQPTTDRILSTDVDATWVWDNKKIGTVYDIAKAADKGIFDNVYNQARETTLTTFANENSPSVQATMFNMATQILEKACSVDSVSYELPNKHYFLIDLKWKGLENDNELFYPSPHPNGLIKCTVVRKEKTKL